(19) United States
(12) Patent Application Publication
(10) Pub. No.: US 2021/0322405 A1
(43) Pub. Date: Oct. 21, 2021

(54) COMPOSITIONS AND METHODS FOR TREATING CANCER

(71) Applicants: Washington University, St. Louis, MO (US); The General Hospital Corporation d/b/a Massachusetts General Hospital, Boston, MA (US); The Broad Institute, Inc., Cambridge, MA (US); Battelle Memorial Institute, Richland, WA (US)

(72) Inventors: Li Ding, St. Louis, MO (US); Song Cao, St. Louis, MO (US); Yige Wu, St. Louis, MO (US); Alla Karpova, St. Louis, MO (US); Liang-Bo Wang, St. Louis, MO (US); Milan Chheda, St. Louis, MO (US); Feng Chen, St. Louis, MO (US); Ramaswamy Govindan, St. Louis, MO (US); Albert Kim, St. Louis, MO (US); Michael Gillette, Boston, MA (US); Steven Carr, 02142, MA (US); Shankha Satpathy, Cambridge, MA (US); Tao Liu, Richland, WA (US); Karin D. Rodland, Richland, WA (US); Richard D. Smith, Richland, WA (US)

(21) Appl. No.: 17/231,273

(22) Filed: Apr. 15, 2021

Related U.S. Application Data
(60) Provisional application No. 63/010,214, filed on Apr. 15, 2020.

(51) Int. Cl. A61K 31/497; A61P 35/00; A61K 31/4985; A61K 31/685; A61K 31/702; A61K 45/06

(52) U.S. Cl. CPC A61K 31/497; A61P 35/00; A61K 45/06; A61K 31/685; A61K 31/702; A61K 31/4985

(57) ABSTRACT

The present disclosure provides for compositions and methods of treating cancer. In some embodiments, PTPN11 is targeted with and anti-PTPN11 drug, such as a Shp2 inhibitor (e.g., SHP099). In some embodiments, other upregulated, hyperphosphorylated, or hyperacetylated target proteins are inhibited or targeted.

### CROSS-REFERENCE TO RELATED APPLICATIONS

This application claims priority from U.S. Provisional Application Ser. No. 63/010,214 filed on 15 Apr. 2020, which is incorporated herein by reference in its entirety.

### STATEMENT REGARDING FEDERALLY SPONSORED RESEARCH OR DEVELOPMENT

This invention was made with government support under CA210972, CA210955, and CA210986 awarded by the National Institutes of Health. The government has certain rights in the invention.

### MATERIAL INCORPORATED-BY-REFERENCE

Not applicable.

### FIELD OF THE INVENTION

The present disclosure generally relates to treatment of cancer.

### SUMMARY OF THE INVENTION

Among the various aspects of the present disclosure is the provision of compositions and methods for treating cancer.

An aspect of the present disclosure provides for a method of inhibiting PTPN11 in a tumor comprising a tumor cell comprising: obtaining or having obtained a tumor cell; optionally detecting upregulated PTPN11 in the tumor cell; or administering a PTPN11 inhibitor (e.g., a Shp2 inhibitor, such as SHP099, TNO155, JAB-3068, RMC-4630, BBP 398, RLY1971) to the tumor cell in an amount effective to inhibit PTPN11.

Another aspect of the present disclosure provides for a method of treating a subject having lung cancer comprising: obtaining or having obtained a tumor sample; detecting upregulation of PTPN11 phosphorylation; or administering an amount of a PTPN11 inhibitor (e.g., a Shp2 inhibitor, such as SHP099, TNO155, JAB-3068, RMC-4630, BBP 398, RLY1971) sufficient to inhibit PTPN11 in a tumor of a subject if PTPN11 phosphorylation is upregulated.

Yet another aspect of the present disclosure provides for a method of treating a subject having brain cancer comprising: obtaining or having obtained a tumor sample; detecting upregulation of PTPN11 or PTPN11 phosphorylation; or administering an amount of a PTPN11 inhibitor (e.g., a Shp2 inhibitor, such as SHP099, TNO155, JAB-3068, RMC-4630, BBP 398, RLY1971) sufficient to inhibit PTPN11 in a tumor of a subject if PTPN11 phosphorylation, and optionally, a PLC inhibitor (e.g., edelfosine, neomycin) if PLCG1 phosphorylation is upregulated.

In some embodiments, upregulated PTPN11 is detected by measuring increased expression levels of PTPN11 compared to a tumor sample not EGFR, ALK1, or PDGFRA-activated.

In some embodiments, the tumor is or the tumor cell is a cell from an EGFR, ALK1, or PDGFRA-activated tumor.

In some embodiments, the PTPN11 inhibitor is a Shp2 inhibitor (e.g., SHP099, TNO155, JAB-3068, RMC-4630, BBP 398, RLY1971).

In some embodiments, if the lung cancer or lung tumor is EGFR mutated- and ALK fusion-driven, the subject or tumor cell is treated with a Shp2 inhibitor (e.g., SHP099, TNO155, JAB-3068, RMC-4630, BBP 398, RLY1971) and an ALK inhibitor (e.g., ceritinib); KRAS mutated, the subject or tumor cell is treated with SOS1 inhibition; KRAS and EGFR mutated, the subject or tumor cell is treated with SOS1 inhibition and PTPN11 (Shp2) inhibitor; EGFR mutated, the subject or tumor cell is treated with a PTPN11 (Shp2) inhibitor and an EGFR tyrosine kinase inhibitor; EGFR mutated, the subject or tumor cell is treated with a modulator or inhibitor of: PHLDA1, PHLDA3, SOX9, CTNND2 (δ-catenin), CDK6, or CDKN2C; EML4-ALK mutated, the subject or tumor cell is treated with a WEE1 inhibitor; immune hot, the subject or tumor cell is treated with a combination of PD-1/PD-L1 blockade and IDO1 inhibitor; KEAP mutated, the subject or tumor cell is treated with a modulator or inhibitor of: TXNRD1, SRXN1, NQO1, ARK1C1, ARK1C3, GPX2, AKR1C2, BAG2, UGHD, ARK1B10, ARK1C4, TALDO1, GCLC, GCLM, UCHL1, AKR1B10, RMND1, PGD, GSR, or CYP4E11; KEAP mutated, the subject or tumor cell is treated with a modulator or inhibitor of: NFE2L2, AKR1C1, AKR1C1, NQO1, NFE2L2, CWC22, or MAP2, KEAP mutated, the subject or tumor cell is treated with a KEAP1/NFE2L2 interaction inhibitor; cancer testis (CT) antigen or neoantigen positive the subject or tumor cell is treated with immunotherapy or an inhibitor or modulator of KIF2C, IGF2BP3, PBK, PIWIL, BRDT, TEX15, or AKAP4, TP53 mutated, the subject or tumor cell is treated with a BRAF inhibitor or a EZH2 inhibitor; Treg upregulated, the subject or tumor cell is treated with anti-CTLA4 therapy; EGFR phosphoprotein positive (enriched), the subject or tumor is treated with a modulator or inhibitor of: WNK1 (non-FDA approved drugs), EGFR (FDA approved drugs), CDK18, CSNK1D, NEK1, PDPK1, RIPK2, PI4KB, STK3, PAK4, DCLK1, DAPK1, NEK4, or STK25, KRAS phosphoprotein positive (enriched), the subject or tumor is treated with a modulator or inhibitor of: SLK, PRKCD (FDA approved drugs), PRKCA, PRKD2, MAP3K2, KIT (FDA approved drugs), MAST3, RPS6KC1, NRBP1, PRKAB2, FN3K, AATK, or NME1; TP53 phosphoprotein positive (enriched), the subject or tumor is treated with an a modulator or inhibitor of: EGFR, RIPK2, DCLK1, CDK12 (FDA approved drugs), BRD2, MELK, BRAF (FDA approved drugs), PKN1, NRBP1, TRIO, TLK2, PRPF4B, or RIOK1; STK11 phosphoprotein positive (enriched), the subject or tumor is treated with an a modulator or inhibitor of: SLK, PKN2, TGFBR1, DYRK1B, or PRPF4B, KEAP phosphoprotein positive (enriched), the subject or tumor is treated with an a modulator or inhibitor of: PAK4, DCLK1, TRIO, TLK2, BRD2, TGFBR1, TRIM28, MAPKAPK5, MAP3K7, DYRK2, CDK7, PAK2, TRIO, TLK2, TRIM28, BUB1B, ITPK1, or TRPM7, or ALK phosphoprotein positive (enriched), the subject or tumor is treated with an a modulator or inhibitor of: PTK7, PRKAG2, WEE1 (FDA approved drugs), LRRK2, NEK6, PTK7, PRKAG2, PKM, or RIPK3, or combinations thereof.

In some embodiments, PTPN11 and PLCG1 are hyperphosphorylated and mediate RAS pathway activation in the tumor.

In some embodiments, the tumor is an EGFR-altered or RB-1 altered tumor.

In some embodiments, if the brain cancer or the tumor is RB1-altered, the subject or tumor cell is treated with an MCM inhibitor (e.g., ciprofloxacin); hypermethylated in MGMT promoter region, the subject or tumor cell is treated with temozolomide chemotherapy and radiotherapy; EGFR-altered having co-upregulation of CDK6 and EGFR, the subject or tumor cell is treated with CDK6 and EGFR inhibitors; BRAF V600E mutated having high H2B acetylation, the subject or tumor cell is treated with UBE2I inhibitors; CDK6 and UBE2I protein upregulated, the subject or tumor cell is treated with UBE2I inhibitors; RTK-altered, the subject is treated with anti-Shp2 therapy; ATRX and IDH1 mutated, the subject or tumor cell is treated with a TNIK inhibitor; immune cold (immune-low), the subject is treated with a modulator or inhibitor of: PTGR2, PDE4D, MAOA, MUC5B, or WFDC2 (HE4); immune hot (immune-high), the subject or tumor cell is treated with a CSF-1 or CSF-1R inhibitor (e.g., CSF-1 receptor inhibitor PLX3397); immune hot (immune-high), the subject or tumor cell is treated with a TAM infiltration inhibiting agent capable of targeting monocyte chemoattractant proteins (MCPs) family members in humans (e.g., CCL2, CCL7, CCL8, CCL13) or a MCP inhibitor; immune hot (immune-high), the subject or tumor cell is treated with a combination of PD-1/PD-L1 blockade and IDO1 inhibitor; immune hot (immune-high), the subject or tumor cell is treated with a modulator or inhibitor of: IDO1, immune checkpoint proteins (e.g., CTLA-4, PD-1, TIM-3), WARS, LCK, CD4, TYMP, or B2M; mesenchymal-like GBM, the subject or tumor cell is treated with anti-angiogenic agents (e.g., bevacizumab) and immunotherapy; mesenchymal GBM having elevated ALOX5 expression, the subject or tumor is treated with GPX4 inhibitor (e.g., RSL3); hyperacetylation of H2B, the subject or tumor cell is treated with CREBBP/EP300 inhibitors; IDH mutated, the subject or tumor cell is treated with a GLUD1 inhibitor; IDH mutated or proneural tumor, having upregulated AKT3, the subject or tumor is treated with an AKT3 inhibitor; PTEN downregulated, the subject or tumor cell is treated with a modulator or inhibitor of: AKT1 or AKT2, proneural and IDH-mutated, having amplified PDGFRA, higher RNA, protein, and phosphosite abundance of PDGFRA, the subject or tumor is treated with a PDGFRA inhibitor; GSK3B, AKT1, or MAPK1 phosphorylation upregulated, the subject or tumor is treated with a GSK3B inhibitor, an AKT1 inhibitor, or a MAPK1 inhibitor, respectively; ERBB2 or SHC1 phosphorylation upregulated, the subject or tumor cell is treated with a ERBB2 inhibitor or SHC1 inhibitor, respectively; GSK3B, AKT1, MAPK1, MAPK3 and EGFR phosphorylation upregulated, the subject or tumor cell is treated with GSK3B, AKT1, MAPK1, MAPK3 and EGFR inhibitors, respectively; ABL1-HDAC2 phosphorylation upregulated, the subject or tumor cell is treated with an ABL1 or HDAC-inhibitor; FLT1, MMP14, ENG, and SERPINE1 (HIF-1 downstream targets) upregulated in mesenchymal tumors, the subject or tumor cell is treated with a modulator or inhibitor of: HIF-1 and downstream targets; long telomere enriched in hyperphosphorylated genes, the subject or tumor cell is treated with a modulator or inhibitor of: RFTN2, EML4 (druggable), MLIP, TNIK (druggable), DEPTOR (druggable), ABLIM3, PLEKHA7, RRAS2 (druggable), LARP4B, NHS, CTNND2, TP53BP1, IRS2 (druggable), GJA1 (druggable), EIF4EBP1(druggable), ZNF423, BLNK, LARP4, AKAP6, PARD3, PDCD4 (druggable), SORBS1, TMPO, HEPACAM, SNTB2, GBF1, SPEG, EDNRA (druggable), TCEAL3, CANX (druggable), CCDC6, ARHGAP45, INPP5D, MAP7D1, or TNS1, long versus short telomere enriched in hyperphosphorylated genes, the subject or tumor is treated with a modulator or inhibitor of: RFTN2, EML4 (druggable), MLIP, TNIK (druggable), DEPTOR (druggable), ABLIM3, PLEKHA7, RRAS2 (druggable), LARP4B, NHS, CTNND2, IRS2 (druggable), EIF4EBP1 (druggable), ZNF423, BLNK, LARP4, AKAP6, PARD3, PDCD4 (druggable), SORBS1, TMPO, HEPACAM, SNTB2, GBF1, SPEG, EDNRA (druggable), TCEAL3, CANX (druggable), CCDC6, ARHGAP45, INPP5D, MAP7D1, or TNS1, or short telomere enriched in hyperphosphorylated genes, the subject or tumor is treated with a modulator or inhibitor of: TP53BP1, IRS2 (druggable), EIF4EBP1 (druggable), PARD3, SORBS1, SPEG, CANX (druggable), SLTM, PRAG1, ZMYND8, RANBP2, GRAMD1A, ADGRL2, BBX, PCF11, TOP2B (druggable), SCAF1, TJP1 (druggable), or FIP1 L1 (druggable); or combinations thereof.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1F: Genomic and proteomic landscape of lung adenocarcinoma (LUAD). (A) Pie chart representations of key demographic and histologic features, along with self-reported smoking status of LUAD patient samples characterized in this study dataset. (B) Patient-centric circos plot representing the multi-platform data generated in this study. The radii (spokes) of rectangular blocks represent both the matched tumor (red) and NAT (blue) samples from a given patient while the concentric circles represent the data types (Me: DNA methylation, Mu: Mutation, C: Copy Number Alteration, Mi: miRNA, R: RNA, P: Proteome, pSTY: Phosphoproteome, acK: Acetylproteome, Md: Metadata, Mu: Mutation) generated from them. White gaps in the schematic represent missing data. The associated panel on the right indicates the number of samples in each of the categories (T: Tumor; NAT: Normal Adjacent Tissue) represented in the circos plot. (C) A summary of data and metadata generated in this study. Rows enumerate the subset of clinical features used for association analyses in the dataset, the platforms used to generate genomic and proteomic data and the corresponding number of data features present in each of the constituent datasets. (D) Top panel: Oncoplot generated with maftools depicting the mutually exclusive driver oncogene somatic mutations in KRAS, EGFR, other RAS/RAF pathway genes and receptor tyrosine kinase gene fusions in the LUAD cohort. Rows represent genes and columns represent samples. Somatic mutations in tumor suppressor genes (NF1, KEAP1, STK11 and TP53) are also depicted. Numbers on the right represent the frequency of a given somatic aberration in the current cohort. Bottom panel: Percentage of transition/transversions noted in each sample is depicted in the barplots. (E) The plot represents samples arranged into four clusters (Cluster-1 to Cluster-4) identified using multi-omics non-negative matrix factorization (NMF) clustering. Within each cluster, samples are sorted by cluster membership scores. Top panel shows the somatic mutation load, followed by various clinical and molecular annotations associated with each sample. Annotation of samples by prior RNAseq-based expression subtypes (TCGA LUAD analysis) is also indicated. The heatmap shows the top 50 differential mRNA transcripts, proteins, phosphoproteins, and acetylated proteins. Expression is scaled from 0-1 for visualization. Representative pathways are illustrated for each molecular species. CIMP: CpG Island Methylator Phenotype; SV: Structural Variant from WGS data. (F) Significantly overrepresented (p-value <0.01, Fisher's exact test) metadata terms (such as driver events or demography) within the set of the most representative cluster members defining the cluster core (membership score >0.5) are shown as pie charts. Each chart represents the proportion of samples within each cluster core that are annotated with the corresponding term. Overall number of samples in each cluster and the number of samples in each cluster core are indicated on top of the chart.

FIG. 2A-FIG. 2G: Novel phosphoproteomic aberrations associated with ALK gene fusions. (A) A Summary of all kinase gene fusions identified from RNAseq analysis. The productive in-frame and loss-of-function frameshift events are indicated in blue and red, respectively. (B) RNA expression, protein abundance and specific phosphosite modifications noted to be outliers in the index fusion event sample relative to all other samples. (C) Boxplot showing outlier expression in tumors with ALK fusion of ALK RNA, protein and the ALK Y1507 phosphosite. Blue: Normal adjacent tissues (NAT); Pink: Tumor samples. Sample IDs of outlier cases are indicated. (D) Boxplot showing overexpression of ALK mRNA observed in fusion-positive (Red) versus -negative (Blue) tumors. The three 5′ partners show comparably high expression in both fusion-positive and -negative tumors as expected. (E) Boxplot showing the phosphorylation of two ALK fusion partners, HMBOX1 and EML4, in the indicated index cases. (F) Scatterplot of significantly regulated phosphosites and their corresponding protein expression in tumors with and without ALK fusion. Phosphosites showing distinct upregulation in ALK fusion samples are highlighted in red. (G) Immunohistochemistry reveals upregulation of both total ALK and the ALK Y1507 phosphosite specifically in the tumor epithelia of ALK fusion-positive samples. No staining was seen in RET or ROS1 fusion samples or in matched NATs (FIG. 9C).

FIG. 3A-FIG. 3G: Impact of copy number alteration (CNA) and DNA methylation (DNA-me) on protein and phosphoprotein expression. (A) Steady state correlation between mRNA and protein abundance with average Spearman's correlation coefficient 0.49 (median=0.52) in 110 tumor samples. mRNA and protein showed 95.3% positive correlation (80% significant correlation, FDR<0.01) across tumors. The associated heatmap shows genes that alter their mRNA-protein correlation between all tumors and their paired NAT samples (n=101) as well as for tumor-NAT pairs with TP53 (n=52), EGFR (n=37), KRAS (n=30), or STK11 mutations (n=18) (total of 243 genes with FDR <0.1). Bottom panel represents enriched biological terms including replication, chromatin modifiers and translational regulators that were associated with these genes. −Log 10 (p-value) is shown in brackets. (B) Correlation plots between CNA and RNA expression and between CNA and protein abundance. Significant (FDR <0.05) positive and negative correlations are indicated in red and green, respectively. CNA-driven cis-effects (consequence of CNA on the same locus) appear as the red diagonal line; trans-effects (consequence of CNA on genes encoded elsewhere) appear as vertical red and green lines. The accompanying histograms show the number of significant (FDR <0.05) cis- and trans-events corresponding to the indicated genomic loci (upward plot) as well as the overlap between CNA-RNA and CNA-protein events (downward plot). (C) The left venn diagram shows the overlap between significant cis-events across RNA, proteome and phosphoproteome. The venn diagram on the right shows the cancer-associated genes with significant cis-effects across multiple data types. (D) Genes with CNA events that show significant similarity (FDR <0.1) between their significant trans-effects (FDR <0.05) and LINCS CMap genomic perturbation profiles. Cyan indicates the number of significant trans-events and blue shows the overlap between trans-events and CMap. (E) Shown are the 120 genes whose DNA methylation was associated with cascading cis-regulation of their mRNA expression, global protein level and phosphopeptide abundance. A subset of 92 genes whose protein abundances significantly differed between tumor and paired NAT are labeled with red crosses. Purple triangles (n=85) indicate the subset for which evidence of cis-regulation was found between individual methylation probes in the genes and their RNA transcript and protein abundances. Bold type highlights a few known cancer genes. (F) Heatmaps of multi-omic data for CLDN18, ANK1 and PTPRCAP across all samples for which methylation data was available (n=109). Gene-level methylation scores, RNA expression levels and protein/phospho-peptide abundances were converted into Z-scores and the tumors samples were ordered by methylation levels. (G) Heatmap showing protein expression of PTPRC complex members in tumors.

FIG. 4A-FIG. 4G: Impact of somatic mutation on the proteogenomic landscape. (A) The cis- and trans-effects of selected mutations (x axis) on the expression of cancer-associated proteins (left) and PTMs (right) (see STAR methods). Cis-effects and trans-effects are indicated as circles and squares, respectively. The red/blue scale represents change in protein or PTM expression and the size represents significance. For example, TP53 mutation was associated with a novel trans-effect showing increased EZH2 protein expression (left) and several previously uncharacterized EZH2 phosphosite changes (right). (B) Scatter plots showing the relationship between KEAP1 protein expression and NFE2L2 phosphorylation (S215 and S433 phosphosites) in the KEAP1-mutant samples. (C) Ribbon/Richardson diagram representing 3D protein structure of KEAP1 (Pink) and NFE2L2 DLG motif (green) interaction. Distribution of various KEAP1 amino acid residues affected by somatic mutations observed in this cohort are indicated. (D, E) Scatter plots showing significance of RNA, protein (green), phosphorylation site (purple), and acetylation site (yellow) abundance changes between KRAS mutant (E) or EGFR mutant (F) and wildtype tumors as determined using the Wilcoxon rank sum test. Proteins and PTMs passing a Benjamini-Hochberg-adjusted p value of <0.05 are indicated by triangles. Black vertical lines indicate the threshold for RNA transcripts passing an adjusted p value of <0.05. (F) Heatmap showing phosphorylation of PTPN11 Y62 in EGFR mutant samples. (G) Heatmap showing the outlier kinases enriched (FDR <0.2) at the phosphoprotein, protein, RNA and CNA levels and their association with mutations in select genes (EGFR, KRAS, TP53, STK11, KEAP1, EML4-ALK). DepMap panels on the left show log 2-transformed relative survival averaged across all available lung cell lines after depletion of the indicated gene (rows) by RNAi or CRISPR. Druggability based on DGIdb (http://www.dgidb.org/) is indicated alongside the availability of FDA-approved drugs. The log-transformed druggability score indicates the sum of PubMed journal articles that support the drug-gene relationship.

FIG. 5A-FIG. 5G: Immune landscape in LUAD. (A) Heatmaps show three consensus clusters (Hot-tumor-enriched (HTE), Cold-tumor-enriched (CTE), and NAT-enriched) based on immune/stromal signatures identified from xCell (http://xCell.ucsf.edu/), together with key associated proteogenomic features. Select clinical and molecular features are shown at the top of the figure. The immune/stromal signatures heatmap panel shows xCell-derived sample-wise relative abundance of various immune and stromal cell types. The pathway heatmap panel shows upregulated pathways in HTE and CTE clusters based on global protein abundance. The expression heatmap panel depicts the RNA and protein levels of various immune markers involved in immune evasion mechanisms. (B) Association between mutation profiles and immune/stromal signatures from xCell. Heatmap shows signed −log 10 (p-values). Only associations significant at FDR <0.05 are shown. (C) xCell scores for conventional dendritic cells (cDC) and macrophages for NAT samples (x-axis) and tumor samples (y-axis). For each sample, we indicate if a cell was significantly infiltrated (xCell p-value <0.05) in both NAT and tumor (black), only in NAT (blue), only in tumor (red), or in neither NAT nor tumor (light-gray). Samples with STK11 mutations are displayed with a triangle. STK11 mutation was found enriched in the subset of samples with infiltration of macrophages and dendritic cells only in NATs (Fisher's exact test FDR <0.1). (D) Box plots show association between STK11 mutation and immune score (ESTIMATE). STK11 mutations are associated with lower immune score irrespective of KRAS mutation status. (E) t-SNE (t-Distributed Stochastic Neighbor Embedding) plot provides a two-dimensional representation of the activation scores of individual tiles representing STK11 and WT tumor samples. Activation scores were derived from a deep learning algorithm for 10,000 tiles from hematoxylin-eosin stained tissue images. Overlaying positive prediction scores on sample points defined distinct clusters for predicted STK11 mutant (orange) and WT (blue) cases. Examples of true positive (red outline) and true negative (black outline) tiles exhibit different histologic features, such that the STK11 wild type tiles correctly recognized by the model harbor abundant inflammatory cells, whereas STK11 mutant tiles showed typical adenocarcinoma characteristics without inflammation. (F) Cluster diagram shows enriched pathways (www.metascape.org) among the 230 proteins significantly associated with the STK11 mutation-enriched cluster. The list was derived from protein-based unsupervised independent component analysis clustering presented in FIG. 12C. Amongst the top 20 clusters, the one representing neutrophil degranulation showed highest significance (Q<10⁻¹⁴). Nodes are aggregated into clusters based on membership similarities, and clusters are named for their most significant node. Node size represents the number of differentially expressed gene products. The top 5 clusters by p-value are highlighted. (G) Scatterplot shows differentially regulated protein and RNA expression (signed −log 10 p-value) in tumors with and without STK11 mutation. Proteins associated with neutrophil degranulation are highlighted in red.

FIG. 6A-FIG. 6B: Environmental and smoking-related molecular signatures. (A) Heatmap showing correlation coefficients between the mutational signatures of LUAD tumor samples and 53 previously-identified signatures of environmental exposure (Kucab et al., 2019). The top panel shows self-reported smoking status, gender, percentages and raw counts of six mutation types, smoking score, canonical DNP status, and fraction of Cosmic signature 4. (B) Heatmaps demonstrate impact of high or low smoking score (HSS; >0.1; LSS; <0.1) on tumors and paired NATs. Tumors were first grouped according to HSS or LSS; the same grouping scheme and annotations were then applied to paired NATs. The heatmaps show protein expression-derived, differentially regulated (FDR <0.05) pathways associated with LSS and HSS, separately in tumors (left) and NATs (right). The pathways are grouped into six panels: (1) Pathways activated in tumors with HSS but suppressed in NATs with HSS; (2) Pathways suppressed in tumors with HSS but activated in NATs with HSS; (3) Pathways activated in both tumors and NATs with HSS; (4) Pathways repressed in tumors and NATs with HSS; (5) Pathways activated in tumors with HSS but showing no detectable change between HSS and LSS in NATs; (6) Pathways suppressed in tumors with HSS, but with no change in NATs. (C) Scatterplot showing direction and significance of pathway-level protein differences between samples with high and low smoking scores (HSS and LSS) in tumors and NATs. Pathways are color-coded according to their pathway group in FIG. 6B. Group 1 pathways are upregulated in HSS in tumors and downregulated in HSS in NATs. Group 2 pathways are downregulated in HSS in tumors and upregulated in HSS in NATs. Groups 3 and 4 are upregulated and downregulated, respectively, in both tumors and NATs. Signed log 10 FDR represents Benjamini-Hochberg corrected p-values from Mann-Whitney-Wilcoxon (MWM) test and the direction (+ or −) indicates activation or suppression (i.e. Wilcoxon rank sum test). (D) HSS vs LSS differential pathway scores in tumors and NATs at the transcriptome level. The group separations clearly defined by protein-based pathways (FIG. 6C) are less evident at the RNA level.

FIG. 7A-FIG. 7F: Proteogenomics summary of tumors and paired NATs. (A) Principal component analysis of protein expression shows distinct separation of tumor samples (n=110, cadet blue) and NATs (n=101, coral). (B) Scatter plots showing median fold-change between tumors and paired NATs at the level of proteome and phosphosite (left) and proteome and acetylome (right). The dashed line shows equivalence with intercept 0. Red triangles highlight sites with minimum 16-fold (4-fold in log 2) site-level overexpression compared to associated protein changes with maximum 4-fold overexpression (i.e. a minimum 4-fold differential). Blue triangles highlight downregulated sites using symmetric parameters. NPM1, PRKCZ, DNMT1, CIT, LIMK2, CDK13, HAT1, and EEF2K were observed to be hyper-phosphorylated, whereas PAK4, PTPN11, PRKCD, MAST4, BAZ1B and ENO1 were hypo-phosphorylated in tumors relative to their protein expression. Hyperacetylated sites include HIST1H2BA (K22/K25), EP300 (K1558), ZNF609 (K157), NCL (K228), ROCK2 (K892), AFF1 (K576), POTEJ (K169), ADSSL1 (K18), and KMT2C (K3860). Only HIBCH (K358) was hypoacetylated in tumors. (C) Proteomics-based tumor biomarker candidates (log 2 fold change (Log 2FC) >2 and FDR <0.01 in ≥80% of tumor-NAT pairs) for 4 frequently mutated genes: TP53 (40/63 shown), EGFR (39), KRAS (37) and STK11 (40/77 shown). Each dot represents a tumor sample. Blue-colored boxplots highlight proteins with overexpression in more than 99% of tumor samples with the associated mutation. Relevant characteristics of the biomarker candidates (enzyme, transcription factor, secreted, transmembrane or observed in plasma), and relevant targeted drugs in clinical trials are shown in the accompanying crossword plot. Panel 1: TP53 mutants showed overexpression of tumorigenic proteins TP53, CCNA2, TOP2A, PLOD2, ANLN, and MMP12. Panel 2: EGFR-related overexpression included MET, enzymes DHCR24, TKTL2, and GLA, and prognostic marker S100B. Panel 3: KRAS mutants overexpressed extracellular glycoproteins and collagens (GREM1, COL1A1, COL12A1, COL5A1, FNDC1, POSTN, LOXL2, ADAMTS2, ADAMTS5, and ADAMTS7), as well as enzymes including MMP1, CEMIP, PFKP, and UHRF1. Panel 4: STK11 mutant tumors were enriched for amino acid metabolism proteins CPS1, ARG2, NNMT, OAT, UBA52, SLC7A5, GPT2, ERO1B, and CKMT1A. (D) Volcano plot showing the enrichment score (x-axis) and associated log p-value (y-axis) of differentially regulated phosphosite-driven signatures between tumors and matched NATs as assessed by PTM-SEA (Krug et al., 2018). The significant (FDR <0.05) signatures are highlighted in shades of brown. The size of the circles shows the overlap between phosphosites detected in our dataset and the phosphosite-specific signatures in PTMsigDB (Krug et al., 2018). (E) Rank plots depicting differential phosphosite-driven signatures between tumor and paired NATs in tumors with mutations in EGFR (N=38) or KRAS (N=33). Residual enrichment scores (y-axis) were calculated between mutated tumors (EGFR or KRAS) and all other tumors in order to highlight tumor and NAT differences in tumors harboring the specific mutation (EGFR or KRAS). (F) Heatmap representing tumor antigens including neoantigens (top panel) and cancer testis (CT) antigens (downloaded from CT database (Almeida et al., 2009)) (bottom panel). The frequency of somatic mutations per sample is shown in the top histogram, followed by predicted neoantigens with the RNA-level and proteome-level evidence in each sample shown in subsequent bar graphs. Clinical annotations include smoking status, driver mutation status, and DNA repair gene (POLE, MLH1, MLH3, MSH3, MSH4, MSH6, BRCA1, BRCA2) representation. ESTIMATE-based immune infiltration score for each sample is shown. The 11 cancer/testis (CT) antigen proteins overexpressed by at least 2-fold in tumors compared to NATs in more than 10% of samples are highlighted in red. Grey color indicates missing value.

FIG. 8A-FIG. 8J: Experimental workflow and data quality metrics. (A) Schematic representation showing sample processing steps. Fresh frozen tumors and their matched normal-adjacent tissues (NATs) were cryopulverized, followed by aliquoting for genomics and proteomics analyses. Cryopulverization promoted uniformity in samples undergoing multi-omics analysis. (B) Schematic representation of the workflow used for proteome, phosphoproteome and acetylproteome analysis. Tandem mass tags (TMT) were used to multiplex 9 tumors and NAT samples and 1 internal reference (pool of all tumors and NATs) that was used to link multiple TMT10 plexes. Matched tumor/NAT pairs were included in the same TMT plex. (C) Pearson similarity matrices showing intra- and inter-plex reproducibility across 4 interspersed CompRef process-replicates for proteome, phosphoproteome and acetylproteome. (D) Bar plot showing consistent numbers of identified and quantified proteins, phosphosites and acetylsites across the 25 plexes used for analyzing 212 tumors and NATs. (E) Principal component analysis (PCA) plot representation of proteome, phosphoproteome and acetylproteome separately for tumors and NATs, colored by TMT plex. (F) Sample-wise Pearson correlation between copy number alteration (CNA) and RNA, and between CNA and Proteome. The dark red-colored diagonal demonstrates the absence of sample swaps. (G) Cophenetic correlation coefficient (y-axis) calculated for a range of factorization ranks (x-axis). The maximal cophenetic correlation coefficient was observed for rank K=4 as shown in red. (H) Silhouette plot for K=4. (I) Non-negative matrix factorization (NMF) clustering applied to individual data types. This set of heatmaps reveals the NMF-based intrinsic structure of the data in the space of proteome, phosphoproteome, acetylproteome, mRNA, miRNA, and CNA data, respectively. Each heatmap shows the fractional membership score for each sample (x-axis) in each cluster (y-axis)—essentially, the strength of a sample's “belongingness” to each of the clusters. The maximal cophenetic correlation coefficient (excluding rank=2) was observed for the rank indicated in red. Proteome and RNA clusters overlap substantially with the multi-omics clusters depicted in FIG. 1E, but divergence is seen in both the phosphoproteome and acetylproteome, and additional substructure in the phosphoproteome. miRNA expression defines 5 clusters; Clusters 1 and 2 show notable enrichment for samples STK11 mutation and ALK fusion, respectively. CNA Cluster 1 contains almost exclusively males, most with KRAS mutations, whereas CNA Cluster 2 is enriched for EGFR mutations and almost devoid of KRAS mutations. (J) Louvain clustering of miRNA paralleled the NMF results in identifying 5 clusters. miRNA cluster 2 was markedly enriched for tumors from multi-omics cluster C1, in turn aligned with proximal-inflammatory RNA signatures, while miRNA cluster 3 was enriched for the STK11-mutant subset of the NMF C3, proximal-proliferative cluster. While the remaining three miRNA clusters had mixed composition, miRNA cluster 5 was markedly enriched for ALK fusion-driven tumors, including all 5 EML4-ALK as well as the HMBOX1-ALK fusions.

FIG. 9A-FIG. 9C: Genomic support for ALK fusions. (A) ALK gene fusion transcript architecture constructed from RNAseq data and fusion evidence for ALK fusion transcripts. Red arrows on the ALK and various 5′ partner genes' schematic diagrams indicate fusion breakpoints observed in the respective index samples. Blue arrows indicate gene orientation and numbers indicate genomic coordinates from hg38 assembly. (B) Identification of the precise genomic breakpoints from whole genome sequencing (WGS) data for ALK gene fusions. WGS evidence supporting the underlying genomic rearrangements in the ALK locus is indicated in red and blue; numbers indicate genomic coordinates from hg38 assembly. Immunohistochemistry reveals upregulation of both total ALK and the ALK Y1507 phosphosite specifically in the tumor epithelia of ALK fusion-positive samples. No staining was seen in RET or ROS1 fusion samples or in matched NATs.

FIG. 10A-FIG. 10D: Multi-omics integration. (A) Density plot showing distribution of sample-wise RNA-protein Spearman correlations separately for tumors and NATs. (B) Correlation plots of CNA vs Phosphoprotein and CNA vs Acetylprotein expression. Significant (FDR <0.05) positive and negative correlations are indicated in red and green, respectively. CNA-driven cis-effects (consequence of CNA on the same locus) appear as the red diagonal line; trans-effects (consequence of CNA on genes encoded elsewhere) appear as vertical red and green lines. The accompanying histograms show the number of significant (FDR <0.05) cis- and trans-events corresponding to the indicated genomic loci (upward plot) as well as the overlap between CNA-RNA and CNA-Protein events (downward plot). (C) Heatmap showing 2 dominant clusters of DNA methylation, defined primarily by tumors and NATs. (D) Consensus clustering of CpG island methylator phenotype (CIMP) defines three stable clusters representing high, intermediate and low CIMP phenotypes.

FIG. 11A-FIG. 11I: Impact of mutation on proteogenomic landscape of tumors. (A) The cis- and trans-effects of select mutated genes on the RNA expression of cancer-associated genes. Cis-effects and trans-effects are indicated as circles and squares respectively. The red/blue scale represents change in RNA expression and the size represents significance. (B) Lollipop plot showing KEAP1 mutations identified in this LUAD cohort. The color of the dots indicate the type of mutation. (C) Box plot showing KEAP1 and NFE2L2 RNA expression in KEAP1 wild-type and mutant samples. Also shown is the downregulation of KEAP1 in KEAP1 mutant samples, seen at the protein but not at the RNA level. (D) Volcano plot showing differentially regulated proteins in KEAP1 wildtype vs mutant samples. These differential proteins underlie the pathway analysis shown in FIG. 4D. (E) Volcano plot showing differentially regulated phosphosites in KEAP1 wildtype vs mutant samples. (F) Pathways enriched among proteins differentially expressed between KEAP1 mutant and wildtype tumors. Significant enrichment of the oxidative stress response supports activation of NRF2 signaling in these samples. (G) Box plot showing unchanged PTPN11 protein and significantly (FDR <0.05) elevated phosphopeptide expression (Y546 and Y584) in ALK fusion relative to wild-type samples. (H) Box plot showing increase in GAB1 in ALK fusion relative to other samples. (I) Protein-level gene-set enrichment (GSEA) pathway comparison of EGFR- and KRAS-driven LUAD tumors showing disparity in complement and clotting cascades, with upregulation of coagulation in KRAS and downregulation in EGFR mutant samples. (J) Heatmaps show the phosphatase, ubiquitinase and deubiquitinase outliers enriched (FDR <0.2) at the phosphoprotein, protein, RNA and CNV levels and their association with mutations in select genes (EGFR, KRAS, TP53, STK11, KEAP1, EML4-ALK). DepMap panels on the left show log 2 transformed relative survival averaged across all available lung cell lines after depletion of the indicated gene (rows) by RNAi or CRISPR. Druggability based on DGIdb (http://www.dgidb.org/) is indicated alongside the availability of FDA-approved drugs. The log-transformed druggability score indicates the sum of PubMed journal articles that support the drug-gene relationship.

FIG. 12A-FIG. 12G: Immune landscape in LUAD. (A) Heatmap of expression levels of proteins most correlated with inferred activation of IFN-g axis. Proteins involved in immune evasion signatures and proteins annotated as drug targets (as defined by: https://www.drugbank.ca) are highlighted by vertical bars on the left side of the figure. (B) The heatmap shows abundance levels (converted to Z-scores) of proteins of the IFN-γ axis pathway (Abril-Rodriguez and Ribas 2017) and the Surfactant Metabolism pathway from Reactome database (Fabregat et al. 2018). The IFN-γ axis pathway determines activation of the adaptive immune system; 11 proteins of IFN-γ axis were detected in global proteomics as presented in the heatmap (magenta vertical bar). The Surfactant Metabolism pathway (turquoise vertical bar) was identified among three top pathways anti-correlated with inferred activation of IFN-γ axis pathway. Thus, upregulation of surfactant proteins was associated with downregulation of IFN-γ axis pathway. Lung surfactant is composed of a complex lipoprotein-like mixture that lines the inner surface of the lung to prevent alveolar collapse at the end of expiration and to regulate pulmonary innate immunity (Whitsett 2014). The original Surfactant pathway is composed of 30 proteins; 14 of those proteins, including 5 of 6 primary surfactant proteins (SFTPD, SFTPB, SFTPC, SFTPA1, SFTPA2) were detected by global proteomics. The surfactant proteins can regulate immune responses by increasing immunosuppression (Pastva et al., 2007, Nayak et al, 2012). Prior studies have shown association between genetic polymorphisms of surfactant proteins and lung carcinoma (Seifart et al., 2005) and bronchopulmonary dysplasia (Pavlovic et al. 2006). In particular, genetic defects in SFTPA2, the gene encoding surfactant protein SP-A2, are associated with lung cancer (Wang et al. 2009). The revealed upregulation of surfactant proteins in CTE lung tumors confirms the association of surfactant proteins with immune suppression mechanisms in lung cancer. (C) Heatmap depicting normalized enrichment scores (NES) of Hallmark gene sets (Liberzon et al. 2015) in each multi-omic cluster. To calculate cluster-specific NES we projected the matrix of multi-omic feature weights (VV) derived by non-negative matrix factorization (NMF) onto gene sets using single-sample Gene Set Enrichment Analysis (ssGSEA) (Barbie et al. 2009). To derive a single weight for each gene measured across multiple omics data types (protein, RNA, phosphosite, acetylsite) we retained the weight with maximal absolute amplitude. Only gene sets significant in at least one cluster are shown (FDR <0.01). (D) Boxplot showing the distribution of the number of non-synonymous somatic mutations in each multi-omic cluster. Numbers inside the box depict the median number of nonsynonymous mutations in each cluster. (E) Flow diagram showing the workflow for developing and testing a deep learning algorithm to identify STK11 mutant samples based on hematoxylin and eosin stained histopathology slides. (I) LUAD tissue histopathology slides were downloaded from The Cancer Imaging Archive (TCIA) database; (II) slides and corresponding per-slide level labels were separated into training (80%), validation (10%), and test sets (10%) at the per-patient level; (III) slides were tiled into 299-by-299-pixel pieces with overlapping areas of 49 pixels from each edge, omitting those with over 30% background. Tiles of each set were packaged into a TFrecord file; (IV) the InceptionV3-architectured convolutional neural network (CNN) was trained from scratch and the best performing model was picked based on validation set performance; (V) the model was applied to the test set, and the per-tile prediction scores were aggregated by slides and shown as heatmaps. The last-layer activations of 10000 randomly sampled tiles were exported for feature visualization on tSNE (FIG. 5E); (VI) counts and statistical metrics (area under ROC, area under PRC, and accuracy) on per-slide and per-tile level were calculated with bootstrapped 95% confidence interval in parentheses. Table: the model achieved per-slide level AUROC of 0.961 and per-tile level AUROC of 0.892 in predicting STK11 mutation. Slide-level predictive accuracy was 94%. (F) To extract pathway-level proteomic features in an unsupervised manner, protein abundance measurements of 110 tumor samples were submitted to independent component analysis following the method proposed in (Liu et al., 2019). One signature (IC_068) showed significant associations with STK11 mutation status (average log 10 nominal P-values within component cluster: −5.7). Average mixing scores for IC_068 represented ‘activity’ of the meta-gene level signature in each of the samples. Raw protein abundance values of genes contributing heavily to the signature (coefficient larger than 3) were shown in a heatmap. (G) Heatmaps showing protein (upper) and RNA expression (lower) of 16 gene products associated with neutrophil degranulation. STK11 and its partner STRADA are also shown.

FIG. 13A-FIG. 13E: Impact of smoking on somatic mutations. (A) Bar plots showing distinct mutational signatures in 110 LUAD tumors, identified using SignatureAnalyzer applied to WGS data. (B) Schematic showing the approach used for determining our revised smoking score. Tumor purity estimates, counts of total mutations, and percentages that are smoking signature mutations and smoking-signature DNPs were used to derive a continuous smoking signature score. (C) Shown are the pairwise cosine similarities of each pair of the substitution signature probability for the 53 environmental mutagen exposures reported in Kucab et al (Kucab et al. 2019). (D) Box plot showing significant difference (p<2.2e-16) in RNA-based stemness scores between tumors and NAT. (E) Box plot showing decrease of RNA-based stemness scores in both tumors and NATs with high smoking score (HSS) compared to corresponding samples with low smoking score (LSS).

FIG. 14A-FIG. 14E: Proteogenomic differences between tumors and matched adjacent normals. (A) Principal component analysis (PCA) plots showing RNA, protein, phosphosite and acetylsite expression in 110 tumor samples (cadet blue) and 101 normal samples (coral). (B) Volcano plots showing differentially regulated RNA, proteins, phosphosites and acetylsites between tumors and NATs. Significantly upregulated sites (FDR<0.01, Log 2 fold-change >1) and downregulated sites (FDR <0.01, Log 2 fold-change <−1) are indicated in red and blue, respectively. Analyses of protein (10,316), RNA (18099), phosphosites (40845) and acetylsites (6984) showed that 14.8%, 8.2%, 13.94%, and 14.11% of these gene products, respectively, were globally upregulated while 26.4%, 13.6%, 15.2%, and 11% were downregulated in tumors (≥2-Fold change, FDR <0.01. (C) Enrichment analysis (GSEA) revealing pathways differentially expressed between tumor and paired NATs. Cell cycle progression, MYC Targets Upregulation, Unfolded Protein Response, Glycolysis and TCA cycle (adjusted p<0.001) were upregulated in tumor samples whereas KRAS Signaling (adjusted p=5×10⁻³), STAT3 Signaling (FDR=7.61×10⁻³), and Muscle Differentiation (p<0.001) were downregulated in tumor samples compared to NATs. (D) Global-LUAD: Using stringent cutoffs for quantitative difference, significance and consistency (log 2-fold change >2, adjusted p<0.01, and differential in ≥90% of all Tumor-NAT pairs) we identified 289 proteins upregulated at the protein level in tumors, 60 of which were supported by RNA and are shown in the figure. HPA proportions indicate the proportion of lung adenocarcinoma sections staining positive for the specific marker in the Human Proteome Atlas. This global tumor/NAT comparison revealed 18 enzymes, 3 transcription factors (SOX4, TCF3, HMGA1), 2 transporters, and 23 secreted, 21 transmembrane, and 22 plasma proteins as candidate biomarkers. GREM1, SOX4, SPINT1, ST14, SPINT2, CTHRC1, KDF1, MDK, SFN, HMGA1, ESRP1, NME1, SERPINH1 and CBX8 are implicated in EMT and metastasis. Highly upregulated metabolic proteins included GFPT1, P4HB, PLOD2, PYCR1, SHMT2, PSAT1, ERO1A, IL4I1, DHFR, and LDHA. Stress-related marker candidates with prognostic significance included ERO1A, DHFR, MANF, HYOU1, LDHA, and CBX8. Remainder of figure: Proteomics-based tumor biomarker candidates (fold change >4 and adjusted p-value <0.01 in ≥80% of tumor/NAT pairs) for 4 frequently mutated genes: TP53 (40/63 shown), EGFR (39), KRAS (37) and STK11 (40/77 shown). Each dot represents a tumor sample. Blue-colored boxplots highlight proteins with overexpression in more than 99% of tumor samples with the associated mutation. HPA proportions indicate the proportion of lung adenocarcinoma sections staining positive for the specific marker in the Human Proteome Atlas. Relevant characteristics of the biomarker candidates (enzyme, transcription factor, secreted, transmembrane or observed in plasma), and relevant targeted drugs in clinical trials are shown in the accompanying crossword plot. Condensed representations of these plots are shown in FIG. 21C. (D) Rank plots depicting differential phosphosite-driven signatures between tumor and paired NATs in tumors with mutations in STK11 or TP53. (E) Residual enrichment scores (y-axis) were calculated between mutated tumors (STK11 or TP53) and all other tumors in order to highlight tumor and NAT differences in tumors harboring the indicated mutation (F).

FIG. 15A-FIG. 15C: Proteogenomic summary of the cohort. (A) Summary of data availability of 10 data types generated in this study by sample. (B) Overview of genetic alterations of significantly mutated genes with 5% and more samples altered. For each sample, we display tumor mutation burden (log₂ WES mutation count), structural variant, gene fusion, and copy number variation per gene. (C) Multi-omics clustering of tumor samples by non-negative matrix factorization (NMF) using copy number variation, gene expression, and protein and phosphoprotein abundance as the features. The features recognized to be characteristic expression for each subtype are shown in the heatmap and underwent pathway enrichment analysis to designate representative pathways. Neuron activity related pathways were enriched in the proneural-like subtype (nmf1), Immune response pathways were enriched in the mesenchymal-like subtype (nmf2), and cell cycle pathways were enriched in the classical-like subtype (nmf3). See also FIG. 22, FIG. 23.

FIG. 16A-FIG. 16I: Cis and trans effect of SMGs on RNA, protein and phosphorylation level and ATRX and IDH1 mutations on telomerase activity and length. (A) The cis and trans effect of significantly mutated genes (Y axis) on RNA and protein level (X axis). The effects on RNA and protein often resemble one another. (B) The cis and trans effect of significantly mutated genes (Y axis) on phosphorylation status (X axis). (C) Effect of TP53 alterations on itself, as well as CDK2 and MDM2. The schematic of the negative/positive feedback loop between CDK2, MDM2 and TP53. (D) Effect of RB1 alterations on itself, as well as CDK2, CDK6, MCM2, MCM4, and MCM6 protein expression. Right panel is a cartoon of the proposed interplay between RB1, MCM2, MCM4, and MCM6 in RB1 altered and wild type. (E) ATRX and IDH1 mutational effect on ATRX RNA and protein levels. IDH1 R132H mutations affect ATRX protein only. (F) Neither ATRX nor IDH1 mutations are associated with reduced DAXX protein expression. (G) TERT RNA expression in proposed telomere genotypes. TERTp mutations are associated with stable TERT expression. (H) Telomere length ratio (WGS tumor telomere length/WGS blood normal telomere length) in proposed telomere genotypes. (I) Potentially druggable proteins whose phosphosites are significantly (outlier analysis FDR <0.2) enriched in tumor samples with long telomeres (telomere ratio >1.2) and short telomeres (telomere ratio <0.8). See also FIG. 24.

FIG. 17A-FIG. 17E: Genetic alterations in RTKs and their effects on the expression of protein and phosphosites of themselves and downstream genes. (A) Genetic alterations—structural variations (SV), fusions, mutations (MUT) and copy number variations (CNV)—in EGFR, PDGFRA, FGFR3 and MET, and their cis effect on RNA, protein and phosphosite expression. (B) Volcano plot showing proteins and phosphosites differentially expressed between EGFR-altered and EGFR wild type samples. (C) Left panel: Proteomic effect of altered EGFR on key genes. Right panel: PTPN11 protein level is not affected by EGFR alterations, while phosphorylation of its Y62 site is increased in EGFR-altered samples. (D) Heatmap showing significant (kinase-substrate analysis FDR <0.1) cis- and trans-regulated sites of EGFR and PDGFRA kinases. Both EGFR and PDGFR regulate phosphorylation of PTPN11, but at different phosphosites. The schematic on the right shows dual regulation of PTPN11 by EGFR and PDGFRA, and the downstream substrates that PTPN11 may dephosphorylate. See also FIG. 25.

FIG. 18A-FIG. 18D: Distinct cell type enrichment, macrophage, and immune marker expression, and epigenetic modification analyses derive two immune subtypes. (A) The immune-high and low subtypes identified by the cell type enrichment with distinct expression patterns in the common macrophage markers, Tumor-Associated Macrophage (TAM) markers, and the immune checkpoints and potential immunotherapy targets on the gene expression and global protein expression levels; distinct methylation patterns are associated with the immune-high and low subtypes in GBM; differential expression between immune-high and immune-low samples based on acetylation, global protein abundance, phosphoprotein abundance, and gene expression (FDR <0.05) and Hallmark, KEGG, Reactome pathway enrichment based on the Fisher exact test (FDR <0.1, only pathways with at least 10 genes observed). (B) Visualization of features captured by a deep learning model. Each dot represents a tile of H&E slides in the test set and they are colored according to the prediction score of the deep learning model (orange means predicted immune-high; blue means predicted immune-low). There are 20,000 sampled tiles from 99 patients were clustered by applying tSNE to their activation maps (a 1250-long vector for each tile) from the last layer of the deep learning model to dimensionally reduced into 2-dimension space. (C) Box plots show immune-high subtype have increased immune scores (ESTIMATE, xCell), low mRNA-based stemness index, and fewer WGS mutation counts (log₂-scaled) compared with immune-low subtype. (D) Bar plots show the enrichment score (immune score, stroma score, microenvironment score, macrophage scores) distribution among two immune subtypes in CPTAC GBM and TCGA GBM. See also FIG. 26.

FIG. 19-FIG. 19E: Histone acetylation association with immune subtypes and pathways activity. (A) Unsupervised clustering of histone protein and site-level acetylation reveals distinct clusters of tumors enriched for H3, H4 acetylation and H2B acetylation. (B) Significant associations between histone acetylation sites and histone acetyltransferase, deacetylases, and bromodomain-containing proteins. (C) Pathways associated with H2B acetylation or with H3, H4 acetylation levels by multiomics subtype. (D) Significant Spearman's correlation between xCell scores and acetylation of histone sites (FDR <0.05). (E) SUMO1 and UBE2I protein expression across samples with high and low H2B acetylation. See also FIG. 27.

FIG. 20A-FIG. 20G: Lipidome and metabolome data map to major metabolic and signaling pathways. (A) Averaged abundance of all lipids detected across four tumor expression subtypes and GTEx normal samples. Lipids are sorted by the total number of chain double bonds and by the total number of carbons in side chains. (B) Lipid Mini-On enrichment analysis of lipid properties upregulated in subtype X versus subtype Y. (C) Diagram shows contribution of enzymes that activate PUFAs (ACSL4 and ACSL6) to the phospholipid pool and connection of PUFA-containing phosphatidylethanolamine (PE) to ferroptosis. DHA—docosahexaenoic acid, AA—arachidonic acid, AdA—adrenic acid. (D) Protein expression of ACSL6, ACSL4 and ALOX5 across tumor expression subtypes and GTEx normal. (E) Schematic diagram of lipid conversion reactions essential for cell signaling. (F) Correlation between diacylglycerols (DG), phosphatidic acid (PA) and phospholipases C (cleaves PIP₂ into DG and IP₃), Akt kinases (interact with PIP₃), Protein kinase C (interacts with DG), and DG kinases (phosphorylates DG to produce PA). (G) IDH1 mutants display elevated abundance of glucose, glycolytic intermediate metabolites and oncometabolite 2-hydroxyglutarate (2-HG), along with reduced abundance of glutamate and serine. See also FIG. 27.

FIG. 21A-FIG. 21C: Summary of pathway alterations and potential therapeutic targets. (A) Three oncogenic pathways frequently altered in GBM. Each gene is annotated with the mutational and CNV frequency, RNA, protein, and phosphoprotein abundance per expression subtype. The bottom bar in each gene annotated the alteration frequency in all tumors across all subtypes. We also summarize the proportion of tumors with genetic alterations (first percentage) and protein and phosphoprotein outlier expression (second percentage) for each pathway, respectively. (B) Dysregulated phosphosignaling in RTK, PI3K, WNT, and NOTCH oncogenic pathways across all tumors. Thickness of a kinase-substrate edge indicates how strong variation in the substrate phosphosite abundance can be explained by the variation of the kinase phosphorylation. The color of the edge indicates the percentage of samples with outlier phosphorylation. Kinases governing phenotypic GBM genes with substantial outliers may be potential therapeutic targets. (C) Sample level outlier status for kinase-substrate pairs shown in FIG. 21B. For kinases (blue) outlier status for four levels of data is shown: CNV, RNA, PRO (protein) and PHO (phosphorylation). For their substrates (green) only PHO outlier status is shown. See also FIG. 28.

FIG. 22A-FIG. 22C. Integrated Proteomic Workflow and Quality Control, Related to FIG. 15. (A) TMT-11 based global proteome, phosphoproteome, and acetylome analysis workflow. The GBM tumors and GTEx normal tissues were analyzed in 11 TMT 11-plex experiments, each with 10 study samples and a common internal reference sample created by pooling all study samples (equal contribution). The TMT11-labeled samples were then fractionated, split (with 5% peptide mass analyzed directly for global proteome), and subjected to tandem enrichment of phosphopeptides and acetylated peptides. Peptides were detected and quantified using information from the TMT-11 MS/MS spectra. (B) Distribution of sequence coverage of the identified proteins with tryptic peptides detected by MS/MS in each TMT-11 plex; whiskers show the 5-95 percentiles. (C) Robust and precise proteomics platforms. Longitudinal performance was tested by repeated proteome, phosphoproteome and acetylome analysis of aliquots of the same patient-derived xenograft QC samples in standalone TMT-11 plexes, along with the GBM study samples; scatter plots and Pearson correlations comparing individual replicate measurements are shown.

FIG. 23A-FIG. 23D. Proteogenomic Characterization and Subtyping of GBM, Related to FIG. 15. (A) Heatmap of multi-omics membership scores of all three nmf subtypes for each tumor. (B) GISTIC2 copy number variations at arm (left) and focal (right) level of all tumors with WGS available. (C) Survival Kaplan-Meier curves of mixed-subtype tumors with low multi-omics membership score versus the rest of the tumors. (D) Pathway enrichment analysis of gene expression with differential DNA methylation in promoter regions across all six DNA methylation subtypes.

FIG. 24A-FIG. 24J. Effect of TP53 and TERTp Mutations, and ALT Phenotype Tumors Relation to Patient Age and Number of Mutations, Related to FIG. 16. (A) Boxplots showing RNA, protein and phosphosite abundance for TP53 and TP53BP1 in TP53 wild type tumors (blue) and TP53-mutated tumors (red). (B) Scatterplot showing TP53 protein expression change in TP53 mutated samples in regard to the mutation position in the p53 protein. (C) Variant allele frequency of mutations in TERTp region found in tumors not carrying TERTp hotspot mutations but expressing TERT RNA (upper panel) and in tumors not carrying TERTp hotspot mutations and no expressing TERT RNA (bottom panel). For the upper panel mutations with >10% VAF are shown, for the bottom panel mutations with >5% VAF are shown. Triangles indicate hotspot genomic position 1295113 and 1295135 (−124 and −146 bp from TSS labeled by the dashed line). (D) Overview of genetic alterations in TERTp hotspot and not hotspot positions, mutations in ATRX, IDH1 and TP53. Telomere length ratio in tumor/blood normal is indicated on top, patient age is indicated in the bottom. (E) TERT RNA expression in TERTp hotspot mutated, not hotspot mutated and TERTp wild type samples. (F) ATRX RNA and DAXX RNA are not correlated with each other. (G) Overrepresentation testing showed enrichment for cell junction pathway in the telomere-long group. (H) Telomere length ratio is correlated with patient age in ATRX and/or IDH1 tumors. (I) Telomere length ratio is correlated with the total count of synonymous and non-synonymous mutations in the coding region in ATRX and/or IDH1 tumors. (J) Age of the patient is correlated with the total count of synonymous and non-synonymous mutations in the coding region in ATRX and/or IDH1 tumors.

FIG. 25A-FIG. 25C. Boxplots of CNV, RNA, Protein and Phosphosite Level in Samples With Different RTK Alterations, Related to FIG. 17. (A) The comparison of CNV, RNA and protein expressions and phosphosite level between EGFR altered and WT samples (upon panel) and PDGFRA altered and WT samples (bottom panel). (B) The comparison of RNA and protein expression of FGFR3 and TACC3 between samples with and without FGFR3-TACC3 fusion and the breakpoints found in FGFR3 gene from RNA-Seq data. Three samples which are protein expression outliers in FGFR3 are marked by large circle. (C) The comparison of PLCG1, GAB1, and GRB2 protein expression and PLCG1-Y783 phosphosite level between EGFR-altered and WT samples.

FIG. 26A-FIG. 26E. Distinct Immune Marker Expression Ascertains Immune Subtypes, Related to FIG. 18. (A) The immune high and low subtypes identified by the cell type enrichment with distinct expression patterns in the common macrophage markers, Tumor-Associated Macrophage (TAM) markers, and the immune checkpoints and potential immunotherapy targets on the gene expression in the TCGA GBM cohort. (B) Single-sample gene-set enrichment analysis (ssGSEA) of each immune subtype among the Hallmark, KEGG, Reactome, and PID gene sets. (C) The overall survival of the two immune groups and stratified by ancestry groups (Asian, Caucasian, Hispanic). (D) Box plots show immune-high subtype have more high-level EGFR amplification and high-level PTEN, CDKN2A, CDKN2B deletions in the immune-low subtype. (E) Experienced pathologists reviewed the immune-low tiles and marked the giant cells. (F) Experienced pathologists reviewed the immune-high tiles and marked the inflammatory cells.

FIG. 27A-FIG. 27K. Relations Between H2B Acetylation and SUMOylation Pathway, Related to FIG. 19; Additional Lipid Abundance Evidence, Related to FIG. 20. (A) Scatterplot showing correlation between acetylated H2B peptide K16K17 and Th1 xCell score. Pearson's correlation value and p-value are shown. (B) Scatterplot showing correlation between UBE2I protein level and averaged H2B acetylation level. Pearson's correlation value and p-value are shown. BRAF mutated samples are labeled in dark orange. (C) Scatterplot showing correlation between UBE2I protein level and CDK6 protein level. Pearson's correlation value and p-value are shown. RB1 mutated samples are labeled in dark orange. (D) Frequency of the side chains observed in the detected lipids. (E) Abundance of phosphatidylcholine PC(22:0/0:0) across expression subtypes and GTEx normal samples. Wilcoxon test p-value is labeled. (F) Scatterplot showing correlation between PLA2G4A protein level and PC(22:0/0:0) abundance. Pearson's correlation value and p-value are shown. (G) Abundance of phosphatidylglycerol PG(22:4/22:6) across expression subtypes and GTEx normal samples. 22:6 is likely DHA. Wilcoxon test p-value is labeled. (H) Abundance of triacylglycerol TG(16:0/20:1/22:6) (also known as triglycerides) across expression subtypes and GTEx normal samples. 22:6 is likely DHA. Wilcoxon test p-value is labeled. (I) Abundance of 22:6-carrying PC, PE and PS across expression subtypes and GTEx normal samples. 22:6 is likely DHA. Wilcoxon test p-value is labeled. (J) PEs carrying 22:4 or 20:4 fatty acids are downregulated in mesenchymal subtype, while PE not containing either of the two FAs is upregulated in IDH1 mutant subtype only. (K) Scatterplot showing correlation between DHA abundance and PI (20:4/22:6) abundance. Pearson's correlation value and p-value are shown.

FIG. 28. Summary of Pathway Alterations of Mesenchymal Tumors Compared to the Other Tumors, Related to FIG. 28. Causal explanations for differentially expressed proteomic and phosphoproteomic profiles in mesenchymal tumors versus the rest of the tumors using CausalPath. We highlighted the immune and hypoxia-related interactions enriched in the graph. Genes controlling the immune response might be a potential therapeutic target.

### DETAILED DESCRIPTION OF THE INVENTION

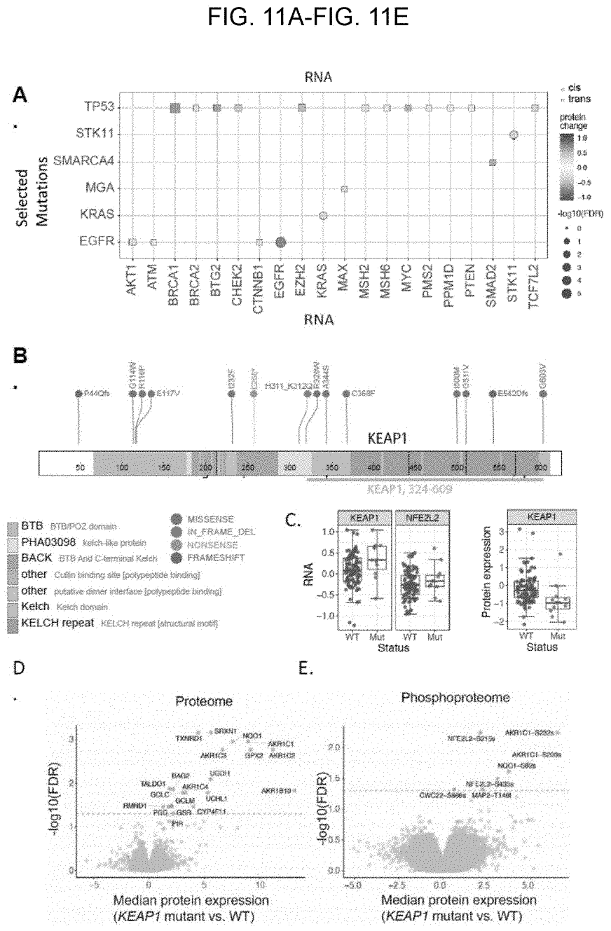

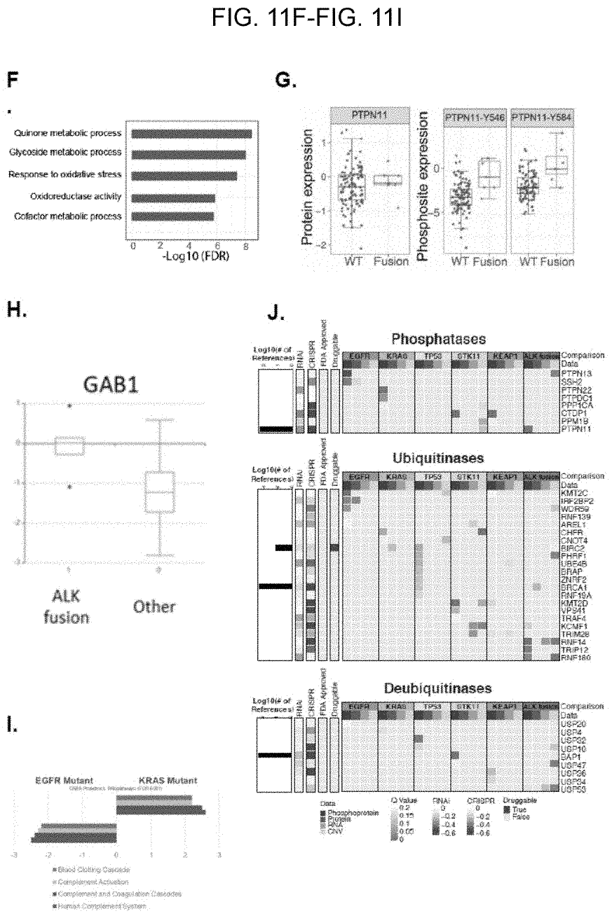

The present disclosure is based, at least in part, on the discovery of drug targets for brain cancer (e.g., glioblastoma multiforme (GBM)) and lung cancer (e.g., lung adenocarcinoma (LUAD)), including refractory GBM and LUAD. Other cancers that can be targeted include breast cancer and pancreatic cancer, though to a slightly lesser degree compared to brain and lung cancers. As shown herein, proteins and phosphosites upregulated in samples with KEAP1 mutations (FIG. 11D and FIG. 11E) were members of the NFE2L2 oncogenic signatures, mostly associated with activation of antioxidant responses that provide cytoprotection to cancer cells (FIG. 11F) (Taguchi and Yamamoto, 2017). These upregulated proteins provide potential therapeutic targets for treating KEAP1 mutant tumors.

As described herein, the data suggests that EGFR mutant- and ALK fusion-driven LUADs would be particularly promising target populations for Shp2 inhibitor therapy (e.g., Shp2 inhibitor, SHP099, in combination with the ALK inhibitor ceritinib). Furthermore, extreme phosphorylation events on important, targetable proteins implied therapeutic possibilities including SOS1 inhibition in KRAS mutant and PTPN11 (Shp2) inhibition in EGFR mutant tumors. Druggable targets discovered herein include EGFR, KRAS, TP53, STK11, KEAP1 and EML4-ALK. Our findings also suggest that the combination of PD-1/PD-L1 blockade with IDO1 inhibitor might increase efficiency of treatment of immune hot tumors in LUAD.

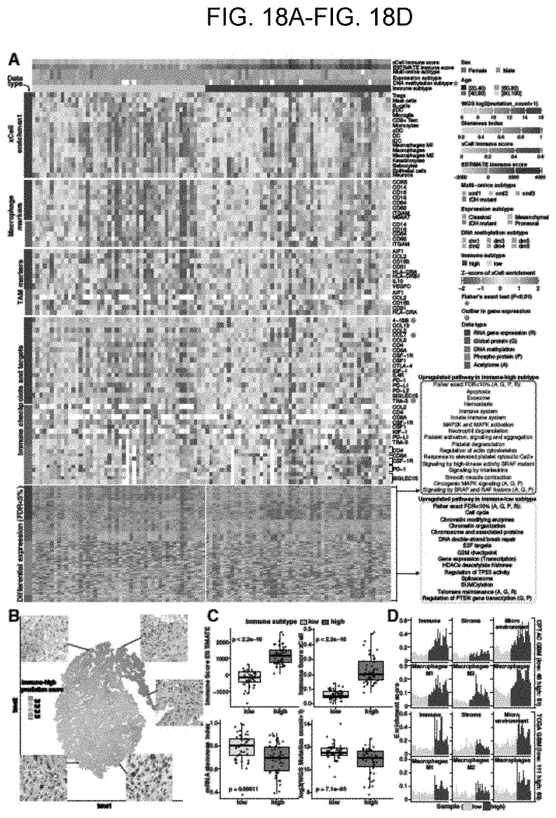

As described herein, in GBM, phosphoproteomic analysis identified PTPN11 and PLCG1 as the principal switches mediating RAS pathway activation, and therefore potential therapeutic targets. Furthermore, upregulated protein or phosphorylation levels of activated MCM genes could be drug targets in RB1-altered samples. Increases in phosphorylation of PTPN11 (Shp2) suggest anti-Shp2 therapies may be effective in RTK-altered samples, regardless of the driver mutation or genomic alteration. Co-upregulation of CDK6 and EGFR suggests combining EGFR and CDK6 inhibitors in EGFR-altered samples. Also, we identified TNIK phosphoprotein as an outlier in the telomere-long group of samples, suggesting that patients with ATRX and IDH1 mutations may benefit from TNIK inhibitors. Additionally, our results support targeting TAMs in the microenvironment as an adjuvant therapy. Since macrophages depend upon CSF-1 (Pyonteck et al., 2013) and expression of both CSF-1 and CSF-1R are high in the immune-high group (FIG. 18A), the use of a CSF-1 inhibitor might be beneficial. Another option might be to inhibit TAM infiltration by targeting MCPs, which mediate macrophage migration and infiltration: four MCP family members (CCL2, CCL7, CCL8, and CCL13) were highly expressed in the immune-high subtype (FIG. 18A). Our data also suggest that anti-angiogenic agents such as bevacizumab combined with immunotherapy may be more beneficial in mesenchymal-like GBM.

Protein Target Modulating Agent

One aspect of the present disclosure provides for targeting (e.g., inhibiting, modulating) of identified target proteins (i.e., upregulated phosphosites/upregulated phosphorylation), their receptors, or their downstream signaling. The present disclosure provides methods of treating or preventing cancer (e.g., LUAD, GBM) based on the discovery of specific proteins, receptors, or pathways that can be targeted based on the tumor protein, phosphoprotein, or acetylation expression, among others.

As described herein, inhibitors of the tumor target protein (e.g., small molecules, antibodies, fusion proteins) can reduce or prevent protein activity or expression. A target protein inhibiting agent can be any agent that can inhibit a target protein, downregulate a target protein or activity, or knockdown the target protein.

The present disclosure provides for targets and therapeutic methods for treating cancer. The below table includes targets and therapy or agents associated with that target, where applicable. The therapeutic/drug can be used in any combination.

TABLE 1
Targets and therapeutic agents.

| | Therapeutic/drug |
|---|---|
| Protein Target or Subtype | |
| (lung cancer) | |
| PTPN11 | Shp2 inhibitor, such as SHP099, TNO155, |
| | JAB-3068, RMC-4630, BBP 398, RLY1971 |
| EGFR mutants and ALK | Shp2 inhibitor (e.g., SHP099, TNO155, |
| fusion | JAB-3068, RMC-4630, BBP 398, RLY1971) and an |
| | ALK inhibitor (e.g., ceritinib) |
| KEAP1 mutants | Inhibitors of TXNRD1, SRXN1, NQO1, |
| | ARK1C1, ARK1C3, GPX2, AKR1C2, BAG2, |
| | UGHD, ARK1B10, ARK1C4, TALDO1, GCLC, |
| | GCLM, UCHL1, AKR1B10, RMND1, PGD, |
| | GSR, CYP4E11, NFE2L2, AKR1C1, Inhibitors |
| | of AKR1C1, NQO1, NFE2L2, CWC22, MAP2, |
| | or KEAP1/NFE2L2 interaction |
| Protein Target or Subtype | |
| (brain cancer) | |
| PTPN11 | Shp2 inhibitor (e.g., SHP099) |
| PLCG1 | PLC inhibitors (e.g., edelfosine, neomycin) |
| RB1-altered | MCM inhibitor (e.g., ciprofloxacin) |
| EGFR-altered having co- | Inhibitors of CDK6 and EGFR |
| upregulation of CDK6 and | |
| EGFR | |
| EGFR-altered and | anti-Shp2 therapy |
| PDGFRA-altered tumors | |
| Tumors with ATRX and | TNIK inhibitors |
| IDH1 mutations | |
| Immune-high tumors | CSF-1 inhibitors, MCP inhibitors |

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀ [Na⁺])+0.41(fraction G/C content)−0.63(% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain | |
|---|---|
| Characteristic | Amino Acid |
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, Tex.; Sigma Aldrich, Mo.; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Genome Editing

As described herein, target protein signals can be modulated (e.g., reduced, eliminated, or enhanced) using genome editing. Processes for genome editing are well known; see e.g. Aldi 2018 Nature Communications 9 (1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of target protein expression by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for cancer to target cells by the removal of target proteins signals.

For example, the methods as described herein can comprise a method for altering a target polynucleotide sequence in a cell comprising contacting the polynucleotide sequence with a clustered regularly interspaced short palindromic repeats-associated (Cas) protein.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating cancer (e.g., LUAD, GBM) in a subject in need of administration of a therapeutically effective amount of a protein target modulating (e.g., inhibiting agent), so as to modulate/inhibit a target protein, activity, expression, or phosphorylation. Lung and brain cancer are the major targets with the agents described herein, but also have a role in breast and pancreatic cancer.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of a protein target modulating agent is, for example, an amount that would cause the desired therapeutic effect in a subject while minimizing undesired side effects. In various embodiments, an effective amount of a protein target modulating agent described herein can substantially inhibit cancer progression, slow the progress of cancer progression, or limit the development of cancer progression.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a protein target modulating agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to modulate a target protein activity, expression, phosphorylation, or amount.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a protein target modulating agent can occur as a single event or over a time course of treatment. For example, a protein target modulating agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for cancer.

A protein target modulating agent can be administered simultaneously or sequentially with another agent, such as a chemotherapeutic agent, radiation therapy, immunotherapy, or another agent. For example, a protein target modulation agent can be administered simultaneously with another agent, such as a chemotherapeutic agent, radiation therapy, immunotherapy, or another agent. Simultaneous administration can occur through administration of separate compositions, each containing one or more of a protein target modulation agent, a chemotherapeutic agent, radiation therapy, immunotherapy, or another agent. Simultaneous administration can occur through administration of one composition containing two or more of a protein target modulation agent, a chemotherapeutic agent, radiation therapy, immunotherapy, or another agent. A protein target modulation agent can be administered sequentially with a chemotherapeutic agent, radiation therapy, immunotherapy, or another agent. For example, a protein target modulation agent can be administered before or after administration of a chemotherapeutic agent, radiation therapy, immunotherapy, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Cancer

Methods and compositions as described herein can be used for the prevention, treatment, or slowing the progression of cancer, tumor growth, or tumor cell proliferation. As shown herein, cancers associated with EGFR, KRAS, TP53, STK11, KEAP1, and EML4-ALK are targeted. Lung and Brain cancers are the major target, with breast and pancreatic cancers to a lesser degree.

Other cancers that could be potentially targeted include, for example, can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sezary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sezary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor. Brain or spinal cord tumors can be acoustic neuroma; astrocytoma, atypical teratoid rhaboid tumor (ATRT); brain stem glioma; chordoma; chondrosarcoma; choroid plexus; CNS lymphoma; craniopharyngioma; cysts; ependymoma; ganglioglioma; germ cell tumor; glioblastoma (GBM); glioma; hemangioma; juvenile pilocytic astrocytoma (JPA); lipoma; lymphoma; medulloblastoma; meningioma; metastatic brain tumor; neurilemmomas; neurofibroma; neuronal & mixed neuronal-glial tumors; non-hodgkin lymphoma; oligoastrocytoma; oligodendroglioma; optic nerve glioma; pineal tumor; pituitary tumor; primitive neuroectodermal (PNET); rhabdoid tumor; or schwannoma. An astrocytoma can be grade I pilocytic astrocytoma, grade II-low-grade astrocytoma, grade III anaplastic astrocytoma, or grade IV glioblastoma (GBM), or a juvenile pilocytic astrocytoma. A glioma can be a brain stem glioma, ependymoma, mixed glioma, optic nerve glioma, or subependymoma.

### Definitions

A control sample or a reference sample as described herein can be a sample from a tumor without a target mutation (e.g., IDH-wild type, EGFR-wildtype), a healthy subject, a group of wild-type samples, or healthy subjects. A reference value can be used in place of a control or reference sample, which was previously obtained from a tumor without a target mutation (e.g., IDH-wild type, EGFR-wildtype), a healthy subject, a group of wild-type samples, or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Up-Regulated PTPN11 Phosphorylation at Multiple Sites in EGFR, ALK, and PDGFRA Activated Lung and Brain Tumors

The following example provides for novel drug targets and methods of treating glioblastoma multiforme (GBM) and lung cancer, including refractory cancers thereof and breast and pancreatic cancer.

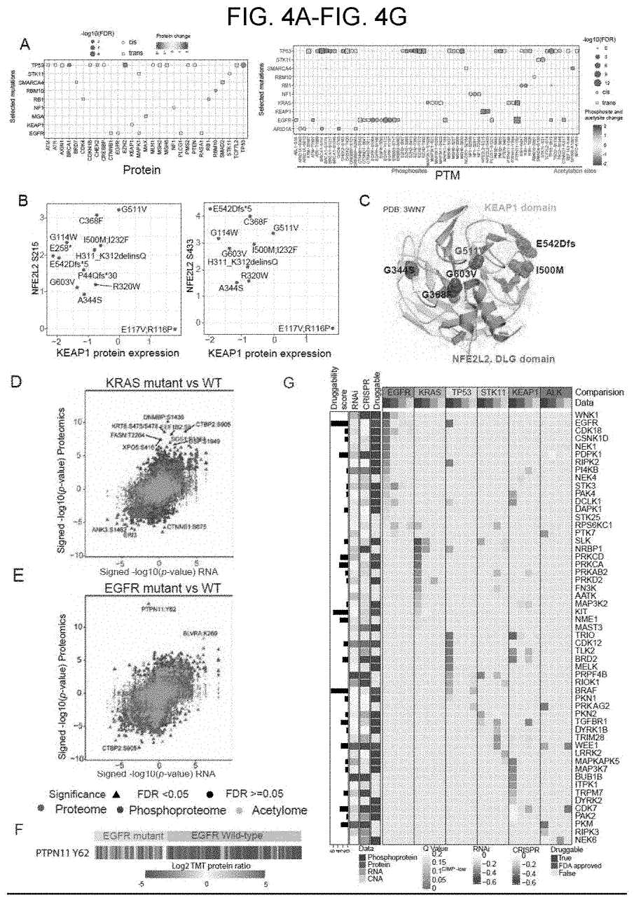

Oncogenic EGFR mutations are associated with upregulation of EGFR RNA and protein expression, and phosphorylation. We also observed strong trans effects in EGFR-mutated samples, e.g., the upregulation of beta-catenin (CTNNB1) in protein and phosphosite level, but not RNA level (FIG. 4A). In phosphosite level, we also found a high PTPN11 Y62 phosphorylation. In addition, EGFR-mutated samples tend to have high acetylation levels in CBFB, KDM6A, and KMT2C.

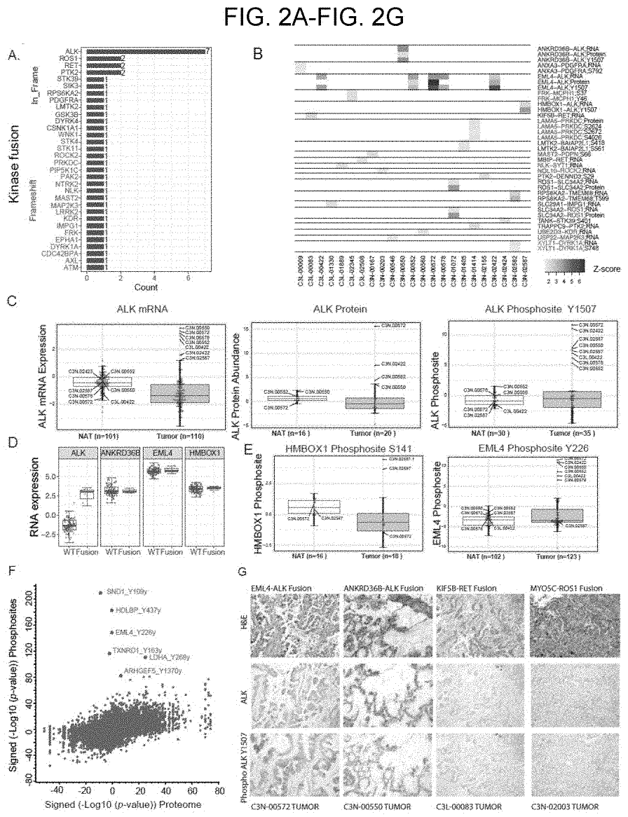

We further studied the trans effect of ALK fusion and found an increased protein expression of PTPRB, and PTPN11 Y546 and Y587 phosphorylation in samples with ALK fusion (FIG. 2D). A previous cell line study showed that ALK fusion can form a complex with PTPN11 and regulates PTPN11.

These results have led to the identification of novel drug targets that may be useful for treating lung and brain cancers as well as breast and pancreatic cancers. Experiments involving a number of mono- and combination therapies are currently being examined in our patient derived xenograft (PDX) models. Provided herein are the combination of new targets (e.g., PTPN11) and treatment modalities.

### Example 2: Proteogenomic Characterization Reveals Therapeutic Vulnerabilities in Lung Adenocarcinoma

To explore the biology of lung adenocarcinoma (LUAD) and identify new therapeutic opportunities, we performed comprehensive proteogenomic characterization of 110 tumors and 101 matched normal adjacent tissues (NATs) incorporating genomics, epigenomics, deep-scale proteomics, phosphoproteomics and acetylomics. Multi-omics clustering revealed four subgroups defined by key driver mutations, place of origin and gender. Proteomic and phosphoproteomic data illuminated biology downstream of copy number aberrations, somatic mutations and fusions, and identified therapeutic vulnerabilities associated with driver events including KRAS, EGFR and ALK fusions. Immune subtyping revealed a complex landscape, reinforced the association of STK11 with immune-cold behavior and underscored a potential immunosuppressive role of neutrophil degranulation. Smoking-associated LUAD showed evidence of other environmental exposures and a field effect in NATs. Matched NATs allowed for the identification of differentially expressed proteins with potential diagnostic and therapeutic utility. This proteogenomics dataset represents a unique public resource for researchers and clinicians seeking to better understand and treat lung adenocarcinoma.

Introduction

Lung cancer is the leading cause of cancer deaths in the United States (Siegel et al., 2019) and worldwide (Bray et al., 2018). Despite therapeutic advances including tyrosine kinase inhibitors and immunotherapy, sustained responses are rare and prognosis remains poor (Herbst et al., 2018), with a 19% overall 5-year survival rate in the United States (Bray et al., 2018) and a worldwide ratio of lung cancer mortality to incidence of 0.87. Adenocarcinoma, the most common lung malignancy, is strongly related to tobacco smoking, but also the subtype most frequently found in individuals who have reported no history of smoking (“never-smokers”) (Subramanian and Govindan, 2007; Sun et al., 2007). The genetics and natural history of lung adenocarcinoma (LUAD) have been shown to be strongly influenced by smoking status, gender, and ethnicity, among other variables (Chapman et al., 2016; Okazaki et al., 2016; Subramanian and Govindan, 2007; Sun et al., 2007). However, contemporary large-scale sequencing efforts have typically been based on cohorts of smokers with limited ethnic diversity. Among the major sequencing studies that have helped to elucidate the genomic landscape of LUAD (Clinical Lung Cancer Genome Project (CLCGP) and Network Genomic Medicine (NGM), 2013; Ding et al., 2008; Imielinski et al., 2012), only The Cancer Genome Atlas (TCGA) measured a small subset of proteins and phosphopeptides, restricted to a 160-protein reversed phase array (Cancer Genome Atlas Research Network, 2014). As the most frequent genomic aberrations in LUAD involve RAS/RAF/RTK pathway genes that lead to cellular transformation mainly by inducing proteomic and phosphoproteomic alterations (Cully and Downward, 2008), global proteogenomic profiling is clearly needed to provide deeper mechanistic insights. Furthermore, while prior molecular characterization has identified a number of oncologic dependencies and facilitated the development of effective inhibitors for LUAD driven by mutant EGFR (Lynch et al., 2004; Paez et al., 2004) and ALK (Kwak et al., 2010), ROS1 (Shaw et al., 2014) and RET fusions (Gautschi et al., 2017; Kohno et al., 2012; Takeuchi et al., 2012), a substantial proportion of LUADs still lack known or currently targetable mutations. To further our understanding of lung adenocarcinoma pathobiology and potential therapeutic vulnerabilities, the National Cancer Institute (NCI)'s Clinical Proteomics Tumor Analysis Consortium (CPTAC) undertook comprehensive genomic, deep-scale proteomic and post-translational modifications (PTM) analyses of paired (patient-matched) LUAD tumor and normal adjacent tissue pairs. Our integrative proteogenomic analyses focused particularly on novel and clinically actionable insights revealed in the proteome and PTMs. These analyses provide a multifaceted view of lung cancer by connecting somatic alterations to downstream signaling, adding significant new biology, further refining LUAD molecular taxonomy, and nominating specific targeted and immunologic therapies. The underlying data represent an exceptional resource for further biological, diagnostic and drug discovery efforts.

Results

Proteogenomic Landscape and Molecular Subtypes of Lung Adenocarcinoma (LUAD)

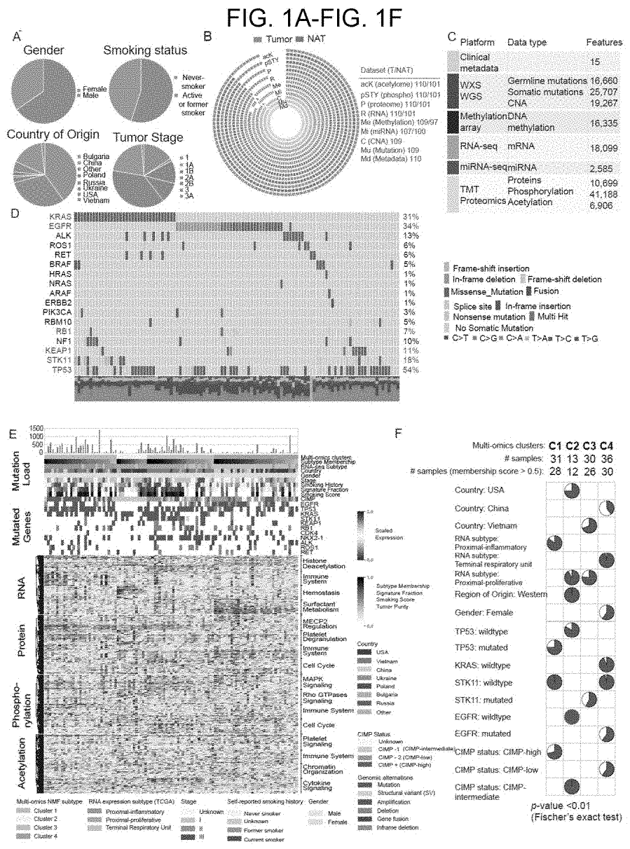

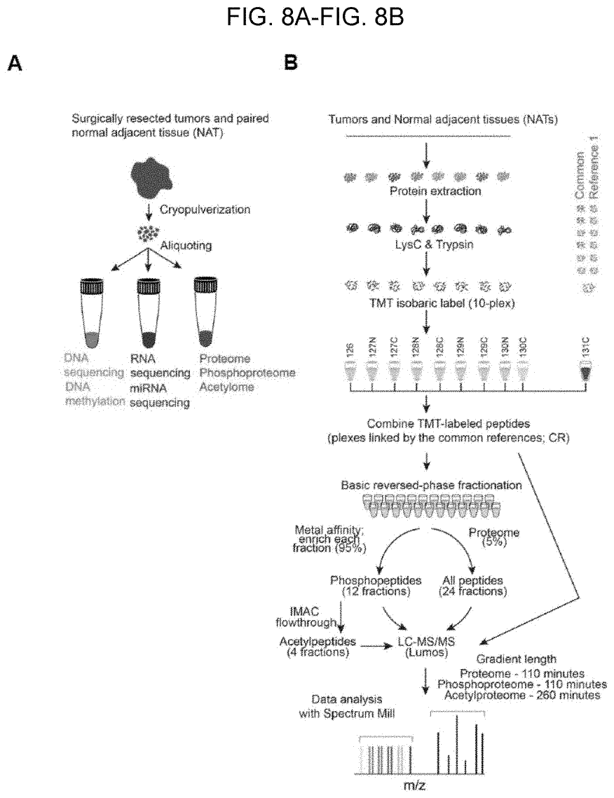

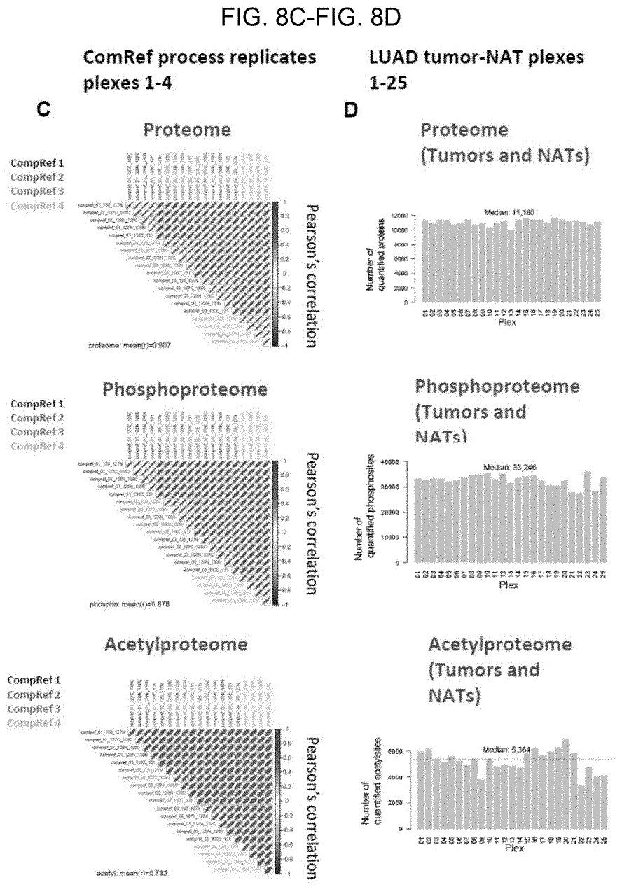

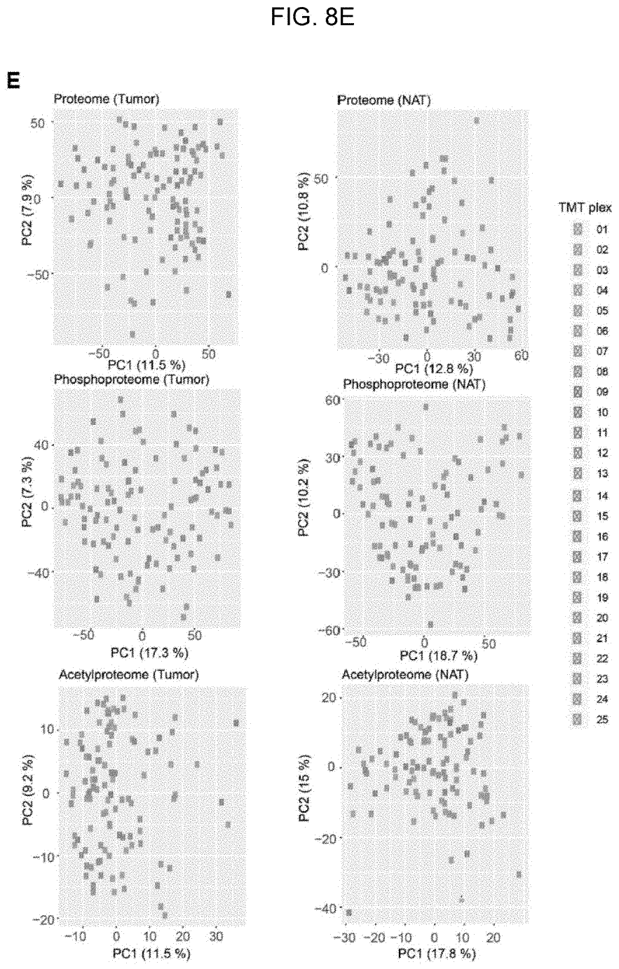

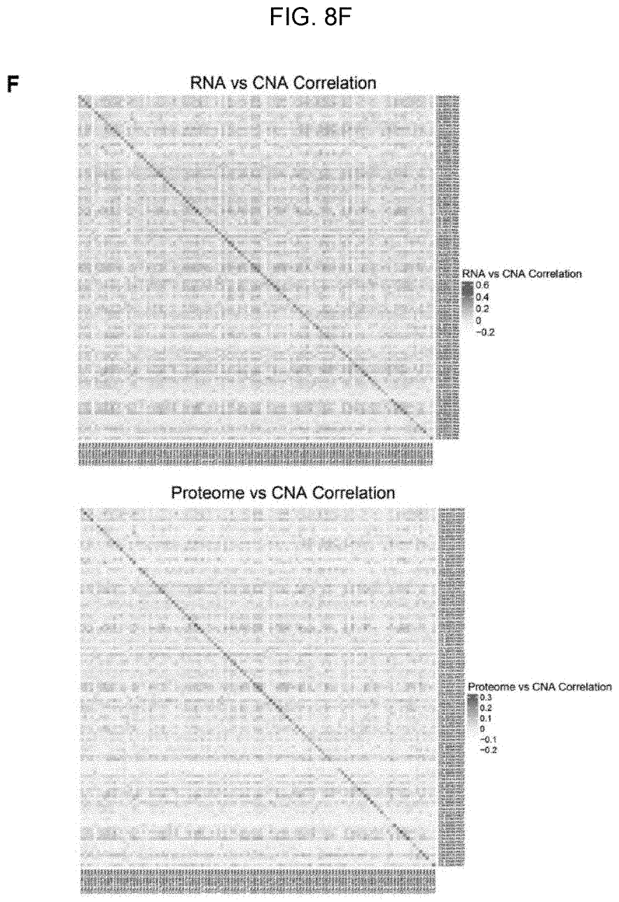

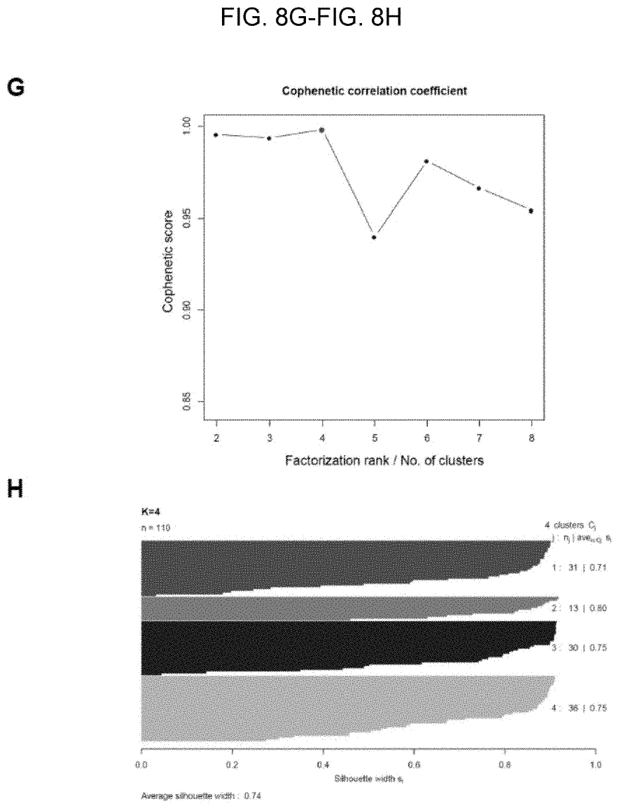

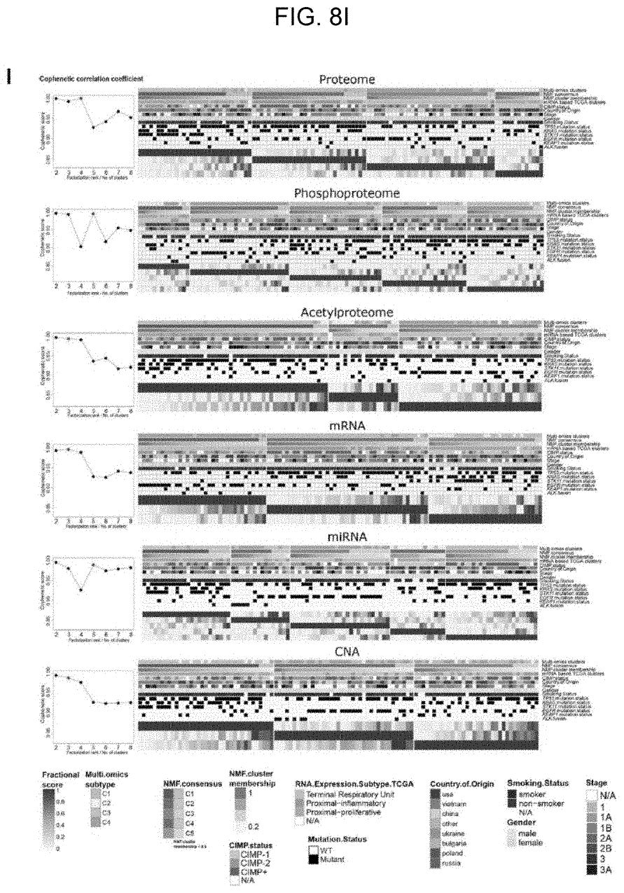

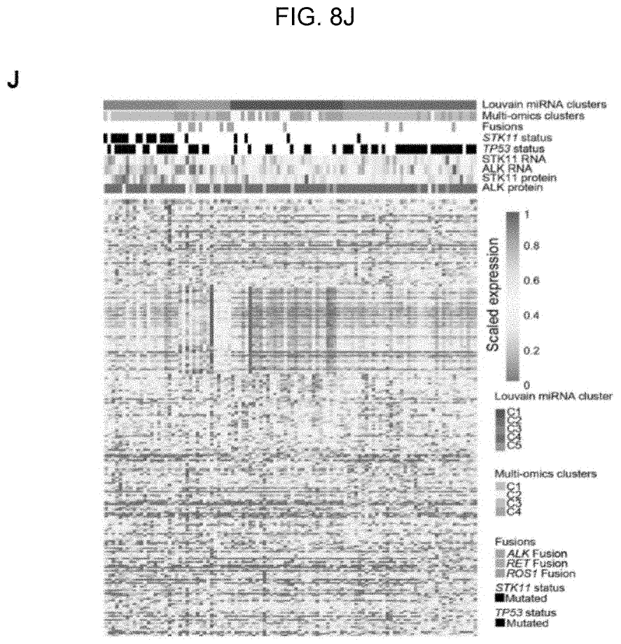

We investigated the proteogenomic landscape of 110 prospectively collected, treatment-naive lung adenocarcinoma (LUAD) tumors and 101 samples of paired normal adjacent tissue (NAT). Strict protocols limited ischemic exposure and controlled pre-analytical variability and sample quality. Unique to this study, the samples represented diverse countries and regions of origin, smoking status and tumor stages, with >40% never-smokers and approximately even divisions between Asian (Chinese and Vietnamese) and non-Asian samples and between early- (stages 1A and 1B) and later-stage (2 and above) disease (FIG. 1A). Each of the tumors and NATs was cryopulverized and aliquoted before undergoing comprehensive proteogenomic characterization, including whole exome (WXS, nominal 150× coverage), whole genome (WGS, nominal 15× coverage), RNA (RNA-seq) and miRNA sequencing (miRNA-seq), array-based DNA methylation analysis, and in-depth proteomic, phosphoproteomic and acetylproteomic characterization, yielding deep-scale proteogenomics datasets (FIG. 1B, FIG. 8A). Tandem mass tags (TMT)-based isobaric labeling was used for precise relative quantification of proteins, serine (S), threonine (T) and tyrosine (Y) phosphorylation, and lysine (K) acetylation (FIG. 8B). Complete suites of proteogenomic data were obtained for 101 tumors and 96 NATs (FIG. 1B). Interspersed full proteomics comparator reference (CompRef) process replicates demonstrated excellent reproducibility (Pearson Correlation, Proteome: R=0.9, Phosphoproteome: R=0.88, Acetylome: R=0.73) and consistent identifications across several months of data acquisition time (FIG. 8C). Robust quality control and stringent criteria for identification and quantification maintained high proteomic data quality across all 25 TMT10-plexes with no apparent batch effect (FIG. 8D-FIG. 8E). Sample-wise correlation between CNA-RNA and CNA-Proteome confirmed the lack of any sample mislabeling (FIG. 8F). After appropriate filtering, we identified 19,267 somatic copy number aberrations (CNA), 6,917 synonymous and 25,707 nonsynonymous somatic mutations, 16,660 genes with germline mutations, 18,099 protein coding transcripts, 10,699 proteins, 41,188 phosphorylation sites and 6,906 acetylated lysine sites (FIG. 1C).

The general landscape of somatic alterations in this study was consistent with prior large-scale profiling efforts including TCGA (Cancer Genome Atlas Research Network, 2014), although with a different distribution likely due to the greater demographic diversity and larger proportion of self-reported never-smokers in the current study (FIG. 1D). Prevalent oncogenic and tumor suppressor mutations included TP53 (54%), EGFR (34%), KRAS (31%), STK11 (18%), KEAP1 (11%), RB1 (7%) and RBM10 (5%). We observed mutations and fusion events for ALK (13%), RET (6%) and ROS (6%), as well as mutations in RAS pathway genes including BRAF, HRAS, NRAS and ARAF (FIG. 1D). In addition, 27 focal amplifications (such as EGFR and NKX2-1) and 24 focal deletions (such as CDKN2A and SMARCA4) with Q value <0.20 were identified (Campbell et al., 2016; Cancer Genome Atlas Research Network, 2014; Weir et al., 2007). Despite the differences in subject demography, the platform used for CNA determination (array-based in TCGA versus WGS and WXS in CPTAC) and sample size, a high degree of overlap was observed between significant focal amplifications (5q35.1, 20p11.21, 4q12, 6p21.1, 17q23.1, 11q13.3, 7p11.2, 19q12, 20q13.33, 12q15, 8q24.21, 5p15.33, 14q13.3) and focal deletions (9p21.3, 9p23, 22q13.32, 21811.2, 19q13.33, 16q24.3) in the CPTAC and TCGA LUAD datasets.

To investigate the intrinsic structure of the proteogenomics data, unsupervised clustering was performed on CNA, RNA, miRNA, protein, phosphosites and acetylsites as individual data types, and on RNA, protein, phosphosites and acetylsites collectively as “multi-omics clustering” (FIG. 1E, FIG. 8G-FIG. 8J). Non-negative matrix factorization (NMF)-based multi-omics clustering revealed 4 stable clusters (C1-4) (FIG. 1E). These overlapped with prior mRNA-based proximal-inflammatory, proximal-proliferative and terminal respiratory unit clusters (Cancer Genome Atlas Research Network, 2014; Wilkerson et al., 2012), but subdivided the second of these into two distinct clusters. The core samples (defined by cluster membership score >0.5) of the 4 multi-omic LUAD subtypes were significantly associated with distinctive clinical and molecular features (Fisher's exact test, p-value <0.01; FIG. 1F). Cluster 1 (C1), aligned with proximal-inflammatory, was enriched for TP53 mutants, STK11 wild type, and CIMP high status; Cluster 2 (C2), a proximal-proliferative subcluster, was distinguished by Westerners (especially from USA), TP53 and EGFR wild type status, and intermediate CIMP status; Cluster 3 (C3), the dominant proximal-proliferative cluster, was enriched for Vietnamese patients and STK11 mutation (including two structural events identified from WGS); and Cluster 4 (C4), aligned with terminal respiratory unit, was enriched for EGFR mutants, female gender and Chinese nationality and essentially devoid of KRAS or STK11 mutations. Most (4 out of 7) of the samples harboring EML4-ALK fusions were assigned to Cluster 4 and lacked mutations in other key driver genes, consistent with a primary role for EML4-ALK in LUAD tumorigenesis (Gao et al., 2018).

To further explore the biology associated with the multi-omics taxonomy, we performed over-representation pathway analysis using differentially regulated genes, proteins, and post-translational modifications (PTMs) in each of the clusters (FIG. 1E) (Zhang et al., 2016). C1/proximal-inflammatory samples were primarily associated with immune signaling across multiple data types. The C2 subset of the proximal-proliferative subtype demonstrated signaling by Rho GTPases, as well as signatures of hemostasis and platelet activation, signaling and degranulation suggestive of systematic disturbances in coagulation homeostasis. The dominant proximal-proliferative subtype in C3 had a distinctive histone deacetylase signature but also upregulation of cell cycle pathways. Finally, the terminal respiratory unit subtype in C4 was distinguished by surfactant metabolism, MAPK1/MAPK3 signaling, MECP2 regulation, and chromatin organization in the acetylproteome. Notably, C1, characterized by increased expression of immune system-related genes, included samples with high non-synonymous mutation burden and high CpG island methylator phenotype (CIMP) status. The pathway enrichment analysis highlights intrinsic differences in both oncogenic signaling and host response across LUAD subtypes.

In an attempt to more directly uncover associations with key demographic variables, we developed a linear model to identify gender-specific markers in the global proteome. After adjustment for known confounding factors including smoking status, region of origin and mutation status of commonly mutated genes (EGFR, KRAS, STK11, TP53 and ALK fusions), the resulting model was able to identify only 22 differentially expressed proteins (FDR <0.05), with no coherent functional annotations. While female smokers have a higher risk for lung cancer (Rivera and Stover, 2004), our data does not suggest any gender-specific mechanisms after accounting for smoking and mutation status.

To explore the pattern of miRNA expression in LUAD, we performed unsupervised Louvain clustering on mature miRNA expression of 107 tumor samples for which miRNA data was available. Five subgroups of LUAD patients were identified by their distinctive miRNA expression profiles (FIG. 8K). Two of the subtypes were markedly enriched for tumors from multi-omics clusters 1 and 4, in turn aligned with proximal-inflammatory and proximal-proliferative RNA signatures, while the remaining three subtypes had mixed composition. One miRNA cluster was markedly enriched for ALK fusion-driven tumors, including all 5 EML4-ALK as well as the HMBOX1-ALK fusions, and featured high expression of miR-494, miR-495, and miR-496. miR-494 is known to play a key role in non-small-cell lung cancer (NSCLC) by downregulating BIM in TRAIL resistance (Romano et al., 2012), while miR-495 has been described as both a tumor suppressor and an oncogene, and is implicated in many cancers including NSCLC (reviewed in (Chen et al., 2017)). The vast majority of patients with STK11 mutation were categorized into another subgroup in which miR-106b-5p, miR-20a-5p, and miR-17-5p were highly expressed. miR-106b-5p has been ascribed protean effects in hepatocellular, renal cell, and other cancers, including promoting stem cell-like properties (Lu et al., 2017; Shi et al., 2018). miR-17-5p has been proposed to modulate cancer cell proliferation (Ao et al., 2019; Chen et al., 2016a; Cloonan et al., 2008), and is associated with poorer overall survival in many cancers, especially in Asian populations (Kong et al., 2018).

Genomic and Epigenetic Impact on Expression Landscape

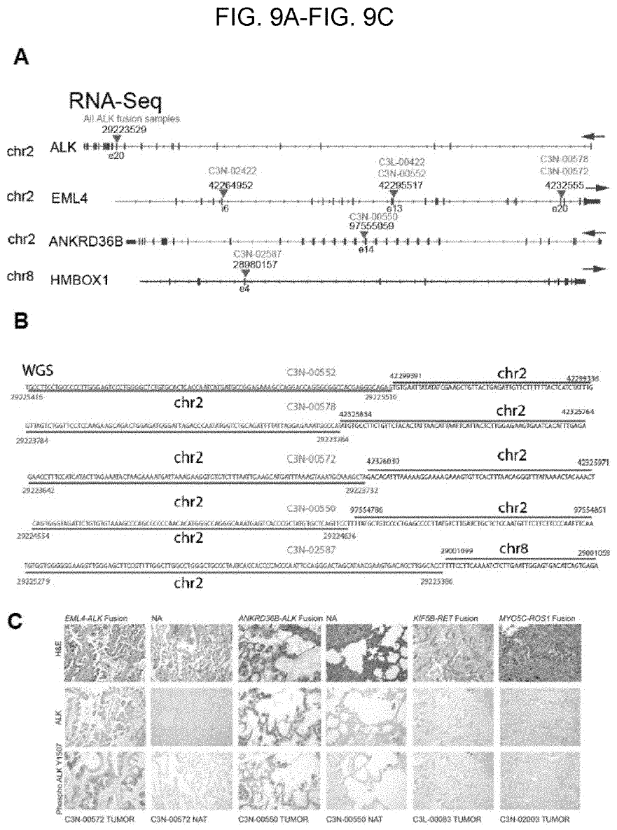

A comprehensive suite of genomics and proteomics data types allowed us to explore in detail the relationships between epigenetic and genomic events and downstream expression of RNA, proteins, and PTMs. Given their importance in LUAD biology, fusions were an area of particular focus. Multiple analysis tools (Star Methods) were used on tumor and NAT RNA-seq data to identify all gene fusions in the cohort. Cross-referencing with a curated kinase fusion database (Gao et al., 2018) allowed identification of all rearrangements involving kinases (FIG. 2A). While ALK, ROS1, RET, and PTK2 gene fusions were the most recurrent events in the cohort, several novel, potentially oncogenic kinase fusions were also discovered. Generally, such oncogenic kinases contained in-frame fusions, while kinases with a tumor suppressive role (such as STK11, STK4, ATM, AXL, FRK, EPHA1) exhibited disruptive out-of-frame events (FIG. 2A). The potential functional significance of several kinase fusions was supported by commensurate differential RNA, protein, and phosphosite expression of the index cases (FIG. 2B). Besides the 7 druggable kinase fusion outliers in ALK, we found instances in ROS1, RET, PRKDC, and PDGFRA in tumor but not paired NAT samples. Investigation of the fusion architecture of the highly recurrent in-frame ALK gene fusions (n=7) identified multiple 5′ partners including the well-established EML4 and novel HMBOX1 and ANKRD36B genes (FIG. 9A). WGS provided definitive structural support by precisely locating the genomic breakpoints in the intron proximal to exon-20 (e20) underlying these gene ALK rearrangements for 5 cases (FIG. 9B). All ALK gene fusion cases showed outlier expression of ALK mRNA and 4/7 showed outlier ALK total protein abundance; however, the most dramatic difference was seen in the specific and significant increase in ALK phosphosite Y1507 for all 7 ALK fusion cases, a likely autophosphorylation event consistent with constitutive activation of the ALK kinase domain in the ALK fusion product (FIG. 2C).

While RNA expression levels of the 5′ partner genes were uniformly high and did not differ between fusion-positive and -negative samples (FIG. 2D), both EML4-Y226 and HMBOX-S141 showed phosphosite enrichment only in the corresponding gene fusion-positive tumor samples (FIG. 2E). To assess phosphorylation of canonical and novel targets by mislocalized ALK fusion proteins (Ducray et al., 2019), we identified all protein phosphorylation events associated with ALK fusion (FIG. 2F). This analysis identified tyrosine phosphorylation of multiple proteins such as SND1, HDLBP, and ARHGEF5 (FIG. 2F). SND1 has previously been described as an oncogene (Jariwala et al., 2017), impacts biological processes such as angiogenesis and invasion, and regulates expression of oncogenic miRNAs (Chidambaranathan-Reghupaty et al., 2018), suggesting a novel role in ALK fusion-mediated tumorigenesis. Finally, to validate our observation of the fusion-specific ALK phosphosite Y1507 and to begin to explore its potential clinical translation, we performed immunohistochemistry (IHC) with commercially available ALK and phospho (Y1507) ALK antibodies. We noted tumor-specific positive staining in all ALK fusion-positive cases (n=5; two tumor samples were not available for analysis), whereas no detectable staining was observed in either paired NATs or samples with ROS1/RET fusions (FIG. 2G, FIG. 9C). The fusion transcript architecture and the specific phosphosite modifications observed by both mass spectrometry and IHC provide incontrovertible evidence of ALK fusion ontogeny and functionality for both EML4 and the novel fusion partners we describe, and greatly expand our knowledge of oncogenic ALK fusion protein signaling.

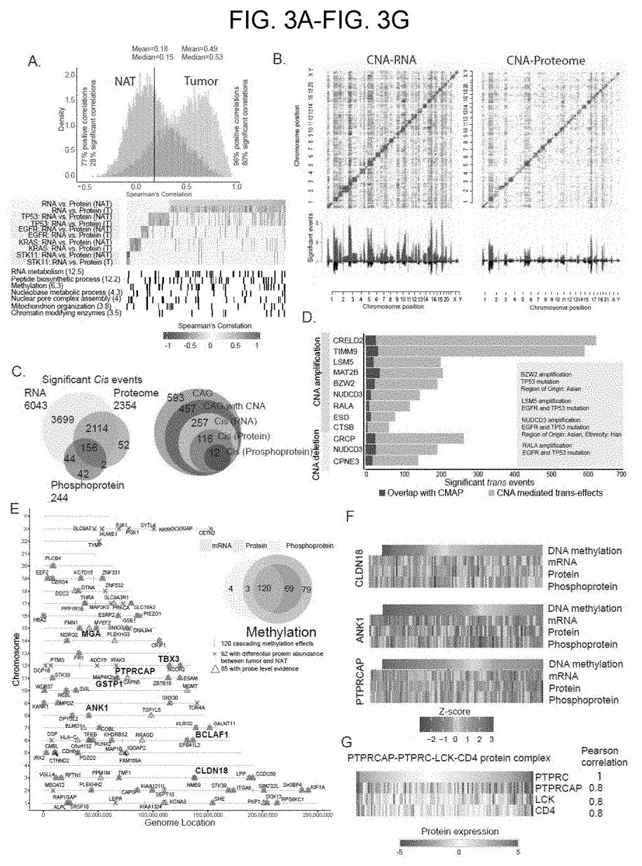

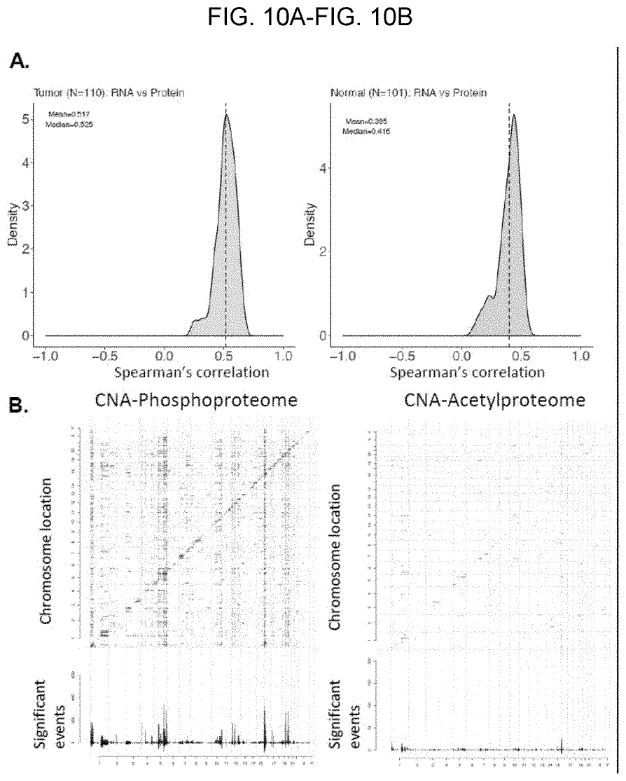

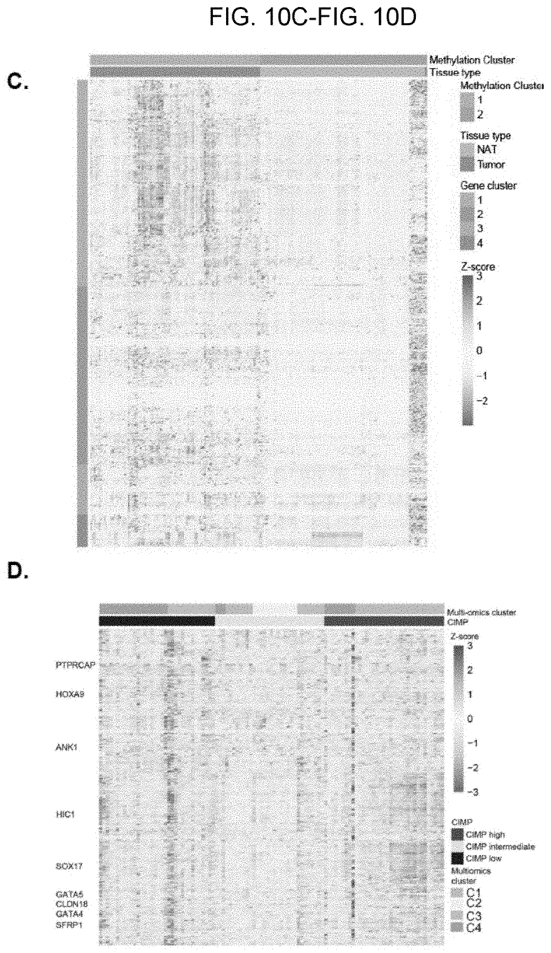

To investigate the relationship between mRNA and protein expression, we performed both sample-wise and gene-wise mRNA-protein correlation. While sample-wise correlations were fairly consistent between tumors and NATs (FIG. 10A), gene-wise mRNA-protein correlations displayed striking differences (FIG. 3A), with tumors showing a median gene-wise mRNA-protein correlation of 0.49 compared to only 0.18 in NATs (FIG. 3A). We identified a total of 227 transcript/protein pairs differentially correlated (FDR <0.01) between tumors and NATs globally or within 4 major mutational subtypes (TP53-mutant (n=52), EGFR-mutant (n=36), KRAS-mutant (n=29), STK11 mutant (n=17) (FIG. 3A middle panel). The identified gene products were markedly enriched for RNA metabolism, peptide biosynthesis, methylation, mRNA splicing, nuclear processing, mitochondrial organization, and chromatin modifiers (p-value <10⁻³, FIG. 3B bottom panel), suggesting tighter or more active translational control of proteins involved in key housekeeping processes in tumors relative to NATs.

The impact of copy number alterations on RNA and protein abundance in both cis and trans has been interrogated and is depicted in FIG. 3B. Loci associated with significant trans-effects (FDR <0.05) are represented below the heatmap. As we have previously observed (Mertins et al., 2016), CNA correlations are broadly comparable but considerably dampened at the protein relative to the RNA level, suggesting that protein-level correlations may facilitate nomination of functionally important genes within copy number-altered regions. This effect is further evidenced in the diminishing number of copy number cis-events that are reflected in RNA and translated to the level of proteins and post-translational modifications (FIG. 3C, FIG. 10A). A total of 6,013, 2,324, 244 and 29 significant positive correlations (cis-effects) were observed for RNA, protein, phosphoproteins and acetylated proteins, respectively, with only 156 significant cis-effects overlapping between RNA, proteome and phosphoproteome (FIG. 3C). A similar trend was observed within 593 cancer-associated genes (CAG) (FIG. 3C). The 12 cancer-associated genes showing significant regulation at RNA, proteome and phosphoprotein levels were CREBBP, KMT2B, PSIP1, AKT2, EGFR, GMPS, IL6ST, IRF6, NFKB2, PHF6, YES1, ZBTB7B. In addition, numerous genes associated with recurrent LUAD-specific CNA events (Campbell et al., 2016) showed dowstream expression effects, including significant cis-regulation at RNA and protein levels for CDK4, RB1, SMAD4, ARID2, MET, ZMYND11 and ZNF217.

To help nominate functionally important genes within copy number-altered regions, we compared protein-level trans-effects to approximately half a million genomic perturbation signatures contained in the Connectivity Map database (Connectivity map) (STAR Methods). Trans-effects significantly paralleled the associated gene perturbation profiles for 12 CNA events (CRELD2, TIMM9, LSM5, MAT2B, BZW2, NUDCD3, RALA, ESD, CTSB, CRCP, CPNE3) (BH, FDR <0.1) (FIG. 3D), of which 4 (BZW2, LSM5, NUDCD3, RALA) showed significant enrichment for specific mutational or demographic features (Fisher's exact test, FDR <0.1); FIG. 3D, inset). Ras-related protein Ral-A (RALA) is a GTPase that has been shown to mediate oncogenic signaling and regulate EGFR and KRAS mutation-mediated tumorigenesis (Gildea et al., 2002; Kashatus, 2013; Peschard et al., 2012). Our data suggests that amplification of RALA may affect the biology of EGFR mutant tumors. The role of basic leucine zipper and W2 domain 2 (BZW2) in LUAD has not been elaborated, but BZW2 stimulates AKT/mTOR/PI3K signaling and cell growth in bladder and hepatocellular carcinoma (Gao et al., 2019; Jin et al., 2019), and has also been shown to interact with EGFR (Foerster et al., 2013). The lysosomal cysteine proteinase cathepsin B (CTSB) has long been described as a marker of poor prognosis in LUAD (Fujise et al., 2000; Inoue et al., 1994) with mechanistic association with metastasis (Erdel et al., 1990; Higashiyama et al., 1993) possibly mediated by the E3 ubiquitin ligase NEDD4 (Shao et al., 2018). In summary, protein-level trans-effects provide mechanistic hypotheses for the tumorigenic impact of copy number alterations, though experimental validation is required.

DNA methylation analyses showed LUAD tumors to be much more highly methylated than their counterpart NATs (p-value <0.0001, Two-sided Wilcoxon rank-sum test) (FIG. 10C). Unsupervised clustering of the tumor methylome revealed CIMP-high, -intermediate, and -low clusters (respectively referred to as CIMP+, -1, and -2) (FIG. 10D), as previously described (Cancer Genome Atlas Research Network, 2014), with CIMP-low clusters nevertheless having focal areas of increased methylation (FIG. 10D). Unlike prior studies, however, deep proteogenomics data allowed examination of methylation effects that cascade through RNA to protein and phosphoprotein levels, increasing their likelihood of functional significance. FIG. 3E shows the landscape of 120 methylation-driven cis-effects associated with coordinate differential expression at the RNA, protein and phosphoprotein levels analyzed using iProFun (Song et al., 2019). The majority of these (85/120, represented by triangles in FIG. 3E) are directly supported by probe-level data in the promoter region of the gene. While many of these are novel, others, including CLDN18, ANK1 and PTPRCAP (FIG. 3F) have strong associations with lung adenocarcinoma biology. CLDN18 is highly expressed in lung alveolar epithelium; its knockdown leads to increased lung parenchyma, expanded lung epithelial progenitor populations, and increased propensity for lung adenocarcinoma development (Zhou et al., 2018). ANK1 promoter CpG islands are unmethylated in normal lung but methylated in more than half of lung adenocarcinomas, irrespective of stage but associated with positive smoking history. ANK1 knockdown affects pathways associated with cell proliferation, movement, and survival, though direct phenotypic effects have not been demonstrated (Tessema et al., 2017). Importantly, however, miR-486-3p and miR-486-5p, both strongly associated with lung adenocarcinoma oncogenesis, are located within ANK1 introns and are co-expressed with their host gene. PTPRCAP (CD45 associated protein), together with the three other members of its supramolecular complex, phosphatase CD45, co-receptor CD4, and kinase LCK, is implicated in regulation of lymphocyte function (Kruglova et al., 2017; Matsuda et al., 1998). While methylation probe positions do not allow us to determine whether the complex partners of PTPRCAP are regulated by methylation, they show coordinate expression at the protein level (FIG. 3G). Notably, PTPRCAP was included in a 5-gene methylation-based immune signature associated with survival in multiple malignancies including lung cancer (Jeschke et al., 2017). Other cancer-related genes with “cascading” methylation effects include BCLAF1, GSTP1, MGA, and TBX3. Loss-of-function MGA mutations were identified as somatic events activating the MYC pathway in lung adenocarcinoma (Cancer Genome Atlas Research Network, 2014), suggesting the MGA promoter methylation identified in our analysis may represent an important event in tumorigenesis. GSTP1 methylation is a potential biomarker for many cancers (Chen et al., 2013; Gurioli et al., 2018), while TBX3 hypermethylation was shown to be associated with prognosis in bladder cancer (Beukers et al., 2015; Kandimalla et al., 2012).

Connecting Driver Mutations to Proteome, Phosphoproteome and Pathways

To identify both broader biological and clinically actionable impacts of selected mutated genes, we examined mutational effects on expression of products of the gene itself (cis-effects), and on expression of other cancer-related gene products (trans-effects) (Cancer Genome Atlas Research Network, 2014; Ding et al., 2008). We identified 11 genes with either cis- or trans-effects in at least one of RNA, protein and phosphoprotein expression (FDR <0.05, Wilcoxon rank-sum test). As shown in FIG. 4A, TP53 and EGFR mutations resulted in their elevated protein and phosphosite expression, whereas STK11, RBM10, RB1, NF1 and KEAP1 mutants showed reduced protein and phosphosite expression.

TP53 showed significant cis-correlation at the level of protein and phosphosites (S315) but not at the level of RNA (FIG. 11A), suggesting post-translational regulation (Mertins et al., 2016). TP53 mutants showed upregulation of protein expression in mismatch repair (MMR) genes such as MLH1, MSH2, MSH6, and PSM2 and genes involved in DNA damage response (DDR) including ATM, ATR, and BRCA1. TP53 mutants also showed significantly elevated EZH2 protein relative to RNA expression (FIG. 4A, FIG. 11A), consistent with the recent observation of elevated EZH2 protein stability in p53 mutant cell lines (Kuser-Abali et al., 2018), and displayed downregulation of protein expression for genes involved in Wnt signaling (e.g. AXIN1 and TCF7L2) (Rother et al., 2004; Sanchez-Vega et al., 2018). Mutation in RB1, another key cell cycle-related gene, was associated with increased CDK4 protein abundance. RB1 mutation has been shown to induce resistance to CDK4/6 inhibitors and CDK4 protein upregulation may contribute to this in RB1-mutated LUAD samples. SMARCA4 mutants had increased protein expression of SMAD2, while STK11 mutation was associated with increased phosphorylation of SMAD4 (S138). SMAD2 and SMAD4 are key elements in the transcriptional regulation of EMT induced by TGF-6 signaling (Xu et al., 2009). EGFR mutant samples showed decreased CTNNB1 expression at the level of RNA but elevated expression both at the level of proteome and phosphoproteome (FIG. 4A, FIG. 11A). CTNNB1 has been shown to play a critical role in EGFR-driven lung adenocarcinoma (Nakayama et al., 2014), and the trans-regulated phosphosite S552 on CTNNB1 induces its transcriptional activity (Fang et al., 2007). In addition to CTNNB1 regulation, we observed increased phosphorylation and acetylation of CTNND1, which has been implicated in NF-KB and RAC1-mediated signaling but not previously described, to our knowledge, in EGFR-mediated LUAD (Mizoguchi et al., 2017; Perez-Moreno et al., 2006).

The cis- and trans-effects identified above (FIG. 4A) helped reveal the detailed regulatory network of the KEAP1/NFE2L2 (NRF2) complex. KEAP1 interacts with NFE2L2 through two distinct binding domains, DLG and ETGE (Canning et al., 2015; Fukutomi et al., 2014), and undergoes conformational change under oxidative stress allowing NFE2L2 to execute the antioxidant response vital to lung cancer progression and metastasis (Lignitto et al., 2019; Wiel et al., 2019). Twelve LUAD tumors harbored KEAP1 mutations, including missense mutations and truncations distributed across the entire length of the protein (FIG. 11B). KEAP1 mutations did not impact RNA expression of KEAP1 or NFE2L2 (FIG. 11C), but resulted in a downregulation of KEAP1 protein expression and phosphorylation of NFE2L2 on S215 and S433 (FDR <0.05, Wilcoxon rank-sum test) (FIG. 11C). While 10 of 12 missense and truncation mutations resulted in both downregulation of KEAP1 protein expression and upregulation of NFE2L2 phosphorylation (FIG. 4B), one BTB domain mutation (G511V) did not downregulate KEAP1 protein expression but had amongst the highest levels of NFE2LE phosphorylation (FIG. 4B), suggesting a novel mechanism of action. Superposition of the site on the KEAP1 crystal structure shows that the G511V mutation falls close to the KEAP1/NFE2L2 binding domain (FIG. 4C), leading to the hypothesis that this mutation functions to disrupt KEAP1-NFE2LE interaction rather than to impact protein stability. The vast majority of proteins and phosphosites upregulated in samples with KEAP1 mutations (FIG. 11D and FIG. 11E) were members of the NFE2L2 oncogenic signatures, mostly associated with activation of antioxidant responses that provide cytoprotection to cancer cells (FIG. 11F) (Taguchi and Yamamoto, 2017). These upregulated proteins provide potential therapeutic targets for treating KEAP1 mutant tumors.

Identification of Therapeutic Strategies from Proteogenomics Analyses

Comparison of global differential regulation of RNA, proteins, phosphosites and acetylsites revealed extreme phosphosite outliers in both KRAS (FIG. 4D) and EGFR (FIG. 4E) mutant tumors. KRAS mutants showed significant upregulation of numerous cancer-associated phosphosites, including SOS1 phosphorylation on S1161. SOS1 is a guanine exchange factor (GEF) that activates KRAS (Vigil et al., 2010), and inhibition of SOS1 and KRAS is an emerging therapeutic strategy for KRAS-mutant cancers (Hillig et al., 2019; O'Bryan, 2019). The observed C-terminal phosphorylation of SOS1 (Kamioka et al., 2010) likely relieves its constitutive interaction with GRB2 (Giubellino et al., 2008) allowing its recruitment to the membrane for KRAS activation in a GRB2-independent manner (Aronheim et al., 1994; Rojas et al., 2011). Interestingly, we also observed C-terminal phosphorylation of another GEF containing protein, DNMBP (also called TUBA), the role of which is not yet established in LUAD or KRAS mutant cancers.

EGFR mutant tumors showed highly significant (FIG. 4E) and remarkably consistent (FIG. 4F) tyrosine phosphorylation of PTPN11 (Shp2) at Y62, but no effect was observed at the RNA or protein levels (FIG. 4E). While prior studies have associated Shp2 phosphorylation with important biological consequences in non-small cell lung cancer cell lines and xenograft models, this is, to our knowledge, the first report of such phosphorylation in a large set of primary treatment-naive lung adenocarcinomas. Shp2, a ubiquitously expressed non-receptor protein tyrosine phosphatase encoded by PTPN11, has two N-terminal Src homology 2 (Sh2) domains, N-SH2 and CSH2, a catalytic PTP domain, and a C-terminal tail with tyrosine phosphorylation sites. In its basal state the protein is inactive in a closed conformation, with the N-SH2 domain binding to the active site of the PTP domain. This oncogenic tyrosine phosphatase (Chan and Feng, 2007) undergoes conformational activation downstream of growth factor receptor and cytokine signaling, regulating cell survival and proliferation chiefly through RAS and ERK activation (Matozaki et al., 2009). Residue Y62 falls in the interface between the N-SH2 and PTP domains, where its phosphorylation is thought to stabilize the active protein conformation (Ren et al., 2010). While Shp2 oncogenic mutations (most of which fall in the interface occupied by Y62) (Vemulapalli et al., 2019) are common in some subtypes of leukemia (Chan and Feng, 2007; Ostman et al., 2006), they are uncommon in solid tumors, where other mechanisms of Shp2 activation predominate. Indeed, mutations were reported in only 1 of 118 primary lung adenocarcinomas and 2 of 65 NSCLC cell lines (Bentires-Alj et al., 2004). In a tissue microarray panel of non-small cell lung cancer cases, however, elevated Shp2-expression was associated with lymph node metastasis, increased VEGFR-2 expression and microvessel density, and decreased survival (Tang et al., 2013). Decreased overall and progression-free survival was also seen in EGFR-positive non-small lung cancer patients with high compared to low Shp2 mRNA expression (Karachaliou et al., 2019).

Notably, ALK fusion-driven tumors also showed outlier phosphorylation of PTPN11, albeit at the C-terminal tyrosine phosphorylation sites Y546 and Y584 (FIG. 11G). Various activating functions have been proposed for these phosphorylation sites, including directly driving the active conformation (Bennett et al., 1994; Lu et al., 2001) and serving as growth factor receptor-bound protein 2 (Grb2) docking sites (Bennett et al., 1994; Cunnick et al., 2002; Okazaki et al., 2013; Vogel and Ullrich, 1996). The Shp2 adaptor protein Grb2-associated binder-1 (GAB1) (Montagner et al., 2005) was also significantly upregulated in ALK fusion-driven tumors (FIG. 11H).

Irrespective of the mode of activation, multiple lines of evidence suggest that Shp2 inactivation will suppress tumorigenesis (Aceto et al., 2012; Prahallad et al., 2015; Ren et al., 2010; Schneeberger et al., 2015), making Shp2 among the highest priority PTP targets for anticancer drug development (Ostman et al., 2006). Shp2 activation has also been identified as a resistance mechanism in ALK-inhibitor resistant ALK-driven patient-derived cell lines, a process overcome by treatment with the Shp2 inhibitor SHP099 in combination with the ALK inhibitor ceritinib (Dardaei et al., 2018). Shp2 inhibitors have shown great promise in preclinical trials (Chen et al., 2016b) and are now in clinical trials from multiple companies. Our data suggest that EGFR mutant- and ALK fusion-driven LUADs would be particularly promising target populations for such therapy.

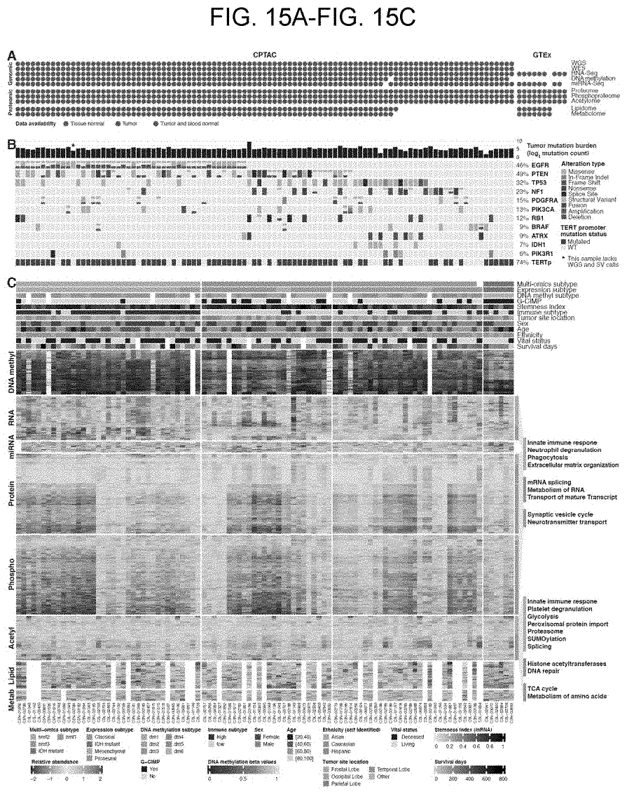

Deep proteogenomics data allow the nomination of specific therapeutic targets (as for SOS1 and Shp2 inhibitors, above) and more general therapeutic strategies. Protein-level pathway comparison of EGFR- and KRAS-driven LUAD tumors showed remarkable disparity in complement and clotting cascades, with upregulation of coagulation in KRAS and downregulation in EGFR mutant samples (FIG. 11I). Of note, signals of hypercoagulability were a particularly prominent feature in the KRAS-dominated NMF cluster C2 (FIG. 15E). The increased risk of venous thromboembolism in patients with primary lung cancer is well-established (Chew et al., 2008) and the associated morbidity and mortality, counterbalanced by bleeding risk associated with systemic anticoagulation, has led to many pharmacological trials of novel anticoagulants and the continuing evolution of recommendations regarding prophylaxis (Key et al., 2019). Our data suggest that such risk-benefit analyses in the lung cancer population should be stratified by mutation type. This concept is supported by a recent study of 605 Chinese patients with newly diagnosed NSCLC, in which the likelihood of confirmed VTE in the first year was 8.3% in those with and 13.2% in those without EGFR mutations (P<0.05; (Dou et al., 2018)).

To systematically nominate druggable targets specific to groups of samples with mutations in major mutant-driven LUADs (EGFR, KRAS, TP53, STK11, KEAP1, ALK fusions), we assessed hyperphosphorylation of kinases as a proxy for abnormal kinase activity (Mertins et al., 2016) (FIG. 4G) and annotated outliers for the degree to which shRNA- or CRISPR-mediated depletion reduced survival and proliferation in lung cancer cell lines (Barretina et al., 2012; Tsherniak et al., 2017). Totals of 11, 10, 13, 5, 14 and 9 significantly hyperphosphorylated kinases (FDR <0.20) were identified in EGFR, KRAS, TP53, STK11, KEAP1 and EML4-ALK mutant samples respectively, the majority of which did not show outlier expression at the level of CNV, RNA or protein expression. EGFR tyrosine kinase inhibitors are standard-of-care therapy in LUAD patients with EGFR mutations (Paez et al., 2004); as expected, we found that hyperphosphorylated EGFR was enriched in the EGFR-mutated subset. Several other kinases that have interactions with FDA-approved drugs were found to be hyperphosphorylated in various mutant samples, including PRKCD in KRAS mutants, BRAF in TP53 mutants, and WEE1 in EML4-ALK fusions. In addition, we identified 27 putatively druggable kinases with known but as yet non-FDA approved inhibitors (Cotto et al., 2018). Similar phosphorylation outlier analyses were performed for phosphatases, ubiquitinases, and deubiquitinases (FIG. 11J), though the role of phosphorylation in these protein classes is less well established. One limitation of outlier analysis is its inability to identify altered expression in a large subset of the samples, e.g., PTPN11 Y62 did not appear as an “outlier” phosphatase because of its relatively uniform high expression across the entire class of EGFR mutant tumors (N=38).

Immune Landscape of Lung Adenocarcinoma

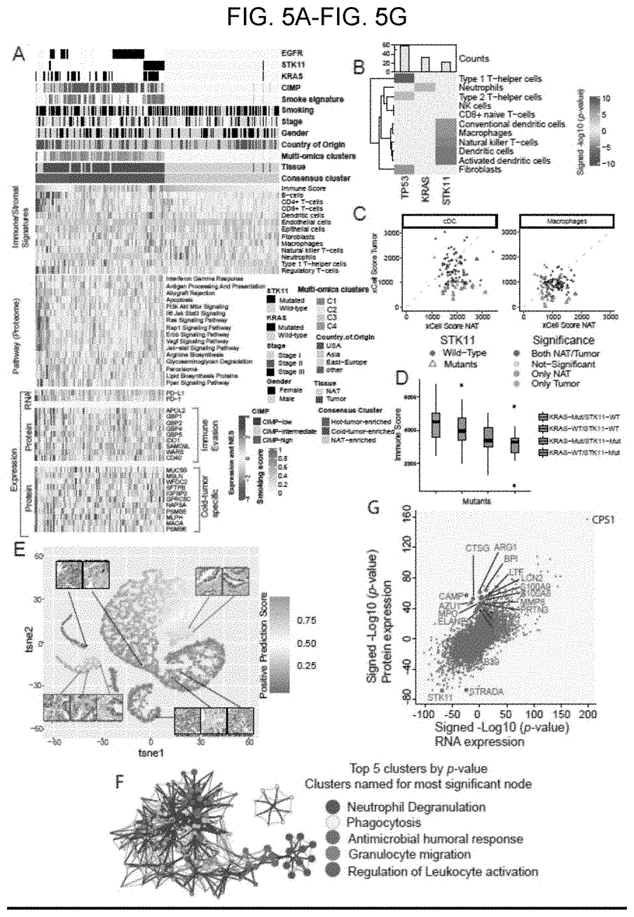

We analyzed the transcriptomic profiles of 110 tumors and 101 normal adjacent tissue samples, and deconvoluted immune and stromal cell gene signatures using xCell (Aran et al., 2017), resulting in the identification of 64 cell types. Consensus clustering identified three major immune clusters: “Hot”-tumor-enriched (HTE), “Cold”-tumor-enriched (CTE) and NAT-enriched clusters (FIG. 5A, upper panel. Associations were observed between immune and NMF clusters, with NMF cluster C1 enriched in HTE and NMF clusters C3 and C4 enriched in CTE immune clusters (p-value <0.0003, Fisher's exact test). HTE tumors were differentiated from CTE tumors by their stronger signatures for B-cells, CD4+ and CD8+ T-cells, dendritic cells and macrophages. The HTE proteome was characterized by upregulation of immune-related pathways such as Interferon Gamma Response, Allograft Rejection, Adaptive Immune System and Antigen Processing and Presentation, as well as by Ras and Rap1 Signaling, ErbB Signaling, EMT, VEGF Signaling, Jak-STAT Signaling, Oncogenic MAPK Signaling and Apoptosis (FIG. 5A, middle panel). PD-1 and PD-L1 were also upregulated in the immune HTE cluster based on both RNA and global protein abundance (FIG. 5A, lower panel). Notably, however, the HTE subtype also revealed the presence of immune inhibitory cells such as regulatory T-cells, and showed protein upregulation of key-markers of T-reg function such as CTLA4 and FOXP3. Cytokines including TGF-beta and IL-10 that are known to enhance the suppressive mechanisms of Tregs were upregulated in HTE tumors based on RNAseq data. As tumors with high levels of Treg infiltration are typically associated with poor prognosis (Shimizu et al., 2010), anti-CTLA4 therapy might be of benefit to this population (Wing et al., 2008).

CTE tumors were characterized by the upregulation of various metabolic pathways such as Arginine Biosynthesis, Butanoate Metabolism, Glycosaminoglycan Metabolism, and Biosynthesis of Unsaturated Fatty Acids. Peroxisome and PPAR Signaling Pathway activities in CTE tumors were only captured by proteomics data and not by RNAseq data (FIG. 5A, middle panel). Several studies have shown that IFNgamma promoter activity can be inhibited by PPAR-gamma activation (Marx et al., 2000), and in particular that suppression of the inflammatory immune response by PPAR-gamma activation may be achieved through induction of immune cell apoptosis. PPARgamma activation was shown to impair T-cell proliferation through an IL-2 dependent mechanism, while PPAR-beta activation was shown to favor oxidation of fatty acids and glucose in developing T-cells (Le Menn and Neels, 2018). As shown in FIG. 5A, CIMP-based clusters were strongly associated with immune clusters, with CIMP-low being enriched in hot tumors. CTE tumors were heterogeneous; a subset shared some dendritic, macrophage and T-cell signatures with the HTE tumors (FIG. 5A, top heatmap), an overlap reinforced in pathway and expression space (FIG. 5A, middle and bottom heatmaps). This also accounts for the immune system upregulation associated with NMF Cluster 1 (FIG. 1E).

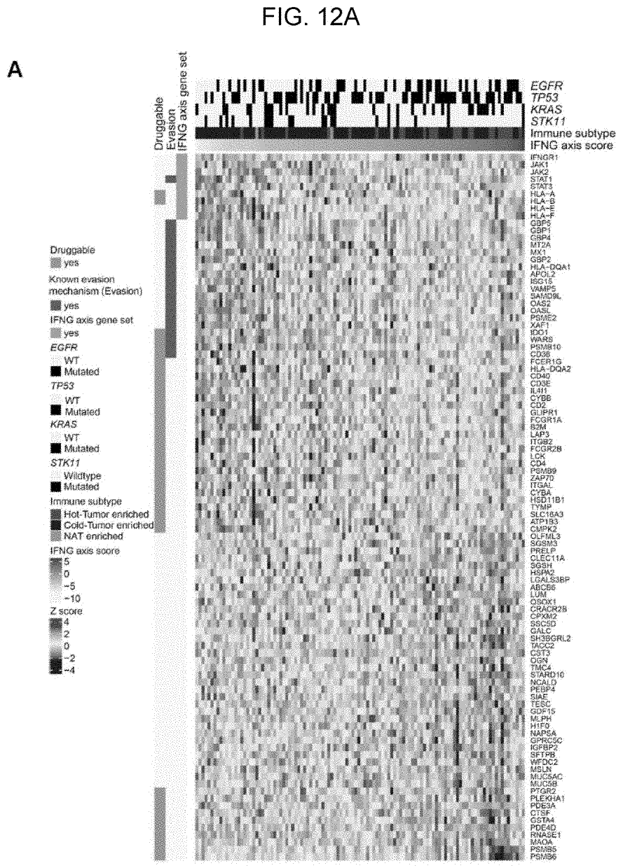

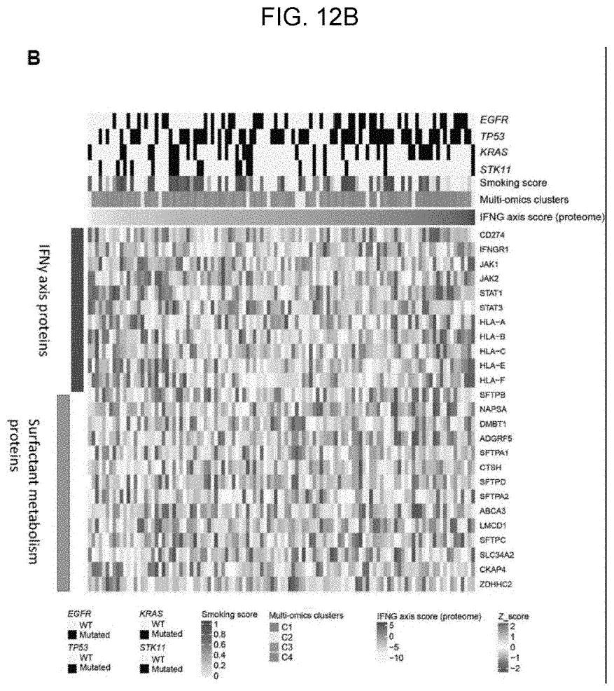

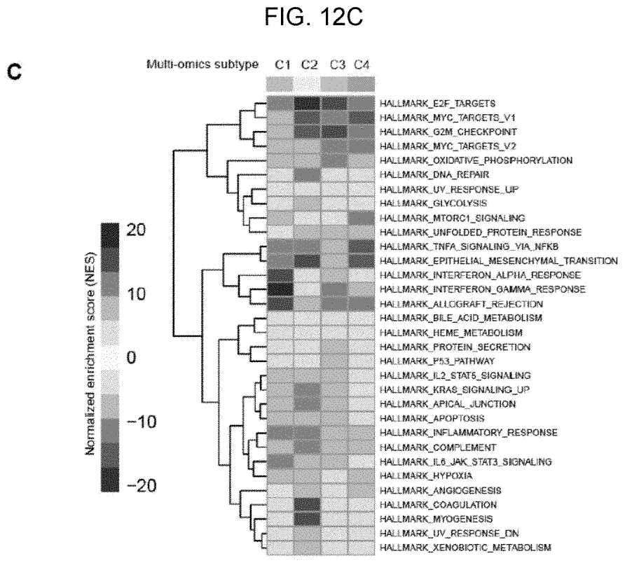

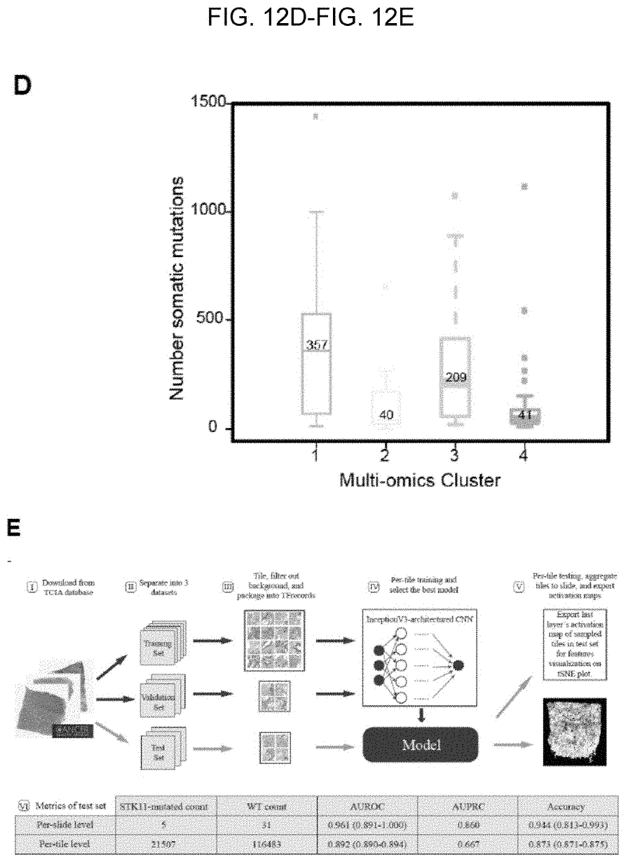

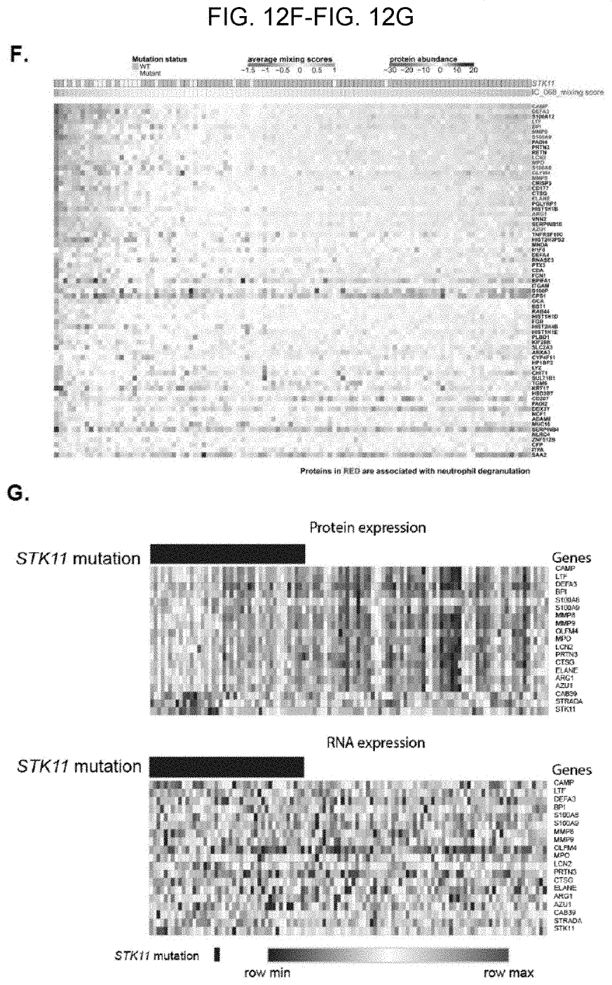

As an orthogonal assessment of the immune landscape of LUAD, we ranked tumors by activity of the IFN-γ axis, which is responsible for activation of the adaptive immune system (Abril-Rodriguez and Ribas, 2017), and assessed regulation of established immune evasion markers (Achyut and Arbab, 2016; Allard et al., 2016a; Liu et al., 2018). We also evaluated markers of “non-responder” signature genes derived from single cell sequencing of melanoma tumors classified by response to PD1/PDL1 therapy (Jerby-Arnon et al., 2018). As shown in FIG. 5A and FIG. 12A, the protein abundance of some important immune evasion markers, including Indoleamine 2, 3-dioxygenase (IDO1), was upregulated in the HTE cluster. IDO1 is a tryptophan catabolic enzyme that catalyzes the conversion of tryptophan into kynurenine (FIG. 5A, lower panel) (Liu et al., 2018). The depletion of tryptophan and the increase in kynurenine are associated with activation of T-regulatory cells (present in the HTE cluster) and myeloid-derived suppressor cells (Achyut and Arbab, 2016), suppressing the functions of effector-T and natural killer cells (Liu et al., 2018). IDO1 plays an important role in angiogenesis and promotion of epithelial-mesenchymal transition in cancer (Zhang et al., 2019a). Recently, combination therapy with an inhibitor of IDO1 and anti-PD-1/PD-L1 treatment was proposed for non-small cell lung cancer (Takada et al., 2019). Our findings suggest that the combination of PD-1/PD-L1 blockade with IDO1 inhibitor might increase efficiency of treatment of immune hot tumors in LUAD (Kozuma et al., 2018a; Takada et al., 2019). Other important immune evasive markers such as WARS, LCK, CD4, TYMP, B2M (upregulated in the HTE cluster) and PTGR2, PDE4D, MAOA (upregulated in the CTE cluster) are shown in FIG. 12A. LCK and CD4 are members of the supramolecular lymphocyte regulatory complex that includes PTPRCAP, one of the proteins our data suggest is regulated by DNA methylation (FIG. 3F, FIG. 3G). Also relevant to immune function is the pulmonary epithelium, a physical barrier that produces antimicrobial mucus and surfactant proteins, facilitates host-microbiota interactions to control mucosal immunity and is critical for tumor development (Whitsett and Alenghat, 2015). We found upregulation of immunosuppressive components of this barrier in the immune CTE cluster of lung tumors, including MUC5B, WFDC2 (HE4) (FIG. 5A, lower panel) (Parikh et al., 2019; Roy et al., 2014) and surfactants SFTPB, DMBT1, SFTPA1, and SFTPD (FIG. 12B) (Nayak et al., 2012; Seifart et al., 2005; Wang et al., 2009).

Notably, the NAT-enriched cluster had immune infiltration signatures that were intermediate between the hot and cold tumor subtypes (FIG. 5A), suggesting bi-directional regulation, with pro-inflammatory mechanisms in HTE and immune-evasive mechanisms in CTE tumors. The most dramatic down-regulation of immune activation was in STK11 mutant tumors, with marked reductions in xCell-derived Dendritic cell, Natural Killer T-cell and Macrophage signatures (FIG. 5B, FDR <0.1). In striking contrast, STK11 mutant-associated NATs were enriched for dendritic cell and macrophage infiltration (FIG. 5C, Fisher's exact test FDR <0.1). ESTIMATE immune scores (Yoshihara et al., 2013), while reduced for all STK11 mutants, were particularly low for those wild-type for KRAS (FIG. 5D). This immune downregulation did not appear to be related to mutation burden, as NMF cluster C3, strongly enriched for STK11 mutants (FIG. 1E), was second only to cluster C1 in somatic mutations (FIG. 12C, FIG. 12D). The immune-cold landscape of STK11 mutant tumors is sufficiently distinctive and pervasive that it proved to be the dominant feature in a deep-learning-based predictive algorithm for determining LUAD mutational status from histopathology (FIG. 5E). Building on prior work (Coudray et al., 2018), 10,000 tiles randomly selected from a holdout test set of ˜140,000 tiles from hematoxylin/eosin-stained LUAD slides representing STK11 mutant and wild type tumors were used to train a predictive model that achieved 94% accuracy at the slide level for predicting STK11 mutation status (FIG. 12E). By examining the layer immediately before the final output layer it was possible to identify critical tiles used by the model for prediction. The defining histopathologic features of STK11 mutant samples related to tumor epithelium, whereas STK11 wild type samples were predominantly characterized by immune cells (FIG. 5C).

In an attempt to understand the mechanisms contributing to the immune-cold phenotype of STK11 mutant tumors, we examined differential RNA, protein and phosphoprotein expression between STK11 WT and mutant samples. Pathway enrichments based on the top 100-300 differential proteins consistently showed neutrophil degranulation to be the signature most strongly associated with STK11 mutation. Notably, neutrophils do not appear to be either specifically enriched or specifically depleted in STK11 mutant tumors (FIG. 5A, FIG. 5B). Nevertheless, even in unsupervised feature extraction (Liu et al., 2019) of proteomic data, the defining feature of an STK-11 enriched cluster was neutrophil degranulation (FIG. 5F, FIG. 12F). All 16 of the measured proteins strongly associated with neutrophil degranulation were coherently overexpressed in STK11 mutants (FIG. 12G, upper panel). This signal was not detectable at the RNA level as the proteins, following translation, are stored in the granules until later release (FIG. 5G and FIG. 12G bottom panel). Most of these proteins, including cathelicidin antimicrobial peptide, lactoferrin, bactericidal permeability increasing protein, matrix metalloproteinases 8 and 9, myeloperoxidase, lipocalin 2, neutrophil elastase and arginase 1, have established (though sometimes divergent) immune modulatory functions. Collectively, they suggest a compelling hypothetical mechanism that may account for some of the immunologic effects of STK11 mutation.

Characterization of Smoking-Related Phenotype in Tumors and NATs

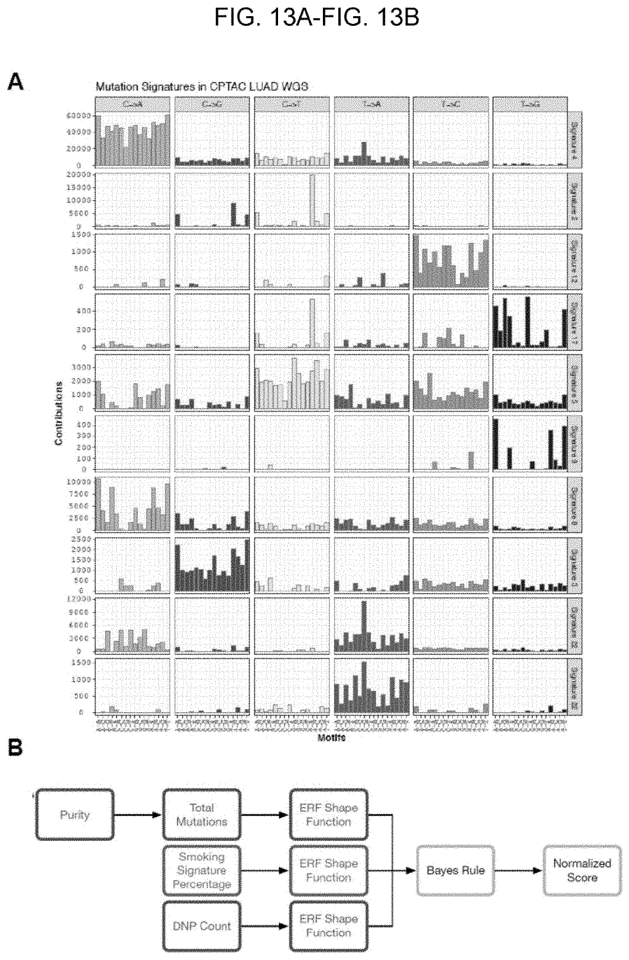

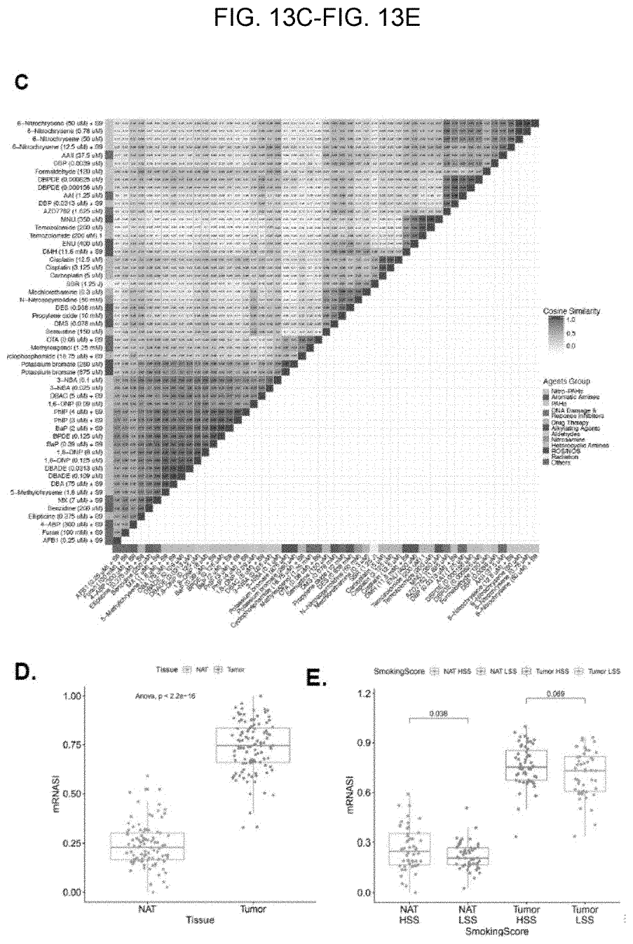

We called mutations for 109 LUAD tumor samples from WGS data using somaticwrapper, and subsequently identified ten distinct mutation signatures using SignatureAnalyzer (Kim et al., 2016) (STAR Methods). As shown in FIG. 13A, these signatures recapitulated the known characteristics of lung cancer mutations, including dominant C>T mutations in the AID/APOBEC family of cytidine deaminases signature (COSMIC mutational signature 2), transcriptional strand bias for T>C substitutions in the ApTpN context in COSMIC mutational signature 5, and C>A transversions in the smoking signature (COSMIC mutational signature 4) (Tate et al., 2019). Many samples had large percentages of smoking signature mutations (52 samples >50%). We further combined two adjacent SNVs into a single di-nucleotide polymorphism (DNP) mutation if they were in the same haplotype (STAR Methods). In total, we found 24,416 DNPs in the LUAD cohort. GG->TT or CC->AA is the dominant DNP type (˜50%) and is associated with smoking status.

In order to better characterize the influence of smoking as a major contributor to lung adenocarcinoma (Cancer Genome Atlas Research Network, 2014), we integrated tumor purity estimates, counts of total mutations, and percentages that are smoking signature mutations and smoking-signature DNPs into a continuous smoking signature score (FIG. 13B) (STAR Methods). Furthermore, we adjusted the scores for those with strong alternative (never-smoking) signatures. Among the 110 samples, we used this WGS-based smoking score (0, 1) to characterize 51 samples (score≤0.1, low smoking score, LSS) as cases with limited/minor smoking influence and 59 samples (score>0.1, high smoking score, HSS) as cases with clear smoking influence for downstream analysis. A linear model for identifying smoking signature high vs low differential markers (similar to the gender-specific effects model described above) found only three significant, unrelated markers (HDGF, GSDMD, ANXA10) (FDR <0.05) after adjustment for known confounders including mutation status, gender and place of origin.

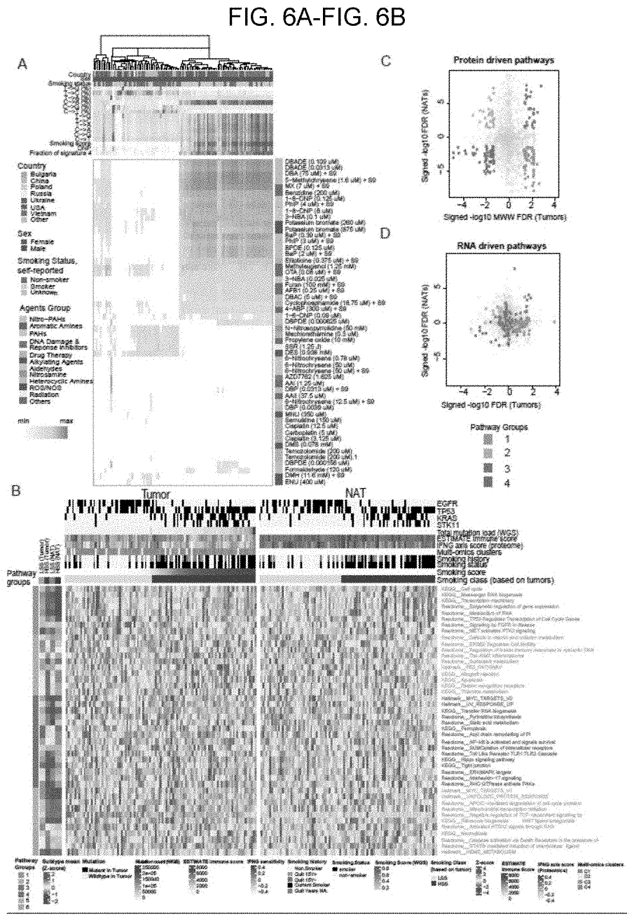

We further investigated links to a broad spectrum of environmental factors based on regression of the 96 possible trinucleotide mutation combinations between the samples in our cohort and the environmental signatures reported by Kucab et al. (Kucab et al., 2019). Briefly, correlations were calculated by least-squares fit, with significance assessed by T-test and subsequent multiple test correction via FDR. A total of 5,830 hypothesis tests were conducted. We found strong correlation (>0.75) of many samples (FIG. 6A) with many of the signatures of polycyclic aromatic hydrocarbons (PAHs) known to be present in cigarette smoke, e.g. DBADE, DBA, and 5-Methylchrysene. Moreover, these cases correlate highly with our smoking score (FIG. 6A) and with self-reported smoker status. Other environmental contributors (Kucab et al., 2019), evidently unrelated to cigarette smoking, were nevertheless also strongly correlated. For example, MX is a chlorine disinfection by-product and a known DNA mutagen suspected of increasing cancer risk when present at sufficient levels in drinking water (McDonald and Komulainen, 2005). Importantly, its signature was highly co-correlated to smoking signatures in the original Kucab et al. set, including coefficients >0.7 with the three PAHs mentioned above. Likewise, Benzidine, a chemical once heavily used in the dyeing industry and suspected to play a role in lung cancer (Tomioka et al., 2016), and PhIP, present in cooked meat and linked to various cancers (Tang et al., 2007), were also highly co-correlated to these PAHs (>0.5 and >0.7, respectively) (FIG. 13C). These observations suggest caution in interpreting these mutational associations and emphasize the importance of comprehensive clinical annotation including details on environmental and occupational exposures and dietary habits.

As previously reported (Malta et al., 2018) for many cancer types, tumors showed significantly higher stemness score compared to NAT (p=2.2 e-16) (FIG. 13D). Within both tumors and NATs, samples with HSS showed higher stemness score than samples with LSS (tumors: t test p=0.069; NATs: t test, p=0.038) (FIG. 13E), consistent with the known field cancerization effect of tobacco stream exposure (Walser et al., 2008).

We grouped tumors based on their smoking scores (HSS >0.1; LSS <0.1) and extended the same annotations and grouping scheme to matched NATs (FIG. 6B). We identified 6 patterns of differential pathway regulation between samples with high and low smoking scores (FIG. 6B). Pathways including cell cycle, mRNA biogenesis, and transcription machinery were reduced in NATs with HSS compared to LSS, but this pattern was reversed in tumors (FIG. 6B, Pathway Group (PG)1). Contrariwise, the AIM2 inflammasome (involved in pyroptosis), P53 pathway activity, and apoptosis were higher in NATs with HSS than with LSS, but lower in tumors with HSS, consistent with smoking-related tumors more effectively inactivating tumor suppressors and overcoming immune surveillance and apoptosis (FIG. 6B PG2). HSS had parallel effects on tumors and NATs in higher MYC target activity, UV response and ferroptosis, and lower Hippo pathway signaling and NF-kB and IL-17 activity (FIG. 6B PG3 and PG4). Finally, pathways including the unfolded protein response and RAS signaling through NTRK2 were higher in tumors but not NATs with HSS, while necroptosis and caspase signaling through death receptors were lower (FIG. 6B PG5 and PG6). Interestingly, the smoking signature-associated pathway-level differences that defined pathway groups 1-4 were more prominent on protein level than that of RNA. (FIG. 6C, D). While independent contributions of smoking score can be detected after controlling for mutational status, it is likely that the smoking score-related differentials described above represent a complex interplay between the direct effects of combustion-related carcinogen exposure and those mediated by mutational differences related to that exposure.

Tumor-NAT Comparisons Reveal Tumorigenic Changes and Biomarker Candidates

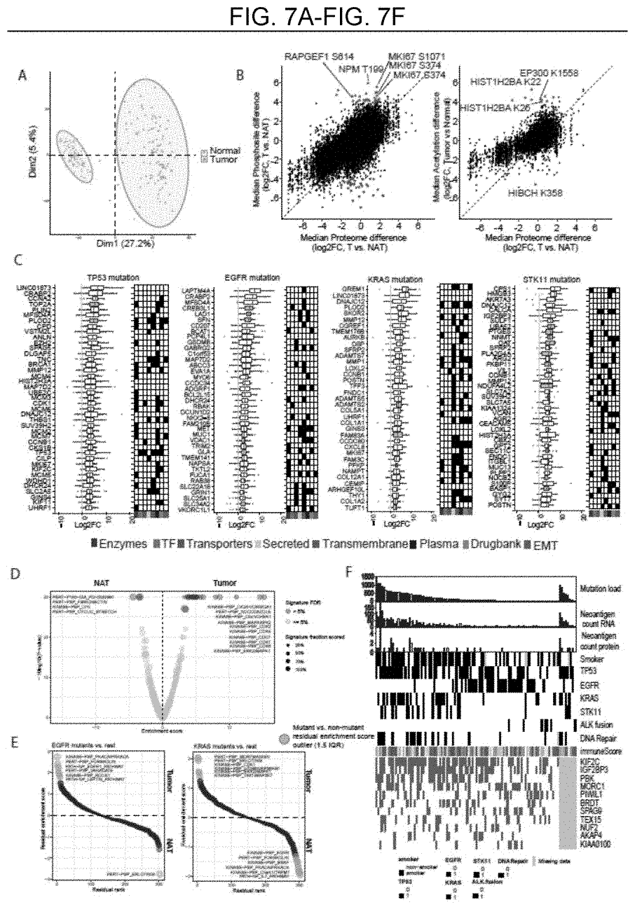

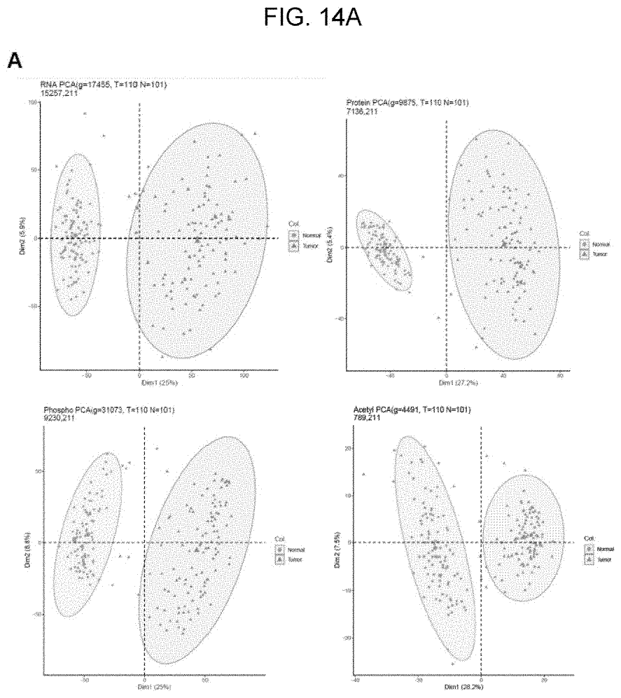

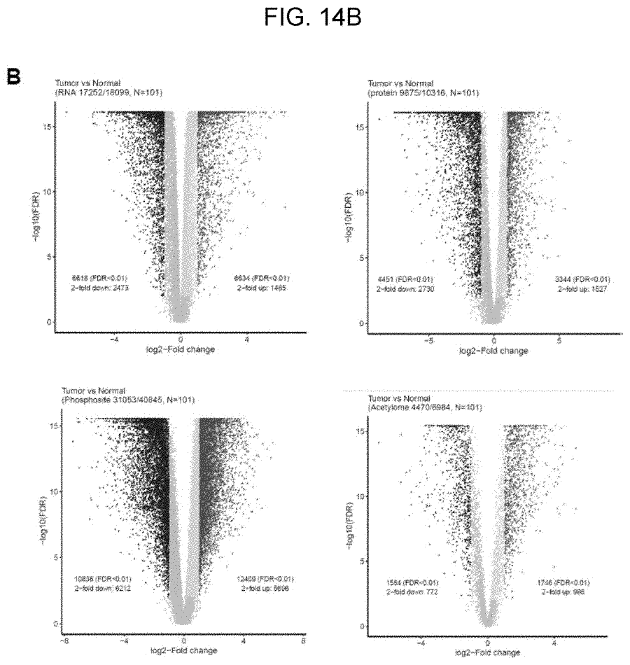

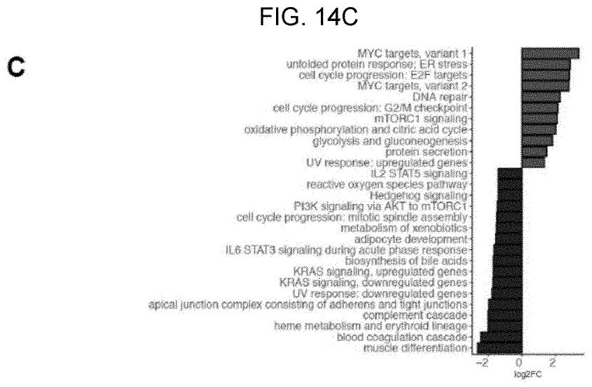

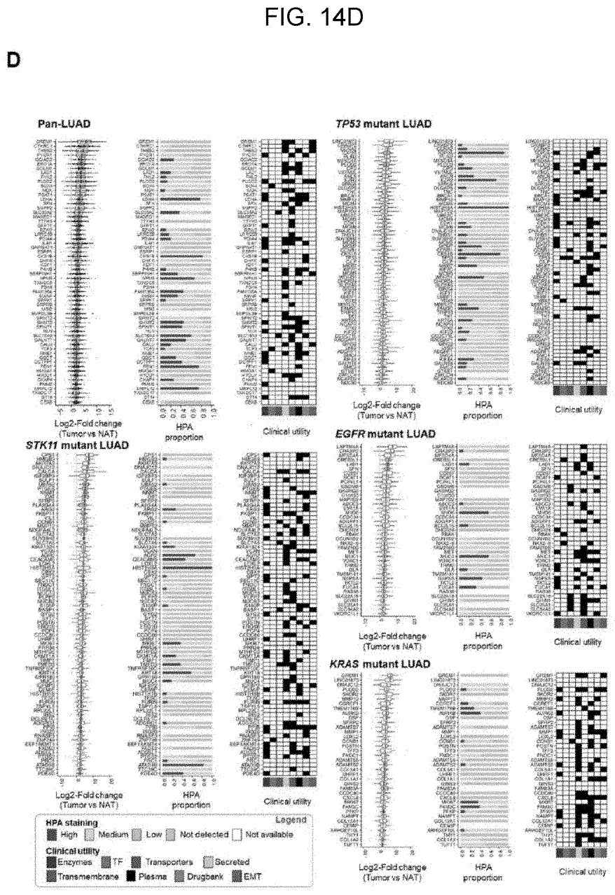

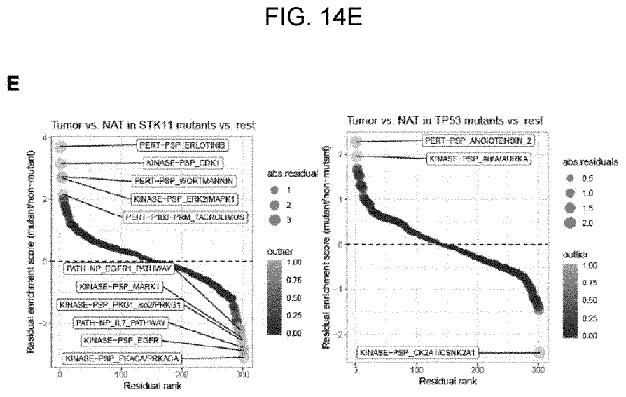

A strength of this study was that proteogenomic profiles were derived for both tumors and paired NATs, presenting a unique opportunity to explore proteogenomic remodeling upon tumorigenesis. Protein-level principal component analysis showed tumor and NAT populations to be completely distinct, with NATs showing a much greater degree of homogeneity (FIG. 7A). This general trend was also seen at the levels of RNA and phosphoproteome, though not in the acetylome (FIG. 14A). Of 10,316 confidently identified and quantified proteins in the global proteome analysis of tumor (n=110) and NAT samples (n=101), 7,795 (76%) showed differential tumor/NAT expression (Benjamini-Hochberg FDR <0.01, Wilcoxon signed rank test). Amongst 4,257 proteins with at least 2-fold differential expression, 64% were upregulated in NAT (FIG. 14B). Enrichment analysis revealed that tumorigenic processes including cell cycle progression, MYC targets upregulation and glycolysis were upregulated in tumor samples (adjusted p<0.001) (FIG. 14C). Although there was an expected significant positive correlation between protein expression and phospho- and acetyl-site abundance (phosphosites: R=0.69, p<2.2×10⁻¹⁶, acetyl-sites: R=0.66, p<2.2×10⁻¹⁶), we observed many sites (70 phosphosites [31-up, 39-down] and 11 acetyl-sites [10-up, 1-down]) for which abundance in tumors was markedly differential relative to associated protein expression, indicating a change in site stoichiometry. In FIG. 7B, sites with a minimum 16-fold up- or down-regulation compared to a maximum 4-fold change in associated protein expression are highlighted as red or blue triangles, for up- and down-regulation, respectively. NPM1 T199 showed the highest level of phosphorylation in tumors (p=1.86×10⁻¹⁵); phosphorylation of the T199 residue is known to be critical for NPM1-mediated DNA damage repair (Koike et al., 2010). Of note, proliferation marker MKI67 phosphorylation was dramatically upregulated in tumors (>45-fold) relative to its protein abundance (3.2-fold change) (FIG. 7B). Acetyl-site regulation included hyper-acetylation of the EP300 substrate, Histone 2B (HIST1H2BA K22/K25, ˜18-40-fold). Interestingly, we also observed significant acetylation of EP300 K1558 (18-fold), a key acetylation site in its activation loop that may be indicative of its activity (Thompson et al., 2004). HIBCH (3-hydroxyisobutyryl-CoA hydrolase), associated with valine metabolism, was the only protein distinctly hypoacetylated in tumors (K358; 24-fold, p=9.12×10⁻¹¹).

Deep proteogenomics characterization of LUAD tumors and paired NATs also provides a powerful dataset for biomarker candidate nomination. For diagnostic purposes, markers upregulated in tumors are generally preferred to those that are downregulated. Using stringent cutoffs for quantitative difference, significance and consistency (log 2-fold change >2, adjusted p<0.01, and differential in ≥90% of all Tumor-NAT pairs), we identified 289 proteins upregulated at the protein level. Sixty of these (FIG. 14D: Pan-LUAD) were also significantly differential at the RNA level, of which 5 (GFPT1, BZW2, PDIA4, P4HB, PMM2; indicated in blue), were upregulated in all tumor samples compared to their paired NATs. The metabolic enzymes GFPT1, PDIA4, P4HB and PMM2 have been previously implicated in lung and other cancer types (Chen et al., 2002; Tufo et al., 2014; Yang et al., 2016). Gremlin 1 (GREM1) protein, highly overexpressed in lung tumor samples (>50-fold, adj p=1.19×10⁻¹⁴) in our study, is known to be a poor prognosis marker in lung cancer (TCGA lung cancer, p<0.001; (Mulvihill et al., 2012), as well as playing an important role in EMT and metastasis processes represented by many other identified marker candidates (FIG. 14D) (Cleynen et al., 2007; Friedman et al., 2004; Tang et al., 2019). Numerous metabolic proteins were also highly upregulated (FIG. 14D, legend). Ovarian cancer immunoreactive antigen domain containing 2 (OCIAD2), highly overexpressed in lung tumors (˜18-fold, adj p=7.67×10⁻¹⁷), is a known poor prognosis marker (Sakashita et al., 2018), as are stress-related marker candidates including DHFR, HYOU1, LDHA, and CBX8 (Fahrmann et al., 2016; Lladó et al., 2009; Takei et al., 2017). While only a few of the metabolic proteins amongst these marker candidates are currently targeted by therapeutics in clinical trials, their marked and consistent differential expression and associations with lung cancer biology and decreased survival support potential utility in early detection and prognostic stratification (Kim et al., 2018; Mulvihill et al., 2012; Sakashita et al., 2018; Wang et al., 2015). Significantly hyperphosphorylated and hyperacetylated sites were identified.

We also explored mutation-specific tumor/NAT differential expression. FIG. 7C (and expanded list FIG. 14D) shows differential proteins (log 2 fold change >2 and FDR <0.01 in >80% of samples) significantly upregulated in TP53-, EGFR-, KRAS- and STK11-mutant phenotypes. Patients with TP53-mutant tumors show high expression of TP53, CCNA2, TOP2A, PLOD2, ANLN, and MMP12, all shown to have roles in tumorigenesis (Chen et al., 2015; Hosgood et al., 2008; Konofaos et al., 2013; Qu et al., 2009; Song et al., 2013). The coordinated high expression of TP53 and TP53 inhibitor MDM2 and MDM4 proteins in TP53 mutant samples suggests either TP53 stabilization or low turnover of TP53 degradation (Quintás-Cardama et al., 2017). Notably, we also observed high protein expression of CDK1 and CCNB1 as well as elevated CDK1 phosphorylation in TP53 mutants, all known to be associated with resistance in preclinical models modulated by p53 status (FIG. 7C, 1st panel) (Schwermer et al., 2015). Significant overexpression of the proto-oncogene MET was noted in EGFR mutants (FIG. 7C, 2nd panel), along with S100B, an established serological marker overexpressed in EGFR mutant lung tumors (Mu et al., 2017). Extracellular glycoproteins, collagens and enzymes were enriched in KRAS mutant tumors (FIG. 7C, 3rd panel), as were the well-described KRAS-associated chemokine CXCL8 and immune target THY1 (Sunaga et al., 2012). STK11 mutant tumors were enriched (FIG. 7C, 4th panel) for amino acid metabolism proteins, which are associated with nitric oxide metabolic processes, suggesting perturbation of the urea cycle in the context of STK11 mutation (Kim et al., 2017; Lam et al., 2019).

Phosphosite-specific pathway analyses (Krug et al., 2018) of the entire population of tumor/NAT pairs showed upregulated phosphosite-driven signatures chiefly of checkpoint control and cell cycle progression in tumors (FIG. 7D) compared to extracellular matrix-focused signatures in paired NATs. Phosphosite-driven signatures differential between tumor and matched NATs in tumors with EGFR (N=38) or KRAS (N=33) mutations yielded near-mirror image plots (FIG. 7D). KRAS mutant tumors showed site-driven activation of pathways downstream of RAS, including MAPK1, as well as of TAK1, the hub at which IL1, TGF-β and Wnt signaling pathways converge (Santoro et al., 2017). Pathways upregulated in EGFR tumors included ROCK1, a Rho-associated protein kinase that has been shown to enhance EGFR activation in some cancer types (Nakashima et al., 2011).

Cancer testis (CT) antigens and tumor neoantigens can serve both diagnostic and therapeutic roles, including as potential cancer vaccine targets. Of 44 CT antigens recurrently over-expressed in tumors (fold-change ≥2), 9 were observed in ≥10% of samples (FIG. 7F). KIF2C was the most ubiquitous, being highly expressed in 63% of samples. Seven of these 9 common CT antigens, KIF2C, IGF2BP3, PBK, PIWIL, BRDT, TEX15, and AKAP4, have been previously associated with lung cancer (Bai et al., 2019; Lei et al., 2015; Loriot et al., 2003; Scanlan et al., 2000; Xie et al., 2018; Zhao et al., 2017), although their specific roles in tumorigenesis and progression are unclear. IGF2BP3 is associated with tumor progression and poor prognosis in colorectal cancer, lung cancer and hepatocellular carcinoma (Jiang et al., 2008; Lochhead et al., 2012; Xu et al., 2012), while AKAP4 has been proposed to be a potential biomarker in non-small cell lung cancer (NSCLC) (Loriot et al., 2003). To our knowledge, MORC1 and NUF2 are novel CT antigens in LUAD tumors, covering 38% and 16% of patients, respectively. To identify additional predicted tumor neoantigens, we also searched for both RNA transcripts and peptides containing evidence of somatic mutations. We identified a total of 2481 mRNA-validated and 49 peptide-validated somatic mutations, corresponding to 104 patients (FIG. 7F). Overall, 97 samples had evidence of either CT antigens or neoantigens, holding promise for the future of immunotherapy-based approaches to LUAD management.

Discussion

In this study, we report the most comprehensive proteogenomic characterization of 110 LUAD tumors and 101 matched NATs to date. Unlike TCGA, which included primarily smoking-related LUAD, our cohort included roughly equal numbers of current or former smokers and never-smokers, as well as a geographically diverse population. Multi-omics unsupervised clustering showed that previously-described terminal respiratory unit and proximal-inflammatory clusters translate to the protein level, while proximal-proliferative samples showed substructure based on TP53 status and place of origin. miRNA taxonomy included clusters enriched for STK11 mutant- and ALK fusion-driven tumors. We observed consistent differential phosphorylation of ALK Y1507 in samples with ALK fusion, in addition to multiple other proteins exclusively regulated at the level of phosphoproteome that underscores their relevance to ALK-associated biology. Both known and novel ALK fusion partners were identified, with differential expression of partners also seen only at the phosphosite level.

The inclusion of deep-scale proteomic and PTM data allowed us to track the downstream signaling consequences of epigenetic and genomic alterations. Among 120 methylation cis-effects that cascaded through RNA to protein and phosphoprotein expression, a number were established but many had no prior association to LUAD biology. Linking protein and phosphosite data to KEAP1 mutations elucidated the KEAP1/NFE2L2 regulatory network, suggesting a novel regulatory mechanism involving hindrance of KEAP1/NFE2L2 interaction. Extreme phosphorylation events on important, targetable proteins implied therapeutic possibilities including SOS1 inhibition in KRAS mutant and PTPN11 (Shp2) inhibition in EGFR mutant tumors. The latter is an especially exciting potential opportunity as the relevant PTPN11 Y62 phosphorylation affected virtually all EGFR mutant tumors and Shp2 inhibitors are already in clinical trials. We also systematically identified and annotated outlier kinases, some unique to major mutational subtypes, many of which have known inhibitors or appear to be druggable. These outliers are also predominantly phosphorylation events, reinforcing the value of post-translational modification analysis. Matched tumor-NAT analysis illuminated elements of oncogenesis, but also allowed us to catalog global-LUAD and driver-specific differential proteins and nominate the most extreme as biomarker candidates and potential drug development targets.

Furthermore, integrated proteogenomics allowed extensive characterization of the immune landscape of LUADs and identification of a number of potential therapeutic vulnerabilities. We highlighted the particular association of STK11 mutation with immune-cold behavior, and implicated neutrophil degranulation as a potential immunosuppressive mechanism in STK11-mutant LUAD. As with many of the findings we present, the neutrophil degranulation signal was evident only in the proteomics space, emphasizing the critical importance of combining proteomic with genomic information to better understand tumor biology. The combination of proteogenomic data, balanced representation of smokers and never-smokers, and paired tumor/NAT analyses enabled us to capture the impact of cancerization not only in tumors, but also in the adjacent tissue samples, including higher stemness scores in tobacco-exposed tissues.

There are inherent limitations to a study of this type. Important annotations based on self report, such as smoking history and other environmental exposures, can be subject to bias; this may account for a degree of discrepancy in our study between our self-reported tobacco use and molecular signature of smoking exposure. The interdependence of variables including mutational status, ethnicity or geography, gender and smoking status require that comparisons based on any one of these be interpreted with caution. Furthermore, given the large number of confounders, efforts to adjust for this by linear modeling are not effective in a dataset of this size, frustrating association analyses such as for gender and smoking effects. This effort shares with all bulk tumor analyses the lack of spatial and cellular resolution that might add orthogonal insights into tumor biology, such as by disambiguating the contributions of tumor epithelium and microenvironment. Approaches geared to more spatially resolved proteogenomics, such as we and others have recently described (Hunt et al., 2019; Satpathy et al., 2019), might add nuance to our understanding of the intricate crosstalk between tumor, immune cells and the microenvironment, while integration of single cell genomics and proteomics would provide additional valuable insights into tumor evolution. The integration of deep-scale proteomic and PTM data nevertheless represents a substantial advance over prior genomics studies of LUAD. We hope that both the specific observations and hypotheses delineated in this manuscript, and the data that underlie them, will be a rich resource for those investigating lung adenocarcinoma and for the larger research community, including for the development of targeted chemo- or immuno-therapies.

Methods

Human Subjects

A total of 111 participants were included in this study collected by 13 different tissue source sites from 8 different countries. This study contained both males (n=73) and females (n=38). Histopathologically defined adult Lung Adenocarcinoma tumors were only considered for analysis, with an age range of 35-80. Institutional review boards at tissue source site reviewed protocols and consent documentation adhering to the Clinical Proteomic Tumor Analysis Consortium (CPTAC) guidelines.

Clinical Data Annotation

Clinical data were obtained from tissue source sites and aggregated by an internal database called the CDR (Comprehensive Data Resource) that synchronizes with the CPTAC DCC. Clinical data can be accessed and downloaded from the DCC (Data Coordinating Center) at https://cptac-data-portal.georgetown.edu/cptac/s/5046. Demographics, histopathologic information, and treatment details were collected. The characteristics of the LUAD cases of CPTAC cohort reflect the general population of Lung Adenocarcinoma patients. The genotypic, clinical, geographical and other associated metadata was collected.

Specimen Acquisition

The tumor, normal adjacent tissue (NAT), and whole blood samples used in this manuscript were prospectively collected for the CPTAC project. Biospecimens were collected from newly diagnosed patients with LUAD who were undergoing surgical resection and had received no prior treatment for their disease, including chemotherapy or radiotherapy. All cases had to be of acceptable LUAD histology but were collected regardless of surgical stage or histologic grade. Cases were staged using the AJCC cancer staging system 7th edition (Edge et al., 2010). The tumor specimens weight ranged between 125 to 715 milligrams. Paired normal tissues were collected from the same patient that tumor was excised. The criteria was that NATs must be free of tumor upon path review. The average tissue mass was found to be 238 mg. For most cases, three to four tumor specimens were collected. Each tissue specimen endured cold ischemia for 40 minutes or less prior to freezing in liquid nitrogen. The specimens were collected with an average total ischemic time of 13 minutes from resection/collection to freezing. Specimens were either flash frozen in liquid nitrogen or embedded in optimal cutting temperature (OCT) medium, with histologic sections obtained from top and bottom portions for review. Each case was reviewed by a board-certified pathologist to confirm the assigned pathology. The top and bottom sections had to contain an average of 50% tumor cell nuclei with less than 20% necrosis. Specimens were shipped overnight from the tissue source sites to the biospecimen core resource (BCR) located at Van Andel Research Institute at Grand Rapids, Mich. using a cryoport that maintained an average temperature of less than −140° C. At the biospecimen core resource, the specimens were confirmed for pathology qualification and prepared for genomic, transcriptomic, and proteomic analyses. Selected specimens were cryopulverized, and material aliquoted for subsequent molecular characterization. Genomic DNA and total RNA were extracted and sent to the genome sequencing centers. The DNA sequencing was performed at the Broad Institute, Cambridge, Mass. and RNA sequencing was performed at the University of North Carolina, Chapel Hill, N.C. Material for proteomic analyses were sent to the Proteomic Characterization Center (PCC) at the Broad Institute, Cambridge, Mass.

Genomics and Transcriptomics Sample Preparation

Our study sampled a single site of the primary tumor from surgical resections, due to the internal requirement to process a minimum of 125 mg of tumor issue and 50 mg of adjacent normal tissue. DNA and RNA were extracted from tumor and adjacent normal specimens in a co-isolation protocol using Qiagen's QIAsymphony DNA Mini Kit and QIAsymphony RNA Kit. Genomic DNA was also isolated from peripheral blood (3-5 mL) to serve as matched normal reference material. The Qubit™ dsDNA BR Assay Kit was used with the Qubit® 2.0 Fluorometer to determine the concentration of dsDNA in an aqueous solution. Any sample that passed quality control and produced enough DNA yield to go through various genomic assays was sent for genomic characterization. RNA quality was quantified using both the NanoDrop 8000 and quality assessed using Agilent Bioanalyzer. A sample that passed RNA quality control and had a minimum RIN (RNA integrity number) score of 7 was subjected to RNA sequencing. Identity match for germline, normal adjacent tissue, and tumor tissue was assayed at the BCR using the Illumina Infinium QC array. This beadchip contains 15,949 markers designed to prioritize sample tracking, quality control, and stratification.

Whole Exome Sequencing (WXS)

Library Construction and In-Solution Hybrid Selection

Library construction was performed as described in (Fisher et al., 2011), with the following modifications: initial genomic DNA input into shearing was reduced from 3 μg to 20-250 ng in 50 μL of solution. For adapter ligation, Illumina paired-end adapters were replaced with palindromic forked adapters, purchased from Integrated DNA Technologies, with unique dual-indexed molecular barcode sequences to facilitate downstream pooling. Kapa HyperPrep reagents in 96-reaction kit format were used for end repair/A-tailing, adapter ligation, and library enrichment PCR. In addition, during the post-enrichment SPRI cleanup, elution volume was reduced to 30 μL to maximize library concentration, and a vortexing step was added to maximize the amount of template eluted. After library construction, libraries were pooled into groups of up to 96 samples. Hybridization and capture were performed using the relevant components of Illumina's Nextera Exome Kit and following the manufacturer's suggested protocol, with the following exceptions. First, all libraries within a library construction plate were pooled prior to hybridization. Second, the Midi plate from Illumina's Nextera Exome Kit was replaced with a skirted PCR plate to facilitate automation. All hybridization and capture steps were automated on the Agilent Bravo liquid handling system

Cluster Amplification and Sequencing

After post-capture enrichment, library pools were quantified using qPCR (automated assay on the Agilent Bravo) using a kit purchased from KAPA Biosystems with probes specific to the ends of the adapters. Based on qPCR quantification, libraries were normalized to 2 nM. Cluster amplification of DNA libraries was performed according to the manufacturer's protocol (Illumina) using exclusion amplification chemistry and flowcells. Flowcells were sequenced utilizing sequencing-by-synthesis chemistry. The flowcells were then analyzed using RTA v.2.7.3 or later. Each pool of whole exome libraries was sequenced on paired 76 cycle runs with two 8 cycle index reads across the number of lanes needed to meet coverage for all libraries in the pool. Pooled libraries were run on HiSeq4000 paired-end runs to achieve a minimum of 150× on target coverage per each sample library. The raw Illumina sequence data were demultiplexed and converted to fastq files; adapter and low-quality sequences were trimmed. The raw reads were mapped to the hg38 human reference genome and the validated bams were used for downstream analysis and variant calling.

Whole Genome Sequencing

Cluster Amplification and Sequencing

An aliquot of genomic DNA (350 ng in 50 μL) was used as the input into DNA fragmentation (aka shearing). Shearing was performed acoustically using a Covaris focused-ultrasonicator, targeting 385 bp fragments. Following fragmentation, additional size selection was performed using a SPRI cleanup. Library preparation was performed using a commercially available kit provided by KAPA Biosystems (KAPA Hyper Prep without amplification module) and with palindromic forked adapters with unique 8-base index sequences embedded within the adapter (purchased from IDT). Following sample preparation, libraries were quantified using quantitative PCR (kit purchased from KAPA Biosystems), with probes specific to the ends of the adapters. This assay was automated using Agilent's Bravo liquid handling platform. Based on qPCR quantification, libraries were normalized to 1.7 nM and pooled into 24-plexes.

Sample pools were combined with HiSeqX Cluster Amp Reagents EPX1, EPX2, and EPX3 into single wells on a strip tube using the Hamilton Starlet Liquid Handling system. Cluster amplification of the templates was performed according to the manufacturer's protocol (Illumina) with the Illumina cBot. Flowcells were sequenced to a minimum of 15× on HiSeqX utilizing sequencing-by-synthesis kits to produce 151 bp paired-end reads. Output from Illumina software was processed by the Picard data processing pipeline to yield BAM files containing demultiplexed, aggregated, aligned reads. All sample information tracking was performed by automated LIMS messaging.

Array Based Methylation Analysis

The MethylationEPIC array uses an 8-sample version of the Illumina Beadchip capturing >850,000 methylation sites per sample. 250 ng of DNA was used for the bisulfite conversation using Infinium MethylationEPIC BeadChip Kit. The EPIC array includes sample plating, bisulfite conversion, and methylation array processing. After scanning, the data was processed through an automated genotype calling pipeline. Data generated consisted of raw idats and a sample sheet.

RNA Sequencing

QA, QC of RNA Analytes and Total RNA-seq Library Construction All RNA analytes were assayed for RNA integrity, concentration, and fragment size. Samples for total RNA-seq were quantified on a TapeStation system (Agilent, Inc. Santa Clara, Calif.). Samples with RINs >8.0 were considered high quality.

Total RNA-seq libraries were generated using 300 nanograms of total RNA using the TruSeq Stranded Total RNA Library Prep Kit with Ribo-Zero Gold and bar-coded with individual tags following the manufacturer's instructions (Illumine). Total RNA Libraries were prepared on an Agilent Bravo Automated Liquid Handling System. Quality control was performed at every step, and the libraries were quantified using a TapeStation system.

Total RNA Sequencing

Indexed libraries were prepared and run on HiSeq4000 paired-end 75 base pairs to generate a minimum of 120 million reads per sample library with a target of greater than 90% mapped reads. The raw Illumina sequence data were demultiplexed and converted to fastq files, and adapter and low-quality sequences were quantified. Samples were then assessed for quality by mapping reads to hg38, estimating the total number of mapped reads, amount of RNA mapping to coding regions, amount of rRNA in the sample, number of genes expressed, and relative expression of housekeeping genes. Samples passing this QA/QC were then clustered with other expression data from similar and distinct tumor types to confirm expected expression patterns. Atypical samples were then SNP typed from the RNA data to confirm source analyte. FASTQ files of all reads were then uploaded to the GDC repository.

miRNA-Seq Library Construction

miRNA-seq library construction was performed from the RNA samples using the NEXTflex Small RNA-Seq Kit (v3, PerkinElmer, Waltham, Mass.) and barcoded with individual tags following the manufacturer's instructions. Libraries were prepared on a Sciclone Liquid Handling Workstation. Quality control was performed at every step, and the libraries were quantified using a TapeStation system and an Agilent Bioanalyzer using the Small RNA analysis kit. Pooled libraries were then size selected according to NEXTflex Kit specifications using a Pippin Prep system (Sage Science, Beverly, Mass.).

miRNA Sequencing

Indexed libraries were loaded on the HiSeq4000 to generate a minimum of 10 million reads per library with a minimum of 90% reads mapped. The raw Illumina sequence data were demultiplexed and converted to FASTQ files for downstream analysis. Resultant data were analyzed using a variant of the small RNA quantification pipeline developed for TCGA (Chu et al., 2016). Samples were assessed for the number of miRNAs called, species diversity, and total abundance. Samples were uploaded to the GDC repository.

Mass Spectrometry Based Proteomics, Phosphoproteomics and Acetylomics.

The protocols below for protein extraction, tryptic digestion, TMT-10 labeling of peptides, peptide fractionation by basic reversed-phase liquid chromatography, phosphopeptide enrichment using immobilized metal affinity chromatography, and LC-MS/MS were performed as previously described in-depth (Mertins et al., 2018). Acetyl-enrichment was performed as described before (Svinkina et al., 2015; Udeshi et al.) with modifications described below.

Protein Extraction and Tryptic Digestion for TMT Analysis

Cryopulverized human lung adenocarcinoma patient tumor samples were homogenized in lysis buffer at a ratio of about 200 μL lysis buffer for every 50 mg wet weight tissue. The lysis buffer consisted of 8 M urea, 75 mM NaCl, 1 mM EDTA, 50 mM Tris HCl (pH 8), 10 mM NaF, phosphatase inhibitor cocktail 2 (1:100; Sigma, P5726) and cocktail 3 (1:100; Sigma, P0044), 2 μg/mL aprotinin (Sigma, A6103), 10 μg/mL Leupeptin (Roche, 11017101001), and 1 mM PMSF (Sigma, 78830). Lysates were centrifuged at 20,000 g for 10 minutes and protein concentrations of the clarified lysates were measured by BCA assay (Pierce). Protein lysates were subsequently reduced with 5 mM dithiothreitol (Thermo Scientific, 20291) for an hour at 37 C and alkylated with 10 mM iodoacetamide (Sigma, A3221) for 45 minutes in the dark at room temperature. Prior to digestion, samples were diluted 4-fold to achieve 2 M urea with 50 mM Tris HCl (pH 8). Digestion was performed with LysC (Wako, 100369-826) for 2 hours and with trypsin (Promega, V511X) overnight, both at a 1:50 enzyme-to-protein ratio and at room temperature. Digested samples were acidified with formic acid (FA; Fluka, 56302) to achieve a final volumetric concentration of 1% (final pH of ˜3), and centrifuged at 1,500 g for 15 minutes to clear precipitated urea from peptide lysates. Samples were desalted on C18 SepPak columns (Waters, 100 mg, WAT036820) and dried down using a SpeedVac apparatus.

Construction of the Common Reference Pool

The proteomic and phosphoproteomic analyses of lung cancer samples were structured as TMT-10 plex experiments. To facilitate quantitative comparison between all samples across experiments, a common reference sample was included in each 10-plex. A common physical, rather than in silico reference was used for this purpose for optimal quantitative precision between TMT-10 experiments. Considerations prior to creating the reference sample were that this sample needed to be of adequate quantity to cover all planned experiments for both the discovery and confirmatory sets with overhead for additional possible experiments. The internal reference includes nearly all the samples analyzed in the TMT experiments, yielding an internal reference that is representative of all the samples in the study. Making the internal reference as representative of the study as a whole was particularly important since by definition only analytes represented in the reference sample would be included in the final ratio-based data analyses.

111 unique tumor samples with 102 paired normal samples were distributed among 2510-plex experiments, with 9 individual samples occupying the first 9 channels of each experiment and the 10th channel being reserved for the reference sample. The first 8 channels of each experiment contained 4 tumor/normal pairs, with each pair of patient samples adjacent to each other. All the tumors were in the C channels and all the normal samples were in the N channels. Of the 25 130C channels, 9 contained unpaired tumors, 4 contained tumor only internal references, 4 had normal only internal references, 2 were internal references from a team in Taiwan (Academia Sinica), 2 were replicate tumor samples, and 4 samples were 2 tumor/normal paired patients, split for the purpose of filling in channels.

To ensure capacity for additional samples or experiments given a target input of 300 μg protein per channel per experiment, 30 mg total was targeted for reference material. To meet these collective requirements, all the samples that had enough material were included in the internal reference. After reserving 300 μg peptide/sample for individual sample analysis, an additional amount of 150 μg for each of the samples with adequate quantities were pooled. In total, 203 samples were selected for the combined tumor/normal internal reference. To make the combined internal reference, tumor only and normal only internal references were first created separately. 103 tumor samples and 100 normal samples were included in the respective internal references. After creating individual IRs, a pool of combined internal reference was made, comprised of 4.8 mg tumor only reference and 4.8 mg normal only reference. The 9.6 mg pooled reference material was divided into 300 μg aliquots and frozen at −80° C. until use. 3.9 mg of tumor only and 3.9 mg of normal only reference pool were set aside for future combined tumor/normal internal references. The remaining tumor only and normal only references were aliquoted into 300 μg amounts, dried down, and stored at −80 C for future use.

Construction and Utilization of the Comparative Reference Sample

As a quality control measure, two “comparative reference” (“CompRef”) samples were generated as previously described (Li et al., 2013; Mertins et al., 2018) and used to monitor the longitudinal performance of the proteome, phosphoproteome, and acetylome workflows throughout the course of the project. Briefly, patient-derived xenograft tumors from established basal (WHIM2) and luminal-B (WHIM16) breast cancer intrinsic subtypes (Li et al., 2013) were raised subcutaneously in 8 week old NOD.Cg-Prkdc^{scid} Il2rg^{tm1Wj1}/SzJ mice (Jackson Laboratories, Bar Harbor, Me.) using procedures reviewed and approved by the institutional animal care and use committee at Washington University in St. Louis. All PDX models are available through the application to the Human and Mouse-Linked Evaluation of Tumors core at http://digitalcommons.wustl.edu/hamlet/. Xenografts were grown in multiple mice, pooled, and cryofractured to provide a sufficient amount of material for the duration of the project. Using the same analysis protocol as the patient samples, four proteome, phosphoproteome, and acetylome process replicates of each of the two xenografts were prepared as described below and run as TMT 10-plex experiments (5 aliquots of each PDX model/plex) at the beginning and end of the 25 patient plexes and interposed after patient plexes 8 and 16. Interstitial samples were evaluated for depth of coverage and for consistency in quantitative comparison between the basal and luminal models.

TMT-10 Labeling of Peptides

300 μg of desalted peptides per sample (based on peptide-level BCA after digestion) were labeled with 10-plex TMT reagents according to the manufacturer's instructions (Thermo Scientific; Pierce Biotechnology, Germany). For each 300 μg peptide aliquot of an individual tumor sample, 2.4 mg of labeling reagent was used. Peptides were dissolved in 300 μL of 50 mM HEPES (pH 8.5) solution and labeling reagent was added in 123 μL of acetonitrile. After 1 h incubation with shaking and after confirming good label incorporation, 24 μL of 5% hydroxylamine was added to quench the unreacted TMT reagents. Good label incorporation was defined as having a minimum of 95% fully labeled MS/MS spectra in each sample, as measured by LC-MS/MS after taking out a 2.8 μg aliquot from each sample and analyzing 1.25 μg. If a sample did not have sufficient label incorporation, additional TMT was added to the sample and another 1 h incubation was performed with shaking. At the time that the labeling efficiency quality control samples were taken out, an additional 4 μg of material from each sample was taken out and combined as a mixing control. After analyzing the mixing control sample by LC-MS/MS, intensity values of the individual TMT reporter ions were summed across all peptide spectrum matches and compared to ensure that the total reporter ion intensity of each sample met a threshold of +/−15% of the internal reference. If necessary, adjustments were made by either labeling additional material or reducing an individual sample's contribution to the mixture, and analyzing a subsequent mixing control, until all samples met the threshold and were thus approximately 1:1:1. Differentially labeled peptides were then mixed (10×300 μg), dried down via vacuum centrifuge, and then quenched, combined sample was subsequently desalted on a 200 mg C18 SepPak column.

Peptide Fractionation by Basic Reversed-Phase Liquid Chromatography (bRPLC)

To reduce sample complexity, peptide samples were separated by high pH reversed phase (RP) separation as described prior. A desalted 3 mg, 10-plex TMT-labeled experiment (based on protein-level BCA prior to digestion) was reconstituted in 900 μL 5 mM ammonium formate (pH 10) and 2% acetonitrile, loaded on a 4.6 mm×250 mm column RP Zorbax 300 A Extend-C18 column (Agilent, 3.5 μm bead size), and separated on an Agilent 1260 Series HPLC instrument using basic reversed-phase chromatography. Solvent A (2% acetonitrile, 4.5 mM ammonium formate, pH 10) and a nonlinear increasing concentration of solvent B (90% acetonitrile, 4.5 mM ammonium formate, pH 10) were used to separate peptides. The 4.5 mM ammonium formate solvents were made by 40-fold dilution of a stock solution of 180 mM ammonium formate, pH 10. To make 1 L of stock solution, add 25 mL of 28% (wt/vol) ammonium hydroxide (28%, density 0.9 g/ml, Sigma-Aldrich) to ˜850 ml of HPLC grade water, then add ˜35 mL of 10% (vol/vol) formic acid (>95% Sigma-Aldrich) to titrate the pH to 10.0; bring the final volume to 1 liter with HPLC grade water. The 96 minute separation LC gradient followed this profile: (min: % B) 0:0; 7:0; 13:16; 73:40; 77:44; 82:60; 96:60. The flow rate was 1 mL/min. Per 3 mg separation, 82 fractions were collected into a 96 deep-well×2 mL plate (Whatman, #7701-5200), with fractions combined in a step-wise concatenation strategy and acidified to a final concentration of 0.1% FA as reported previously. An additional 14 fractions were collected from the 96 deep-well plate for fraction A, which are the early eluting fractions that tend to contain multi-phosphorylated peptides. 5% of the volume of each of the 24+A proteome fractions was allocated for proteome analysis, dried down, and re-suspended in 3% MeCN/0.1% FA (MeCN; acetonitrile) to a peptide concentration of 0.25 μg/μL for LC-MS/MS analysis. The remaining 95% of concatenated 24 fractions were further combined into 12 fractions, with fraction A as a separate fraction. These 13 fractions were then enriched for phosphopeptides as described below.

Phosphopeptide Enrichment Using Immobilized Metal Affinity Chromatography (IMAC)

Ni-NTA agarose beads were used to prepare Fe³⁺-NTA agarose beads. In each phosphoproteome fraction, ˜237.5 μg peptides (based on peptide-level BCA after digestion with uniformly-distributed fractionation presumed) were reconstituted in 475 μL 80% MeCN/0.1% TFA (trifluoroacetic acid) solvent and incubated with 10 μL of the IMAC beads for 30 minutes on a shaker at RT. After incubation, samples were briefly spun down on a tabletop centrifuge; clarified peptide flow-throughs were separated from the beads; and the beads were reconstituted in 200 μL IMAC binding/wash buffer (80 MeCN/0.1% TFA) and loaded onto equilibrated Empore C18 silica-packed stage tips (3M, 2315). Samples were then washed twice with 50 μL of IMAC binding/wash buffer and once with 50 μL 1% FA, and were eluted from the IMAC beads to the stage tips with 3×70 μL washes of 500 mM dibasic sodium phosphate (pH 7.0, Sigma S9763). Stage tips were then washed once with 100 μL 1% FA and phosphopeptides were eluted from the stage tips with 60 μL 50% MeCN/0.1% FA. Phosphopeptides were dried down and re-suspended in 9 μL 50% MeCN/0.1% FA for LC-MS/MS analysis, where 4 μL was injected per run.

Acetylpeptide Enrichment

Acetylated lysine peptides were enriched using an antibody against Acetyl-Lysine motif (CST PTM-SCAN Catalogue No. 13416). IMAC eluents were concatenated into 4 fractions (˜750 μg peptides per fraction) and dried down using a SpeedVac apparatus. Peptides were reconstituted with 1.4 ml of IAP buffer (5 mM MOPS pH 7.2, 1 mM Sodium Phosphate (dibasic), 5 mM NaCl) per fraction and incubated for 2 hours at 4° C. with pre-washed (4 times with IAP buffer) agarose beads bound to acetyl-lysine motif antibody. Peptide bound beads were washed 4 times with ice-cold PBS followed by elution with 100 ul of 0.15% TFA. Eluents were desalted using C18 stage-tips and eluted with 50% ACN and dried down. Acetylpeptides were-suspended in 7 ul of 0.1% FA and 3% ACN and 4 ul was injected per run.

LC-MS/MS for TMT Global Proteome, Phosphoproteome and Acetylome Analysis

Liquid Chromatography:

Online separation was done with a nanoflow Proxeon EASY-nLC 1200 UHPLC system (Thermo Fisher Scientific). In this set up, the LC system, column, and platinum wire used to deliver electrospray source voltage were connected via a stainless-steel cross (360 μm, IDEX Health & Science, UH-906x). The column was heated to 50° C. using a column heater sleeve (Phoenix-ST) to prevent over-pressuring of columns during UHPLC separation. Each peptide fraction, ˜1 μg (based on protein-level BCA prior to digestion with uniformly-distributed fractionation presumed), the equivalent of 12% of each global proteome sample in a 2 ul injection volume, or 50% of each phosphoproteome sample in a 4 μl injection volume, was injected onto an in-house packed 22 cm×75 μm diameter C18 silica picofrit capillary column (1.9 μm ReproSil-Pur C18-AQ beads, Dr. Maisch GmbH, r119.aq; Picofrit 10 um tip opening, New Objective, PF360-75-10-N-5). Mobile phase flow rate was 200 nL/min, comprised of 3% acetonitrile/0.1% formic acid (Solvent A) and 90% acetonitrile/0.1% formic acid (Solvent B). The 110-minute LC-MS/MS method consisted of a 10-min column-equilibration procedure; a 20-min sample-loading procedure; and the following gradient profile: (min:% B) 0:2; 1:6; 85:30; 94:60; 95;90; 100:90; 101:50; 110:50 (the last two steps at 500 nL/min flow rate). For acetylome analysis, same LC and column setup was used with the exception of gradient length. A 260-minute LC-MS/MS method was used with the following gradient profile: (min:% B) 0:2; 1:6; 235:30; 244:60; 245;90; 250:90; 251:50; 260:50 (the last two steps at 500 nL/min flow rate).

Mass Spectrometry for Proteome Analysis:

Samples were analyzed with a benchtop Q Exactive HF-X mass spectrometer (Thermo Fisher Scientific) equipped with a nanoflow ionization source (James A. Hill Instrument Services, Arlington, Mass.). Data-dependent acquisition was performed using Q Exactive HF-X Orbitrap v 2.9 software in positive ion mode at a spray voltage of 1.5 kV. MS1 Spectra were measured with a resolution of 60,000, an AGC target of 3e6 and a mass range from 350 to 1800 m/z. The data-dependent mode cycle was set to trigger MS/MS on up to the top 20 most abundant precursors per cycle at an MS2 resolution of 45,000, an AGC target of 5e4, an isolation window of 0.7 m/z, a maximum injection time of 105 msec, and an HCD collision energy of 31%. Peptides that triggered MS/MS scans were dynamically excluded from further MS/MS scans for 45 sec. Peptide match was set to preferred for monoisotopic peak determination, and charge state screening was enabled to only include precursor charge states 2-6, with an intensity threshold of 9.5e4. Advanced precursor determination feature (APD) (Myers et al., 2018) was turned off using a software patch provided to us by Thermo Fisher Scientific allowing us to turn APD off in the tune file, Tune version 2.9.0.2926 (later versions of Exactive Tune 2.9 sp2 for the HFX have this option as standard).

Mass Spectrometry for Phosphoproteome and Acetylome Analysis:

Samples were analyzed with a benchtop Orbitrap Fusion Lumos mass spectrometer (Thermo Fisher Scientific) equipped with a NanoSpray Flex NG ion source. Data-dependent acquisition was performed using Xcalibur Orbitrap Fusion Lumos v3.0 software in positive ion mode at a spray voltage of 1.8 kV. MS1 Spectra were measured with a resolution of 60,000, an AGC target of 4e5 and a mass range from 350 to 1800 m/z. The data-dependent mode cycle time was set at 2 seconds with a MS2 resolution of 50,000, an AGC target of 6e4, an isolation window of 0.7 m/z, a maximum injection time of 105 msec, and an HCD collision energy of 36%. Peptide mode was selected for monoisotopic peak determination, and charge state screening was enabled to only include precursor charge states 2-6, with an intensity threshold of 1e4. Peptides that triggered MS/MS scans were dynamically excluded from further MS/MS scans for 45 sec, with a +/−10 ppm mass tolerance. Perform dependent scan on single charge state per precursor only was enabled for proteome analysis and disabled for acetylome analysis.

Immunohistochemistry

Total ALK and phospho-ALK (Y1507) immunostainings were performed on representative tumor and matched benign adjacent tissues from the available cases which contained ALK, ROS1 or RET gene fusions. The antibodies used include anti-ALK primary rabbit monoclonal antibody (ALK(D5F3) XP, Cell Signaling Technology, cat #3633 at 1 in 250 dilution) and anti-phospho ALK rabbit monoclonal antibody (D6F1V, Cell Signaling Technology, cat #14678 at 1:500 dilution). Briefly, 5-micron formalin fixed, paraffin sections were rehydrated and a heat induced epitope retrieval was performed with citrate buffer (pH 6). Incubation with the respective antibodies were carried out overnight at 4 degrees followed by buffer washes. For total-ALK, post incubation with secondary antibody was done for half an hour and for phospho-ALK (Y1507), post incubation was done initially with amplifier antibody (goat anti-rabbit IgG) for 15 minutes followed by secondary for 30 minutes. After buffer washes for total-Alk the signal was developed using DAB Peroxidase Substrate Kit (SK-4100; Vector laboratories) and for phospho-ALK using equal volumes of ImmPACT DAB EqV Reagent 1 (chromogen) and ImmPACT DAB EqV Reagent 2 (Diluent) for 5 minutes. Slides were counterstained with 50% Hematoxylin for 2 minutes, dehydrated, and cover-slipped. IHC was assessed for nuclear and cytoplasmic expression on tumor cells and the background was assessed in NATs (R.M. and R.M.).

Quantification and Statistical Analysis

Genomic Data Analysis

Copy Number Calling

Copy-number analysis was performed jointly leveraging both whole-genome sequencing (WGS) and whole-exome sequencing data of the tumor and germline DNA, using CNVEX (https://github.com/mctp/cnvex). CNVEX uses whole-genome aligned reads to estimate coverage within fixed genomic intervals, and whole-genome and whole-exome variant calls to compute B-allele frequencies at variable positions (we used TNScope germline calls). Coverages were computed in 10 kb bins, and the resulting log coverage ratios between tumor and normal samples were adjusted for GC bias using weighted LOESS smoothing across mappable and non-blacklisted genomic intervals within the GC range 0.3-0.7, with a span of 0.5 (the target, blacklist, and configuration files are provided with CNVEX). The adjusted log coverage-ratios (LR) and B-allele frequencies (BAF) were jointly segmented by custom algorithm based on Circular Binary Segmentation (CBS). Alternative probabilistic algorithms were implemented in CNVEX, including algorithms based on recursive binary segmentation (RBS) (Gey and Lebarbier, 2008), and dynamic programming (Bellman, 1961), as implemented in the R-package jointseg (Pierre-Jean et al., 2014). For the CBS-based algorithm, first LR and mirrored BAF were independently segmented using CBS (parameters alpha=0.01, trim=0.025) and all candidate breakpoints collected. The resulting segmentation track was iteratively “pruned” by merging segments which had similar LR, BAFs and short lengths. For the RBS and DP based algorithms joint-breakpoints were “pruned” using a statistical model selection method (Lebarbier, 2005). For the final set of CNV segments, we chose the CBS-based results as they did not require specifying a prior on the number of expected segments (K) per chromosome arm, were robust to unequal variances between the LR and BAF tracks, provided empirically the best to the underlying data.

Somatic Variant Calling

We called somatic variants for GDC aligned WXS bams by using the somaticwrapper pipeline (https://github.com/ding-lab/somaticwrapper), which includes four different callers, i.e., Strelka v.2 (Saunders et al., 2012), MUTECT v1.7 (Cibulskis et al., 2013), VarScan v.2.3.8 (Koboldt et al., 2012), and Pindel v.0.2.5 (Ye et al., 2009). We kept SNVs called by any 2 callers among MUTECT v1.7, VarScan v.2.3.8, and Strelka v.2 and indels called by any 2 callers among VarScan v.2.3.8, Strelka v.2, and Pindel v.0.2.5. For the merged SNVs and indels, we applied a 14× and 8× coverage cutoff for tumor and normal, separately. We also filtered SNVs and indels by a minimal variant allele frequency (VAF) of 0.05 in tumors and a maximal VAF of 0.02 in normal samples. Finally, we filtered any SNV which was within 10 bp of an indel found in the same tumor sample.

In step 13 of somaticwrapper pipeline, it combined adjacent SNVs into DNP (di nucleotide polymorphisms) by using COCOON: As input, COCOON takes a MAF file from standard variant calling pipeline. First, it extracts variants within a 2 bp window as DNP candidate sets. Next, if the corresponding BAM files used for variant calling are available, it extracts the reads (denoted as n_t) spanning all candidate DNP locations in each variant set, and then counts the number of reads with all the co-occurring variants (denoted as n_c) to calculate co-occurrence rate (r_c=n_c/n_t); If r_c≥0.8, the nearby SNVs will be combined into DNP and it also updates annotation for the DNPs from the same codon based on the transcript and coordinates information in the MAF file. Among a total 32,624 somatic variants identified from somaticwrapper pipeline, there are 442 DNPs, in which 223 are falling in the dominated smoking-related DNP type

(CC->AA or GG->TT).

GISTIC and MutSig Analysis

Genomic Identification of Significant Targets in Cancer (GISTIC2.0) algorithm (Mermel et al., 2011) was used to identify significantly amplified or deleted focal-level and arm level events, with q values that were smaller than 0.25 considered significant. The following parameters were used:
Amplification Threshold=0.1
Deletion Threshold=0.1
Cap Values=1.5
Broad Length Cutoff=0.98
Remove X-Chromosome=0
Confidence Level=0.99
Join Segment Size=4
Arm Level Peel Off=1
Maximum Sample Segments=2000
Gene GISTIC=1

Each gene of every sample is assigned a thresholded copy number level that reflects the magnitude of its deletion or amplification. These are integer values ranging from −2 to 2, where 0 means no amplification or deletion of magnitude greater than the threshold parameters described above. Amplifications are represented by positive numbers: 1 means amplification above the amplification threshold; 2 means amplification larger than the arm level amplifications observed in the sample. Deletions are represented by negative numbers: −1 means deletion beyond the threshold; −2 means deletions greater than the minimum arm-level copy number observed in the sample.

The somatic variants were filtered through a panel of normals to remove potential sequencing artifacts and undetected germline variants. MutSig2CV (Lawrence et al. 2014) was run on these filtered results to evaluate the significance of mutated genes and estimate mutation densities of samples. These results were constrained to genes in the Cancer Gene Census (Sondka et al. 2018), with false discovery rates (q values) recalculated. Genes of q value <0.1 were declared significant.

RNA Quantification and Analysis

RNA Quantification

Transcriptome data have been analyzed as described previously (Robinson et al., 2017), using the Clinical RNA-seq Pipeline (CRISP) developed at the University of Michigan (https://github.com/mcieslik-mctp/crisp-build). Briefly, raw sequencing data was trimmed, merged using BBMap, and aligned to GRCh38 using STAR. The resulting BAM files were analyzed for expression using feature counts against a transcriptomic reference based on Gencode 26. The resulting gene-level counts for protein-coding genes were upper-quartile normalized and transformed into RPKMs using edgeR and further log 2 transformed. Genes quantified in fewer than 30% of all samples were removed from the data matrix. Data rows of redundant gene symbols were aggregated by calculating the average log 2(RPKM).

For integrative multi-omics subtyping we normalized each gene by the median log 2(RPKM) across all tumors (gene-centering) and applied the same per-sample normalization strategy used to normalize proteomics data tables (see below: Two-component normalization of TMT ratio distributions).

miRNA-Seq Data Analysis and Unsupervised Clustering

miRNA-seq FASTQ files were downloaded from CPTAC GDC API (https://docs.gdc.cancer.gov). TPM (Transcripts per million) values of mature miRNA and precursor miRNA were reported after adapter trimming, quality check, alignment, annotation, reads counting. (https://github.com/ding-lab/CPTAC_miRNA/blob/master/cptac_mima_analysis.md). The mature miRNA expression was calculated irrespective of its gene of origin by summing the expression from its precursor miRNAs.

Unsupervised miRNA expression subtype identification was performed on mature miRNAs expression (log 2 TPM) from 106 LUAD patients using Louvain clustering. (https://doi.org/10.5281/zenodo.595481). The expression of top 50 differentially expressed miRNAs from each miRNA-based subtype was shown in the heatmap. For consistency, miRNA expression, RNA expression and protein expression were scaled to 0-1.

Proteomics Data Analysis: Protein-Peptide Identification, Phosphosite/Acetyl Site Localization, and Quantification

Spectrum Quality Filtering and Database Searching

All MS data were interpreted using the Spectrum Mill software package v7.0 pre-release (Agilent Technologies, Santa Clara, Calif.) co-developed by Karl Clauser of the Carr laboratory (https://www.broadinstitute.org/proteomics). Similar MS/MS spectra acquired on the same precursor m/z within +/−45 sec were merged. MS/MS spectra were excluded from searching if they failed the quality filter by not having a sequence tag length >0 (i.e., minimum of two masses separated by the in-chain mass of an amino acid) or did not have a precursor MH+ in the range of 800-6000. MS/MS spectra were searched against a RefSeq-based sequence database containing 41,457 proteins mapped to the human reference genome (hg38) obtained via the UCSC Table Browser (https://genome.ucsc.edu/cgi-bin/hgTables) on Jun. 29, 2018, with the addition of 13 proteins encoded in the human mitochondrial genome, 264 common laboratory contaminant proteins, and 553 non-canonical small open reading frames. Scoring parameters were ESI-QEXACTIVE-HCD-v2, for whole proteome datasets, and ESI-QEXACTIVE-HCD-v3, for phosphoproteome datasets. All spectra were allowed +/−20 ppm mass tolerance for precursor and product ions, 30% minimum matched peak intensity, and “trypsin allow P” enzyme specificity with up to 4 missed cleavages. Allowed fixed modifications included carbamidomethylation of cysteine and selenocysteine. TMT labeling was required at lysine, but peptide N-termini were allowed to be either labeled or unlabeled. Allowed variable modifications for whole proteome datasets were acetylation of protein N-termini, oxidized methionine, deamidation of asparagine, hydroxylation of proline in PG motifs, pyro-glutamic acid at peptide N-terminal glutamine, and pyro-carbamidomethylation at peptide N-terminal cysteine with a precursor MH+ shift range of −18 to 97 Da. For the phosphoproteome dataset the allowed variable modifications were revised to allow phosphorylation of serine, threonine, and tyrosine, allow deamidation only in NG motifs, and disallow hydroxylation of proline with a precursor MH+ shift range of −18 to 272 Da. For the acetylome dataset the allowed variable modifications were revised to allow acetylation of lysine, allow deamidation only in NG motifs, and disallow hydroxylation of proline with a precursor MH+ shift range of −400 to 70 Da.

PSM Quality Filtering, Protein Grouping, and Localization of Phosphosites and Acetylsites

Identities interpreted for individual spectra were automatically designated as confidently assigned using the Spectrum Mill autovalidation module to use target-decoy based false discovery rate (FDR) estimates to apply score threshold criteria. For the whole proteome dataset thresholding was done in 3 steps: at the peptide spectrum match (PSM) level, the protein level for each TMT-plex, and the protein level for all 25 TMT-plexes. For the phosphoproteome and acetylome datasets thresholding was done in two steps: at the PSM and variable modification (VM) site levels.

In step 1 for all datasets, PSM-level autovalidation was done first and separately for each TMT-plex experiment consisting of either 25 LC-MS/MS runs (whole proteome), 13 LC-MS/MS runs (phosphoproteome), or 4 LC-MS/MS runs (acetylome) using an auto-thresholds strategy with a minimum sequence length of 7; automatic variable range precursor mass filtering; and score and delta Rank1-Rank2 score thresholds optimized to yield a PSM-level FDR estimate for precursor charges 2 through 4 of <0.8% for each precursor charge state in each LC-MS/MS run. To achieve reasonable statistics for precursor charges 5-6, thresholds were optimized to yield a PSM-level FDR estimate of <0.4% across all runs per TMT-plex experiment (instead of per each run), since many fewer spectra are generated for the higher charge states.

In step 2 for the whole proteome dataset, protein-polishing autovalidation was applied separately to each TMTplex experiment to further filter the PSMs using a target protein-level FDR threshold of zero. The primary goal of this step was to eliminate peptides identified with low scoring PSMs that represent proteins identified by a single peptide, so-called “one-hit wonders”. After assembling protein groups from the autovalidated PSMs, protein polishing determined the maximum protein level score of a protein group that consisted entirely of distinct peptides estimated to be false-positive identifications (PSMs with negative delta forward-reverse scores). PSMs were removed from the set obtained in the initial peptide-level autovalidation step if they contributed to protein groups that had protein scores below the maximum false-positive protein score. Step 3 was then applied, consisting of protein-polishing autovalidation across all TMT plexes together using the protein grouping method expand subgroups, top uses shared to retain protein subgroups with either a minimum protein score of 25 or observation in at least 4 TMT plexes. The primary goal of this step was to eliminate low scoring proteins that were infrequently detected in the sample cohort. As a consequence of these two protein-polishing steps each identified protein reported in the study was comprised of multiple peptides, unless a single excellent scoring peptide was the sole match and that peptide was observed in at least 4 TMT-plexes. In calculating scores at the protein level and reporting the identified proteins, peptide redundancy was addressed in Spectrum Mill as follows. The protein score was the sum of the scores of distinct peptides. A distinct peptide was the single highest scoring instance of a peptide detected through an MS/MS spectrum. MS/MS spectra for a particular peptide may have been recorded multiple times (e.g. as different precursor charge states, in adjacent bRP fractions, modified by deamidation at Asn or oxidation of Met, or with different phosphosite localization), but were still counted as a single distinct peptide. When a peptide sequence of >8 residues was contained in multiple protein entries in the sequence database, the proteins were grouped together and the highest scoring one and its accession number were reported. In some cases when the protein sequences were grouped in this manner there were distinct peptides that uniquely represent a lower scoring member of the group (isoforms, family members, and different species). Each of these instances spawned a subgroup. Multiple subgroups were reported, counted towards the total number of proteins, and given related protein subgroup numbers (e.g. 3.1 and 3.2 for group 3, subgroups 1 and 2). For the whole proteome datasets the above criteria yielded false discovery rates (FDR) for each TMT-plex experiment of <0.6% at the peptide-spectrum match level and <0.8% at the distinct peptide level. After assembling proteins with all the PSMs from all the TMT-plex experiments together the aggregate FDR estimates were 0.57% at the at the peptide-spectrum match level, 2.6% at the distinct peptide level, and <0.01% (1/11,015) at the protein group level. Since the protein level FDR estimate neither explicitly required a minimum number of distinct peptides per protein nor adjusted for the number of possible tryptic peptides per protein, it may underestimate false positive protein identifications for large proteins observed only on the basis of multiple low scoring PSMs.

In step 2 for the phosphoproteome and acetylome datasets, variable modification (VM) site polishing autovalidation was applied across all 25 TMT plexes to retain all VM-site identifications with either a minimum id score of 8.0 or observation in at least 4 TMT plexes. The intention of the VM site polishing step is to control FDR by eliminating unreliable VM site-level identifications, particularly low scoring VM sites that are only detected as low scoring peptides that are also infrequently detected across all of the TMT plexes in the study. In calculating scores at the VM-site level and reporting the identified VM sites, redundancy was addressed in Spectrum Mill as follows. A VM-site table was assembled with columns for individual TMT-plex experiments and rows for individual VM-sites. PSMs were combined into a single row for all non-conflicting observations of a particular VM-site (e.g. different missed cleavage forms, different precursor charges, confident and ambiguous localizations, and different sample-handling modifications). For related peptides neither observations with a different number of VM-sites nor different confident localizations were allowed to be combined. Selecting the representative peptide from the combined observations was done such that once confident VM-site localization was established, higher identification scores and longer peptide lengths were preferred. While a Spectrum Mill identification score was based on the number of matching peaks, their ion type assignment, and the relative height of unmatched peaks, the VM site localization score was the difference in identification score between the top two localizations. The score threshold for confident localization, >1.1, essentially corresponded to at least 1 b or y ion located between two candidate sites that has a peak height >10% of the tallest fragment ion (neutral losses of phosphate from the precursor and related ions as well as immonium and TMT reporter ions were excluded from the relative height calculation). The ion type scores for b-H₃PO₄, y-H₃PO₄, b-H₂O, and y-H₂O ion types were all set to 0.5. This prevented inappropriate confident localization assignment when a spectrum lacked primary b or y ions between two possible sites but contained ions that could be assigned as either phosphate-loss ions for one localization or water loss ions for another localization. VM-site polishing yielded 65,103 phosphosites with an aggregate FDR of 0.74% at the phosphosite level. In aggregate, 71% of the reported phosphosites in this study were fully localized to a particular serine, threonine, or tyrosine residue. VM-site polishing yielded 13,480 acetylsites with an aggregate FDR of 0.89% at the acetylsite level. In aggregate, 99% of the reported acetylsites in this study were fully localized to a particular lysine residue.

Quantitation Using TMT Ratios

Using the Spectrum Mill Protein/Peptide Summary module a protein comparison report was generated for the proteome dataset using the protein grouping method expand subgroups, top uses shared (SGT). For the phosphoproteome and acetylome datasets a Variable Modification site comparison report limited to either phospho or acetyl sites, respectively, was generated using the protein grouping method unexpand subgroups. Relative abundances of proteins and VM-sites were determined in Spectrum Mill using TMT reporter ion intensity ratios from each PSM. TMT reporter ion intensities were corrected for isotopic impurities in the Spectrum Mill Protein/Peptide summary module using the afRICA correction method which implements determinant calculations according to Cramer's Rule (Shadforth et al., 2005) and correction factors obtained from the reagent manufacturer's certificate of analysis (https://www.thermofisher.com/order/catalog/product/90406) for TMT10_lot number SE240163. A protein-level, phosphosite-level, or acetylsite-level TMT ratio is calculated as the median of all PSM level ratios contributing to a protein subgroup, phosphosite, or acetylsite. PSMs were excluded from the calculation that lacked a TMT label, had a precursor ion purity <50% (MS/MS has significant precursor isolation contamination from co-eluting peptides), or had a negative delta forward-reverse identification score (half of all false-positive identifications). Lack of TMT label led to exclusion of PSMs per TMT plex with a range of 3.5 to 4.6% for the proteome, 1.2 to 3.9% for the phosphoproteome, and 1.3 to 6.6% for the acetylome datasets. Low precursor ion purity led to exclusion of PSMs per TMT plex with a range of 1.2 to 1.6% for the proteome, 2.0 to 2.9% for the phosphoproteome, and 4.6 to 7.5% for the acetylome datasets.

Two-Component Normalization of TMT Ratio Distributions

It was assumed that for every sample there would be a set of unregulated proteins or phosphosites that have abundance comparable to the reference sample. In the normalized sample, these proteins, phosphosites, or acetylsites should have a log TMT ratio centered at zero. In addition, there were proteins, phosphosites, and acetylsites that were either up- or down-regulated compared to the reference. A normalization scheme was employed that attempted to identify the unregulated proteins and phosphosites, and centered the distribution of these log-ratios around zero in order to nullify the effect of differential protein loading and/or systematic MS variation. A 2-component Gaussian mixture model-based normalization algorithm was used to achieve this effect. The two Gaussians (μi1,1) and N(μi2,σi2) for a sample i were fitted and used in the normalization process as follows: the mode mi of the log-ratio distribution was determined for each sample using kernel density estimation with a Gaussian kernel and Shafer-Jones bandwidth. A two-component Gaussian mixture model was then fit with the mean of both Gaussians constrained to be mi, i.e., μi1=μi2=mi. The Gaussian with the smaller estimated standard deviation σi=min (\hat{σ}1i,\hat{σ}2 i) was assumed to represent the unregulated component of proteins/phosphosites/acetylsites, and was used to normalize the sample. The sample was standardized using (mi,) by subtracting the mean mi from each protein/phosphosite/acetylsite and dividing by the standard deviation σi.

Comparative Reference Sample—Xenograft Considerations for Identification and Quantitation

To better dissect the tumor/stroma (human/mouse) origin of orthologous proteins in the CompRef xenograft samples, a few divergences were made in the data analysis described above. The sequence database used for searching MS/MS spectra was expanded to include 30,608 mouse proteins, mapped to the mouse reference genome (mm10) obtained via the UCSC Table Browser (https://genome.ucsc.edu/cgi-bin/hgTables) on the same date as the corresponding human reference genome Jun. 29, 2018, along with the addition of 13 proteins encoded in the mouse mitochondrial genome. For the proteome dataset autovalidation step 3 consisted of protein-polishing autovalidation across all 4 TMT plexes together used the protein grouping method unexpand subgroups, to retain protein groups with either a minimum protein score of 25 or observation in at least 2 TMT plexes. The subsequent protein comparison report generated for the proteome dataset employed the subgroup-specific (SGS) protein grouping option, which omitted peptides that are shared between subgroups, and included only subgroup specific peptide sequences toward each subgroup's count of distinct peptides and protein level TMT quantitation. If evidence for BOTH human and mouse peptides from an orthologous protein were observed, then peptides that cannot distinguish the two (shared) were ignored. However, the peptides shared between species were retained if there was specific evidence for only one of the species, thus yielding a single subgroup attributed to only the single species consistent with the specific peptides. Furthermore, if all peptides observed for a protein group were shared between species, thus yielding a single subgroup composed of indistinguishable species, then all peptides were retained. For the proteome dataset, only PSM's from subgroup-specific peptide sequences contributed to the protein level quantitation. A protein detected with all contributing PSM's shared between human and mouse was considered to be human. For the phosphoproteome and acetylome datasets, a phosphosite or acetylsite was considered to be mouse if the contributing PSM's were distinctly mouse and human if they were either distinctly human or shared between human and mouse.

Systems Biology Analysis

Sample Exclusion

We wanted to ensure that poor quality or questionable samples are not included in the final dataset. To achieve this goal, we performed principal component analysis (PCA) on the RNAseq, global proteome and phosphosite expression data. In the input to PCA, we excluded any genes, proteins and phosphosites (in the respective datasets) missing in 50% or more of the samples. For each dataset, we plotted the 95% confidence ellipse in the PC1 vs PC2 plot for the tumor and normal groups. Any samples falling outside these ellipses were deemed to be outliers. Samples that were outliers in all three datasets (RNAseq, proteome and phosphosite) and had inconsistent pathology reviews were excluded. Only sample C3N.00545 satisfied all exclusion criteria was removed from the final dataset.

Dataset Filtering

Genes (RNAseq), proteins (global proteome), phosphosites and acetyl sites present in fewer than 30% of samples (i.e., missing in >70% of samples) were removed from the respective datasets. Furthermore:
Proteins were required to have at least two observed TMT ratios in >25% of samples in order to be included in the proteome dataset. Phosphosites and acetyl sites were required to have at least one observed TMT ratio in >25% of samples.
Proteins, phosphosites and acetyl sites were required to have TMT ratios with an overall standard deviation >0.5 across all the samples where they were observed. This ensures that a small number of proteins, phosphosites and acetyl sites that do not vary much over the set of samples are excluded to minimize noise.

Replicate samples in the dataset are merged by taking the mean of the respective expression values or ratios.

Some of the filtering steps were modified for specific analysis in the study. For many of the marker selection and gene set enrichment analyses, at least 50% of samples were required to have non-missing values for proteins/phosphosites/acetyl sites, since missing values are imputed, and excessive missing values can result in poor imputation. Alternate filtering has been noted in descriptions of the relevant methods.

Unsupervised Subtyping using Non-Negative Matrix Factorization (NMF)

We used non-negative matrix factorization (NMF) implemented in the NMF R-package (Gaujoux and Seoighe, 2010) to perform unsupervised clustering of tumor samples and to identify proteogenomic features (proteins, phosphosites, acetylation sites and RNA transcripts) that show characteristic expression patterns for each cluster. Briefly, given a factorization rank k (where k is the number of clusters), NMF decomposes a p×n data matrix V into two matrices Wand H such that multiplication of Wand H approximates V. Matrix H is a k×n matrix whose entries represent weights for each sample (1 to N) to contribute to each cluster (1 to k), whereas matrix W is a p×k matrix representing weights for each feature (1 to p) to contribute to each cluster (1 to k). Matrix H was used to assign samples to clusters by choosing the k with maximum score in each column of H. For each sample we calculated a cluster membership score as the maximal fractional score of the corresponding column in matrix H. We defined a “cluster core” as the set of samples with cluster membership score >0.5. Matrix W containing the weights of each feature to a certain cluster was used to derive a list of representative features separating the clusters using the method proposed in (Kim and Park, 2007).

Preprocessing of Data Tables:

To enable integrative multi-omics clustering we enforced all data types (and converted if necessary) to represent ratios to either a common reference measured in each TMT plex (proteome, phosphoproteome, acetylome) or an in-silico common reference calculated as the median abundance across all samples (mRNA, see “RNA Quantification”). All data tables where then concatenated and filtered to contain a maximum of 30% missing values across all tumors. The remaining missing values were imputed via k-nearest neighbor (kNN) imputation implemented in the impute R-package (DOI: 10.18129/B9.bioc.impute) using the 5 nearest neighbors. To remove uninformative features from the dataset prior to NMF clustering we removed features with the lowest standard deviation (bottom 5^{th} percentile) across all samples. Each row in the data matrix was further scaled and standardized such that all features from different data types were represented as z-scores.

Since NMF requires a non-negative input matrix we converted the z-scores in the data matrix into a non-negative matrix as follows:
1) Create one data matrix with all negative numbers zeroed.
2) Create another data matrix with all positive numbers zeroed and the signs of all negative numbers removed.
3) Concatenate both matrices resulting in a data matrix twice as large as the original, but with positive values only and zeros and hence appropriate for NMF.

Determination of Factorization Rank:

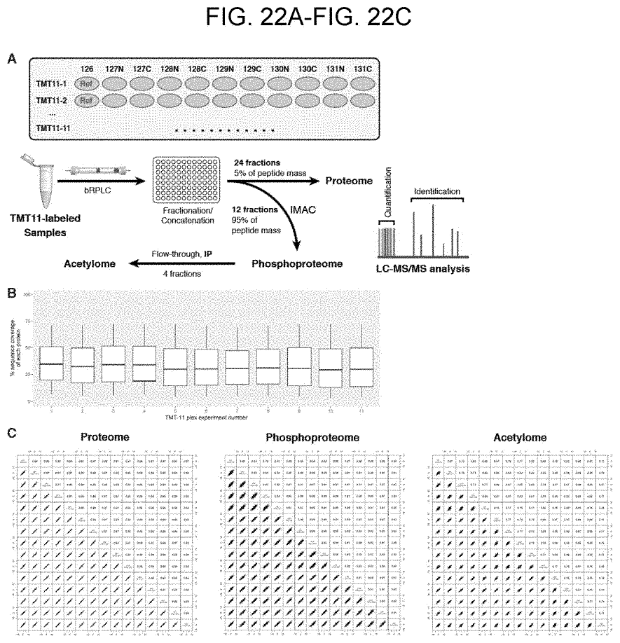

The resulting matrix was then subjected to NMF analysis leveraging the NMF R-package (Gaujoux and Seoighe, 2010) and using the factorization method described in (Brunet et al., 2004). To determine the optimal factorization rank k (number of clusters) for the multi-omic data matrix we tested a range of clusters between k=2 and 8. For each k we factorized matrix V using 50 iterations with random initializations of Wand H. To determine the optimal factorization rank we calculated cophenetic correlation coefficients measuring how well the intrinsic structure of the data is recapitulated after clustering and chose the k with maximal cophenetic correlation for cluster numbers between k=3 and 8. (FIG. 22G).

Final NMF Clustering:

Having determined the optimal factorization rank k, in order to achieve robust factorization of the multi-omics data matrix V, we repeated the NMF analysis using 200 iterations with random initializations of Wand H and performed the partitioning of samples into clusters as described above. Due to the non-negative transformation applied to the z-scored data matrix as described above matrix W of feature weights contained two separate weights for positive and negative z-scores of each features, respectively. In order to revert the non-negative transformation and to derive a single signed weight for each feature, we first normalized each row in matrix W by dividing by the sum of feature weights in each row, aggregated both weights per feature and cluster by keeping the maximal normalized weight and multiplication with the sign of the z-score the initial data matrix. Thus, the resulting transformed version of matrix W_{signed} contained signed cluster weights for each feature in the input matrix.

Functional Characterization of Clustering Results by Single Sample Gene Set Enrichment Analysis (ssGSEA):

For each cluster we calculated normalized enrichment scores (NES) of cancer-relevant gene sets by projecting the matrix of signed multi-omic feature weights (W_{signed}) onto hallmark pathway gene sets (Liberzon et al., 2015) using ssGSEA (Barbie et al., 2009). To derive a single weight for each gene measured across multiple omics data types (protein, RNA, phosphorylation site, acetylation site) we retained the weight with maximal absolute amplitude. We used the ssGSEA implementation available on https://github.com/broadinstitute/ssGSEA2.0 using the following parameters:
gene.set.database=“h.all.v6.2.symbols.gmt”
sample.norm.type=“rank”
weight=1
statistic=“area.under.RES”
output.score.type=“NES”
nperm=1000
global.fdr=TRUE
min.overlap=5
correl.type=“z.score”

Association Between Clusters and Clinical Variables:

To test the association of the resulting clusters to clinical variables we used Fisher's exact test (R function fisher.test) to test for overrepresentation in the set of samples defining the cluster core as described above. The following variables were included in the analysis: RNA.Expression.Subtype.TCGA, Region.of.Origin, Stage, Gender, Smoking. Status (self reported), TP53.mutation.status, KRAS.mutation.status, STK11.mutation.status, EGFR. mutation. status, KEAP1.mutation.status, ALK. fusion, CIMP.status.

Pipeline Implementation and Availability:

The entire workflow described above has been implemented as a module for Broad's Cloud platform Terra (https://app.terra.bio/). The docker containers encapsulating the source code and required R-packages for NMF clustering and ssGSEA have been submitted to Dockerhub (broadcptac/pgdac_mo_nmf:9, broadcptac/pgdac_ssgsea:5). The source code for ssGSEA is available on GitHub: https://github.com/broadinstitute/ssGSEA2.0.

Unsupervised Clustering by RNA Expression Data and Pathway Over-Representation Analysis

Starting with RNA expression data for the CPTAC LUAD cohort, the top 5,000 most variable genes were subjected to clustering using ConsensusClusterPlus (Wilkerson and Hayes, 2010). The resulting three clusters were mapped to TCGA RNA expression subtypes (Cancer Genome Atlas Research Network, 2014; Wilkerson et al., 2012) by associating enriched clinical features and gene mutations. The association of subtype and features were compared using Fisher's exact test.

To designate the representative pathways of multi-omics subtypes, we used the Wilcoxon rank sum test to select the top 250 differentially expressed features (mRNA, proteins and phosphosites), or features with p-value less than 0.05 (acetylsites) for each subtype. We then performed hierarchical clustering on these 1000 features and 573 acetylsites. Each set of clustered features underwent pathway enrichment analysis using Reactome (Fabregat et al., 2017). Pathways with p-value smaller than 0.05 were manually reviewed and highlighted in FIG. 1E. For visualization purpose, only the top 50 differentially expressed features for each subtype were displayed. In total, 200 features were shown for each data type in heatmap.

Fusion Detection and Analysis

Structural variants in WGS samples were called with Manta 1.3.2, retaining variants where sample site depth is less than 3× the median chromosome depth near one or both variant breakends, somatic score is greater than 30, and for small variants (<1000 bases) in the normal sample, the fraction of reads with MAPQ0 around either breakend does not exceed 0.4.

Fusions in RNA-Seq samples were called using three callers: STAR-Fusion, EricScript, and Integrate, with fusions reported by at least 2 callers or reported by STAR-Fusion being retained. Fusions present in the following databases were then excluded: 1) uncharacterized genes, immunoglobin genes, mitochondrial genes, etc., 2) fusions from the same gene or paralog genes, and 3) fusions reported in TCGA normal samples, GTEx tissues, and non-cancer cell studies. Finally, normal fusions were filtered out from the tumor fusions.

mRNA and Protein Correlation in Tumor and NATs

To compare mRNA expression and protein abundance across samples we focused on the RNA data with 18,099 genes, and global proteome with 10,316 quantified proteins respectively. Only genes or proteins with <50% NAs (missing values) were considered for the analysis, and protein IDs were mapped to gene names. In total, 9,616 genes common to both RNA and proteome data spanning 110 tumor samples were used in the analysis. The analyses were carried out on normalized data—RNA data were log 2 transformed, upper quartile normalized RPKM values, which were median-centered by row (i.e. gene); proteome data was two-component normalize as described earlier. Correlation was calculated by Spearman's correlation method using cor.test (Bioconductor, version 3.5.2) function in R. Both correlation coefficient and p-value were computed. Further, adjusted p-value was calculated using the Benjamini-Hochberg procedure. Similarly, mRNA-protein correlation among NAT samples were carried out with overlapping genes over the 101 NAT samples.

To identify genes that reverse their direction in tumors relative to NATs, i.e negative RNA-Protein correlation in NATs compared to positive RNA-protein correlation in tumors or vice-versa, we selected significant (Benjamini-Hochberg multiple test, FDR <0.1) mRNA-protein pairs in NATs and Tumors respectively, that changed direction of correlation (negative correlation to positive correlation or vice-versa) Significant genes identified in the global tumor-NAT comparison and individual mutant categories were merged together and shown in FIG. 3A with corresponding correlation coefficient. For paired tumor-NAT analysis, we considered 101 out of 110 samples for which, we have paired NATs, out of which 52, 36, 29, 17 samples had TP53, EGFR, KRAS and STK11 mutation respective.

CNA Driven Cis and Trans Effects

Correlations between copy number alterations (CNA) and RNA, proteome, phosphoproteome and acetylome (with proteome and PTM data mapped to genes, by choosing the most variable protein isoform/PTM site as the gene-level representative) were determined using Pearson correlation of common genes present in CNA-RNA-proteome (9,341 genes), CNA-RNA-phosphoproteome (5,244 genes) and CNA-RNA-acetylome (1,313 genes). In addition, p-values (corrected for multiple testing using Benjamini-Hochberg FDR) for assessing the statistical significance of the correlation values were also calculated. CNA trans-effects for a given gene were determined by identifying genes with statistically significant (FDR <0.05) positive or negative correlations.

CMAP Analysis

Candidate genes driving response to copy number alterations were identified using large-scale Connectivity Map (CMAP) queries. The CMAP (Lamb et al., 2006; Subramanian et al., 2017) is a collection of about 1.3 million gene expression profiles from cell lines treated with bioactive small molecules (˜20,000 drug perturbagens), shRNA gene knockdowns (˜4,300) and ectopic expression of genes. The CMAP dataset is available on GEO (Series GSE92742). For this analysis, we use the Level 5 (signatures from aggregating replicates) TouchStone dataset with 473,647 total profiles, containing 36,720 gene knock-down profiles, with measurements for 12,328 genes. See https://clue.io/GEO-guide for more information.

To identify candidate driver genes, proteome profiles of copy number-altered samples were correlated with gene knockdown mRNA profiles in the above CMAP dataset, and enrichment of up/down-regulated genes was evaluated. Normalized log 2 copy number values less than −0.3 defined deletion (loss), and values greater than +0.3 defined copy number amplifications (gains). In the copy number-altered samples (separately for CNA amplification and CNA deletion), the trans-genes (identified by significant correlation in “CNA driven cis and trans effects” above) were grouped into UP and DOWN categories by comparing the protein ratios of these genes to their ratios in the copy number neutral samples (normalized log 2 copy number between −0.3 and +0.3). The lists of UP and DOWN trans-genes were then used as queries to interrogate CMAP signatures and calculate weighted connectivity scores (WTCS) using the single-sample GSEA algorithm (Krug et al., 2018). The weighted connectivity scores were then normalized for each perturbation type and cell line to obtain normalized connectivity scores (NCS). See (Subramanian et al., 2017) for details on WTCS and NCS. For each query we then identified outlier NCS scores—a score is considered an outlier if it lies beyond 1.5 times the interquartile range of score distribution for the query. The query gene is a candidate driver if (i) the score outliers are statistically cis-enriched (Fisher test with BH-FDR multiple testing correction) and (ii) the gene has statistically significant and positive cis-correlation.

For a gene to be considered for inclusion in a CMAP query it needed to i) have a copy number change (amplification or deletion) in at least 15 samples; ii) have at least 20 significant trans genes; and iii) be on the list of shRNA knockdowns in the CMAP. 501 genes satisfied these conditions and resulted in 737 queries (CNA amplification and deletion combined) which were tested for enrichment. Twelve (12) candidate driver genes were identified with Fisher test FDR <0.1, using this process.

In order to ensure that the identified candidate driver genes were not a random occurrence, we performed a permutation test to determine how many candidate driver genes would be identified with random input (Mertins et al., 2016). For the 737 queries used, we substituted the bona-fide trans-genes with randomly chosen genes, and repeated the CMAP enrichment process. To determine FDR, each permutation run was treated as a Poisson sample with rate λ, counting the number of identified candidate driver genes. Given the small n (=10) and λ, a Score confidence interval was calculated (Barker, 2002) and the mid-point of the confidence interval used to estimate the expected number of false positives. Using 10 random permutations, we determined the overall false discovery rate to be FDR=0.13, with a 95% CI of (0.06, 0.19).

To identify how many trans-correlated genes for all candidate regulatory genes could be directly explained by gene expression changes measured in the CMAP shRNA perturbation experiments, knockdown gene expression consensus signature z-scores (knockdown/control) were used to identify regulated genes with α=0.05, followed by counting the number of trans-genes in this list of regulated genes.

To obtain biological insight into the list of candidate driver genes, we performed (i) enrichment analysis on samples with extreme CNA values (amplification or deletion) to identify statistically enriched sample annotation subgroups; and (ii) GSEA on cis/trans-correlation values to find enriched pathways.

Defining Cancer Associated Genes (CAG)

Cancer associated genes were compiled from list of genes defined by Bailey et al. (Bailey et al., 2018) and also cancer associated genes listed in Mertins et al. (Mertins et al., 2016) and adapted from Vogelsteain et al. (Vogelstein et al., 2013). A list of genes were generated.

DNA Methylation Data Preprocessing

Raw methylation image files were downloaded from CPTAC DCC (See data availability). We calculated and analyzed methylated (M) and unmethylated (U) intensities for LUAD samples as described previously (Fortin et al., 2014). We flagged locus as NA where probes did not meet a detection p-value of 0.01. Probes with MAF more than 0.1 were removed, and samples with more than 85% NA values were removed. Resulting beta values of methylation were utilized for subsequent analysis.

Classification of Samples with CpG Island Methylator Phenotype (CIMP)

To classify the tumor samples into the CpG island methylator phenotypes (CIMP), we performed consensus clustering of the methylation data. Specifically, we first generated the gene-level methylation score, by taking the averaged beta values of all probes harboring in the islands of promotor or 5 UTR regions of the gene. Then, we considered all genes that were hypermethylated in tumor, i.e. the gene-level methylation score >0.2, transformed the score into M-values (Du et al. 2010), normalized the transformed score, and then imputed the missing values as zero (mean of normalized data). Then we performed consensus clustering 1000 times, each taking 80% of the samples and all genes, and calculated the consensus matrix (probabilities of two samples clustering together) for each predetermined number of clusters K. In each value of K, we visualized the consensus matrix using hierarchical clustering with Pearson correlation as the distance metric. Finally, we determined the optimal number of clusters by considering the relative change in area under the consensus cumulative density function (CDF) curve. In the end, three distinct clusters were identified, one was hypermethylated with mean M value 0.3, and two were hypomethylated with mean M value −0.17 and −0.18, respectively. We labeled these three clusters as CIMP high, CIMP intermediate, and CIMP low groups.

iProFun Based Cis Association Analysis

We used iProFun, an integrative analysis tool to identify multi-omic molecular quantitative traits (QT) perturbed by DNA-level variations. In comparison with analyzing each molecular trait separately, the joint modeling of multi-omics data via iProFun provided enhanced power for detecting significant cis-associations shared across different omics data types; and it also achieved better accuracy in inferring cis-associations unique to certain type(s) of molecular trait(s). Specifically, we considered three functional molecular quantitative traits (mRNA expression levels, global protein abundances, and phosphopeptide abundances) for their associations with DNA methylation. We also adjusted for copy number alterations measured by log ratios, copy number alterations measured by b-allele frequency and somatic mutations when assessing the associations.

Data and Preprocessing:

We analyzed the tumor sample data from 101 cases in the current cohort collected by CPTAC. The mRNA expression levels measured with RNA-seq were available for 19,267 genes, the global protein abundance measurements were available for 10,316 genes, and the phosphopeptide abundance was available for 41,188 peptides from 7650 genes. The log ratios and b-allele frequency of copy number alterations using a segmentation method combining whole genome sequencing and whole exome sequencing was obtained for 19,267 and 19,267 genes, respectively. The DNA methylation levels (beta values) averaging the CpG islands located in the promoter and 5′ UTR regions were available for 16,479 genes. Somatic mutations were called using whole exome sequencing (See Somatic variant calling section above).

Proteomics and phosphoproteomics data were preprocessed with TMT outlier filtering and missing data imputation to increase number of features in the Cis Association Analysis. Due to the quantification of extreme small values on spectrum level, some extreme values with either positive or negative sign generated after log 2 transform of the TMT ratios. We believe those extreme values will have unstable impact on imputation of the data set since missing value are dependent with the observed values of same samples or same protein/phosphosite. To identify TMT ratio outliers with extreme values, we performed an inter TMT plex t-test for each individual protein/phosphosite. For each protein/phosphosite, the TMT ratios of samples within a single TMT-plex were compared against the TMT ratios of samples in all the other 24 TMT-plexes using a spearman two-sample t-test assuming equal variance. In proteomics 344 TMT were identified as outliers with inter TMT t-test p value lower than 10e-6, 3053 data points (0.122% of all observations) were removed from the data sets. And in phosphoproteomics 729 TMT were identified as outliers with inter TMT t-test p value lower than 10e-7, 6458 data points (0.088% of all observations) were removed from the data sets. Imputation was performed after outlier filtering. We selected proteins/phosphosites with missing rate less than 50%, and imputed with an algorithm tailored for proteomics data: We used DreamAI tool for imputation. (https://github.com/WangLab-MSSM/DreamAI)

The mRNA expression levels, global protein and phosphoprotein abundances were also normalized on each gene/phosphosite, to align the median to 0 and standard deviation to 1. To account for potential confounding factors, we considered age, gender, tumor purity, smoking status and country of origin. Tumor purity was determined using ESTIMATE (Yoshihara et al., 2013) from RNA-seq data.

iProFun Procedure:

The iProFun procedure was applied to a total of 4992 genes measured across all six data types (mRNA, global protein, phosphoprotein, CNA-Ir, CNA-baf, DNA methylation) for their cis regulatory patterns in tumors. Specifically, for the 4992 genes, we considered the following three regressions:

mRNA ˜CNV (Ir)+CNV (baf)+methy+covariates,

global ˜CNV (Ir)+CNV (baf)+methy+covariates, and

phosphor ˜CNV (Ir)+CNV (baf)+methy+covariates.

The association summary statistics of methy were applied to iProFun to call posterior probabilities of belonging to each of the eight possible configurations (“None”, “mRNA only” “global only”, “phosphor only” “mRNA & global”, “mRNA & phosphor”, “global & phospho” and “all three”) and to determine significance associations.

The significant genes need to pass three criteria: (1) the satisfaction of biological filtering procedure, (2) posterior probabilities >75%, and (3) empirical false discovery rate (eFDR)<5%. Specifically, we used the following biological filtering criterion for DNA methylations:only DNA methylations with negative associations with all the types of molecular QTs, were considered for significance call. Secondly, a significance was called only if the posterior probabilities >75% of a predictor being associated with a molecular QT, by summing over all configurations that are consistent with the association of interest. For example, the posterior probability of a DNA methylation being associated with mRNA expression levels was obtained by summing up the posterior probabilities in the following four association patterns—“mRNA only”, “mRNA & global”, “mRNA & phosphor” and “all three”, all of which were consistent with DNA methylation being associated with mRNA expression. Lastly, we calculated empirical FDR via 100 permutations per molecular QTs by shuffling the label of the molecular QTs, and requested empirical FDR (eFDR) <5% by selecting a minimal cutoff value of alpha such that 75%<alpha<100%. The eFDR is calculated by:

eFDR=(Averaged No. of genes with posterior probabilities >alpha in permuted data)/(Averaged No. of genes with posterior probabilities >alpha in original data).

Among all the genes whose methylation levels were significantly associated with all three molecular traits, FIG. 3E annotated those whose protein abundances significantly differ between tumor and NAT, protein clusters, and immune clusters.

Differential Abundance Analysis

RNA, protein, and PTM abundance were compared between mutated and wildtype tumor samples using the Wilcoxon rank-sum test. P-values were adjusted within a data type using the Benjamini-Hochberg method. Signed −log 10 (p-value) was used to indicate quantitative differences between mutated and wildtype tumors where signs “+” and “−” indicated upregulated and downregulated mRNA, proteins, phosphosites, and acetyl sites, respectively.

Deriving Mutation Based Signature

Non-negative matrix factorization algorithm (NMF) was used in deciphering mutation signatures in cancer somatic mutations stratified by 96 base substitutions in tri-nucleotide sequence contexts. To obtain a reliable signature profile, we used somaticwrapper to call mutations from WGS data. SignatureAnalyzer exploited the Bayesian variant of NMF algorithm and enabled an inference for the optimal number of signatures from the data itself at a balance between the data fidelity (likelihood) and the model complexity (regularization) (Kasar et al., 2015; Kim et al., 2016; Tan and Févotte, 2013). After decomposing into three signatures, signatures are compared against known signatures derived from COSMIC (Tate et al., 2019) and cosine similarity is calculated to identify best match.

Continuous Smoking Score

We also sought to integrate count of total mutations, t, percentage that are signature mutations, c, and count of DNPs, n, into a continuous score, 0<S <1, to quantify the degree of confidence that a sample is associated with smoking signature. We refer to these quantities as the data, namely D=C∩T∩N, and use A and A′ to indicate smoking signature and lack thereof, respectively. In a Bayesian framework, it is readily shown that a suitable form is S=1/(1+R), where R is the ratio of the joint probability of A′ and D to the joint probability of A and D. For example, the latter can be written P(A)·P(C|A)·P(T|A)·P(N|A) and the former similarly, where each term of the former is the complement of its respective term in this expression. Common risk statistics are invoked as priors, i.e. P(A)=0.9 (Walser et al., 2008).

We consider S to be a score because rigorous conditioned probabilities are difficult to establish. (For example, the data types themselves are not independent of one another and models using common distributions like the Poisson do not recapitulate realistic variances.) Instead, we adopt a data-driven approach of estimating contributions of each data type based on 2-point fitting of the extremes using shape functions based on the Gaussian error function, erf. The general model for data type G is P(G|A)=[x·erf (g/y)+1]/(x+2), with the resulting fitted values being the following: for total mutations G=T and (x,y)=(4028, 1000) when g=t; for percentage that are signature mutations G=C and (x,y)=(200, 50) when g=c; and for number of DNPs G=N and (x,y)=(30, 4) when g=n. Each of these parametric combinations adds significant weight above a linear contribution as the count for its respective data type increases above the average. For example, for g/y≈0.6, weights for each data type are around 50% higher than their corresponding linear values would be.

The shape function for T includes an expected-value correction for purity, u. (Correction for C is implicit, as it is a percentage of T.) Namely, assuming mutation-calling does not capture all mutations because of impurities, t is taken as the observed number of mutations divided by a purity shape function, f, where f 1. Although one might model f according to common characteristics of mutation callers, e.g. close to 100% sensitivity for pure samples and very low calling rate for low variant allele fractions (VAFs), the purity estimates for these data are based on RNA-Seq and are not highly correlated with total mutation counts. Consequently, we use a weaker, linear shape function, f=0.3·u+0.7, which does not strongly impact the adjustment of low-purity samples.

Determination of Sternness Score

Stemness scores were calculated as previously described (Malta et al., 2018).

To calculate the sternness scores based on mRNA expression, we built a predictive model using one-class logistic regression (OCLR) (Sokolov et al., 2016) on the pluripotent stem cell samples (ESC and iPSC) from the Progenitor Cell Biology Consortium (PCBC) dataset (Daily et al., 2017; Salomonis et al., 2016). For mRNA expression-based signatures, to ensure compatibility with the CPTAC LUAD cohort, we first mapped the gene names from Ensembl IDs to Human Genome Organization (HUGO), dropping any genes that had no such mapping. The resulting training matrix contained 12,945 mRNA expression values measured across all available PCBC samples. To calculate mRNA based sternness index (mRNASi) we used RPKM mRNA expression values for all CPTAC LUAD tumors and NAT samples. (uq-rpkm-log 2-NArm-row-norm.gct) to generate the mRNASi (mRNA sternness index) for each sample. We used the function TCGAanalyze_Stemness from the package TCGAbiolinks (Colaprico et al., 2016) and following our previously-described workflow (Ho et al., 1987), with “stemSig” argument set to PCBC_stemSig.

Immune Subtyping and Downstream Analysis

Identification Based on Cell Type Composition:

The abundance of 64 different cell types for lung tumors and NAT samples were computed via xCell (Aran et al., 2017). For this analysis, log 2 (UQ) RPKM expression values were utilized. The final score computed by xCell of different cell types for all tumor and NAT samples were generated. Consensus clustering was derived based on only cells which were detected in at least 5 patients (adjusted p-value <1%). Based on xCell signatures, consensus clustering was performed in order to identify groups of samples with the same immune/stromal characteristics. Consensus clustering was performed using the R packages ConsensusClusterPlus (Monti et al., 2003; Wilkerson and Hayes, 2010). Specifically, 80% of the original samples were randomly subsampled without replacement and partitioned into 3 major clusters using the K-Means algorithm.

Estimation of Tumor Purity, Stromal and Immune Scores:

Besides xCell, we utilized ESTIMATE (Yoshihara et al., 2013) to infer immune and stromal scores based on RNA-seq data. To infer tumor purity, TSNet was utilized (Petralia et al., 2018).

DEG and Pathway Analysis:

ssGSEA (Barbie et al., 2009) was utilized to obtain pathway scores based on RNAseq and global proteomics data. For this purpose, the R package GSVA (Hänzelmann et al., 2013) was utilized. Then, a wilcoxon test was performed to find pathways differentially expressed between Cold-Tumor and Hot-Tumor subgroups. P-values were adjusted via Benjamini-Hochberg procedure. The genes/proteins and pathways differentially expressed based on RNAseq and global proteomics abundance were discovered.

Mutations Associated to xCell Signatures:

Raw xCells signatures were modeled as a linear function of mutation status. For this analysis, only mutations with more than 15 mutated samples across all 211 tumor samples were considered (i.e., 67 genes). P-values were adjusted for multiple comparison using Benjamini-Hochberg correction.

Immune Evasive Mechanisms:

Immune evasion, wherein tumor cells employ multiple mechanisms to evade anti-tumor immune response, is a fundamental process driving tumor cell survival and evolution. Immune checkpoint blockade therapy has emerged as a treatment strategy for cancer patients, based on harnessing the anti-tumor immune response genes (Abril-Rodriguez and Ribas, 2017). However, a significant number of patients have failed to respond to immunomodulation strategies such as checkpoint inhibitors, likely due to tumor-specific immunosuppressive mechanisms and incomplete restoration of adaptive immunity (Achyut and Arbab, 2016; Allard et al., 2016b; Jerby-Arnon et al., 2018; Kozuma et al., 2018b). We assume that the failure of immune therapy is caused by two basic reasons: (i) the insufficient activation of the immune response, and, (ii) the evolutionarily selected mechanisms of immune evasion. We also assume that activation of adaptive immune system and sensitivity to checkpoint therapy entirely depends on upregulation or downregulation of IFN-γ axis—a pathways of 15 genes, which is composed of proteins expressed primarily in cancer cells—IFN-γ receptors (IFNGR1, IFNGR2); JAK/STAT-signaling component (JAK1, JAK2, STAT1, STAT3, IRF1); antigen presenting (HLA-A, HLA-B, HLA-C, HLA-E, HLA-F, HLA-G); and, checkpoint proteins (PD-L1/PD1). Thus, non-responder tumors are either those which are invisible to immune cells because of suppressed IFN-γ axis, or those with IFN-γ axis activated along with activated immune evasion that prevent leukocyte-driven cancer cell death. Following this idea, we proposed a general protocol for revealing proteins involved in immune evasion and determining potential targets for combination therapy. First, we inferred relative activation of IFN-γ axis pathway across tumors. To this end, we assessed the probability of pathway proteins to occupy by random the observed (or higher) positions in a list of tumor proteins ranked by abundance levels from the top to the bottom and, similarly, probability to occupy the same positions (or lower) by random in a list of abundance levels ranked from the bottom to the top. These two probabilities were computed as geometrically averaged P values assessed for proteins of each pathway by Fisher's exact test; these probabilities assess significance of “overrepresentation” of pathway proteins in the top and at the bottom of the ranked list of tumor proteins. The inferred pathway activation score was defined as the negative log of the ratio of the above two probabilities. this score is positive when pathway proteins largely occupy positions in the top half of the list, and it is negative, when pathway proteins are largely at the bottom. Secondly, we determined proteins which are significantly upregulated with inferred activation of IFN-γ axis and have known immune evasion role (markers of MDSC (Achyut and Arbab, 2016), adenosine signaling signature (Allard et al., 2016b), IDO1 pathway (Kozuma et al., 2018b; Liu et al., 2018; Takada et al., 2019; Zhang et al., 2019b) or have potential therapeutic value as targets of drugs from Drug Bank (Frolkis et al., 2010; Jewison et al., 2014).

Deep Learning Analysis to Identify Histological Features

Tissue histopathology slides were first downloaded from The Cancer Imaging Archive (TCIA) database. The slides and their corresponding per-slide level labels were then separated into a training (80%), a validation (10%), and a test set (10%) at per-patient level. Each slide was then tiled into 299-by-299-pixel pieces with overlapping area of 49 pixels from each edge, omitting those with over 30% background. Tiles of each set were packaged into a TFrecord file. Then, the InceptionV3-architectured convolutional neural network (CNN) was trained from scratch and the best performing model was picked based on validation set performance. The performance of the model was evaluated by statistical metrics (area under ROC, area under PRC, and accuracy) on per-slide and per-tile level. Lastly, the trained model was applied to the test set, and the per-tile prediction scores were aggregated by slides and shown as heatmaps. 10000 tiles were randomly selected for visualization from the test set of 137990 tiles cropped from 36 slides of 11 individual patients. The test data were propagated through the trained model to obtain positive prediction scores, the probability of being a STK11 mutation positive case estimated by the deep learning model. Additionally, for each test example, activation scores of the fully-connected layer immediately before output layer, a vector of 2048 elements were extracted as representation of the input sample in perspective of the predictive model. The activation scores of 10000 sample tiles were further reduced to two-dimensional representations by tSNE. Overlay of positive prediction scores on sample points show distinct clusters for predicted positive (orange) and predictive negative (blue) cases. Examples of true positive (red outline) and true negative (black outline) tiles exhibit different histologic features, such that the STK11 mutated tiles correctly recognized by the model harbors abundant inflammatory cells, and STK11 wild type tiles showed typical adenocarcinoma characteristics.

Independent Component Analysis (ICA)

As previously described (Liu et al., 2019), ICA was run 100 times with random initial values on 110 tumor samples. In each run, 110 independent components (equal to the number of samples) were extracted to obtain as much information as possible. All components were then pooled and grouped into 110 clusters using K-medoids method and Spearman correlation as dissimilarity measure. Each independent component (and a sample point submitted to the clustering algorithm) was a vector comprised with weights of all genes in the original data. Genes that contributed heavily to a component were assigned large coefficients that could serve as a pathway-level molecular signature. Consistent clusters of independent components would exhibit large intra-group homogeneity (average silhouette width>0.8) and are comprised of members generated in different runs (>90), indicating that similar signals were extracted recurrently when the algorithm was initiated from different values. The centroids of the clusters were considered as representative of a stable signature, and mean mixing scores (activity of each signature over all samples) of each cluster were used to represent the activity levels of corresponding signature in each sample. To investigate the correlation between blindly extracted features and known clinical characteristics, the corresponding mixing scores for all members of a component cluster were regressed against 46 clinical variables, and the count of significant correlations (P<1.9810-6, linear regression, P value controlled for multiple testing at the 0.01 level) indicated association between the particular molecular signature and clinical variable pair. Signatures showed high percentage of significant correlations for all members and large average −log 10(p-value) values within the cluster were considered to be associated with the clinical feature. Genes heavily weighted in the cluster centroid coefficients vector may thus shed light on molecular mechanisms underlying the clinical feature. One highly consistent signature (average cluster silhouette width 0.97, 100 members produced by 100 different runs) was found to be significantly associated with STK11 mutation status, with an average −log P value of 5.7.

Calculating Mutation-Based Cis- and Trans-Effects

We examined the cis- and trans-effects of 18 mutations that were significant in a previous large-scale TCGA LUAD study (Cancer Genome Atlas Research Network, 2014) on the RNA, proteome, and phosphoproteome of cancer-related genes (Bailey et al., 2018). After excluding silent mutations, samples were separated into mutated and wild type groups. We used the Wilcoxon rank-sum test to report differentially expressed features (RNA, proteins, phosphosites and acetyl sites) between the two groups. Differentially enriched features passing an FDR <0.05 cut-off were separated into two categories based on cis- and trans-effects.

Multi-Omic Outlier Analysis

We calculated the median and interquartile range (IQR) values for phosphopeptide, protein, gene expression and copy number alterations of known kinases (N=701), phosphatases (N=135), E3 ubiquitin ligases (N=377) and de-ubiquitin ligases (N=87) using TMT-based global phosphoproteomic and proteomic data, RNA-Seq expression data or CNA data. Outliers were defined as any value higher than the median plus 1.5× IQR. Phosphopeptide data was aggregated into genes by summing outlier and non-outlier values per sample. Outlier counts were used to determine enriched genes in a group of samples at each data level. First, genes without an outlier value in at least 10% of samples in the group of interest were filtered out. Additionally, only genes where the frequency of outliers in the group of interest was higher than the frequency in the outgroup were considered in the analysis. The group of interest was compared to the rest of the samples using a Fisher's exact test on the count of outlier and non-outlier values per group. Resulting p-values were corrected for multiple comparisons using the Benjamini-Hochberg correction. Druggability was determined for each gene using the drug-gene interaction database (DGIdb) (Cotto et al., 2018). The mean impact of shRNA or CRISPR mediated depletion of each gene on survival and proliferation in lung cancer cell lines was also visualized based on previous studies (Barretina et al., 2012; Tsherniak et al., 2017).

Pathway Analysis in Tumors and NATs with High and Low Smoking Scores.

In the set of tumor samples, the high smoking score (HSS) subset consists of 58 samples, while low smoking score (LSS) subset contains 49 samples. There are 52 NAT samples with paired HSS tumor samples, and 46 NAT samples with paired LSS tumor samples.

We used gene sets of molecular pathways from KEGG (Kanehisa and Goto, 2000), Hallmark (Liberzon et al., 2015) and Reactome (Croft et al., 2014) databases to compute single sample gene set enrichment scores (Barbie et al., 2009) for each sample. To compute pathway HSS vs LSS differential scores, for both tumor and NAT, we ran two one-sided Wilcoxon rank-sum tests (greater than, and lesser than) on HSS vs LSS sets of samples and performed Benjamini-Hochberg correction on computed p-values (P.adj). The differential score (Q) is obtained as signed log 10(P.adj) from the lower of the two p-values derived from two one-sided Wilxocon rank-sum tests. The sign “+” and “−” indicated upregulated and downregulated pathways respectively, in HSS. Differential scores were computed for both proteome (for the set of 7136 proteins with no missing values) and transcriptome (18099 genes).

To select the six groups of pathways with characteristic HSS vs LSS proteome behavior in tumor and NAT, we used the FDR <0.05 for differential behavior and FDR >0.3 for the absence of differential behavior. For specific pathway groups, this amounted to the following conditions: group 1: Q(Tumor) >1.301 & Q(NAT)<−1.301; group 2: Q(Tumor)<−1.301 & Q(NAT) >1.301; group 3: Q(Tumor) >1.301 & Q(NAT) >1.301; group 4: Q(Tumor)<−1.301 & Q(NAT)<−1.301; group 5: Q(Tumor) >1.301 & |Q(NAT)|<0.523; group 6: Q(Tumor)<-1.301 & |Q(NAT)|<0.523.

Tumor-NAT Related Analysis

Principal Component Analysis (PCA):

Principal component analysis was performed on RNA (18099), protein (10165), phophosites (40845), and acetyl-sites (6984) dataset using factoextra (Bioconductor, version 1.0.5) package in R (3.1.2). Features with no variance were removed.

Tumor Vs Normal Differential Proteomic Analysis:

TMT-based global proteomic data were used to perform differential proteome analysis between tumor and NAT samples. A Wilcoxon rank sum test was performed to determine differential abundance of proteins between tumor and NAT samples. Proteins with log 2-fold change >2 and Benjamini-Hochberg adjusted p-value <0.01 were considered to be tumor-associated proteins. For each tumor-associated protein, we obtained immunohistochemistry-based staining score in lung tumors from the Human Protein Atlas (HPA, https://www.proteinatlas.org), in which tumor-specific staining is reported in four levels, i.e. high, medium, low, and not detected. The protein specific annotations such as protein class, found in plasma, or ontology were obtained from HPA, Uniprot and GO. Proteins with known function or class like transcription factors, enzymes, transporters, and transmembranes were classified. Plasma proteins represent the proteins found in plasma, whereas secreted were secreted/exported outside cell. The FDA approved drug targeting the protein or under clinical trial were annotated. Considering the role of epithelial-to-mesenchymal transition (EMT) in metastasis proteins overlapping with hallmarks of EMT geneset were shown separately. Differential results were used to perform enrichment analysis using GSEA (Subramanian et al., 2005) implemented in WebGestalt (Wang et al., 2017). Similar analysis was performed on phosphoproteome and acetylome to detect tumor-specific phosphosites and acetyl-sites respectively.

Mutant Phenotype Specific Protein Biomarkers:

Four driver mutant phenotypes considered for analysis were TP53 (n=52), EGFR (n=36), KRAS (n=29), and STK11 (n=17). A Wilcoxon rank sum test was performed between tumor and paired NAT samples using only samples with mutations. Similar analysis was performed on samples with wild-type (WT) phenotype only (TP53_{WT}=49, EGFR_{WT}=65, KRAS_{WT}=72, STK11_{WT}=84). Differentially expressed proteins in a given mutant phenotype were selected based on >4-fold difference and Benjamini-Hochberg adjusted p-value <0.01. Further, mutant specific proteins were filtered using log 2 (median difference between Mutant and Wildtype) >1.5 to remove noise from corresponding WT samples. The filtered proteins were nominated as mutant-specific biomarkers if its expression is upregulated in 80% of tumor samples compared to matched normal samples. The fold change between tumor and matched normal are shown in heatmap for identified protein biomarkers in each mutant phenotype.

Phosphorylation-Driven Signature Enrichment Analysis

Based on the results of the Tumor-NAT related analysis described above, we performed phosphosite-specific signature enrichment analysis (PTM-SEA) (Krug et al., 2018) to identify dysregulated phosphorylation-driven pathways in tumors compared to its paired normal adjacent tissue (NAT). To adequately account for both magnitude and variance of measured phosphosite abundance we used p-values derived from application of the Wilcoxon rank-sum test to phosphorylation data as ranking for PTM-SEA. To that end p-values were log-transformed and signed according to the fold change (signed p-value) such that a large positive values indicated tumor-specific phosphosite abundance and large negative values NAT-specific phosphosite abundance.

log Pₛᵢₜₑ=−log₁₀(p-valueₛᵢₜₑ)*sign(log₂(fold changeₛᵢₜₑ)))

PTM-SEA relies on site-specific annotation provided by PTMsigDB and thus a single site-centric data matrix data is required such that each row corresponds to a single phosphosite. We note that in this analysis the data matrix comprised of a single data column (log transformed and signed p-values of the tumor vs. NAT comparison) and each row represents a confidently localized phosphosites assigned by Spectrum Mill software.

We employed the heuristic introduced in (Krug et al., 2018) to deconvolute multiply phosphorylated peptides to separate data points (log-transformed and signed p-values). Briefly, phosphosites measured on different phospho-proteoform peptides were resolved by using the p-value derived from the least modified version of the peptide. For instance, if a site T4 measured on a doubly phosphorylated (T4, S8) peptide (PEPtIDEsR) was also measured on a mono-phosphorylated version (PEPtIDESR), we assign the p-value derived from the mono-phosphorylated peptide proteoform to T4, and the p-value derived from PEPtIDEsR to S8. If only the doubly phosphorylated proteoform was present in the dataset, we assigned the same p-value to both sites T4 and S8.

We queried the PTM signatures database (PTMsigDB) v1.9.0 downloaded from http://prot-shiny-vm.broadinstitute.org:3838/ptmsigdb-app/using the flanking amino acid sequence (+/−7 aa) as primary identifier. We used the implementation of PTM-SEA available on GitHub (https://github.com/broadinstitute/ssGSEA2.0) using the command interface R-script (ssgsea-cli.R). The following parameters were used to run PTM-SEA:

weight: 1

statistic: “area.under.RES”

output.score.type: “NES”

nperm: 1000

min.overlap: 5

correl.type: “rank”

The sign of the normalized enrichment score (NES) calculated for each signature corresponds to the sign of the tumor-NAT log fold change. P-values for each signature were derived from 1,000 random permutations and further adjusted for multiple hypothesis testing using the method proposed by Benjamini & and Hochberg (Benjamini and Hochberg, 1995). Signatures with FDR-corrected p-values <0.05 were considered to be differential between tumor and NAT.

For mutational subtype analysis (EGFR, KRAS, TP53, STK11) we derived a residual enrichment score between mutated and WT samples by separately applying PTM-SEA to mutated and WT samples to derive signature enrichment scores from which we then calculated the residuals via linear regression (mut non-mut). From the resulting distribution of residual enrichment scores we identified outliers using the +/−1.5*IQR definition used in box and whisker plots.

Variant Peptide Identification and Neoantigen Prediction

We used NeoFlow (https://github.com/bzhanglab/neoflow) for neoantigen prediction. Specifically, Optitype (Szolek et al., 2014) was used to find human leukocyte antigens (HLA) in the WXS data. Then we used netMHCpan (Jurtz et al., 2017) to predict HLA peptide binding affinity for somatic mutation-derived variant peptides with a length between 8-11 amino acids. The cutoff of IC₅₀ binding affinity was set to 150 nM. HLA peptides with binding affinity higher than 150 nM were removed. Variant identification was also performed at both mRNA and protein levels using RNA-Seq data and MS/MS data, respectively. To identify variant peptides, we used a customized protein sequence database approach (Wang et al., 2012). We derived customized protein sequence databases from matched WXS data and then performed database searching using the customized databases for individual TMT experiments. We built a customized database for each TMT experiment based on somatic variants from WXS data. We used Customprodbj (https://github.com/bzhanglab/customprodbj) for customized database construction. MS-GF+ was used for variant peptide identification for all global proteome, phosphorylation data. Results from MS-GF+ were filtered with 1% FDR at PSM level. Remaining variant peptides were further filtered using PepQuery (http://www.pepquery.org) (Wen et al., 2019) with the p-value cutoff <=0.01. The spectra of variant peptides were annotated using PDV (http://www.zhang-lab.org/) (Li et al., 2019).

Cancer Testes (CT) Antigen Prediction

CT antigens were downloaded from the CTdatabase (Almeida et al., 2009). CT antigens with a 2×-fold increase in tumor from adjacent normal in at least 10% of the samples were highlighted.

Data Availability

Proteomics raw datasets are publicly available though the CPTAC data portal https://cptac-data-portal.georgetown.edu/cptac/s/5046

The genomics (WGS, WXS, RNA-seq, miRNA-seq, methylation-array) datasets are available with dbGaP Study Accession: phs001287.v4.p3

https://www.ncbi.nlm.nih.gov/projects/gap/cgi-bin/study.cgi?study_id=phs001287.v4.p3

In addition, all processed data matrices will also be available at LinkedOmics (Vasaikar et al., 2018) (http://www.linkedomics.org) upon publication, where computational tools are available for further exploration of this dataset.

Additional Resources

CPTAC program website; detailing program initiatives, investigators, and datasets are available at https://proteomics.cancer.gov/programs/cptac.

### REFERENCES

Abril-Rodriguez, G., and Ribas, A. (2017). SnapShot: Immune Checkpoint Inhibitors. Cancer Cell 31, 848-848.e1.
Aceto, N., Sausgruber, N., Brinkhaus, H., Gaidatzis, D., Martiny-Baron, G., Mazzarol, G., Confalonieri, S., Quarto, M., Hu, G., Balwierz, P. J., et al. (2012). Tyrosine phosphatase SHP2 promotes breast cancer progression and maintains tumor-initiating cells via activation of key transcription factors and a positive feedback signaling loop. Nat. Med. 18, 529-537.
Achyut, B. R., and Arbab, A. S. (2016). Myeloid cell signatures in tumor microenvironment predicts therapeutic response in cancer. Onco. Targets. Ther. 9, 1047-1055.
Allard, D., Allard, B., Gaudreau, P.-O., Chrobak, P., and Stagg, J. (2016a). CD73-adenosine: a next-generation target in immuno-oncology. Immunotherapy 8, 145-163.
Allard, D., Allard, B., Gaudreau, P.-O., Chrobak, P., and Stagg, J. (2016b). CD73-adenosine: a next-generation target in immuno-oncology. Immunotherapy 8, 145-163.
Almeida, L. G., Sakabe, N. J., deOliveira, A. R., Silva, M. C. C., Mundstein, A. S., Cohen, T., Chen, Y.-T., Chua, R., Gurung, S., Gnjatic, S., et al. (2009). CTdatabase: a knowledge-base of high-throughput and curated data on cancer-testis antigens. Nucleic Acids Res. 37, D816-D819.
Ao, X., Jiang, M., Zhou, J., Liang, H., Xia, H., and Chen, G. (2019). lincRNA-p21 inhibits the progression of non-small cell lung cancer via targeting miR-17-5p. Oncol. Rep. 41, 789-800.
Aran, D., Hu, Z., and Butte, A. J. (2017). xCell: digitally portraying the tissue cellular heterogeneity landscape. Genome Biol. 18, 220.
Aronheim, A., Engelberg, D., Li, N., al-Alawi, N., Schlessinger, J., and Karin, M. (1994). Membrane targeting of the nucleotide exchange factor Sos is sufficient for activating the Ras signaling pathway. Cell 78, 949-961.
Bai, Y., Xiong, L., Zhu, M., Yang, Z., Zhao, J., and Tang, H. (2019). Co-expression network analysis identified KIF2C in association with progression and prognosis in lung adenocarcinoma. Cancer Biomark. 24, 371-382.
Bailey, M. H., Tokheim, C., Porta-Pardo, E., Sengupta, S., Bertrand, D., Weerasinghe, A., Colaprico, A., Wendl, M. C., Kim, J., Reardon, B., et al. (2018). Comprehensive Characterization of Cancer Driver Genes and Mutations. Cell 173, 371-385.e18.
Barbie, D. A., Tamayo, P., Boehm, J. S., Kim, S. Y., Moody, S. E., Dunn, I. F., Schinzel, A. C., Sandy, P., Meylan, E., Scholl, C., et al. (2009). Systematic RNA interference reveals that oncogenic KRAS-driven cancers require TBK1. Nature 462, 108-112.
Barker, L. (2002). A comparison of nine confidence intervals for a Poisson parameter when the expected number of events is ≤5. Am. Stat. 56, 85-89.
Barretina, J., Caponigro, G., Stransky, N., Venkatesan, K., Margolin, A. A., Kim, S., Wilson, C. J., Lehár, J., Kryukov, G. V., Sonkin, D., et al. (2012). The Cancer Cell Line Encyclopedia enables predictive modelling of anticancer drug sensitivity. Nature 483, 603-607.
Benjamini, Y., and Hochberg, Y. (1995). Controlling the False Discovery Rate: A Practical and Powerful Approach to Multiple Testing. J. R. Stat. Soc. Series B Stat. Methodol. 57, 289-300.
Bennett, A. M., Tang, T. L., Sugimoto, S., Walsh, C. T., and Neel, B. G. (1994). Protein-tyrosine-phosphatase SHPTP2 couples platelet-derived growth factor receptor beta to Ras. Proc. Natl. Acad. Sci. U.S.A. 91, 7335-7339.
Bentires-Alj, M., Paez, J. G., David, F. S., Keilhack, H., Halmos, B., Naoki, K., Maris, J. M., Richardson, A., Bardelli, A., Sugarbaker, D. J., et al. (2004). Activating mutations of the noonan syndrome-associated SHP2/PTPN11 gene in human solid tumors and adult acute myelogenous leukemia. Cancer Res. 64, 8816-8820.
Beukers, W., Kandimalla, R., Masius, R. G., Vermeij, M., Kranse, R., van Leenders, G. J., and Zwarthoff, E. C. (2015). Stratification based on methylation of TBX2 and TBX3 into three molecular grades predicts progression in patients with pTa-bladder cancer. Mod. Pathol. 28, 515-522.
Bray, F., Ferlay, J., Soerjomataram, I., Siegel, R. L., Torre, L. A., and Jemal, A. (2018). Global cancer statistics 2018: GLOBOCAN estimates of incidence and mortality worldwide for 36 cancers in 185 countries. CA Cancer J. Clin. 68, 394-424.
Brunet, J.-P., Tamayo, P., Golub, T. R., and Mesirov, J. P. (2004). Metagenes and molecular pattern discovery using matrix factorization. Proc. Natl. Acad. Sci. U.S.A. 101, 4164-4169.
Campbell, J. D., Alexandrov, A., Kim, J., Wala, J., Berger, A. H., Pedamallu, C. S., Shukla, S. A., Guo, G., Brooks, A. N., Murray, B. A., et al. (2016). Distinct patterns of somatic genome alterations in lung adenocarcinomas and squamous cell carcinomas. Nat. Genet. 48, 607-616.
Cancer Genome Atlas Research Network (2014). Comprehensive molecular profiling of lung adenocarcinoma. Nature 511, 543-550.
Canning, P., Sorrell, F. J., and Bullock, A. N. (2015). Structural basis of Keap1 interactions with Nrf2. Free Radic. Biol. Med. 88, 101-107.
Chan, R. J., and Feng, G.-S. (2007). PTPN11 is the first identified proto-oncogene that encodes a tyrosine phosphatase. Blood 109, 862-867.
Chapman, A. M., Sun, K. Y., Ruestow, P., Cowan, D. M., and Madl, A. K. (2016). Lung cancer mutation profile of EGFR, ALK, and KRAS: Meta-analysis and comparison of never and ever smokers. Lung Cancer 102, 122-134.
Chen, C., Lu, Z., Yang, J., Hao, W., Qin, Y., Wang, H., Xie, C., and Xie, R. (2016a). MiR-17-5p promotes cancer cell proliferation and tumorigenesis in nasopharyngeal carcinoma by targeting p21. Cancer Med. 5, 3489-3499.
Chen, G., Gharib, T. G., Huang, C.-C., Thomas, D. G., Shedden, K. A., Taylor, J. M. G., Kardia, S. L. R., Misek, D. E., Giordano, T. J., Iannettoni, M. D., et al. (2002). Proteomic analysis of lung adenocarcinoma: identification of a highly expressed set of proteins in tumors. Clin. Cancer Res. 8, 2298-2305.
Chen, H., Wang, X., Bai, J., and He, A. (2017). Expression, regulation and function of miR-495 in healthy and tumor tissues. Oncol. Lett. 13, 2021-2026.
Chen, R., Ren, S., Meng, T., Aguilar, J., and Sun, Y. (2013). Impact of glutathione-S-transferases (GST) polymorphisms and hypermethylation of relevant genes on risk of prostate cancer biochemical recurrence: a meta-analysis. PLoS One 8, e74775.
Chen, T., Sun, Y., Ji, P., Kopetz, S., and Zhang, W. (2015). Topoisomerase IIα in chromosome instability and personalized cancer therapy. Oncogene 34, 4019-4031.
Chen, Y.-N. P., LaMarche, M. J., Chan, H. M., Fekkes, P., Garcia-Fortanet, J., Acker, M. G., Antonakos, B., Chen, C. H.-T., Chen, Z., Cooke, V. G., et al. (2016b). Allosteric inhibition of SHP2 phosphatase inhibits cancers driven by receptor tyrosine kinases. Nature 535, 148-152.
Chew, H. K., Davies, A. M., Wun, T., Harvey, D., Zhou, H., and White, R. H. (2008). The incidence of venous thromboembolism among patients with primary lung cancer. J. Thromb. Haemost. 6, 601-608.
Chidambaranathan-Reghupaty, S., Mendoza, R., Fisher, P. B., and Sarkar, D. (2018). The multifaceted oncogene SND1 in cancer: focus on hepatocellular carcinoma. Hepatoma Res 4, 10-20517.
Chu, A., Robertson, G., Brooks, D., Mungall, A. J., Birol, I., Coope, R., Ma, Y., Jones, S., and Marra, M. A. (2016). Large-scale profiling of microRNAs for The Cancer Genome Atlas. Nucleic Acids Res. 44, e3.
Cibulskis, K., Lawrence, M. S., Carter, S. L., Sivachenko, A., Jaffe, D., Sougnez, C., Gabriel, S., Meyerson, M., Lander, E. S., and Getz, G. (2013). Sensitive detection of somatic point mutations in impure and heterogeneous cancer samples. Nat. Biotechnol. 31, 213-219.
Cleynen, I., Huysmans, C., Sasazuki, T., Shirasawa, S., Van de Ven, W., and Peeters, K. (2007). Transcriptional control of the human high mobility group A1 gene: basal and oncogenic Ras-regulated expression. Cancer Res. 67, 4620-4629.
Clinical Lung Cancer Genome Project (CLCGP), and Network Genomic Medicine (NGM) (2013). A genomics-based classification of human lung tumors. Sci. Transl. Med. 5, 209ra153.
Cloonan, N., Brown, M. K., Steptoe, A. L., Wani, S., Chan, W. L., Forrest, A. R. R., Kolle, G., Gabrielli, B., and Grimmond, S. M. (2008). The miR-17-5p microRNA is a key regulator of the G1/S phase cell cycle transition. Genome Biol. 9, R127.
Colaprico, A., Silva, T. C., Olsen, C., Garofano, L., Cava, C., Garolini, D., Sabedot, T. S., Malta, T. M., Pagnotta, S. M., Castiglioni, I., et al. (2016). TCGAbiolinks: an R/Bioconductor package for integrative analysis of TCGA data. Nucleic Acids Res. 44, e71.
Cotto, K. C., Wagner, A. H., Feng, Y.-Y., Kiwala, S., Coffman, A. C., Spies, G., Wollam, A., Spies, N. C., Griffith, 01., and Griffith, M. (2018). DGIdb 3.0: a redesign and expansion of the drug-gene interaction database. Nucleic Acids Res. 46, D1068-D1073.
Coudray, N., Ocampo, P. S., Sakellaropoulos, T., Narula, N., Snuderl, M., Fenyö, D., Moreira, A. L., Razavian, N., and Tsirigos, A. (2018). Classification and mutation prediction from non-small cell lung cancer histopathology images using deep learning. Nat. Med. 24, 1559-1567.
Croft, D., Mundo, A. F., Haw, R., Milacic, M., Weiser, J., Wu, G., Caudy, M., Garapati, P., Gillespie, M., Kamdar, M. R., et al. (2014). The Reactome pathway knowledgebase. Nucleic Acids Res. 42, D472-D477.
Cully, M., and Downward, J. (2008). SnapShot: Ras Signaling. Cell 133, 1292-1292.e1.
Cunnick, J. M., Meng, S., Ren, Y., Desponts, C., Wang, H.-G., Djeu, J. Y., and Wu, J. (2002). Regulation of the mitogen-activated protein kinase signaling pathway by SHP2. J. Biol. Chem. 277, 9498-9504.
Daily, K., Ho Sui, S. J., Schriml, L. M., Dexheimer, P. J., Salomonis, N., Schroll, R., Bush, S., Keddache, M., Mayhew, C., Lotia, S., et al. (2017). Molecular, phenotypic, and sample-associated data to describe pluripotent stem cell lines and derivatives. Sci Data 4, 170030.
Dardaei, L., Wang, H. Q., Singh, M., Fordjour, P., Shaw, K. X., Yoda, S., Kerr, G., Yu, K., Liang, J., Cao, Y., et al. (2018). SHP2 inhibition restores sensitivity in ALK-rearranged non-small-cell lung cancer resistant to ALK inhibitors. Nat. Med. 24, 512-517.
Ding, L., Getz, G., Wheeler, D. A., Mardis, E. R., McLellan, M. D., Cibulskis, K., Sougnez, C., Greulich, H., Muzny, D. M., Morgan, M. B., et al. (2008). Somatic mutations affect key pathways in lung adenocarcinoma. Nature 455, 1069-1075.
Dou, F., Li, H., Zhu, M., Liang, L., Zhang, Y., Yi, J., and Zhang, Y. (2018). Association between oncogenic status and risk of venous thromboembolism in patients with non-small cell lung cancer. Respir. Res. 19, 88.
Ducray, S. P., Natarajan, K., Garland, G. D., Turner, S. D., and Egger, G. (2019). The Transcriptional Roles of ALK Fusion Proteins in Tumorigenesis. Cancers 11.
Erdel, M., Trefz, G., Spiess, E., Habermaas, S., Spring, H., Lah, T., and Ebert, W. (1990). Localization of cathepsin B in two human lung cancer cell lines. J. Histochem. Cytochem. 38, 1313-1321.
Fabregat, A., Sidiropoulos, K., Viteri, G., Forner, O., Marin-Garcia, P., Arnau, V., D'Eustachio, P., Stein, L., and Hermjakob, H. (2017). Reactome pathway analysis: a high-performance in-memory approach. BMC Bioinformatics 18, 142.
Fahrmann, J. F., Grapov, D., Phinney, B. S., Stroble, C., DeFelice, B. C., Rom, W., Gandara, D. R., Zhang, Y., Fiehn, O., Pass, H., et al. (2016). Proteomic profiling of lung adenocarcinoma indicates heightened DNA repair, antioxidant mechanisms and identifies LASP1 as a potential negative predictor of survival. Clin. Proteomics 13, 31.
Fang, D., Hawke, D., Zheng, Y., Xia, Y., Meisenhelder, J., Nika, H., Mills, G. B., Kobayashi, R., Hunter, T., and Lu, Z. (2007). Phosphorylation of beta-catenin by AKT promotes beta-catenin transcriptional activity. J. Biol. Chem. 282, 11221-11229.
Fisher, S., Barry, A., Abreu, J., Minie, B., Nolan, J., Delorey, T. M., Young, G., Fennell, T. J., Allen, A., Ambrogio, L., et al. (2011). A scalable, fully automated process for construction of sequence-ready human exome targeted capture libraries. Genome Biol. 12, R1.
Foerster, S., Kacprowski, T., Dhople, V. M., Hammer, E., Herzog, S., Saafan, H., Bien-Möller, S., Albrecht, M., Völker, U., and Ritter, C. A. (2013). Characterization of the EGFR interactome reveals associated protein complex networks and intracellular receptor dynamics. Proteomics 13, 3131-3144.
Fortin, J.-P., Labbe, A., Lemire, M., Zanke, B. W., Hudson, T. J., Fertig, E. J., Greenwood, C. M., and Hansen, K. D. (2014). Functional normalization of 450k methylation array data improves replication in large cancer studies. Genome Biol. 15, 503.
Friedman, R. S., Bangur, C. S., Zasloff, E. J., Fan, L., Wang, T., Watanabe, Y., and Kalos, M. (2004). Molecular and immunological evaluation of the transcription factor SOX-4 as a lung tumor vaccine antigen. J. Immunol. 172, 3319-3327.
Frolkis, A., Knox, C., Lim, E., Jewison, T., Law, V., Hau, D. D., Liu, P., Gautam, B., Ly, S., Guo, A. C., et al. (2010). SMPDB: The Small Molecule Pathway Database. Nucleic Acids Res. 38, D480-D487.
Fujise, N., Nanashim, A., Taniguchi, Y., Matsuo, S., Hatano, K., Matsumoto, Y., Tagawa, Y., and Ayabe, H. (2000). Prognostic impact of cathepsin B and matrix metalloproteinase-9 in pulmonary adenocarcinomas by immunohistochemical study. Lung Cancer 27, 19-26.
Fukutomi, T., Takagi, K., Mizushima, T., Ohuchi, N., and Yamamoto, M. (2014). Kinetic, thermodynamic, and structural characterizations of the association between Nrf2-DLGex degron and Keap1. Mol. Cell. Biol. 34, 832-846.
Gao, H., Yu, G., Zhang, X., Yu, S., Sun, Y., and Li, Y. (2019). BZW2 gene knockdown induces cell growth inhibition, G1 arrest and apoptosis in muscle-invasive bladder cancers: A microarray pathway analysis. J. Cell. Mol. Med. 23, 3905-3915.
Gao, Q., Liang, W.-W., Foltz, S. M., Mutharasu, G., Jayasinghe, R. G., Cao, S., Liao, W.-W., Reynolds, S. M., Wyczalkowski, M. A., Yao, L., et al. (2018). Driver Fusions and Their Implications in the Development and Treatment of Human Cancers. Cell Rep. 23, 227-238.e3.
Gaujoux, R., and Seoighe, C. (2010). A flexible R package for nonnegative matrix factorization. BMC Bioinformatics 11, 367.
Gautschi, O., Milia, J., Filleron, T., Wolf, J., Carbone, D. P., Owen, D., Camidge, R., Narayanan, V., Doebele, R. C., Besse, B., et al. (2017). Targeting RET in Patients With RET-Rearranged Lung Cancers: Results From the Global, Multicenter RET Registry. J. Clin. Oncol. 35, 1403-1410.
Gildea, J. J., Harding, M. A., Seraj, M. J., Guiding, K. M., and Theodorescu, D. (2002). The role of Ral A in epidermal growth factor receptor-regulated cell motility. Cancer Res. 62, 982-985.
Giubellino, A., Burke, T. R., Jr, and Bottaro, D. P. (2008). Grb2 signaling in cell motility and cancer. Expert Opin. Ther. Targets 12, 1021-1033.
Gurioli, G., Martignano, F., Salvi, S., Costantini, M., Gunelli, R., and Casadio, V. (2018). GSTP1 methylation in cancer: a liquid biopsy biomarker? Clin. Chem. Lab. Med. 56, 702-717.
Hänzelmann, S., Castelo, R., and Guinney, J. (2013). GSVA: gene set variation analysis for microarray and RNA-seq data. BMC Bioinformatics 14, 7.
Herbst, R. S., Morgensztern, D., and Boshoff, C. (2018). The biology and management of non-small cell lung cancer. Nature 553, 446-454.
Higashiyama, M., Doi, O., Kodama, K., Yokouchi, H., and Tateishi, R. (1993). Cathepsin B expression in tumour cells and laminin distribution in pulmonary adenocarcinoma. J. Clin. Pathol. 46, 18-22.
Hillig, R. C., Sautier, B., Schroeder, J., Moosmayer, D., Hilpmann, A., Stegmann, C. M., Werbeck, N. D., Briem, H., Boemer, U., Weiske, J., et al. (2019). Discovery of potent SOS1 inhibitors that block RAS activation via disruption of the RAS-SOS1 interaction. Proc. Natl. Acad. Sci. U.S.A. 116, 2551-2560.
Ho, E. E., Atwood, J. R., and Meyskens, F. L., Jr (1987). Methodological development of dietary fiber intervention to lower colon cancer risk. Prog. Clin. Biol. Res. 248, 263-281.
Hosgood, H. D., 3rd, Menashe, I., Shen, M., Yeager, M., Yuenger, J., Rajaraman, P., He, X., Chatterjee, N., Caporaso, N. E., Zhu, Y., et al. (2008). Pathway-based evaluation of 380 candidate genes and lung cancer susceptibility suggests the importance of the cell cycle pathway. Carcinogenesis 29, 1938-1943.
Hunt, A. L., Bateman, N. W., Hood, B. L., Conrads, K. A., Zhou, M., Litzi, T. J., Oliver, J., Mitchell, D., Gist, G., Blanton, B., et al. (2019). Extensive Intratumor Proteogenomic Heterogeneity Revealed by Multiregion Sampling in a High-Grade Serous Ovarian Tumor Specimen.
Imielinski, M., Berger, A. H., Hammerman, P. S., Hernandez, B., Pugh, T. J., Hodis, E., Cho, J., Suh, J., Capelletti, M., Sivachenko, A., et al. (2012). Mapping the hallmarks of lung adenocarcinoma with massively parallel sequencing. Cell 150, 1107-1120.
Inoue, T., Ishida, T., Sugio, K., and Sugimachi, K. (1994). Cathepsin B expression and laminin degradation as factors influencing prognosis of surgically treated patients with lung adenocarcinoma. Cancer Res. 54, 6133-6136.
Jariwala, N., Rajasekaran, D., Mendoza, R. G., Shen, X.-N., Siddiq, A., Akiel, M. A., Robertson, C. L., Subler, M. A., Windle, J. J., Fisher, P. B., et al. (2017). Oncogenic Role of SND1 in Development and Progression of Hepatocellular Carcinoma. Cancer Res. 77, 3306-3316.
Jerby-Arnon, L., Shah, P., Cuoco, M. S., Rodman, C., Su, M.-J., Melms, J. C., Leeson, R., Kanodia, A., Mei, S., Lin, J.-R., et al. (2018). A Cancer Cell Program Promotes T Cell Exclusion and Resistance to Checkpoint Blockade. Cell 175, 984-997.e24.
Jeschke, J., Bizet, M., Desmedt, C., Calonne, E., Dedeurwaerder, S., Garaud, S., Koch, A., Larsimont, D., Salgado, R., Van den Eynden, G., et al. (2017). DNA methylation-based immune response signature improves patient diagnosis in multiple cancers. J. Clin. Invest. 127, 3090-3102.
Jewison, T., Su, Y., Disfany, F. M., Liang, Y., Knox, C., Maciejewski, A., Poelzer, J., Huynh, J., Zhou, Y., Arndt, D., et al. (2014). SMPDB 2.0: big improvements to the Small Molecule Pathway Database. Nucleic Acids Res. 42, D478-D484.
Jiang, Z., Lohse, C. M., Chu, P. G., Wu, C.-L., Woda, B. A., Rock, K. L., and Kwon, E. D. (2008). Oncofetal protein IMP3: a novel molecular marker that predicts metastasis of papillary and chromophobe renal cell carcinomas. Cancer 112, 2676-2682.
Jin, X., Liao, M., Zhang, L., Yang, M., and Zhao, J. (2019). Role of the novel gene BZW2 in the development of hepatocellular carcinoma. J. Cell. Physiol.
Jurtz, V., Paul, S., Andreatta, M., Marcatili, P., Peters, B., and Nielsen, M. (2017). NetMHCpan-4.0: Improved Peptide-MHC Class I Interaction Predictions Integrating Eluted Ligand and Peptide Binding Affinity Data. J. Immunol. 199, 3360-3368.
Kamioka, Y., Yasuda, S., Fujita, Y., Aoki, K., and Matsuda, M. (2010). Multiple decisive phosphorylation sites for the negative feedback regulation of SOS1 via ERK. J. Biol. Chem. 285, 33540-33548.
Kandimalla, R., van Tilborg, A. A. G., Kompier, L. C., Stumpel, D. J. P. M., Stam, R. W., Bangma, C. H., and Zwarthoff, E. C. (2012). Genome-wide analysis of CpG island methylation in bladder cancer identified TBX2, TBX3, GATA2, and ZIC4 as pTa-specific prognostic markers. Eur. Urol. 61, 1245-1256.
Kanehisa, M., and Goto, S. (2000). KEGG: kyoto encyclopedia of genes and genomes. Nucleic Acids Res. 28, 27-30.
Karachaliou, N., Cardona, A. F., Bracht, J. W. P., Aldeguer, E., Drozdowskyj, A., Fernandez-Bruno, M., Chaib, I., Berenguer, J., Santarpia, M., Ito, M., et al. (2019). Integrin-linked kinase (ILK) and src homology 2 domain-containing phosphatase 2 (SHP2): Novel targets in EGFR-mutation positive non-small cell lung cancer (NSCLC). EBioMedicine 39, 207-214.
Kasar, S., Kim, J., Improgo, R., Tiao, G., Polak, P., Haradhvala, N., Lawrence, M. S., Kiezun, A., Fernandes, S. M., Bahl, S., et al. (2015). Whole-genome sequencing reveals activation-induced cytidine deaminase signatures during indolent chronic lymphocytic leukaemia evolution. Nat. Commun. 6, 8866.
Kashatus, D. F. (2013). Ral GTPases in tumorigenesis: emerging from the shadows. Exp. Cell Res. 319, 2337-2342.
Key, N. S., Khorana, A. A., Kuderer, N. M., Bohlke, K., Lee, A. Y. Y., Arcelus, J. I., Wong, S. L., Balaban, E. P., Flowers, C. R., Francis, C. W., et al. (2019). Venous Thromboembolism Prophylaxis and Treatment in Patients With Cancer: ASCO Clinical Practice Guideline Update. J. Clin. Oncol. JC01901461.
Kim, H., and Park, H. (2007). Sparse non-negative matrix factorizations via alternating non-negativity-constrained least squares for microarray data analysis. Bioinformatics 23, 1495-1502.
Kim, J., Mouw, K. W., Polak, P., Braunstein, L. Z., Kamburov, A., Kwiatkowski, D. J., Rosenberg, J. E., Van Allen, E. M., D'Andrea, A., and Getz, G. (2016). Somatic ERCC2 mutations are associated with a distinct genomic signature in urothelial tumors. Nat. Genet. 48, 600-606.
Kim, J., Hu, Z., Cai, L., Li, K., Choi, E., Faubert, B., Bezwada, D., Rodriguez-Canales, J., Villalobos, P., Lin, Y.-F., et al. (2017). CPS1 maintains pyrimidine pools and DNA synthesis in KRAS/LKB1-mutant lung cancer cells. Nature 546, 168-172.
Kim, K. M., An, A. R., Park, H. S., Jang, K. Y., Moon, W. S., Kang, M. J., Lee, Y. C., Ku, J. H., and Chung, M. J. (2018). Combined expression of protein disulfide isomerase and endoplasmic reticulum oxidoreductin 1-a is a poor prognostic marker for non-small cell lung cancer. Oncol. Lett. 16, 5753-5760.
Koboldt, D. C., Zhang, Q., Larson, D. E., Shen, D., McLellan, M. D., Lin, L., Miller, C. A., Mardis, E. R., Ding, L., and Wilson, R. K. (2012). VarScan 2: somatic mutation and copy number alteration discovery in cancer by exome sequencing. Genome Res. 22, 568-576.
Kohno, T., Ichikawa, H., Totoki, Y., Yasuda, K., Hiramoto, M., Nammo, T., Sakamoto, H., Tsuta, K., Furuta, K., Shimada, Y., et al. (2012). KIF5B-RET fusions in lung adenocarcinoma. Nat. Med. 18, 375-377.
Koike, A., Nishikawa, H., Wu, W., Okada, Y., Venkitaraman, A. R., and Ohta, T. (2010). Recruitment of phosphorylated NPM1 to sites of DNA damage through RNF8-dependent ubiquitin conjugates. Cancer Res. 70, 6746-6756.
Kong, W., Cheng, Y., Liang, H., Chen, Q., Xiao, C., Li, K., Huang, Z., and Zhang, J. (2018). Prognostic value of miR-17-5p in cancers: a meta-analysis. Onco. Targets. Ther. 11, 3541-3549.
Konofaos, P., Kontzoglou, K., Parakeva, P., Kittas, C., Margari, N., Giaxnaki, E., Pouliakis, M., Kouraklis, G., and Karakitsos, P. (2013). The role of ThinPrep cytology in the investigation of ki-67 index, p53 and HER-2 detection in fine-needle aspirates of breast tumors. J. BUON 18, 352-358.
Kozuma, Y., Takada, K., Toyokawa, G., Kohashi, K., Shimokawa, M., Hirai, F., Tagawa, T., Okamoto, T., Oda, Y., and Maehara, Y. (2018a). Indoleamine 2,3-dioxygenase 1 and programmed cell death-ligand 1 co-expression correlates with aggressive features in lung adenocarcinoma. European Journal of Cancer 101, 20-29.
Kozuma, Y., Takada, K., Toyokawa, G., Kohashi, K., Shimokawa, M., Hirai, F., Tagawa, T., Okamoto, T., Oda, Y., and Maehara, Y. (2018b). Indoleamine 2,3-dioxygenase 1 and programmed cell death-ligand 1 co-expression correlates with aggressive features in lung adenocarcinoma. Eur. J. Cancer 101, 20-29.
Krug, K., Mertins, P., Zhang, B., Hornbeck, P., Raju, R., Ahmad, R., Szucs, M., Mundt, F., Forestier, D., Jane-Valbuena, J., et al. (2018). A curated resource for phosphosite-specific signature analysis. Mol. Cell. Proteomics.
Kruglova, N. A., Meshkova, T. D., Kopylov, A. T., Mazurov, D. V., and Filatov, A. V. (2017). Constitutive and activation-dependent phosphorylation of lymphocyte phosphatase-associated phosphoprotein (LPAP). PLoS One 12, e0182468.
Kucab, J. E., Zou, X., Morganella, S., Joel, M., Nanda, A. S., Nagy, E., Gomez, C., Degasperi, A., Harris, R., Jackson, S. P., et al. (2019). A Compendium of Mutational Signatures of Environmental Agents. Cell 177, 821-836.e16.
Kuser-Abali, G., Gong, L., Yan, J., Liu, Q., Zeng, W., Williamson, A., Lim, C. B., Molloy, M. E., Little, J. B., Huang, L., et al. (2018). An EZH2-mediated epigenetic mechanism behind p53-dependent tissue sensitivity to DNA damage. Proc. Natl. Acad. Sci. U.S.A. 115, 3452-3457.
Kwak, E. L., Bang, Y.-J., Camidge, D. R., Shaw, A. T., Solomon, B., Maki, R. G., Ou, S.-H. I., Dezube, B. J., Janne, P. A., Costa, D. B., et al. (2010). Anaplastic lymphoma kinase inhibition in non-small-cell lung cancer. N. Engl. J. Med. 363, 1693-1703.
Lam, S.-K., Yan, S., Xu, S., U, K.-P., Cheng, P. N.-M., and Ho, J. C.-M. (2019). Endogenous arginase 2 as a potential biomarker for PEGylated arginase 1 treatment in xenograft models of squamous cell lung carcinoma. Oncogenesis 8, 18.
Lamb, J., Crawford, E. D., Peck, D., Modell, J. W., Blat, I. C., Wrobel, M. J., Lerner, J., Brunet, J.-P., Subramanian, A., Ross, K. N., et al. (2006). The Connectivity Map: using gene-expression signatures to connect small molecules, genes, and disease. Science 313, 1929-1935.
Lei, B., Qi, W., Zhao, Y., Li, Y., Liu, S., Xu, X., Zhi, C., Wan, L., and Shen, H. (2015). PBK/TOPK expression correlates with mutant p53 and affects patients' prognosis and cell proliferation and viability in lung adenocarcinoma. Hum. Pathol. 46, 217-224.
Le Menn, G., and Neels, J. G. (2018). Regulation of Immune Cell Function by PPARs and the Connection with Metabolic and Neurodegenerative Diseases. Int. J. Mol. Sci. 19.
Li, K., Vaudel, M., Zhang, B., Ren, Y., and Wen, B. (2019). PDV: an integrative proteomics data viewer. Bioinformatics 35, 1249-1251.
Li, S., Shen, D., Shao, J., Crowder, R., Liu, W., Prat, A., He, X., Liu, S., Hoog, J., Lu, C., et al. (2013). Endocrine-therapy-resistant ESR1 variants revealed by genomic characterization of breast-cancer-derived xenografts. Cell Rep. 4, 1116-1130.
Liberzon, A., Birger, C., Thorvaldsdóttir, H., Ghandi, M., Mesirov, J. P., and Tamayo, P. (2015). The Molecular Signatures Database (MSigDB) hallmark gene set collection. Cell Syst 1, 417-425.
Lignitto, L., LeBoeuf, S. E., Homer, H., Jiang, S., Askenazi, M., Karakousi, T. R., Pass, H. I., Bhutkar, A. J., Tsirigos, A., Ueberheide, B., et al. (2019). Nrf2 Activation Promotes Lung Cancer Metastasis by Inhibiting the Degradation of Bach1. Cell 178, 316-329.e18.
Liu, M., Wang, X., Wang, L., Ma, X., Gong, Z., Zhang, S., and Li, Y. (2018). Targeting the IDO1 pathway in cancer: from bench to bedside. J. Hematol. Oncol. 11, 100.
Liu, W., Payne, S. H., Ma, S., and Fenyö, D. (2019). Extracting Pathway-level Signatures from Proteogenomic Data in Breast Cancer Using Independent Component Analysis. Mol. Cell. Proteomics 18, S169-S182.
Lladó, V., Terés, S., Higuera, M., Alvarez, R., Noguera-Salva, M. A., Halver, J. E., Escribá, P. V., and Busquets, X. (2009). Pivotal role of dihydrofolate reductase knockdown in the anticancer activity of 2-hydroxyoleic acid. Proc. Natl. Acad. Sci. U.S.A. 106, 13754-13758.
Lochhead, P., Imamura, Y., Morikawa, T., Kuchiba, A., Yamauchi, M., Liao, X., Qian, Z. R., Nishihara, R., Wu, K., Meyerhardt, J. A., et al. (2012). Insulin-like growth factor 2 messenger RNA binding protein 3 (IGF2BP3) is a marker of unfavourable prognosis in colorectal cancer. Eur. J. Cancer 48, 3405-3413.
Loriot, A., Boon, T., and De Smet, C. (2003). Five new human cancer-germline genes identified among 12 genes expressed in spermatogonia. Int. J. Cancer 105, 371-376.
Lu, J., Wei, J.-H., Feng, Z.-H., Chen, Z.-H., Wang, Y.-Q., Huang, Y., Fang, Y., Liang, Y.-P., Cen, J.-J., Pan, Y.-H., et al. (2017). miR-106b-5p promotes renal cell carcinoma aggressiveness and stem-cell-like phenotype by activating Wnt/β-catenin signalling. Oncotarget 8, 21461-21471.
Lu, W., Gong, D., Bar-Sagi, D., and Cole, P. A. (2001). Site-specific incorporation of a phosphotyrosine mimetic reveals a role for tyrosine phosphorylation of SHP-2 in cell signaling. Mol. Cell 8, 759-769.
Lynch, T. J., Bell, D. W., Sordella, R., Gurubhagavatula, S., Okimoto, R. A., Brannigan, B. W., Harris, P. L., Haserlat, S. M., Supko, J. G., Haluska, F. G., et al. (2004). Activating mutations in the epidermal growth factor receptor underlying responsiveness of non-small-cell lung cancer to gefitinib. N. Engl. J. Med. 350, 2129-2139.
Malta, T. M., Sokolov, A., Gentles, A. J., Burzykowski, T., Poisson, L., Weinstein, J. N., Kamińska, B., Huelsken, J., Omberg, L., Gevaert, O., et al. (2018). Machine Learning Identifies Stemness Features Associated with Oncogenic Dedifferentiation. Cell 173, 338-354.e15.
Marx, N., Mach, F., Sauty, A., Leung, J. H., Sarafi, M. N., Ransohoff, R. M., Libby, P., Plutzky, J., and Luster, A. D. (2000). Peroxisome proliferator-activated receptor-gamma activators inhibit IFN-gamma-induced expression of the T cell-active CXC chemokines IP-10, Mig, and I-TAC in human endothelial cells. J. Immunol. 164, 6503-6508.
Matozaki, T., Murata, Y., Saito, Y., Okazawa, H., and Ohnishi, H. (2009). Protein tyrosine phosphatase SHP-2: a proto-oncogene product that promotes Ras activation. Cancer Sci. 100, 1786-1793.
Matsuda, A., Motoya, S., Kimura, S., McInnis, R., Maizel, A. L., and Takeda, A. (1998). Disruption of lymphocyte function and signaling in CD45-associated protein-null mice. J. Exp. Med. 187, 1863-1870.
McDonald, T. A., and Komulainen, H. (2005). Carcinogenicity of the chlorination disinfection by-product MX. J. Environ. Sci. Health C Environ. Carcinog. Ecotoxicol. Rev. 23, 163-214.
Mermel, C. H., Schumacher, S. E., Hill, B., Meyerson, M. L., Beroukhim, R., and Getz, G. (2011). GISTIC2.0 facilitates sensitive and confident localization of the targets of focal somatic copy-number alteration in human cancers. Genome Biol. 12, R41.
Mertins, P., Mani, D. R., Ruggles, K. V., Gillette, M. A., Clauser, K. R., Wang, P., Wang, X., Qiao, J. W., Cao, S., Petralia, F., et al. (2016). Proteogenomics connects somatic mutations to signalling in breast cancer. Nature 534, 55-62.
Mertins, P., Tang, L. C., Krug, K., Clark, D. J., Gritsenko, M. A., Chen, L., Clauser, K. R., Clauss, T. R., Shah, P., Gillette, M. A., et al. (2018). Reproducible workflow for multiplexed deep-scale proteome and phosphoproteome analysis of tumor tissues by liquid chromatography-mass spectrometry. Nat. Protoc. 13, 1632-1661.
Mizoguchi, T., Ikeda, S., Watanabe, S., Sugawara, M., and Itoh, M. (2017). Mib1 contributes to persistent directional cell migration by regulating the Ctnnd1-Rac1 pathway. Proc. Natl. Acad. Sci. U.S.A. 114, E9280-E9289.
Montagner, A., Yart, A., Dance, M., Perret, B., Salles, J.-P., and Raynal, P. (2005). A novel role for Gab1 and SHP2 in epidermal growth factor-induced Ras activation. J. Biol. Chem. 280, 5350-5360.
Mu, S., Ma, H., Shi, J., and Zhen, D. (2017). The expression of S100B protein in serum of patients with brain metastases from small-cell lung cancer and its clinical significance. Oncol. Lett. 14, 7107-7110.
Mulvihill, M. S., Kwon, Y.-W., Lee, S., Fang, L. T., Choi, H., Ray, R., Kang, H. C., Mao, J.-H., Jablons, D., and Kim, I.-J. (2012). Gremlin is overexpressed in lung adenocarcinoma and increases cell growth and proliferation in normal lung cells. PLoS One 7, e42264.
Myers, S. A., Klaeger, S., Satpathy, S., Viner, R., Choi, J., Rogers, J., Clauser, K., Udeshi, N. D., and Carr, S. A. (2018). Evaluation of Advanced Precursor Determination for Tandem Mass Tag (TMT)-Based Quantitative Proteomics across Instrument Platforms. J. Proteome Res.
Nakashima, M., Adachi, S., Yasuda, I., Yamauchi, T., Kawaguchi, J., Hanamatsu, T., Yoshioka, T., Okano, Y., Hirose, Y., Kozawa, O., et al. (2011). Inhibition of Rho-associated coiled-coil containing protein kinase enhances the activation of epidermal growth factor receptor in pancreatic cancer cells. Mol. Cancer 10, 79.
Nakayama, S., Sng, N., Carretero, J., Weiner, R., Hayashi, Y., Yamamoto, M., Tan, A. J., Yamaguchi, N., Yasuda, H., Li, D., et al. (2014). β-catenin contributes to lung tumor development induced by EGFR mutations. Cancer Res. 74, 5891-5902.
Nayak, A., Dodagatta-Marri, E., Tsolaki, A. G., and Kishore, U. (2012). An Insight into the Diverse Roles of Surfactant Proteins, SP-A and SP-D in Innate and Adaptive Immunity. Frontiers in Immunology 3.
O'Bryan, J. P. (2019). Pharmacological targeting of RAS: Recent success with direct inhibitors. Pharmacol. Res. 139, 503-511.
Okazaki, I., Ishikawa, S., Ando, W., and Sohara, Y. (2016). Lung Adenocarcinoma in Never Smokers: Problems of Primary Prevention from Aspects of Susceptible Genes and Carcinogens. Anticancer Res. 36, 6207-6224.
Okazaki, T., Chikuma, S., Iwai, Y., Fagarasan, S., and Honjo, T. (2013). A rheostat for immune responses: the unique properties of PD-1 and their advantages for clinical application. Nat. Immunol. 14, 1212-1218.
Ostman, A., Hellberg, C., and Böhmer, F. D. (2006). Protein-tyrosine phosphatases and cancer. Nat. Rev. Cancer 6, 307-320.
Paez, J. G., Janne, P. A., Lee, J. C., Tracy, S., Greulich, H., Gabriel, S., Herman, P., Kaye, F. J., Lindeman, N., Boggon, T. J., et al. (2004). EGFR mutations in lung cancer: correlation with clinical response to gefitinib therapy. Science 304, 1497-1500.
Parikh, K., Antanaviciute, A., Fawkner-Corbett, D., Jagielowicz, M., Aulicino, A., Lagerholm, C., Davis, S., Kinchen, J., Chen, H. H., Alham, N. K., et al. (2019). Colonic epithelial cell diversity in health and inflammatory bowel disease. Nature 567, 49-55.
Perez-Moreno, M., Davis, M. A., Wong, E., Pasolli, H. A., Reynolds, A. B., and Fuchs, E. (2006). p120-catenin mediates inflammatory responses in the skin. Cell 124, 631-644.
Peschard, P., McCarthy, A., Leblanc-Dominguez, V., Yeo, M., Guichard, S., Stamp, G., and Marshall, C. J. (2012). Genetic deletion of RALA and RALB small GTPases reveals redundant functions in development and tumorigenesis. Curr. Biol. 22, 2063-2068.
Petralia, F., Wang, L., Peng, J., Yan, A., Zhu, J., and Wang, P. (2018). A new method for constructing tumor specific gene co-expression networks based on samples with tumor purity heterogeneity. Bioinformatics 34, i528-i536.
Prahallad, A., Heynen, G. J. J. E., Germano, G., Willems, S. M., Evers, B., Vecchione, L., Gambino, V., Lieftink, C., Beijersbergen, R. L., Di Nicolantonio, F., et al. (2015). PTPN11 Is a Central Node in Intrinsic and Acquired Resistance to Targeted Cancer Drugs. Cell Rep. 12, 1978-1985.
Qu, P., Du, H., Wang, X., and Yan, C. (2009). Matrix metalloproteinase 12 overexpression in lung epithelial cells plays a key role in emphysema to lung bronchioalveolar adenocarcinoma transition. Cancer Res. 69, 7252-7261.
Quintás-Cardama, A., Hu, C., Qutub, A., Qiu, Y. H., Zhang, X., Post, S. M., Zhang, N., Coombes, K., and Kornblau, S. M. (2017). p53 pathway dysfunction is highly prevalent in acute myeloid leukemia independent of TP53 mutational status. Leukemia 31, 1296-1305.
Ren, Y., Chen, Z., Chen, L., Fang, B., Win-Piazza, H., Haura, E., Koomen, J. M., and Wu, J. (2010). Critical role of Shp2 in tumor growth involving regulation of c-Myc. Genes Cancer 1, 994-1007.
Rivera, M. P., and Stover, D. E. (2004). Gender and lung cancer. Clin. Chest Med. 25, 391-400.
Rojas, J. M., Oliva, J. L., and Santos, E. (2011). Mammalian son of sevenless Guanine nucleotide exchange factors: old concepts and new perspectives. Genes Cancer 2, 298-305.
Romano, G., Acunzo, M., Garofalo, M., Di Leva, G., Cascione, L., Zanca, C., Bolon, B., Condorelli, G., and Croce, C. M. (2012). MiR-494 is regulated by ERK1/2 and modulates TRAIL-induced apoptosis in non-small-cell lung cancer through BIM down-regulation. Proc. Natl. Acad. Sci. U.S.A. 109, 16570-16575.
Rother, K., Johne, C., Spiesbach, K., Haugwitz, U., Tschöp, K., Wasner, M., Klein-Hitpass, L., Möröy, T., Mössner, J., and Engeland, K. (2004). Identification of Tcf-4 as a transcriptional target of p53 signalling. Oncogene 23, 3376-3384.
Roy, M. G., Livraghi-Butrico, A., Fletcher, A. A., McElwee, M. M., Evans, S. E., Boerner, R. M., Alexander, S. N., Bellinghausen, L. K., Song, A. S., Petrova, Y. M., et al. (2014). Muc5b is required for airway defence. Nature 505, 412-416.
Sakashita, M., Sakashita, S., Murata, Y., Shiba-Ishii, A., Kim, Y., Matsuoka, R., Nakano, N., Sato, Y., and Noguchi, M. (2018). High expression of ovarian cancer immunoreactive antigen domain containing 2 (OCIAD2) is associated with poor prognosis in lung adenocarcinoma. Pathol. Int. 68, 596-604.
Salomonis, N., Dexheimer, P. J., Omberg, L., Schroll, R., Bush, S., Huo, J., Schriml, L., Ho Sui, S., Keddache, M., Mayhew, C., et al. (2016). Integrated Genomic Analysis of Diverse Induced Pluripotent Stem Cells from the Progenitor Cell Biology Consortium. Stem Cell Reports 7, 110-125.
Sanchez-Vega, F., Mina, M., Armenia, J., Chatila, W. K., Luna, A., La, K. C., Dimitriadoy, S., Liu, D. L., Kantheti, H. S., Saghafinia, S., et al. (2018). Oncogenic Signaling Pathways in The Cancer Genome Atlas. Cell 173, 321-337.e10.
Santoro, R., Carbone, C., Piro, G., Chiao, P. J., and Melisi, D. (2017). TAK-ing aim at chemoresistance: The emerging role of MAP3K7 as a target for cancer therapy. Drug Resist. Updat. 33-35, 36-42.
Satpathy, S., Jaehnig, E. J., Krug, K., Kim, B.-J., Saltzman, A. B., Chan, D., Holloway, K. R., Anurag, M., Huang, C., Singh, P., et al. (2019). Microscaled Proteogenomic Methods for Precision Oncology.
Saunders, C. T., Wong, W. S. W., Swamy, S., Becq, J., Murray, L. J., and Cheetham, R. K. (2012). Strelka: accurate somatic small-variant calling from sequenced tumor-normal sample pairs. Bioinformatics 28, 1811-1817.
Scanlan, M. J., Altorki, N. K., Gure, A. O., Williamson, B., Jungbluth, A., Chen, Y. T., and Old, L. J. (2000). Expression of cancer-testis antigens in lung cancer: definition of bromodomain testis-specific gene (BRDT) as a new CT gene, CT9. Cancer Lett. 150, 155-164.
Schneeberger, V. E., Ren, Y., Luetteke, N., Huang, Q., Chen, L., Lawrence, H. R., Lawrence, N. J., Haura, E. B., Koomen, J. M., Coppola, D., et al. (2015). Inhibition of Shp2 suppresses mutant EGFR-induced lung tumors in transgenic mouse model of lung adenocarcinoma. Oncotarget 6, 6191-6202.
Schwermer, M., Lee, S., Köster, J., van Maerken, T., Stephan, H., Eggert, A., Monk, K., Schulte, J. H., and Schramm, A. (2015). Sensitivity to cdk1-inhibition is modulated by p53 status in preclinical models of embryonal tumors. Oncotarget 6, 15425-15435.
Seifart, C., Lin, H.-M., Seifart, U., Plagens, A., DiAngelo, S., Von Wichert, P., and Floros, J. (2005). Rare SP-A alleles and the SP-A1-6A4 allele associate with risk for lung carcinoma. Clinical Genetics 68, 128-136.
Shadforth, I. P., Dunkley, T. P. J., Lilley, K. S., and Bessant, C. (2005). i-Tracker: for quantitative proteomics using iTRAQ. BMC Genomics 6, 145.
Shao, G., Wang, R., Sun, A., Wei, J., Peng, K., Dai, Q., Yang, W., and Lin, Q. (2018). The E3 ubiquitin ligase NEDD4 mediates cell migration signaling of EGFR in lung cancer cells. Mol. Cancer 17, 24.
Shaw, A. T., Ou, S.-H. I., Bang, Y.-J., Camidge, D. R., Solomon, B. J., Salgia, R., Riely, G. J., Varella-Garcia, M., Shapiro, G. I., Costa, D. B., et al. (2014). Crizotinib in ROS1-rearranged non-small-cell lung cancer. N. Engl. J. Med. 371, 1963-1971.
Shi, D.-M., Bian, X.-Y., Qin, C.-D., and Wu, W.-Z. (2018). miR-106b-5p promotes stem cell-like properties of hepatocellular carcinoma cells by targeting PTEN via PI3K/Akt pathway. Onco. Targets. Ther. 11, 571-585.
Shimizu, K., Nakata, M., Hirami, Y., Yukawa, T., Maeda, A., and Tanemoto, K. (2010). Tumor-Infiltrating Foxp3 Regulatory T Cells are Correlated with Cyclooxygenase-2 Expression and are Associated with Recurrence in Resected Non-small Cell Lung Cancer. Journal of Thoracic Oncology 5, 585-590.
Siegel, R. L., Miller, K. D., and Jemal, A. (2019). Cancer statistics, 2019. CA Cancer J. Clin. 69, 7-34.
Sokolov, A., Paull, E. O., and Stuart, J. M. (2016). ONE-CLASS DETECTION OF CELL STATES IN TUMOR SUBTYPES. Pac. Symp. Biocomput. 21, 405-416.
Song, N., Liu, B., Wu, J.-L., Zhang, R.-F., Duan, L., He, W.-S., and Zhang, C.-M. (2013). Prognostic value of HMGB3 expression in patients with non-small cell lung cancer. Tumour Biol. 34, 2599-2603.
Song, X., Ji, J., Gleason, K. J., Yang, F., Martignetti, J. A., Chen, L. S., and Wang, P. (2019). Insights into Impact of DNA Copy Number Alteration and Methylation on the Proteogenomic Landscape of Human Ovarian Cancer via a Multi-omics Integrative Analysis. Mol. Cell. Proteomics 18, S52-S65.
Subramanian, J., and Govindan, R. (2007). Lung cancer in never smokers: a review. J. Clin. Oncol. 25, 561-570.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc. Natl. Acad. Sci. U.S.A. 102, 15545-15550.
Subramanian, A., Narayan, R., Corsello, S. M., Peck, D. D., Natoli, T. E., Lu, X., Gould, J., Davis, J. F., Tubelli, A. A., Asiedu, J. K., et al. (2017). A Next Generation Connectivity Map: L1000 Platform and the First 1,000,000 Profiles. Cell 171, 1437-1452.e17.
Sun, S., Schiller, J. H., and Gazdar, A. F. (2007). Lung cancer in never smokers—a different disease. Nat. Rev. Cancer 7, 778-790.
Sunaga, N., Imai, H., Shimizu, K., Shames, D. S., Kakegawa, S., Girard, L., Sato, M., Kaira, K., Ishizuka, T., Gazdar, A. F., et al. (2012). Oncogenic KRAS-induced interleukin-8 overexpression promotes cell growth and migration and contributes to aggressive phenotypes of non-small cell lung cancer. Int. J. Cancer 130, 1733-1744.
Svinkina, T., Gu, H., Silva, J. C., Mertins, P., Qiao, J., Fereshetian, S., Jaffe, J. D., Kuhn, E., Udeshi, N. D., and Carr, S. A. (2015). Deep, Quantitative Coverage of the Lysine Acetylome Using Novel Anti-acetyl-lysine Antibodies and an Optimized Proteomic Workflow. Mol. Cell. Proteomics 14, 2429-2440.
Szolek, A., Schubert, B., Mohr, C., Sturm, M., Feldhahn, M., and Kohlbacher, O. (2014). OptiType: precision HLA typing from next-generation sequencing data. Bioinformatics 30, 3310-3316.
Taguchi, K., and Yamamoto, M. (2017). The KEAP1-NRF2 System in Cancer. Front. Oncol. 7, 85.
Takada, K., Kohashi, K., Shimokawa, M., Haro, A., Osoegawa, A., Tagawa, T., Seto, T., Oda, Y., and Maehara, Y. (2019). Co-expression of 001 and PD-L1 in lung squamous cell carcinoma: Potential targets of novel combination therapy. Lung Cancer 128, 26-32.
Takei, N., Yoneda, A., Sakai-Sawada, K., Kosaka, M., Minomi, K., and Tamura, Y. (2017). Hypoxia-inducible ERO1α promotes cancer progression through modulation of integrin-β1 modification and signalling in HCT116 colorectal cancer cells. Sci. Rep. 7, 9389.
Takeuchi, K., Soda, M., Togashi, Y., Suzuki, R., Sakata, S., Hatano, S., Asaka, R., Hamanaka, W., Ninomiya, H., Uehara, H., et al. (2012). RET, ROS1 and ALK fusions in lung cancer. Nat. Med. 18, 378-381.
Tan, V. Y. F., and Fèvotte, C. (2013). Automatic relevance determination in nonnegative matrix factorization with the β-divergence. IEEE Trans. Pattern Anal. Mach. Intell. 35, 1592-1605.
Tang, B., Tian, Y., Liao, Y., Li, Z., Yu, S., Su, H., Zhong, F., Yuan, G., Wang, Y., Yu, H., et al. (2019). CBX8 exhibits oncogenic properties and serves as a prognostic factor in hepatocellular carcinoma. Cell Death Dis. 10, 52.
Tang, C., Luo, D., Yang, H., Wang, Q., Zhang, R., Liu, G., and Zhou, X. (2013). Expression of SHP2 and related markers in non-small cell lung cancer: a tissue microarray study of 80 cases. Appl. Immunohistochem. Mol. Morphol. 21, 386-394.
Tang, D., Liu, J. J., Rundle, A., Neslund-Dudas, C., Savera, A. T., Bock, C. H., Nock, N. L., Yang, J. J., and Rybicki, B. A. (2007). Grilled meat consumption and PhIP-DNA adducts in prostate carcinogenesis. Cancer Epidemiol. Biomarkers Prev. 16, 803-808.
Tate, J. G., Bamford, S., Jubb, H. C., Sondka, Z., Beare, D. M., Bindal, N., Boutselakis, H., Cole, C. G., Creatore, C., Dawson, E., et al. (2019). COSMIC: the Catalogue Of Somatic Mutations In Cancer. Nucleic Acids Res. 47, D941-D947.
Tessema, M., Yingling, C. M., Picchi, M. A., Wu, G., Ryba, T., Lin, Y., Bungum, A. O., Edell, E. S., Spira, A., and Belinsky, S. A. (2017). ANK1 Methylation regulates expression of MicroRNA-486-5p and discriminates lung tumors by histology and smoking status. Cancer Lett. 410, 191-200.
Thompson, P. R., Wang, D., Wang, L., Fulco, M., Pediconi, N., Zhang, D., An, W., Ge, Q., Roeder, R. G., Wong, J., et al. (2004). Regulation of the p300 HAT domain via a novel activation loop. Nat. Struct. Mol. Biol. 11, 308-315.
Tomioka, K., Saeki, K., Obayashi, K., and Kurumatani, N. (2016). Risk of Lung Cancer in Workers Exposed to Benzidine and/or Beta-Naphthylamine: A Systematic Review and Meta-Analysis. J. Epidemiol. 26, 447-458.
Tsherniak, A., Vazquez, F., Montgomery, P. G., Weir, B. A., Kryukov, G., Cowley, G. S., Gill, S., Harrington, W. F., Pantel, S., Krill-Burger, J. M., et al. (2017). Defining a Cancer Dependency Map. Cell 170, 564-576.e16.
Tufo, G., Jones, A. W. E., Wang, Z., Hamelin, J., Tajeddine, N., Esposti, D. D., Martel, C., Boursier, C., Gallerne, C., Migdal, C., et al. (2014). The protein disulfide isomerases PDIA4 and PDIA6 mediate resistance to cisplatin-induced cell death in lung adenocarcinoma. Cell Death Differ. 21, 685-695.
Udeshi, N. D., Mani, D. C., Satpathy, S., Fereshetian, S., Gasser, J. A., Svinkina, T., Ebert, B. L., Mertins, P., and Carr, S. A. UbiFast, a rapid and deep-scale ubiquitylation profiling approach for biology and translational research.
Vasaikar, S. V., Straub, P., Wang, J., and Zhang, B. (2018). LinkedOmics: analyzing multi-omics data within and across 32 cancer types. Nucleic Acids Res. 46, D956-D963.
Vemulapalli, V., Chylek, L., Erickson, A., and LaRochelle, J. (2019). Time resolved quantitative phosphoproteomics reveals distinct patterns of SHP2 dependence in EGFR signaling. bioRxiv.
Vigil, D., Cherfils, J., Rossman, K. L., and Der, C. J. (2010). Ras superfamily GEFs and GAPs: validated and tractable targets for cancer therapy? Nat. Rev. Cancer 10, 842-857.
Vogel, W., and Ullrich, A. (1996). Multiple in vivo phosphorylated tyrosine phosphatase SHP-2 engages binding to Grb2 via tyrosine 584. Cell Growth Differ. 7, 1589-1597.
Vogelstein, B., Papadopoulos, N., Velculescu, V. E., Zhou, S., Diaz, L. A., Jr, and Kinzler, K. W. (2013). Cancer genome landscapes. Science 339, 1546-1558.
Walser, T., Cui, X., Yanagawa, J., Lee, J. M., Heinrich, E., Lee, G., Sharma, S., and Dubinett, S. M. (2008). Smoking and lung cancer: the role of inflammation. Proc. Am. Thorac. Soc. 5, 811-815.
Wang, D., Hao, T., Pan, Y., Qian, X., and Zhou, D. (2015). Increased expression of SOX4 is a biomarker for malignant status and poor prognosis in patients with non-small cell lung cancer. Mol. Cell. Biochem. 402, 75-82.
Wang, J., Vasaikar, S., Shi, Z., Greer, M., and Zhang, B. (2017). WebGestalt 2017: a more comprehensive, powerful, flexible and interactive gene set enrichment analysis toolkit. Nucleic Acids Res. 45, W130-W137.
Wang, X., Slebos, R. J. C., Wang, D., Halvey, P. J., Tabb, D. L., Liebler, D. C., and Zhang, B. (2012). Protein identification using customized protein sequence databases derived from RNA-Seq data. J. Proteome Res. 11, 1009-1017.
Wang, Y., Kuan, P. J., Xing, C., Cronkhite, J. T., Torres, F., Rosenblatt, R. L., DiMaio, J. M., Kinch, L. N., Grishin, N. V., and Garcia, C. K. (2009). Genetic defects in surfactant protein A2 are associated with pulmonary fibrosis and lung cancer. Am. J. Hum. Genet. 84, 52-59.
Weir, B. A., Woo, M. S., Getz, G., Perner, S., Ding, L., Beroukhim, R., Lin, W. M., Province, M. A., Kraja, A., Johnson, L. A., et al. (2007). Characterizing the cancer genome in lung adenocarcinoma. Nature 450, 893-898.
Wen, B., Wang, X., and Zhang, B. (2019). PepQuery enables fast, accurate, and convenient proteomic validation of novel genomic alterations. Genome Res. 29, 485-493.
Whitsett, J. A., and Alenghat, T. (2015). Respiratory epithelial cells orchestrate pulmonary innate immunity. Nat. Immunol. 16, 27-35.
Wiel, C., Le Gal, K., Ibrahim, M. X., Jahangir, C. A., Kashif, M., Yao, H., Ziegler, D. V., Xu, X., Ghosh, T., Mondal, T., et al. (2019). BACH1 Stabilization by Antioxidants Stimulates Lung Cancer Metastasis. Cell 178, 330-345.e22.
Wilkerson, M. D., and Hayes, D. N. (2010). ConsensusClusterPlus: a class discovery tool with confidence assessments and item tracking. Bioinformatics 26, 1572-1573.
Wilkerson, M. D., Yin, X., Walter, V., Zhao, N., Cabanski, C. R., Hayward, M. C., Miller, C. R., Socinski, M. A., Parsons, A. M., Thorne, L. B., et al. (2012). Differential pathogenesis of lung adenocarcinoma subtypes involving sequence mutations, copy number, chromosomal instability, and methylation. PLoS One 7, e36530.
Wing, K., Onishi, Y., Prieto-Martin, P., Yamaguchi, T., Miyara, M., Fehervari, Z., Nomura, T., and Sakaguchi, S. (2008). CTLA-4 control over Foxp3+ regulatory T cell function. Science 322, 271-275.
Xie, K., Zhang, K., Kong, J., Wang, C., Gu, Y., Liang, C., Jiang, T., Qin, N., Liu, J., Guo, X., et al. (2018). Cancer-testis gene PIWIL1 promotes cell proliferation, migration, and invasion in lung adenocarcinoma. Cancer Med. 7, 157-166.
Xu, J., Lamouille, S., and Derynck, R. (2009). TGF-beta-induced epithelial to mesenchymal transition. Cell Res. 19, 156-172.
Xu, Q.-W., Zhao, W., Wang, Y., Sartor, M. A., Han, D.-M., Deng, J., Ponnala, R., Yang, J.-Y., Zhang, Q.-Y., Liao, G.-Q., et al. (2012). An integrated genome-wide approach to discover tumor-specific antigens as potential immunologic and clinical targets in cancer. Cancer Res. 72, 6351-6361.
Yang, C., Peng, P., Li, L., Shao, M., Zhao, J., Wang, L., Duan, F., Song, S., Wu, H., Zhang, J., et al. (2016). High expression of GFAT1 predicts poor prognosis in patients with pancreatic cancer. Sci. Rep. 6, 39044.
Ye, K., Schulz, M. H., Long, Q., Apweiler, R., and Ning, Z. (2009). Pindel: a pattern growth approach to detect break points of large deletions and medium sized insertions from paired-end short reads. Bioinformatics 25, 2865-2871.
Yoshihara, K., Shahmoradgoli, M., Martínez, E., Vegesna, R., Kim, H., Torres-Garcia, W., Trevino, V., Shen, H., Laird, P. W., Levine, D. A., et al. (2013). Inferring tumour purity and stromal and immune cell admixture from expression data. Nat. Commun. 4, 2612.
Zhang, H., Liu, T., Zhang, Z., Payne, S. H., Zhang, B., McDermott, J. E., Zhou, J.-Y., Petyuk, V. A., Chen, L., Ray, D., et al. (2016). Integrated Proteogenomic Characterization of Human High-Grade Serous Ovarian Cancer. Cell 166, 755-765.
Zhang, W., Zhang, J., Zhang, Z., Guo, Y., Wu, Y., Wang, R., Wang, L., Mao, S., and Yao, X. (2019a). Overexpression of Indoleamine 2,3-Dioxygenase 1 Promotes Epithelial-Mesenchymal Transition by Activation of the IL-6/STAT3/PD-L1 Pathway in Bladder Cancer. Translational Oncology 12, 485-492.
Zhang, W., Zhang, J., Zhang, Z., Guo, Y., Wu, Y., Wang, R., Wang, L., Mao, S., and Yao, X. (2019b). Overexpression of Indoleamine 2,3-Dioxygenase 1 Promotes Epithelial-Mesenchymal Transition by Activation of the IL-6/STAT3/PD-L1 Pathway in Bladder Cancer. Transl. Oncol. 12, 485-492.
Zhao, W., Lu, D., Liu, L., Cai, J., Zhou, Y., Yang, Y., Zhang, Y., and Zhang, J. (2017). Insulin-like growth factor 2 mRNA binding protein 3 (IGF2BP3) promotes lung tumorigenesis via attenuating p53 stability. Oncotarget 8, 93672-93687.
Zhou, B., Flodby, P., Luo, J., Castillo, D. R., Liu, Y., Yu, F.-X., McConnell, A., Varghese, B., Li, G., Chimge, N.-O., et al. (2018). Claudin-18-mediated YAP activity regulates lung stem and progenitor cell homeostasis and tumorigenesis. J. Clin. Invest. 128, 970-984.

### Example 3: Proteogenomic and Metabolomic Characterization of Human Glioblastoma

This example describes the analysis of protein phosphorylation identified key signaling intermediates in the RTK/RAS pathway (PTPN11 and PLCG1) common to multiple RTK genomic alterations, potentially offering therapeutic targets for different oncogenic drivers in GBM. Also described herein is the identification of distinct immune-high and immune-low phenotypes in GBM, driven by tumor-associated macrophage markers, and associated with distinct epigenetic modifications and histone acetylation patterns. Furthermore, phosphoproteomics identified potential druggable interactions based on kinase-substrate pathway analysis, as well as novel phosphoprotein targets associated with the regulation of telomere length. Additionally, identification of metabolic changes in IDH mutants were found to facilitate the accumulation of oncometabolite 2-HG and changes in the level of lipid second messengers (e.g., DG) associated with PLCG1 and AKT1 expression.

Glioblastoma (GBM) is the most aggressive central nervous system cancer; with a median survival time of less than 2 years. Understanding the underlying molecular mechanisms of pathogenesis in order to improve the diagnosis and treatment of patients suffering from this deadly disease has motivated us to conduct a first-of-its-kind multi-omics investigation of 99 treatment-naive GBMs including proteogenomics, posttranslational modifications (PTMs) and metabolomics. Phosphoproteomic analysis identified PTPN11 and PLCG1 as the principal switches mediating RAS pathway activation, and therefore potential therapeutic targets. Additional novel targets were identified for EGFR-altered and RB-1 altered tumors. We identified two immune subtypes of GBM based on differential gene and protein expression of macrophage and immune checkpoint markers, and also an association with epigenetic modifications of CSF-1R, PD-1, and CD4. Acetylation of Histone H2B is a key component of classical-type and immune-low GBM, driven largely by BRDs, CREBBP, and EP300. Additionally, lipid abundances vary across subtypes, consistent with the expression of proteins involved in lipid signaling, an insight that can only be revealed by multi-omics data. Metabolomic analysis also identified changes in glycolysis/TCA metabolic cascade in IDH mutants potentially driving the upregulation of oncometabolite 2-HG. Overall, this work points to new additional therapeutic avenues to stratify patients for appropriate and effective treatment.

Introduction

Glioblastoma (GBM) is the most common primary malignant brain tumor with an incidence of 3.22 per 100,000 persons in the United States (˜11,800 newly diagnosed patients per year), a median survival of less than 2 years from diagnosis (Delgado-Lopez and Corrales-Garcia, 2016; Ostrom et al., 2019) and limited treatment options (Lim et al., 2018; Reifenberger et al., 2017). The Cancer Genome Atlas (TCGA) (Brennan et al., 2013; The Cancer Genome Atlas Research Network, 2008) and focused studies (Yan et al., 2009) have reshaped the World Health Organization classification of nervous system tumors (Louis et al., 2016), which now includes hallmark molecular features (Louis et al., 2017). GBM is categorized as either IDH-wild type (˜90%) or IDH-mutant (˜10%). Many IDH-mutant tumors arise from lower grade tumors (secondary GBMs) and are associated with younger age at diagnosis and better prognosis (Ohgaki and Kleihues, 2013). TCGA surveyed 206 GBMs with genomic and transcriptomic profiling (The Cancer Genome Atlas Research Network, 2008), extending the subclassifications developed by Aldape and colleagues (Phillips et al., 2006; Verhaak et al., 2010). The current view is that IDH-wild type GBMs fall into three distinct subclasses: proneural, classical, and mesenchymal, based on genomic alterations and gene expression signatures (Brennan et al., 2013; Ceccarelli et al., 2016; Noushmehr et al., 2010; The Cancer Genome Atlas Research Network, 2008; Verhaak et al., 2010; Wang et al., 2017). Tumor microenvironment may also play an important role in GBM pathogenesis, where tumor-associated macrophages (TAMs) comprise the majority of the immune population (Charles et al., 2012; Hambardzumyan et al., 2016; Quail and Joyce, 2017). TAMs originate from tissue-resident microglia and bone marrow-derived macrophages (BMDMs), with evidence suggesting that they facilitate tumor proliferation, survival, and migration (Hambardzumyan et al., 2016). M2-type macrophages, typically thought of as immunosuppressive, dominate and may serve as a microenvironmental advantage to the tumor (Kennedy et al., 2013).

Despite extensive molecular and immunological characterization of GBM, surgical resection followed by concurrent chemotherapy and radiotherapy remain standard of care (Stupp et al., 2005; Hegi et al., 2005; Perry et al., 2017), with the recent addition of tumor treating fields (Stupp et al., 2017); while targeted therapies have been disappointing. Several promising immunotherapies have been proposed, including checkpoint inhibitors, vaccines, Chimeric antigen receptor (CAR) T-cell therapy, and viral therapy, though none have yet demonstrated therapeutic efficacy in phase 3 clinical trials (Lim et al., 2018; McGranahan et al., 2019; Reifenberger et al., 2017). Importantly, the current state of the art is to treat all GBM patients uniformly upon diagnosis; subtype notwithstanding, no treatment has been found to work in a pre-specified subset of patients based on transcriptomic differences. Hence, there is an urgent need for a more nuanced understanding of tumor cells beyond gene expression, and the tumor microenvironment in a manner that is predictive of precision therapy efficacy. To further investigate GBM biology and inform new therapeutic options, for the first time, we integrated proteogenomic and metabolomic data from 10 experimental platforms for 99 treatment-naive GBM tumors, and 10 unmatched normal brain samples obtained from the Genotype Tissue Expression (GTEx) project (GTEx Consortium, 2013). Besides standard whole genome, whole exome, and RNA sequencing (WGS/WES/RNA-Seq), quantitative proteome, phosphoproteome, and acetylome data obtained for all 109 samples using isobaric peptide labeling (11-plexed tandem mass tags; TMT-11) and liquid chromatography-tandem mass spectrometry (LC-MS/MS), 97, 106, and 82 samples were also characterized by DNA methylation array, miRNA-seq, and lipidome and metabolome quantification using LC-MS/MS and gas chromatography mass spectrometry (GC-MS), respectively (FIG. 15A). Herein, we report newly discovered immune-based subtypes and findings on the regulation of histone H2B by bromodomain proteins and acetyltransferases, differential lipid abundance across subtypes, and the metabolic cascades contributing to the upregulation of oncometabolite 2-hydroxyglutarate (2-HG) in IDH mutants. Our comprehensive phosphoproteomic and metabolomic investigation of 99 GBM cases provided an unprecedented opportunity for discovering novel therapeutic possibilities by disrupting co-regulated kinase-substrate interactions, redirecting macrophages to reverse their tumor-promoting function, and intervening in onco-metabolic cascades. Our high quality, comprehensive datasets and findings provide a rich resource for the cancer research community provides an opportunity to significantly advance our understanding of the molecular biology of GBM and assist in appropriately stratifying patients for the most suitable treatments.

Results

Proteogenomic and Metabolomic Features Delineate Molecular Subtypes of Glioblastoma

We extensively characterized the proteogenomic landscape of 99 prospectively collected, treatment-naive glioblastomas (GBMs) (55 males and 44 females) and 10 unmatched normal brain samples (5 males and 5 females) from the GTEx project. Samples represented diverse countries of origin and ethnicities from the United States, China, Poland, and Russia. Our cohort was comprised of 34 frontal, 34 temporal, 12 parietal, 5 occipital lobe tumors, and 14 tumors involving multiple lobes or deep midline structures (e.g., thalamic gliomas); the GTEx samples were all from the frontal lobe. We observed that more tumors occurred in the frontal lobe in male than in female in our samples (22 [40%] vs. 12 [27%]), but the difference was not statistically significant (Fisher's exact test p=0.21). The age at initial diagnosis ranged from 24 to 88 years old, including 10 adolescents and young adults (AYA; 18 to 39 yr). Ninety-three cases have available survival data, for which the median and 2-year survival rates were 16.3 months (497 days) and 33.3%, respectively. There were six IDH1 R132H hotspot mutant cases in our cohort, which showed earlier disease onset (median 47 yr) than IDH wild type cases (median 59 yr) (t-test p=0.055), consistent with previous reports (Popov et al., 2013; Yan et al., 2009). We also detected one additional non-hotspot IDH1 R222C mutation. No 1DH2 mutants were observed.

All tumors and GTEx normals were first homogenized via cryopulverization and aliquoted for 10 different assays, comprising WES, WGS, RNA-seq, and miRNA sequencing (miRNA-seq), DNA methylation microarray, and proteome, phosphoproteome, acetylome, lipidome, and metabolome (FIG. 15A, FIG. 22A-FIG. 22C; STAR Methods). Protein expression, phosphorylation and acetylation were analyzed using an integrated TMT labeling-based quantitative proteomics workflow (FIG. 22A). The relative abundances of proteins and post-translational modification (PTM) sites across the tumor and normal tissue samples were quantified using a universal reference strategy (Mertins et al., 2016; Zhang et al., 2016a) at a stringent 1% false discovery rate (FDR) cutoff at the protein level. Protein identification and quantification results, along with normalization methods, were carefully evaluated to confirm data quality (STAR Methods, FIG. 22A-FIG. 22C). The metabolome and lipidome levels were measured using established label-free methods by GC-MS and LC-MS, respectively (STAR Methods). Genomic properties of our cohort were comparable to those of the TCGA GBM cohort (Brennan et al., 2013; The Cancer Genome Atlas Research Network, 2008) (FIG. 15B), for example, key known drivers were represented. We identified a large number of structural variants (SV) in key cancer genes, including EGFR, PTEN, PDGFRA, and NF1. EGFR mutations often co-occurred with EGFR SV and amplification events (p<0.01). In the EGFR-wild type group, an enrichment of genetic alterations of tumor suppressors, such as TP53 (p<0.01), NF1 (p<0.01), RB1 (p=0.03), and ATRX (p=0.03) was seen. We also observed that PIK3CA alterations were mutually exclusive with PIK3R1. Since previous studies reported recurrent TERT promoter (TERTp) mutations in GBM (Killela et al., 2013; Nonoguchi et al., 2013; Vinagre et al., 2013), we used both WES and WGS to identify TERTp mutations with variant allele frequency (VAF) >5%. As a result, 74% of GBM samples were identified with C>T substitutions in either of the two TERTp hotspot mutations (NM_198253.2: c.-124C>T [C228T; chr5:1295113G>A] and c.-146C>T [C250T; chr5:1295135G>A]) (FIG. 15B). Furthermore, we applied GISTIC2 (Mermel et al., 2011) to assess focal and arm-level copy number variations (CNVs) using WGS data (FIG. S23B), obtaining similar CNV patterns as TCGA (Brennan et al., 2013). Briefly, 88 out of 97 tumors possessed chr7 amplification or chr10 deletion at the arm level. In addition, a high portion of samples had focal amplification in FGFR3, PDGFRA, EGFR, and CDK4, and deletions in QKI, CDKN2A/B/C, PTEN, RB1, and NF1.

It has previously been demonstrated that epigenetic modifications could influence the development and therapeutic response of gliomas (Dong and Cui, 2019). By profiling tumor-specific DNA methylation at the probe level, we identified six DNA methylation subtypes, including two distinct G-CIMP subtypes (dm2 and dm6, STAR Methods). Interestingly, dm6 is an IDH1 mutant-specific subtype characterized by a chromatin organization signature, while dm2 tumors demonstrated molecular signatures associated with transcription and mRNA splicing (FIG. 3D). Twenty eight out of 90 tumors (31%) with sufficient data coverage showed hypermethylation in the MGMT promoter region and significantly decreased MGMT RNA and protein abundances (Welch's t-test p=8.6e-11 and 3.1e-07, respectively). MGMT promoter methylation suggests these patients would benefit from temozolomide chemotherapy and radiotherapy (Bady et al., 2012; Hegi et al., 2005). To nominate cancer drivers, iProFun (Song et al., 2019) was used to examine genes whose CNAs and/or DNA methylations have cis-associations with either some or all three types of molecular traits (CNA/methylation, mRNA and protein abundances). We found the promoter hypomethylation of NR2F2 (COUP-TFII) was negatively correlated with its RNA and protein expression. NR2F2 is a transcription factor promoting angiogenesis (Pereira et al., 1999; Qin et al., 2010) and associated with ventral telencephalic stem cell origin (Molnar et al., 2019; Paredes et al., 2016; Pletikos et al., 2014). NR2F2 showed significantly higher protein abundance in temporal tumors than in frontal and parietal tumors (FDR <0.1), which could be a potential temporal-specific marker for tumor associated neural stem cells.

Next, we explored the pattern of miRNA expression in GBM to identify new targets by performing unsupervised Louvain clustering on mature miRNA expression of the 98 tumor samples (STAR Methods). Among the five subgroups of GBM patients identified by their distinctive miRNA expression profiles, three showed significant enrichment for tumor-intrinsic transcriptional subtypes (IDH mutant, proneural, and classical) (Wang et al., 2017), while the remaining two subtypes had mixed composition. One miRNA cluster was markedly enriched for classical subtype tumors and featured high expression of miR-128-5p, miR-204-5p, and miR-183-5p. miR-128-5p is known to promote glioma tumorigenesis (Xue et al., 2016). Due to the role that circular RNAs (circRNAs) play in the development and progression of cancer, we further analyzed and identified 3,670 circular RNA (circRNA) from the RNA-seq data (STAR Methods), among which 375 were consistently observed in over 50% of the tumor samples. The two circRNAs with the highest average abundance in tumor samples were circVCAN and circPTN, which have been previously reported to be over-expressed in GBM tumors versus normal tissues (Song et al., 2016).

To understand the intrinsic molecular characteristics across GBMs, we performed unsupervised multi-omics clustering using CNV, RNA, protein, and phosphosite abundance, revealing 3 stable clusters with distinct genetic alterations and expression signatures (FIG. 15C). We also performed gene expression based subtyping from the latest TCGA classification to our cohort (expression subtype), which classified the tumors as classical, mesenchymal, and proneural ((Wang et al., 2017); STAR Methods). Our multi-omics subtypes and expression subtypes showed high concordance (χ² test; p<1e-6). Based on the enrichment of the expression subtypes, multi-omics subtypes of IDH-wild type tumors were named respectively: cluster nmf1 (proneural-like; N=29), cluster nmf2 (mesenchymal-like; N=37), and cluster nmf3 (classical-like; N=26). Although the multi-omics classification and previous RNA-based classification were strongly correlated, there were 27 tumors (29%) that were re-classified as a different subtype according to the multi-omics. The multi-omics clustering leverages a more complete view of cellular physiology, because it contains data on protein and protein phosphorylation, in addition to RNA. Therefore, these clusters likely represent a more phenotypically homogenous grouping than the RNA-only classification. The representative biological functions of each multi-omics cluster were annotated by pathway enrichment analysis on RNA, protein, and phosphosite abundance. The proneural-like cluster was enriched for synaptic vesicle cycle and neurotransmission transport; active synaptic pathways in the proneural-like cluster might suggest the synaptic integration of GBM (Venkataramani et al., 2019; Venkatesh et al., 2019). The mesenchymal-like cluster, on the other hand, was enriched for innate immune response, including neutrophil degranulation, phagocytosis, and extracellular matrix (ECM) organization. Lastly, the classical-like cluster was enriched for mRNA splicing and metabolism of RNA. Among the 2,249 multi-omics features, most features were based on protein or phosphosite (89.0%) and only 17 features were shared with the gene-expression based features. While distinct features were identified by the two classification approaches, the enriched pathways for nmf1/proneural and nmf2/mesenchymal were consistent. We identified 47 protein and phosphosite features (33 genes) associated with cancer drivers (Bailey et al., 2018), including RHOB and GNAQ in nmf1, PIK3CG and PTPRC in nmf2, and DNMT3A and SETD2 in nmf3.

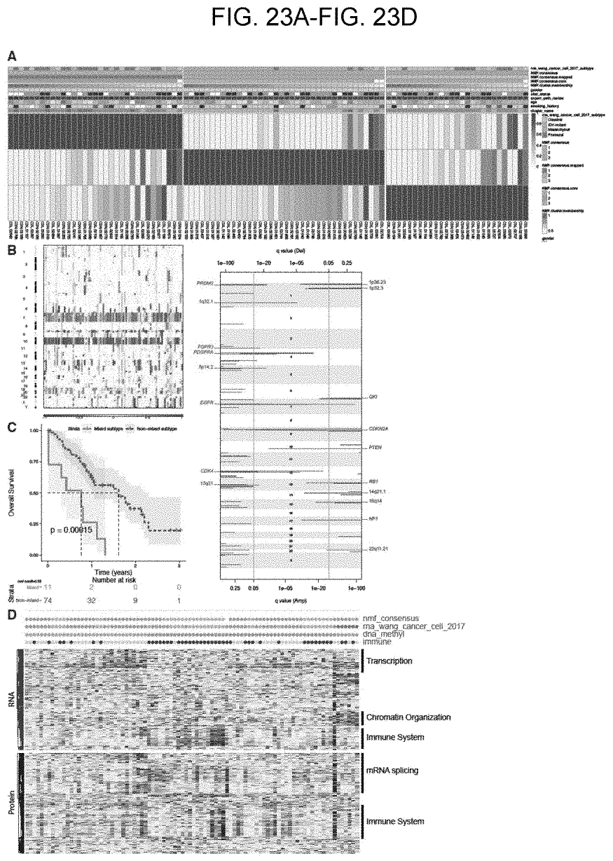

We then associated the tumor subtypes with various clinical data in order to identify molecular features associated with clinical outcome such as prognosis. In our cohort, mesenchymal-like tumors occurred less frequently in the parietal lobe than classical-like and proneural-like tumors (χ² test; p=0.027). Notably, we found 12 tumors with mixed subtypes, showing high multi-omics membership scores in two or more clusters (STAR Methods) and these were associated with a worse prognosis (11 with available survival information; log-rank test p=0.00015; FIG. 23C) in comparison with the tumors of non-mixed subtypes (excluding IDH1 mutants). This corroborates the observation that GBMs with high intratumoral heterogeneity may be more aggressive (Sottoriva et al., 2013). We also identified pathways enriched in each multi-omics cluster at the acetylation level. For instance, the proneural-like cluster showed higher abundance of acetylated proteins involved in TCA cycle and metabolism of amino acids; mesenchymal-like cluster was enriched for innate immune system activation, peroxisomal protein import and glycolysis; and finally, the classical-like subtype was enriched for acetylation of chromatin modifiers and DNA repair proteins.

This CPTAC study is the first of its kind to add another layer of comprehensive lipidome and metabolome datasets to enhance the understanding of tumor biology. We identified more than 300 lipids to be differentially abundant across the three multi-omics subtypes (Wilcoxon test, FDR <0.05, FIG. 15C). Interestingly, the mesenchymal-like subtype demonstrated elevated abundance of triacylglycerols (TGs), as well as depleted levels of phosphatidylcholines (PCs) and other types of phospholipids. As for metabolites, the proneural-like cluster exhibited significantly increased levels of creatinine and homocysteine, and reduced levels of L-cysteine and palatinitol (Wilcoxon test, FDR ≤0.05).

Driver Genetic Alterations Influence Major Oncogenic Processes

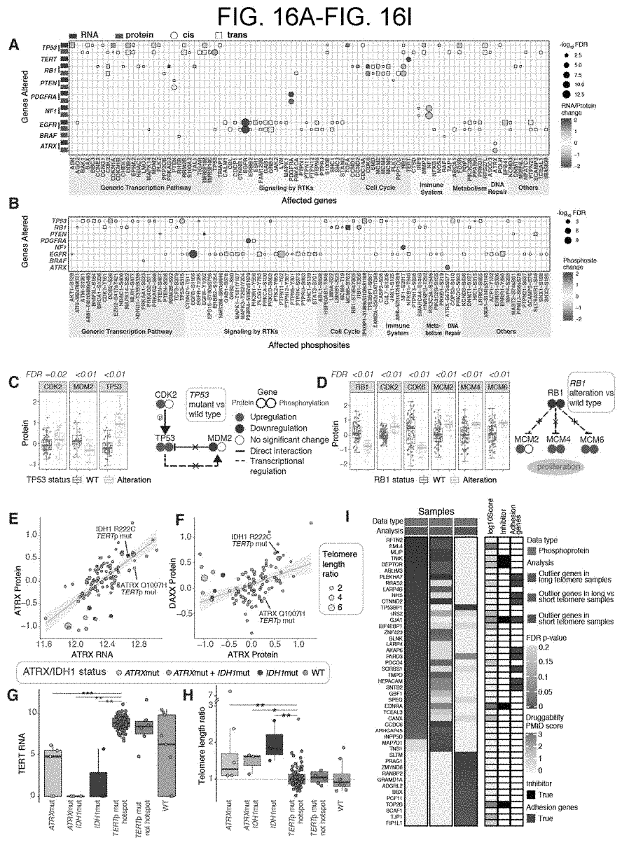

In order to comprehensively understand the breadth of genomic influences on GBM, we characterized the impact of major genetic alterations (mutations, CNVs, fusions, and SVs) on RNA and protein expression and phosphorylation levels in the context of significantly mutated genes (SMGs) (Brennan et al., 2013) (FIG. 16A-FIG. 16B). Strong cis effects were observed for EGFR and PDGFRA, with significant increases in RNA and protein expression and increased phosphorylation at S1166 and S1067/S1070, respectively. At the trans level, we found elevated protein expression of CTNNB1 (β-catenin), a known contributor to lung cancer progression (Nakayama et al., 2014) and high phosphorylation of both PTPN11 (known as Shp2) at Y62 and PLCG1 at Y783 in tumors with EGFR amplification and/or mutation (FIG. 16A-FIG. 16B). The observation that β-catenin protein is increased while the mRNA is decreased illustrates the importance of using both mRNA and protein data.

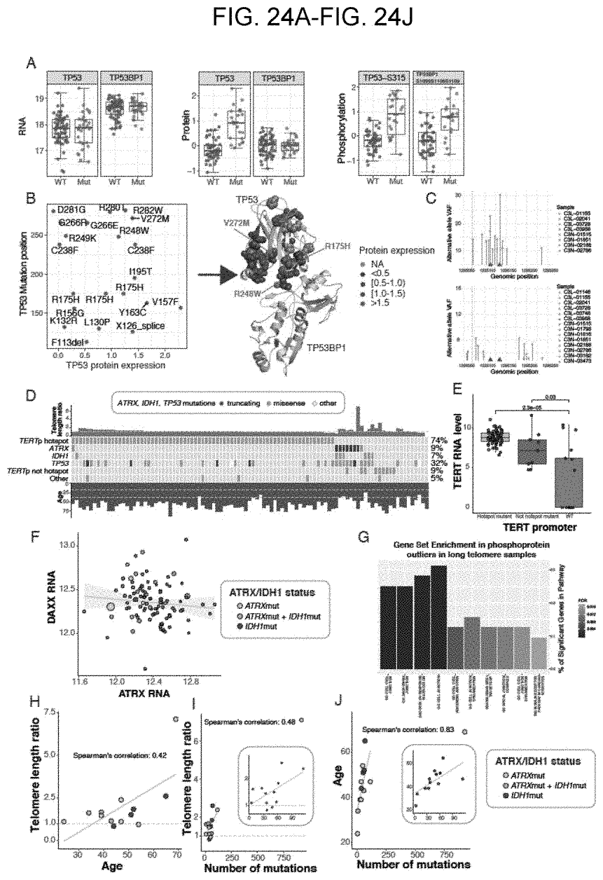

Samples with RB1, NF1, PTEN, and ATRX alterations showed downregulated RNA, protein, and phosphorylation levels, but mutated TP53 showed increased protein expression only, consistent with the known stabilizing effect of most TP53 mutations on protein turnover. Notably, we found that interferon regulatory factor 8 (IRF8) was upregulated at both protein and RNA levels in neurofibromin 1 (NF1)-altered (mutated or deleted) samples (FIG. 16A-FIG. 16B). NF1-altered samples have decreased NF1 RNA, protein and phosphorylation expression. IRF8 is an important transcriptional factor that controls microglial motility (Masuda et al., 2014). Microglia interact with GBM cells and contribute to tumor growth (Neil et al., 1988). Thus, NF-1 genetic aberration has an impact on tumor growth as demonstrated at both RNA and protein levels. In addition, we found that TERT RNA expression is upregulated in samples with TERT promoter mutations. We could not evaluate TERT promoter mutation effects on protein because its protein expression was not detected using current analytical platform. The TP53 binding protein TP53BP1 is more highly phosphorylated, but its RNA and protein expression levels are indistinguishable in TP53-mutated versus wild type samples (FIG. 24A). Phosphorylation levels for both TP53 at S315 and TP53BP1 at S1099, S1106, and S1109 were correlated with TP53 protein expression (Pearson's r=0.89 and 0.53, respectively). Besides known TP53 mutational hotspots at R175, R248, and R273 (Baugh et al., 2018), we found additional TP53 missense mutations and one splice-site mutation, Y126_splice, all with high TP53 protein expression (FIG. 24B). Mapping these mutations to the crystal structure demonstrates that events associated with high TP53 protein expression (>0.5 in relative abundance) are widely distributed within its DNA-binding domain (FIG. 24B).

We also detected high CDK2 at the protein level and low MDM2 at both the RNA and protein levels in TP53-mutated samples (32% of all tumors; FIG. 16A and FIG. 16C). It is known that CDK2 phosphorylates TP53 at S315 (Price et al., 1995), while MDM2 is a principal antagonist of TP53 through a post-transcriptional ubiquitin-dependent mechanism (Moll and Petrenko, 2003). Our data suggest that mutated TP53 fails to stimulate the expression of MDM2, resulting in a reduction of MDM2 RNA and protein expression in TP53-mutated samples (Moll and Petrenko, 2003). Consequently, the low MDM2 expression boosts protein and phosphorylation levels at S315 of TP53. Since this autoregulatory feedback loop from MDM2 to TP53 acts at the post-transcriptional level, as expected, we did not observe any change in TP53 RNA expression between mutated versus wild type samples.

Furthermore, we observed a strong trans effect of RB1 on CDK2, CDK6 and the mini-chromosome maintenance complex proteins including MCM2, MCM4, and MCM6 (FIG. 16A). RB1-altered (mutated or deleted) samples (12% in the cohort) showed significantly downregulated RB1 and upregulated MCM2, MCM4, and MCM6 protein expression (FIG. 16D). RB1 inhibits MCM2, MCM4, and MCM6 activity through negative feedback loop (Simon and Schwacha, 2014), and high MCM2 expression is associated with high cell proliferation in GBM (Hoelzinger et al., 2005). The upregulation of MCM genes suggests an intriguing mechanism of cell proliferation in RB1-altered GBM that may be amenable to treatment with an MCM inhibitor like ciprofloxacin, which penetrates the blood-brain barrier (Lipman et al., 2000; Simon et al., 2013).

We identified that 74% of primary GBM tumors carried TERTp hotspot mutations, resulting in an increase of TERT RNA expression (FIG. 16G and FIG. 24E). An additional 9% of tumors carrying non-hotspot mutations in TERTp also displayed elevated TERT expression, although with considerably more variance. Interestingly, the non-hotspot mutations were observed in close proximity to hotspot positions (within 40 nucleotides), unlike any TERTp substitutions in cases having no TERT expression, which are ≤40 nucleotides upstream or downstream of hotspot positions (FIG. 24C). Hotspot TERTp mutations were reported to be mutually exclusive with ATRX loss associated with alternative lengthening of telomeres phenotype (ALT) (Liu et al., 2019a). In the present study, we found that ATRX mutations were also mutually exclusive with TERTp hotspot mutations and appeared to co-occur with TP53 and IDH1 R132H mutations (FIG. 24D). In light of these nine ATRX mutants, we detected significantly diminished ATRX RNA and protein levels, suggesting the mutations resulted in loss-of-function (FIG. 16E). The remaining missense ATRX mutant, Q1007H, residing outside of the SNF2 and helicase domains, had normal RNA and protein expression, suggesting this mutation is likely non-functional.

We noticed that other mutations can also cause alterations in ATRX protein levels, observing significantly decreased ATRX protein in IDH1 R132H mutants, even though the ATRX RNA level itself remained unchanged (FIG. 16E). Tumors with both ATRX and IDH1 hotspot mutations have the lowest ATRX protein abundances in the entire cohort. These observations suggest either that mutant IDH1 reduces ATRX protein expression through an unknown mechanism, or there is selective pressure in IDH1 mutant tumors for mechanisms that promote low ATRX protein and its associated ALT mechanism. In addition, loss of ATRX did not affect RNA and protein expression of its complex partner DAXX (FIG. 16F), suggesting DAXX is stabilized by mechanisms independent of ATRX. ATRX and DAXX were moderately correlated at the protein level (Spearman's r=0.48, p<10e-5, FIG. 24F), but not at the gene expression level.

ATRX and/or IDH1 mutants displayed longer telomeres than TERTp mutants and wild type (average telomere length ratio was 1.94, 1.15 and 1.08, respectively; FIG. 16H), suggesting ATRX and/or IDH1 R132H tumors indeed have the ALT phenotype characterized by longer telomeres (Mukherjee et al., 2018; Philip et al., 2018). On the contrary, TERTp mutations, both hotspot and non-hotspot, did not result in longer telomeres in tumors. It appeared the telomerase maintains telomeres at roughly the same length over multiple cell division cycles, as opposed to ALT mechanism by which telomeres can become significantly extended. In addition, total mutation counts in patients with ATRX and/or IDH1 mutations were moderately correlated with telomere length ratio (Spearman's r=0.48, p=0.12;) and strongly with patient age (Spearman's r=0.84, p<10e-5, FIG. 24H-FIG. 24J), suggesting that telomere length might indicate tumor age in these patients; however, future studies with larger sample sizes are needed to investigate this hypothesis.

Next, we evaluated the effect of long telomeres (WGS telomere ratio >1.2) and short telomeres (WGS telomere ratio <0.8) on protein expression and phosphorylation levels. We applied an outlier analysis (STAR Methods) to find hyper-phosphorylated genes enriched in tumors with lengthened telomeres. FIG. 16I shows outlier genes that were significantly enriched at the phosphosite level in either long telomeres, or short telomeres, or long versus short telomere groups with druggability annotation from DGIdb (Cotto et al., 2018). A number of outlier gene products in the telomere-long group were cell adhesion related proteins, which were not seen in the telomere-short group. Overrepresentation testing showed enrichment for cell junction pathway in the telomere-long group (FIG. 24G). We also identified potential druggable proteins that might be targeted in the telomere long-group (TNIK, DEPTOR, GJA1, EDNRA), which consisted mostly of ATRX and/or IDH1 mutant tumors and TOP2B protein for telomere-short group. We further applied a deep learning model based on 20,000 sampled tiles of images of hematoxylin-eosin (H&E) staining. Human pathological review of these images determined that the model captured low cellularity, but more vascular structures in telomere-long cases. On the contrary, the telomere-short cases had higher cellularity and less vascularity, while the telomere-normal cases demonstrated an intermediate phenotype. Taken together, these results provide an interesting connection between extended telomeres, lower tumor cellularity and higher vascularity possibly indicated by higher phosphorylation of adhesion proteins, and raises the possibility of novel therapeutic directions for such tumors.

RTK Signaling Cascades are Activated in GBM

Genomic loci associated with receptor tyrosine kinases (RTKs) like EGFR, PDGFRA, and MET are frequently amplified in GBMs (Brennan et al., 2013), with subsequent activation of the oncogenic RAS pathway (Sanchez-Vega et al., 2018) to promote cell proliferation. We systematically explored the genetic alterations, including SVs, fusions, and CNAs, in RTKs and their effects on RNA, protein, and phosphosite levels in multiple components of these pathways, providing an increasingly detailed view of the interconnections between these components.

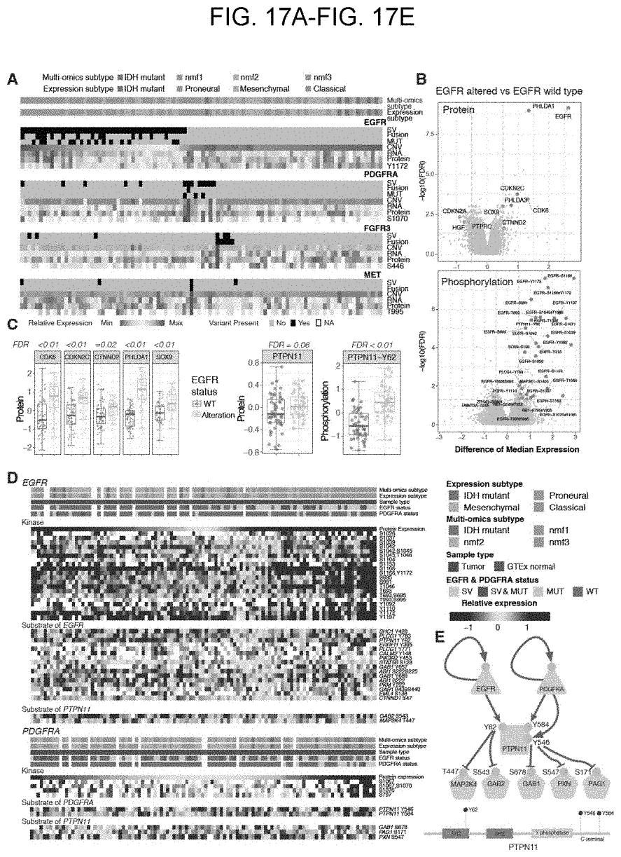

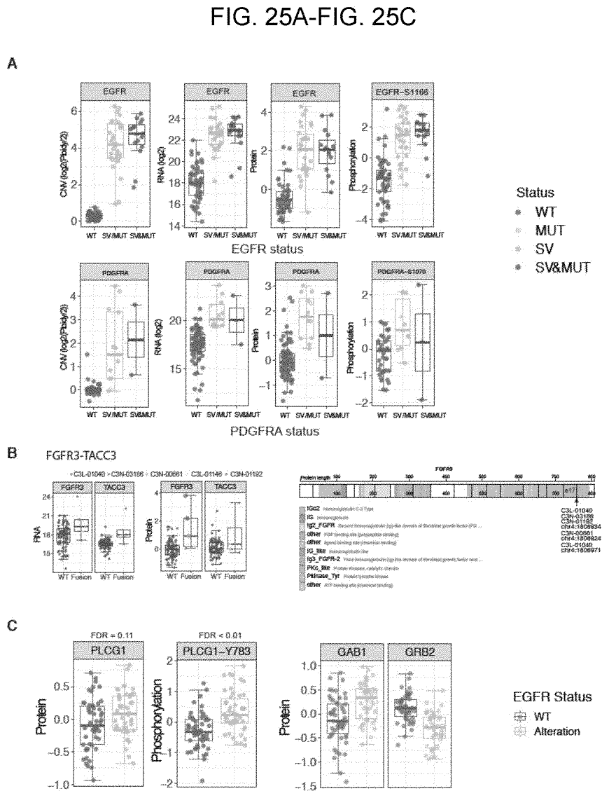

In this regard, we discovered 45 tumors with EGFR SVs, all having copy number amplifications, suggesting high concordance between SV and CNV (FIG. 17A). Interestingly, all samples with mutated EGFR overlap with an SV event. These EGFR-altered tumors have correspondingly high RNA, protein, and phosphorylation levels at Y1172. We did not find expression differences between samples having a sole SV event versus those with a dual mutation and SV event (FIG. 25A), suggesting EGFR upregulation in GBM is largely due to SV, as associated with CNV amplification, rather than mutationally driven, which is different from that in other tumor types, such as lung cancer (Cancer Genome Atlas Research Network, 2014). We also found nine samples in which EGFR SV co-occurred with PDGFRA or FGFR3 SV, while 13 samples with either PDGFRA or FGFR3 alteration did not show any alterations in EGFR. For PDGFRA, two of three mutations overlap with SV events (FIG. 25A). Only one sample with mutation in PDGFRA had high PDGFRA RNA and protein expression. In addition, we found five samples with FGFR3-TACC3 fusions with intact FGFR3 kinase domain (FIG. 25B), and MET SV and amplification co-occurred with PDGFRA SV in one sample.

Interestingly, differential protein and phosphosite levels between EGFR-altered and EGFR-wild type groups demonstrated novel downstream signaling hubs in classical-type GBM. Pleckstrin homology-like domain family A member proteins (PHLDA1 and PHLDA3), transcription factor SOX9, cell adhesion protein CTNND2 (δ-catenin), and cell cycle proteins (CDK6 and CDKN2C) were highly upregulated at the protein level in EGFR-altered samples, together with high autophosphorylation levels of several EGFR sites, and enhanced SOX9 S199 phosphorylation (FIG. 17B-FIG. 17C). These are remarkable findings, given that high PHLDA1 promotes tumor growth (Liu et al., 2019b), EGFR-SOX9 signaling has been implicated in the transition from chronic injury to urothelial cancer (Ling et al., 2011), and SOX9 enhances prostate cancer invasion (Wang et al., 2008). Our finding of higher SOX9 expression in EGFR-altered samples is consistent with a previous study (Liu et al., 2015). The identification of a novel phosphosite at S199 provides an additional resource for studying the causal role of SOX9 in GBM, suggesting a new therapeutic opportunity. In addition, high levels of CDK6 in EGFR-altered samples supports the hypothesis of using CDK6 and EGFR inhibitors for this subset of patients, which have been effective in treating esophageal squamous cell carcinoma with an acceptable toxicity profile (Zhou et al., 2017a). This kind of rational combination strategy should be further tested in a clinical trial.

Several phosphosites were observed as being differentially regulated in tumors with EGFR-alterations (FIG. 17B, bottom). For instance, high levels of PTPN11-Y62, PLCG1-Y783, RB1-S795Y805, SOX9-S199, MAP3K1-S1408, and specific EGFR sites were observed in EGFR-altered samples. Notably, total protein levels of PTPN11 (Shp2) were comparable between the two groups, which suggests that its activity is mainly regulated by phosphorylation (FIG. 17C). PTPN11-Y62 exists at the interface between the N-terminal Src homology 2 (N-SH2) and phosphatase (PTP) domains. PTPN11-Y62 phosphorylation activates PTPN11 by inducing a conformational change (Ren et al., 2010), which in turn, promotes cell proliferation through the activation of the RAS pathway (Sanchez-Vega et al., 2018). To our knowledge, this is the first observation that PTPN11-Y62 is upregulated in EGFR-altered GBMs.

Another potentially important regulatory phosphorylation event in EGFR-altered samples is PLCG1 (known as PLCγ1). There was no significant difference in PLCγ1 protein expression (FDR=0.11), but Y783 phosphorylation was significantly higher in EGFR-altered versus EGFR-wild type samples (FDR <0.01; FIG. 25C). PLCγ1, a group C phospholipase, plays a vital role in actin reorganization and cell migration. In other systems, RTKs are known to phosphorylate PLCγ1 at Y783, which in turn binds to its own C-terminal SH2 (cSH2) domain, activating PLCγ1 (Poulin et al., 2005). Activated PLCγ1 increases cell proliferation, migration, and invasiveness in breast cancer (Sala et al., 2008), cutaneous T-cell lymphomas (Vague et al., 2014), and angiosarcomas (Kunze et al., 2014). Our findings now suggest that PLCγ1 is also activated in GBM through EGFR-mediated phosphorylation of PLCγ1 at Y783.

To further explore the role of phosphorylation-dependent regulation in EGFR-altered samples, we performed a focused kinase-substrate (K-S) study for EGFR and PDGFRA by investigating correlations between EGFR and PDGFRA protein expression and the phosphorylation levels of their substrates using a regression model (STAR Methods). Consistent with the above analyses for differentially regulated phosphorylation sites, we found high phosphorylation levels of EGFR, PTPN11, and PLCγ1 associated with high EGFR protein expression. Notably, the K-S analysis identified high levels of GAB1 phosphorylation at Y689 and Y657 that aligned well with high EGFR expression, and two additional PTPN11 phosphosites at Y546 and Y584 in accordance with high PDGFRA expression (FIG. 17D-FIG. 17E). Previous work has shown two similar PTPN11 phosphosites at the C-terminus in lung cancer samples with ALK fusions (Voena et al., 2007).

PTPN11 inactivation suppresses lung tumors in a mouse model (Schneeberger et al., 2015) and has been explored as an anti-cancer mechanism for drug development (Fodor et al., 2018; Ostman et al., 2006). Here, activation of PTPN11 through EGFR or PDGFRA related phosphorylation suggests a potential therapeutic opportunity for using an anti-PTPN11 drug like SHP099 (Chen et al., 2016) in RTK-altered glioblastomas.

PTPN11 (Shp2), GAB1, and GRB2 form a complex and are co-regulated by RTKs to activate the RAS pathway (Montagner et al., 2005). FIG. 16A and FIG. 25C showed that the activation status of EGFR is associated with upregulated GAB1 and downregulated GRB2 protein expression, consistent with the previous finding that it is GAB1, not GBR2, that promotes tumor progression and that GRB2 behaves functionally more like a tumor suppressor (Seiden-Long et al., 2008).

Distinct Immune Marker Expression and Epigenetic Events Characterize GBM Immune Subtypes

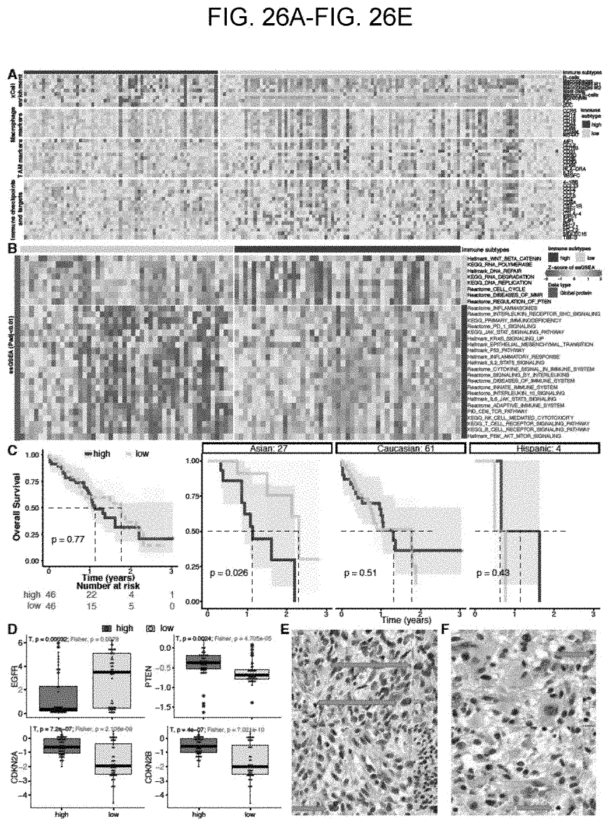

Using the normalized cell-type enrichment score to perform consensus clustering (STAR Methods), we identified two distinct immune-based GBM subtypes. xCell returned higher immune scores for most of the 51 samples in the cluster on the right-side (FIG. 18A), designated as the immune-high group. The other cluster of 48 samples belongs to the immune-low group (FIG. 18A). The immune-high group largely overlaps with the nmf2 multi-omics subtype, and mesenchymal gene expression subtype. The two immune subtypes were also identified and confirmed when we re-analyzed the TCGA GBM transcriptome data (FIGS. 18D and 26A).

The immune-high cluster showed a higher enrichment for microglia, macrophages, monocytes, B cells, dendritic cells, CD4, CD8, among others; and common macrophage markers (e.g., CD14, CD16) had high expression at both gene and protein levels (FIG. 18A). The major non-neoplastic cell population in the GBM microenvironment includes TAMs, which can facilitate tumor growth by their proangiogenic and immunosuppressive properties. Their features align with the so-called M2 phenotype, which promotes wound healing and suppresses adverse immune responses (Chen and Hambardzumyan, 2018).

To further study the importance of TAMs in GBM, we used a pool of TAM marker genes (FIG. 18A), which showed higher overall gene and protein expression in the immune-high cluster. For example, AIF1, which encodes the TAM-specific protein IBA1, is particularly high in this cluster (FIG. 18A) (Kaffes et al., 2019). Gene and protein expression levels of negative regulatory immune checkpoints (e.g., CTLA-4, PD-1, and TIM-3) were also significantly higher in the immune-high group (FIG. 18A). 4-1BB, CCL7, and TIM-3 were captured in the outlier analysis on the gene expression level (FIG. 18A). We also found substantially more high-level amplifications in EGFR and deep deletions in PTEN, CDKN2A, CDKN2B in the immune-low group (FIG. 26D). The DNA methylation dm3 group was strongly associated with the immune-high group showing the immune-related signature (Fisher test p=6.5e-07). Immune-related genes showed distinct methylation patterns in the immune-high group. For example, the promoter regions of CD4, CD8A, CSF-1R, PD-1, and SIGLEC15 were hypomethylated, as detected by multiple probes (FIG. 18A). This highlights the very first observation of distinct epigenetic modifications associated with immune-high and immune-low subtypes in GBM and will enable further clarification for immunotherapy trial design.

To understand the biological differences between the two GBM immune subtypes, we investigated differential abundance at protein and gene expression, and phosphorylation and acetylation levels (FDR=0.05) for overrepresented pathways (Hallmark, KEGG, Reactome). Pathway enrichment was based on an FDR=0.1 threshold and limited to pathways having at least 10 genes observed in each data type. Pathways upregulated across the four data types in the immune-high subtype included immune system, innate immune system, neutrophil degranulation, signaling by interleukins, and others as indicated (FIG. 18A). The unique availability of protein abundance also captured the pathways that were missed by gene expression such as signaling by BRAF and RAF fusions (FIG. 18A). Cell cycle, telomere maintenance, and regulation of PTEN gene transcription and so on were upregulated in the immune-low subtype (FIG. 18A). We validated the pathway enrichment analysis by performing single-sample gene-set enrichment analysis (ssGSEA) of each immune subtype (FIG. 26B).

In addition, the immune-high subtype was strongly associated with high immune scores (ESTIMATE and xCell), low mRNA stemness index, and WGS mutation counts (log₂-scaled) (STAR Methods, FIG. 18C). The high-level copy number amplifications in EGFR, and deep deletions in CDKN2A and CDKN2B were enriched in the immune-low subtype (FIG. 18A and FIG. 26D). The overall survival of the two immune groups were compared in FIG. 26C. Although the overall survival between the two immune subtypes was not significantly different (p=0.77), the median survival of the immune-high subtype (1.13 years) was shorter than that of the immune-low subtype (1.77 years), suggesting that immune-high group reflects a more suppressive or exhausted microenvironment. Markers such as C3, HIF1A, PD-L1, TIM-3 associated with inhibition of killing tumor cells were highly expressed in the immune-high subtype at both the gene expression and protein levels. In Asians, this difference was significant (p=0.026) (FIG. 26C). We further visualized the features captured by a deep learning model from 20,000 sampled tiles from H&E staining. Pathological review of these tiles in the immune-high and immune-low clusters based on t-distributed Stochastic Neighbor Embedding (t-SNE) plotting (FIG. 18B, FIG. 26E-FIG. 26F) found: 1) a substantial number of giant cells (an uncommon subtype of glioblastoma) exist in immune-low tiles but few reside in immune-high tiles; 2) inflammatory cell fractions of ˜5% and ˜20% in immune-low and immune-high tiles, respectively; and 3) microcystic changes and a few vascular structures in immune-high tiles that were absent in immune-low tiles. These features support the immune-high and immune-low classification of GBM, where immune-high group is characterized by the larger fraction of the inflammatory cells and vascular structures.

Compared to the immune-low subtype, the immune-high subtype had increased levels of immune checkpoint proteins (e.g., CTLA-4, PD-1, TIM-3), macrophage-specific cytokines (e.g., CSF-1) (Pyonteck et al., 2013), and monocyte chemoattractant protein (MCP) family members in humans (CCL2, CCL7, CCL8, and CCL13) which play an essential role in mediating monocyte migration and tissue infiltration (Chen and Hambardzumyan, 2018). This differential expression of key immune modulators suggests a strategy for improved selection of patients for immunotherapies.

Differential Acetylation of Histone is Associated with Subtypes and Pathways

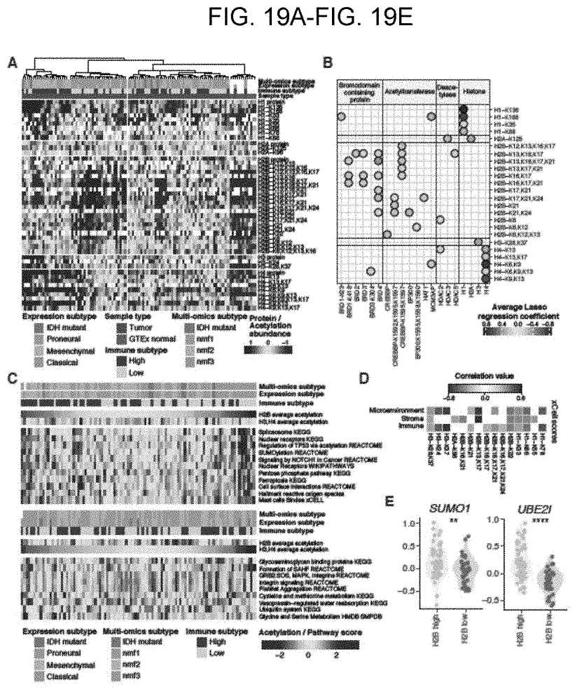

We detected over 30 acetylation sites on histones H1, H2A, H2B, H3.3 and H4. Unsupervised clustering of these sites across 99 tumor and 10 GTEx normal samples identified subsets of tumors with differentially acetylated histones H1, H2B, H3.3, and H4 (FIG. 19A). Association of acetylation and induced gene expression has been established for histones H3 and H4, but little is known for H2A and H2B acetylation, even though multiple acetylation sites have been identified. Both protein and acetylation levels of histones H1, H3.3, and H4 correlated among each other in our cohort, while acetylation of H2B and H2A did not. The association between H1 and H3/H4 acetylation we observed may indicate synergistic regulation of overall chromatin architecture and accessibility. Conversely, H2A and H2B, components of another dimer of the nucleosome, did not show strong correlation in their protein or acetylation abundances. We found that histone acetylation was generally upregulated in tumors compared to GTEx normal samples, with a subset of tumors having elevated H1, H3, and H4 acetylation, while a different cluster of tumors exhibited significantly increased acetylation of H2B N-terminal sites (FIG. 19A). To further investigate this apparent acetylation-based tumor stratification, we explored the associations with gene expression, tumor subtypes and pathway activity.

To determine potential histone acetyltransferases (HATs) and deacetylases (HDACs) that might control global histone acetylation in GBMs, we performed a Lasso linear regression using acetylation of each histone site as dependent variables and protein expression vectors of the main HATs, HDACs, bromodomain-containing proteins (BRDs) and the protein expression vector of the corresponding histone as independent variables. Additionally, we included acetylation abundance vectors of HATs, HDACs, and BRDs as supplementary dependent variables, since acetylation of a lysine-rich autoacetylation loop in the HAT domain of EP300 protein upregulates its HAT activity (Thompson et al., 2004). A close paralog of EP300, acetyltransferase CREBBP shares a highly conserved autoacetylation loop, suggesting that acetylation of the same domain of CREBBP is also crucial for its activity. Using Lasso regression, we discovered multiple positive associations between HATs and BRDs and H2B acetylation sites. In particular, CREBBP-K1592, K1595, and K1597 acetylation levels and histone H2B-K12, K13, K16, K17, K21 showed substantial associations, suggesting the potential regulation of H2B acetylation by a hyperacetylated CREBBP protein. CREBBP protein level had a single significant correlation with H2B acetylated peptide H2B-K6, K12, K13, while EP300 protein and acetylation each showed association with only one H2B acetylated peptide, K21,K24 (FIG. 19B). These observations suggest that hyperacetylation of H2B in the subset of tumors may be reversed by inhibition of CREBBP/EP300 (Garcia-Carpizo et al., 2018). In addition to HATs, H2B acetylation sites correlated with protein and acetylation abundance of BRD1, BRD3, and BRD4 proteins, which are known to bind acetylated histones and mediate gene transcription (LeRoy et al., 2008). However, the ability of CREBBP, EP300, BRD1, BRD3 and/or BRD4 to bind H2B specifically for the regulation on its acetylation has yet to be established. Acetylation of H1, H2A, H3, and H4 histones has been associated mainly with the levels of corresponding histone proteins, suggesting their acetylation might not be under active regulation in GBM.

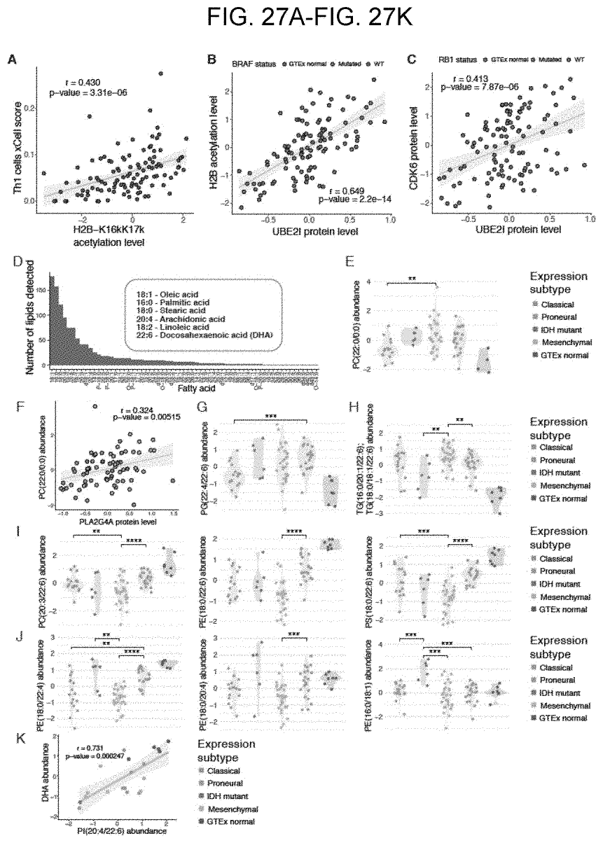

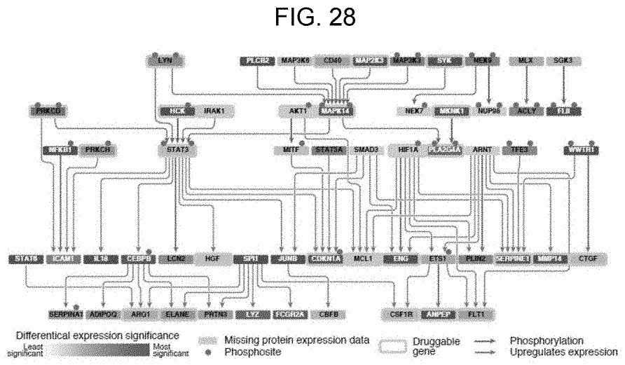

Acetylation of histones was found to be correlated with xCell-derived stromal and immune scores (FIG. 19D). For example, H2B demonstrated significant negative correlation with these scores, while other histones showed positive association with the microenvironment enrichment score. However, H2B acetylation positively correlated with Th1 xCell enrichment score, which was enriched in the immune-low cluster of GBM tumors (FIG. 19A and FIG. 27A). In addition to correlating histone acetylation with immune signatures, we explored the association of all known pathways with histone acetylation levels. We scored each pathway from KEGG, Reactome, WikiPathways, Hallmark, and HMDB SMPDB databases (STAR Methods), identifying pathways strongly correlated with H2B, H3, or H4 average acetylation level. Spliceosome, SUMOylation, and Nuclear receptors pathways were correlated significantly with high H2B acetylation, while a number of pathways related to immune infiltration, such as ferroptosis, mast cells, and reactive oxygen species pathways had negative correlations (FIG. 19C). Notably, positive association between H2B acetylation and SUMOylation pathway activity can be attributed to the fact that a number of transcription factors can be SUMOylated, which is recognized by CREBBP/EP300 (Rosonina et al., 2017) that we found to have a potential impact on H2B acetylation. SUMOylation has recently been explored as a potential therapeutic target for cancer treatment. Depletion of SUMO E2 conjugating enzyme encoded by UBE21 drastically decreases proliferation of BRAF mutant or KRAS mutant malignant cell lines (Yu et al., 2015). Here, we observed that SUMO1 and UBE21 were significantly upregulated in H2B hyperacetylated tumors (FIG. 19E). The combination of BRAF V600E mutations and high H2B acetylation observed in three patients with the subsequently increased expression of UBE21, may hint the use of UBE2I inhibitors for treating those patients (FIGS. 19E and 27B). Besides transcription factors, SUMOylation targets cell cycle regulators such as CDK6 (Fox et al., 2019). Inhibition of SUMOylation results in the decrease of CDK6 protein level in GBM (Bellail et al., 2014; Bernstock et al., 2017), which is consistent with the current data, where UBE2I protein correlated moderately with CDK6 (Pearson's r=0.413, FIG. 27C). Thus, tumors with high CDK6 and UBE2I protein level may also benefit from UBE2I inhibitors, except for RB1 mutants observed to have low CDK6 expression in our data. Our data overall showed a negative association between SUMOylation, and the Pentose Phosphate Pathway and HIF-1 alpha activity pathways (FIG. 19C). This result addresses a current controversy in the field: several studies have demonstrated that SUMOylation of HIF-1 alpha reduces its transcriptional activity (Berta et al., 2007; Kang et al., 2010), while one study showed topotecan, a SUMOylation inhibitor, decreases the activity of G6PD and HIF-1 alpha level in dose-dependent manner (Bernstock et al., 2017). Our data clearly support the former finding.

Lipid Composition and Metabolomic Changes Correlate with GBM Subtypes

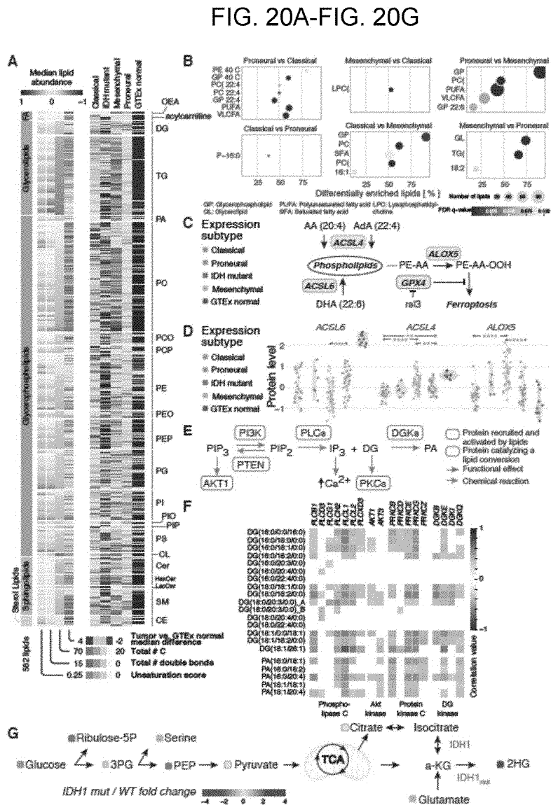

Using LC-MS/MS, we quantified 582 lipids (Fahy et al., 2007) in 75 tumor and 7 GTEx normal samples, including phosphatidylethanolamine (PE), phosphatidylcholine (PC), phosphatidylserine (PS), phosphatidylglycerol (PG) and phosphatidylinositol (PI). Besides glycerophospholipids, we identified triacylglycerols (TG), sphingomyelins (SM), ceramides (Cer), and cholesteryl esters (CE) (FIG. 20A). From the lipidome data, we found that oleic, palmitic, stearic, arachidonic, linoleic, and docosahexaenoic acids (DHA) were incorporated into a large number of lipids (FIG. 27D). The mesenchymal subtype shows high abundance of TGs compared to other subtypes and GTEx normal samples (FIG. 20A). Similar to GTEx normals, the IDH1 mutant subtype exhibited lower abundance of TGs with very long polyunsaturated fatty acids 22 carbons, VLC PUFA), while long-chained (13-21 carbons, LC) TGs were as abundant as those in mesenchymal subtype. PCs were downregulated in the mesenchymal subtype, while monoacyl-PCs were slightly upregulated compared to classical and proneural subtypes (FIG. 27E), consistent with upregulation of PLA2G4A (Pearson's r=0.32, p<0.01, FIG. 27F). We also evaluated the enrichment for certain features in lipids significantly upregulated in one subtype versus another (Wilcoxon test FDR <0.05) using Lipid Mini-On (Clair et al., 2019). The proneural subtype was enriched for glycerophospholipids with long-chain (LC) PUFAs 22:4 and 22:6 compared to classical and mesenchymal subtypes. Mesenchymal subtype, on the other hand, showed enrichment in TGs and lipids with 18:2 fatty acid (likely linoleic acid) (FIG. 20B). Excessive TGs usually accumulate in lipid droplets, which have been linked to the increased chemotherapy resistance (Cotte et al., 2018), suggesting that mesenchymal subtype may be more resistant to chemotherapy treatment.

Next, we investigated the connection between differential abundance of 22:4- and 22:6-containing lipids and proteins involved in the metabolism of those lipids, since they play neuroprotective and anti-cancer roles (Bhagat and Das, 2015; Laviano et al., 2013; Mayurasakorn et al., 2011; Murray et al., 2015). One connection we observed was between 22:6 (likely DHA) and ACSL6 (an acyl-coA synthetase) (FIG. 20C), which is essential for incorporating DHA into phospholipids (Fernandez et al., 2018). Tumor samples had drastically diminished protein expression of ACSL6 (FIG. 20D) and increased content of DHA-containing PGs (FIG. 27G) and TGs (FIG. 27H), while other PE, PC, PS lipids were downregulated (FIG. 27I). In addition, the proneural subtype demonstrated elevated expression of ACSL6 and phospholipids carrying DHA compared to mesenchymal, but decreased abundance of TGs and PGs carrying the same fatty acid. Some of the PG lipids identified in this study are a structural isomer, bis(monoacylglycero)phosphate (BMP). BMP lipids are located to late endosomes and lysosomes and known to be enriched in DHA (Akgoc et al., 2015; Bouvier et al., 2009). Interestingly, a single phosphatidylinositol PI (20:4/22:6) abundance was found to be correlated with free DHA abundance (Pearson's r=0.73, p<0.001), and also to be upregulated in the proneural subtype compared to mesenchymal (FIG. 27K). With respect to DHA metabolism, the proneural subtype appeared to be the most similar while the mesenchymal subtype is the least similar to GTEx normals.

Mesenchymal GBMs have an intriguing profile related to ferroptosis, a form of controlled necrotic cell death, involving peroxidation of LC PUFA phospholipids in the membrane, leading to oxidative degradation of lipids. We identified upregulation of the ferroptosis pathway in mesenchymal GBMs by our H2B acetylation-related pathway analysis (FIG. 19C). For example, ferroptosis-related proteins ACSL4 and ALOX5 were found to be significantly upregulated exclusively in the mesenchymal subtype (FIG. 20D). ACSL4 is an essential protein in this pathway responsible for incorporation of arachidonic acid (AA—20:4) and adrenic acid (AdA—22:4) into PEs (Doll et al., 2017; Kagan et al., 2017); ALOX5 (arachidonate 5-lipoxygenase) catalyzes the oxidation of PUFAs (Gaschler and Stockwell, 2017). Upregulation of ACSL4 and ALOX5 might be an indication of higher content of oxidized PEs in the mesenchymal subtype. In addition, downregulation of intact PE with PUFAs was observed in the mesenchymal subtype (FIG. 27J), attributable to possibly higher content of oxidized PE in this subtype. However, we were unable to detect any oxidized lipids using the current LC-MS/MS approach, a potential limitation of the interpretation of ferroptosis pathway activity. Ferroptosis is negatively regulated by GPX4 (Kagan et al., 2017; Miess et al., 2018), which is not differentially expressed across tumor subtypes. Together, these observations suggest the intriguing possibility that mesenchymal GBMs may be sensitive to GPX4 inhibitors, such as RSL3 (Lu et al., 2017).

Next, we examined the potential connection of lipids to signaling mediated by diacylglycerols (DGs) (FIG. 20E) across subtypes. FIG. 20F shows the correlation coefficients between all DG detected and proteins producing DGs from phospholipids (phospholipases C), proteins being recruited by DGs (protein kinases C), proteins utilizing DGs to form phosphatidic acid (PA) (DG kinases), and Akt kinases being activated by PIP₃ created by PI3K kinase from PIP₂. We found a significant direct correlation between DGs and AKT1, PLCD3, and PLCG1 protein expression (PLCG1 phosphorylation was affected by EGFR alterations [FIG. 16B]). We also observed a significant correlation between PA abundance and DGKE and DGKQ kinase expression. Notably, protein kinases C, usually recruited by DGs, showed only negative correlation with total DGs levels, indicating that their activity mediated by DG binding might not be captured at this level of analysis.

Finally, we explored metabolic alterations in IDH1 mutant tumors by examining our metabolome dataset. We performed statistical analysis for each of the detected metabolites in IDH1 mutants versus IDH1 wild type tumors, and identified 2-HG as the most highly abundant metabolite in IDH1 mutant tumors (median log₂ FC=3.62, FDR <0.05). We also found other differentially present metabolites with p<0.05, although they do not pass the FDR cutoff. Since 2-HG is the most proximal metabolite to mutant IDH1 and is not further metabolized, it is likely that 2-HG accumulates more readily than other metabolites. As other metabolites are subject to constant flux as they are metabolized, we analyzed all metabolites passing p<0.05 cutoff and included them in the pathway diagram in FIG. 20G. Interestingly, a number of metabolites involved in glycolysis showed increased abundance in IDH1 mutants, while serine and glutamate levels were reduced. Glutamate may contribute to alpha-ketoglutarate levels and then to 2-HG levels via GLUD1- and IDH1-catalyzed reactions. Supporting this hypothesis is the negative correlation between GLUD1 protein expression and glutamate abundance (Pearson's r=−0.29, p<0.01). Therefore, the inhibition of GLUD1 that may decrease 2-HG level appears to be an interesting therapeutic opportunity for IDH mutants.

Key Oncogenic Pathways and Therapeutic Opportunities in GBM

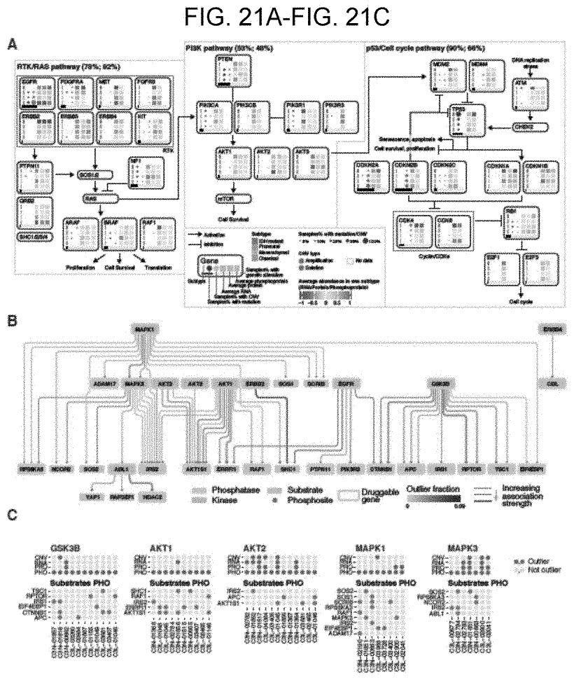

We constructed an integrated proteogenomic map involving three known oncogenic signaling pathways in GBM (Brennan et al., 2013; The Cancer Genome Atlas Research Network, 2008). Specifically, we summarized genetic alterations (mutations and CNVs) and the RNA, protein, and phosphosite levels per expression subtype (FIG. 21A, STAR Methods). Interestingly, we found that the expression outlier percentage was much higher than the genetic alteration rate in RTK pathways, which implies that there may be additional dysregulation through signal transduction. Moreover, all tumors harbored at least one genetic alteration or outlier expression in at least one of the three pathways, suggesting glioblastoma pathogenesis requires at least one of these three pathways to be functionally driven.

Despite similar pathway-level alterations, we observed that tumors of different subtypes utilized different genes in the same pathway. In the RTK/RAS pathway, classical tumors predominantly showed amplified EGFR, while proneural and IDH-mutant tumors showed amplified PDGFRA, both resulting in higher RNA, protein, and phosphosite abundance of EGFR and PDGFRA, respectively. For mesenchymal tumors, we observed upregulated MET and downregulated NF1 protein abundance. In the PI3K pathway, we found that proneural, mesenchymal, and classical tumors showed lower expression of PTEN due to mutations and deletions, which potentially prompted upregulation of AKT1 and AKT2 through the level of phosphatidylinositol-3,4,5-trisphosphate (PIP₃) (not detected in our lipidome data). In contrast, AKT3 expression was relatively higher in IDH-mutant and proneural tumors, which might be explained by the active expression AKT3 in adult brains (Easton et al., 2005) and higher proportion of neurons in the two subtypes based on the xCell deconvolution result. In the p53/cell cycle pathway, we observed amplification and higher expression of MDM2 in mesenchymal and MDM4 in proneural and classical tumors, respectively. We also observed deletion and lower expression of CDKN2A/B in all tumors except for the IDH mutants. Interestingly, TP53 was upregulated in all tumors, suggesting the mixed effect of MDM2/4 and CDKN2A/B and mutations in TP53 itself led to loss of tumor suppressive function (Ham et al., 2019; Zhang et al., 2018). Deletions in CDKN2A/B and amplifications in CDK4 led to elevated CDK4 protein abundance in all tumors. Additionally, CDK6 was highly expressed only in classical tumors. Upregulation of CDK4/6 promotes G1/S progression in cell cycle (Sherr et al., 2016).

Next, we performed extensive kinase-substrate analysis using approximately 250 kinases and their known substrates (STAR Methods) to identify significant and potentially druggable pairs in all GBMs. We found approximately 2400 positively associated kinase-substrate pairs and 50 negatively associated phosphatase-substrate pairs. Overlaying these pairs with phosphosite outlier analysis and druggability information from DGIdb (Cotto et al., 2018) and DEPO (Sun et al., 2018), we sought to identify promising therapeutic targets for GBM patients. Phosphosite outlier analysis (STAR Methods) supported a substantial number of interactions for GSK3B, AKT1, MAPK1, MAPK3 and EGFR (FIG. 21B). Validating this analysis, we found that GSK3B phosphorylation showed positive association with phosphorylation of its downstream proteins involved in mTOR signaling, such as RPTOR and TSC1, and Wnt signaling, such as CTNNB1 and APC. Another player in mTOR signaling, AKT1S1, had many significant connections with AKT1, AKT2 and AKT3 kinases. Interestingly, EGFR was also found to phosphorylate CTNNB1 S33 at the N-terminal, which is known to mediate CTNNB1 proteasomal degradation (Park et al., 2004). The outlying phosphorylation status of GSK3B, AKT1 and MAPK1 is rarely associated with any events at the CNV, RNA or protein levels (FIG. 21C), suggesting that phosphorylation exclusively allows for identification of patients who might benefit from the inhibition of those kinases. Notably, we observed the highest percentage of outliers in kinase-substrate pair of ERBB2 and SHC1 (3 samples), wherein the latter serves as an adaptor protein to pass phosphorylation signaling downstream, eventually driving cell survival and cytoskeletal reorganization. In addition, the MAP kinase cascade demonstrated associations with diverse proteins including the druggable ABL1 kinase, which in turn had a strong association with HDAC2 deacetylase with ˜5% of outliers. We found high HDAC S422 phosphorylation, which was reported to coincide with its functional activity (Eom and Kook, 2015), suggesting HDAC-inhibitor treatment can be applied to patients with outlying ABL1-HDAC2 phosphorylation.

Immunotherapy has unfortunately failed in multiple phase 3 studies in the primary and recurrent GBM settings. However, in each of these studies, patients who do respond have very durable responses, suggesting we need better ways to stratify patients for these types of therapies. To address this and find new immunological targets, we further explored the possible therapeutic targets for the immune active tumors by testing for differential protein and phosphoprotein interactions in mesenchymal tumors over the rest of the tumors using CausalPath (Babur et al., 2018) (FIG. 28A; STAR Methods). We discovered that the hypoxia pathway was particularly activated in mesenchymal tumors. While we did not directly detect HIFIA (HIF-1a) and ARNT (HIF-1β) in the proteome data, a significant number of HIF-1 downstream targets were activated (network permutation p=0.0012). The upregulation of FLT1, MMP14, ENG, and SERPINE1 indicated the promoted angiogenesis in mesenchymal tumors. Importantly, HIF-1 and its downstream targets are druggable. We also observed complex regulations on macrophage activation and polarization through upregulation of STAT3, CEBPB, and SPI1. FCGR2A (CD32), CBFB, and JUNB highly expressed in macrophages were detected (Arlauckas et al., 2018). Markers like SERPINE1, ICAM1, HCK, and ARG1 promoted different macrophage polarization. While SERPINE1 (PAI-1) was shown to promote macrophage M2 polarization via STAT3 phosphorylation (Kubala et al., 2018), we also discovered makers like ICAM1 that promote M1 polarization (Audesirk et al., 1989). HCK and ARG1 are murine macrophage M2 and M1 polarization markers (Arlauckas et al., 2018; Meng and Lowell, 1997), though their functions in human macrophage are less studied. We found ITGAL and ITGA4 were highly expressed in the immune active tumors which are BMDM-specific markers in the mouse model (Bowman et al., 2016). The TAM phenotypes did not fall into the traditional classification of M1 and M2 macrophages, lending support to the concept that decisive M1 and M2 states may not exist in vivo (Quail and Joyce, 2017). Both hypoxia and macrophage polarization further elevated inflammatory response, including ELANE, IL18, CD40, PLCB2, ACLY, FLII, and IRAK1.

Discussion

The ability to integrate, for the first time, a myriad of information including protein abundance, post-translational modifications, small molecule metabolites, and lipid abundance with traditional genomics, clinical and imaging data has unlocked novel molecular and biological insights into GBM. We discovered two immune subtypes that exhibit differences in macrophage marker gene expression and immune checkpoint markers which are associated with epigenetic modifications of cancer-relevant proteins. We also uncovered previously unknown structural variants in cancer driver genes, strong cis and trans effects in protein and phosphorylation levels in samples with genetic alterations, phosphoproteins related to cell adhesion that are enriched in the telomere-long group of tumors, and differences in DG content consistent with altered DG metabolism and signaling in tumor cells. Our data also indicated that upregulated histone H2B acetylation might be driven by bromodomain proteins and acetyltransferases. Lastly, we revealed a metabolic cascade contributing to upregulation of oncometabolite 2-HG specifically in IDH mutants. By integrating omics platforms beyond standard genomic data, our work clearly demonstrates that new biological insights can be gained by adding multiple layers of molecular information, which would not otherwise be accessible.

Importantly, several of these findings have potential therapeutic implications. For example, upregulated protein or phosphorylation levels of activated MCM genes could be drug targets in RB1-altered samples. Increases in phosphorylation of PTPN11 (Shp2) suggest anti-Shp2 therapies may be effective in RTK-altered samples, regardless of the driver mutation or genomic alteration. Co-upregulation of CDK6 and EGFR suggests combining EGFR and CDK6 inhibitors in EGFR-altered samples. Also, we identified TNIK phosphoprotein as an outlier in the telomere-long group of samples, suggesting that patients with ATRX and IDH1 mutations may benefit from TNIK inhibitors. Together, these new discoveries gained from integrative omics exemplifies what precision oncology means to a GBM patient once these findings have been validated in larger cohorts. Such insights and options are beyond what genomics alone can provide. Additionally, our results support targeting TAMs in the microenvironment as an adjuvant therapy (de Groot et al., 2019; Thomas et al., 2019). Since macrophages depend upon CSF-1 (Pyonteck et al., 2013) and expression of both CSF-1 and CSF-1R are high in the immune-high group (FIG. 18A), the use of a CSF-1 inhibitor might be beneficial. Although a clinical trial with the CSF-1 receptor inhibitor PLX3397 did not show clear benefit for GBM patients (Butowski et al., 2016), this might be attributable to a lack of appropriate patient stratification, which our results now inform. Another option might be to inhibit TAM infiltration by targeting MCPs, which mediate macrophage migration and infiltration: four MCP family members (CCL2, CCL7, CCL8, and CCL13) were highly expressed in the immune-high subtype (FIG. 18A).

Despite the explosion of new molecular data, most GBM patients are still treated using a standard of care regimen (Stupp et al., 2005), with no genomic or transcriptional subtype-based therapies yet demonstrating efficacy. The field may be suffering from inadequate patient stratification and/or choices of the wrong targets due to a lack of complete understanding at the functional level. Improvements in these domains may help mitigate the high response variability with the Stupp regimen in GBM patients. Our results leverage genomic and transcriptomic subtypes toward this goal, but also provide deeper biological insight into genomic and transcriptomic hallmarks. For example, classical GBM, driven by EGFR genomic alterations, has a markedly different phosphoproteomic state (with targetable ramifications) than other GBMs. Similarly, from a metabolomic view, architectures of mesenchymal-like GBMs differ substantially from other subtypes and have specific metabolic vulnerabilities not present in other GBMs. Tumor microenvironment analysis suggests different biologies in immune-high and immune-low tumors, with implications for immunotherapy. Immune checkpoint blockade has largely failed in GBM trials, however, patients who responded with substantial durability belonged predominantly to the immune-high group, suggesting the need for better patient stratification. These patients may require additional immune checkpoints that target macrophage-lineages, not T-cells. Our data also suggest that anti-angiogenic agents such as bevacizumab combined with immunotherapy may be more beneficial in mesenchymal-like GBM and future trials should take this into consideration.

We are mindful that the relatively small number of patients enrolled for this study and the limited data on hallmark features, such as MGMT methylation and TERT protein expression, will not yet allow us to fully explore GBM heterogeneity. However, by demonstrating the clinical insight gained from even this modest sized cohort, we believe that future molecular analyses of GBM can no longer rely on genomics alone and should henceforth include a broader multi-omics perspective. We envision extending our strategy to study recurrent GBM tumors with matched primary tumors for understanding the mechanism of disease progression. Further, we plan to characterize adolescents and young adults (AYA), a cohort whose outcomes are generally better than older-onset cases (Leibetseder et al., 2013), but for which epidemiological differences of survival between/DH-wild type and/DH-mutant have likewise been noted (Hafeez et al., 2019). The approach will also likely help resolve the still-puzzling sex-based epidemiological differences in incidence and outcome that are observed across all ages in GBM (Ostrom et al., 2018, 2019) and which are now being investigated with imaging and selected genomic data (Yang et al., 2019). This in turn will advance clinical trial development with new targets and better patient stratification. Together, we hope these advances will lead to new, effective personalized treatments for patients.

TABLE 2
Key Resources Table

| | | |
|---|---|---|
| REAGENT or RESOURCE | SOURCE | IDENTIFIER |

Antibodies
Bacterial and Virus Strains
Biological Samples

| | | |
|---|---|---|
| Primary tumor and normal tissue | This paper | See Methods: Experimental |
| samples | | Model and Subject Details |
| Patient-derived xenograft tissue | Washington University in St. | See Methods: Method Details |
| samples | Louis | |

Chemicals, Peptides, and Recombinant Proteins

| | | |
|---|---|---|
| 4-(2-hydroxyethyl)-1- | Sigma | Catalog: H3375 |
| piperazineethanesulfonic acid | | |
| (HEPES) | | |
| Acetic Acid, glacial | Sigma | Catalog: AX0074-6 |
| Acetonitrile, HPLC grade | J. T. Baker | Catalog: 9829-03 |
| Acetonitrile anhydrous | Sigma | Catalog: 271004 |
| Ammonium hydroxide solution | Sigma | Catalog: 338818 |
| Aprotinin | Sigma | Catalog: A6103 |
| Dithiothreitol | Thermo Scientific | Catalog: 20291 |
| Ethylenediaminetetraacetic acid | Sigma | Catalog: E7889 |
| Formic acid | Sigma | Catalog: 0507 |
| Iodoacetamide | Sigma | Catalog: A3221 |
| Iron (III) chloride | Sigma | Catalog: 451649 |
| HPLC Grade Water | J. T. Baker | Catalog: 4218-03 |
| Hydroxylamine Solution 50% | Sigma | Catalog: 467804 |
| Leupeptin | Roche | Catalog: 11017101001 |
| Lysyl Endopeptidase | Wako Chemicals | Catalog 129-02541 |
| Methanol, HPLC grade | Fluka | Catalog: 34966 |
| Ni-NTA Superflow Agarose Beads | Qiagen | Catalog: 30410 |
| Phenylmethylsulfonyl fluoride | Sigma | Catalog: 93482 |
| Phosphatase Inhibitor Cocktail 2 | Sigma | Catalog: P5726 |
| Phosphatase Inhibitor Cocktail 3 | Sigma | Catalog: P0044 |
| Potassium phosphate dibasic | Sigma | Catalog: P3786 |
| Potassium phosphate monobasic | Sigma | Catalog: P9791 |
| PUGNAc | Sigma | Catalog: A7229 |
| Reversed-phase tC18 SepPak | Waters | Catalog: WAT054925 |
| Sequencing grade modified | Promega | Catalog: V517 |
| trypsin | | |
| Sodium butyrate | Sigma | Catalog: 303410 |
| Sodium chloride | Sigma | Catalog: S7653 |
| Sodium fluoride | Sigma | Catalog: S7920 |
| Tris | Sigma | Catalog: T2694 |
| (hydroxymethyl)aminomethane | | |
| hydrochloride pH 8.0 | | |
| Trifluoroacetic acid | Sigma | Catalog: 91707 |
| Urea | Sigma | Catalog: U0631 |

Critical Commercial Assays

| | | |
|---|---|---|
| BCA Protein Assay Kit | ThermoFisher Scientific | Catalog: A53225 |
| Infinium MethylationEPIC Kit | Illumina | Catalog: WG-317-1003 |
| TMT-11 reagent kit | ThermoFisher Scientific | Catalog: A34808 |
| TruSeq Stranded Total RNA | Illumina | Catalog: RS-122-2301 |
| Library Prep Kit with Ribo-Zero | | |
| Gold | | |
| PTMScan ® Acetyl-Lysine Motif | Cell Signaling | Catalog: 13416 |
| [Ac—K] Kit | | |

Deposited Data
Experimental Models: Cell Lines
Experimental Models: Organisms/Strains
Oligonucleotides
Recombinant DNA
Software and Algorithms

| | | |
|---|---|---|
| Ascore v1.0.6858 | (Beausoleil et al., 2006) | https://github.com/PNNL-Comp-Mass-Spec/AScore |
| MASIC | (Monroe et al., 2008) | https://github.com/PNNL-Comp-Mass-Spec/MASIC |
| MS-GF+ v9981 | (Kim and Pevzner, 2014) | https://github.com/MSGFPlus/msgfplus |
| mzRefinery | (Gibbons et al., 2015) | https://omics.pnl.gov/software/mzrefinery |
| R v3.6 | R Development Core Team | https://www.R-project.org |
| Bioconductor v3.9 | (Huber et al., 2015) | https://bioconductor.org/ |
| Tidyverse | (Wickham et al., 2019) | https://www.tidyverse.org/ |
| Python v3.7 | Python Software Foundation | https://www.python.org/ |
| Bioconda | (The Bioconda Team et al., 2018) | https://bioconda.github.io/ |
| Snakemake v5.6 | (Koster and Rahmann, 2012) | https://snakemake.readthedocs.io/ |
| BIC-Seq2 | (Xi et al., 2016) | http://compbio.med.harvard.edu/BIC-seq/ |
| GISTIC2 v2.0.22 | (Mermel et al., 2011) | https://github.com/broadinstitute/gistic2 |
| xCell v1.2 | (Aran et al., 2017) | http://xcell.ucsf.edu/ |
| Strelka v2.9.2 | (Kim et al., 2018) | https://github.com/lllumina/strelka |
| VarScan v2.3.8 | (Koboldt et al., 2012) | https://dkoboldt.github.io/varscan/ |
| Pindel v0.2.5 | (Ye et al., 2009) | https://github.com/genome/pindel |
| MuTect v1.1.7 | (Cibulskis et al., 2013) | https://github.com/broadinstitute/mutect |
| TinDaisy v1.0 | Li Ding Lab | https://github.com/ding-lab/TinDaisy |
| somaticwrapper v1.3 | Li Ding Lab | https://github.com/ding-lab/somaticwrapper |
| Samtools v1.2 | (Li et al., 2009) | https://www.htslib.org/ |
| GATK v4.0.0.0 | (McKenna et al., 2010) | https://github.com/broadgsa/gatk |
| bam-readcount v0.8 | McDonnell Genome Institute | https://github.com/genome/bam-readcount |
| germlinewrapper v1.1 | Li Ding Lab | https://github.com/ding-lab/germlinewrapper |
| Manta v1.6.0 | (Chen et al., 2016) | https://github.com/Illumina/manta |
| DELLY v0.8.1 | (Rausch et al., 2012) | https://github.com/dellytools/delly |
| Telseq v0.0.1 | (Ding et al., 2014) | https://github.com/zd1/telseq |
| HTSeq v0.11.2 | (Anders et al., 2015) | https://github.com/simon-anders/htseq |
| EricScript v0.5.5 | (Benelli et al., 2012) | https://sites.google.com/site/bioericscript/ |
| INTEGRATE v0.2.6 | (Zhang et al., 2016b) | https://sourceforge.net/projects/integrate-fusion/ |
| STAR-Fusion v1.5.0 | (Haas et al., 2019) | https://github.com/STAR-Fusion/STAR-Fusion |
| BWA v0.7.17-r1188 | (Li and Durbin, 2009) | http://bio-bwa.sourceforge.net/ |
| CIRI V2.0.6 | (Gao et al., 2015) | https://sourceforge.net/projects/ciri/ |
| RSEM v1.3.1 | (Li and Dewey, 2011) | https://deweylab.github.io/RSEM/ |
| Bowtie2 v2.3.3 | (Langmead and Salzberg, 2012) | http://bowtie-bio.sourceforge.net/bowtie2/index.shtml |
| R-rollup | (Polpitiya et al., 2008) | https://omics.pnl.gov/software/danter |
| MODMatcher | (Yoo et al., 2014) | https://github.com/integrativenetworkbiology/Modmatcher |
| ConsensusClusterPlus v1.48.0 | (Wilkerson and Hayes, 2010) | https://bioconductor.org/packages/ConsensusClusterPlus/ |
| louvain-igraph v0.6.1 | (Blondel et al., 2008) | https://doi.org/10.5281/zenodo.1054103 |
| TCGAbiolinks v2.11.1 | (Colaprico et al., 2016) | http://bioconductor.org/packages/TCGAbiolinks/ |
| iProFun | (Song et al., 2019) | https://github.com/songxiaoyu/iProFun |
| BlackSheep | (Blumenberg et al., 2019) | https://github.com/ruggleslab/blackSheep |
| xCell | (Aran et al., 2017) | http://xcell.ucsf.edu/ |
| MoonlightR v1.12.0 | (Colaprico et al., 2020) | http://bioconductor.org/packages/MoonlightR |
| CausalPath v.7c5b934 | (Babur et al., 2018) | https://github.com/PathwayAndDataAnalysis/causalpath |

Other

| | | |
|---|---|---|
| RefSeq (downloaded from UCSC | (O'Leary et al., 2016) | https://www.ncbi.nlm.nih.gov/refseg/; |
| Genome Browser on 2018 Jun. 29) | | https://genome.ucsc.edu/cgi-bin/hgTables: RRID: SCR_003496 |
| GENCODE v22 (download from | (Frankish et al., 2019) | https://www.gencodegenes.org/; |
| GDC Reference Files) | | https://gdc.cancer.gov/about-data/data-harmonization-and-generation/ |
| | | gdc-reference-files |
| gnomAD v2.1 | (Karczewski et al., 2019) | https://gnomad.broadinstitute.org/ |
| The 1000 Genomes Project | (The 1000 Genomes Project | https://www.internationalgenome.org/ |
| | Consortium, 2015) | |
| OmniPath (downloaded on 2018 Mar. 29) | (Türei et al., 2016) | http://omnipathdb.org/ |
| DEPOD (downloaded on 2018 | (Duan et al., 2015) | http://depod.bioss.uni-freiburg.de/ |
| Mar. 29) | | |
| CORUM (downloaded on 2018 | (Ruepp et al., 2010) | https://mips.helmholtz-muenchen.de/corum/ |
| Jun. 29) | | |
| SIGNOR v2.0 (downloaded on | (Licata et al., 2019) | https://signor.uniroma2.it/ |
| 2018 Oct. 29) | | |
| Reactome (downloaded on 2018 | (Fabregat et al., 2018) | https://reactome.org/ |
| Nov. 1) | | |
| NetworKIN 3.0 | (Horn et al., 2014) | https://networkin.info/ |

Experimental Model and Subject Details

Specimens and Clinical Data

Tumor and germline blood samples from 99 qualified cases were collected from 10 tissue source sites in strict accordance to the CPTAC-3 protocol with an informed consent from the patients. No adjacent tissue was collected as part of this study, however, 10 normal samples from the frontal cortex were used in the analysis from the GTEx project (https://gtexportal.org/). This study contained both males (n=55) and females (n=44) from 6 different countries. Histopathologically defined adult glioblastoma tumors were only considered for analysis, with an age range of 24-88. Clinical data were obtained from the tissue source sites and reviewed for correctness and completeness of data.

Sample Processing

The CPTAC Biospecimen Core Resource (BCR) at the Pathology and Biorepository Core of the Van Andel Research Institute in Grand Rapids, Mich. manufactured and distributed biospecimen kits to the Tissue Source Sites (TSS) located in the US, Europe, and Asia. Each kit contains a set of pre-manufactured labels for unique tracking of every specimen respective to TSS location, disease, and sample type, used to track the specimens through the BCR to the CPTAC proteomic and genomic characterization centers.

Tissue specimens averaging 200 mg were snap-frozen by the TSS within a 30 minute cold ischemic time (CIT) (CIT average=13 minutes) and an adjacent segment was formalin-fixed paraffin-embedded (FFPE) and H&E stained by the TSS for quality assessment to meet the CPTAC GBM requirements. Routinely, several tissue segments for each case were collected. Tissues were flash frozen in liquid nitrogen (LN₂) then transferred to a liquid nitrogen freezer for storage until approval for shipment to the BCR.

Specimens were shipped using a cryoport that maintained an average temperature of under −140° C. to the BCR with a time and temperature tracker to monitor the shipment. Receipt of specimens at the BCR included a physical inspection and review of the time and temperature tracker data for specimen integrity, followed by barcode entry into a biospecimen tracking database. Specimens were again placed in LN₂ storage until further processing. Acceptable GBM tumor tissue segments were determined by TSS pathologists based on the percent viable tumor nuclei (>60%), total cellularity (>50%), and necrosis (<50%). Segments received at the BCR were verified by BCR and Leidos Biomedical Research (LBR) pathologists and the percent of total area of tumor in the segment was also documented. Additionally, disease-specific working group pathology experts reviewed the morphology to clarify or standardize specific disease classifications and correlation to the proteomic and genomic data.

Specimens selected for the discovery set were determined on the maximal percent in the pathology criteria and best weight. Specimens were pulled from the biorepository using an LN₂ cryocart to maintain specimen integrity and then cryopulverized. The cryopulverized specimen was divided into aliquots for DNA (30 mg) and RNA (30 mg) isolation and proteomics (50 mg) for molecular characterization. Nucleic acids were isolated and stored at −80° C. until further processing and distribution; cryopulverized protein material was returned to the LN₂ freezer until distribution. Shipment of the cryopulverized segments used cryoports for distribution to the proteomic characterization centers and shipment of the nucleic acids used dry ice shippers for distribution to the genomic characterization centers; a shipment manifest accompanied all distributions for the receipt and integrity inspection of the specimens at the destination. The DNA sequencing was performed at the Broad Institute, Cambridge, Mass. and RNA sequencing was performed at the University of North Carolina, Chapel Hill, N.C. Material for proteomic analyses was sent to the Proteomic Characterization Center (PCC) at Pacific Northwest National Laboratory (PNNL), Richland, Wash.

Method Details

Sample Processing for Genomic DNA and Total RNA Extraction

Our study sampled a single site of the primary tumor from surgical resections, due to the internal requirement to process a minimum of 125 mg of tumor issue and 50 mg of adjacent normal tissue. DNA and RNA were extracted from tumor and blood normal specimens in a co-isolation protocol using Qiagen's QIAsymphony DNA Mini Kit and QIAsymphony RNA Kit. Genomic DNA was also isolated from peripheral blood (3-5 mL) to serve as matched normal reference material. The Qubit™ dsDNA BR Assay Kit was used with the Qubit® 2.0 Fluorometer to determine the concentration of dsDNA in an aqueous solution. Any sample that passed quality control and produced enough DNA yield to go through various genomic assays was sent for genomic characterization. RNA quality was quantified using both the NanoDrop 8000 and quality assessed using Agilent Bioanalyzer. A sample that passed RNA quality control and had a minimum RIN (RNA integrity number) score of 7 was subjected to RNA sequencing. Identity match for germline, normal adjacent tissue, and tumor tissue was assayed at the BCR using the Illumina Infinium QC array. This beadchip contains 15,949 markers designed to prioritize sample tracking, quality control, and stratification.

Whole Exome Sequencing

Library Construction

Library construction was performed as described in (Fisher et al., 2011), with the following modifications: initial genomic DNA input into shearing was reduced from 3 μg to 20-250 ng in 50 μL of solution. For adapter ligation, Illumina paired-end adapters were replaced with palindromic forked adapters, purchased from Integrated DNA Technologies, with unique dual-indexed molecular barcode sequences to facilitate downstream pooling. Kapa HyperPrep reagents in 96-reaction kit format were used for end repair/A-tailing, adapter ligation, and library enrichment PCR. In addition, during the post-enrichment SPRI cleanup, elution volume was reduced to 30 μL to maximize library concentration, and a vortexing step was added to maximize the amount of template eluted.

In-Solution Hybrid Selection

After library construction, libraries were pooled into groups of up to 96 samples. Hybridization and capture were performed using the relevant components of Illumina's Nextera Exome Kit and following the manufacturer's suggested protocol, with the following exceptions. First, all libraries within a library construction plate were pooled prior to hybridization. Second, the Midi plate from Illumina's Nextera Exome Kit was replaced with a skirted PCR plate to facilitate automation. All hybridization and capture steps were automated on the Agilent Bravo liquid handling system.

Preparation of Libraries for Cluster Amplification and Sequencing

After post-capture enrichment, library pools were quantified using qPCR (automated assay on the Agilent Bravo) using a kit purchased from KAPA Biosystems with probes specific to the ends of the adapters. Based on qPCR quantification, libraries were normalized to 2 nM.

Cluster Amplification and Sequencing

Cluster amplification of DNA libraries was performed according to the manufacturer's protocol (Illumina) using exclusion amplification chemistry and flowcells. Flowcells were sequenced utilizing sequencing-by-synthesis chemistry. The flowcells were then analyzed using RTA v.2.7.3 or later. Each pool of whole exome libraries was sequenced on paired 76 cycle runs with two 8 cycle index reads across the number of lanes needed to meet coverage for all libraries in the pool. Pooled libraries were run on HiSeq 4000 paired-end runs to achieve a minimum of 150× on target coverage per each sample library. The raw Illumina sequence data were demultiplexed and converted to fastq files; adapter and low-quality sequences were trimmed. The raw reads were mapped to the hg38 human reference genome and the validated BAMs were used for downstream analysis and variant calling.

PCR-Free Whole Genome Sequencing

Preparation of Libraries for Cluster Amplification and Sequencing

An aliquot of genomic DNA (350 ng in 50 μL) was used as the input into DNA fragmentation (aka shearing). Shearing was performed acoustically using a Covaris focused-ultrasonicator, targeting 385 bp fragments. Following fragmentation, additional size selection was performed using a SPRI cleanup. Library preparation was performed using a commercially available kit provided by KAPA Biosystems (KAPA Hyper Prep without amplification module) and with palindromic forked adapters with unique 8-base index sequences embedded within the adapter (purchased from IDT). Following sample preparation, libraries were quantified using quantitative PCR (kit purchased from KAPA Biosystems), with probes specific to the ends of the adapters. This assay was automated using Agilent's Bravo liquid handling platform. Based on qPCR quantification, libraries were normalized to 1.7 nM and pooled into 24-plexes.

Cluster Amplification and Sequencing (HiSeq X)

Sample pools were combined with HiSeq X Cluster Amp Reagents EPX1, EPX2, and EPX3 into single wells on a strip tube using the Hamilton Starlet Liquid Handling system. Cluster amplification of the templates was performed according to the manufacturer's protocol (Illumina) with the Illumina cBot. Flowcells were sequenced to a minimum of 15× on HiSeq X utilizing sequencing-by-synthesis kits to produce 151 bp paired-end reads. Output from Illumina software was processed by the Picard data processing pipeline to yield BAMs containing demultiplexed, aggregated, aligned reads. All sample information tracking was performed by automated LIMS messaging.

Illumina Infinium MethylationEPIC BeadChip Array

The MethylationEPIC array uses an 8-sample version of the Illumina Beadchip capturing >850,000 DNA methylation sites per sample. 250 ng of DNA was used for the bisulfite conversation using Infinium MethylationEPIC BeadChip Kit. The EPIC array includes sample plating, bisulfite conversion, and methylation array processing. After scanning, the data was processed through an automated genotype calling pipeline. Data generated consisted of raw idats and a sample sheet.

RNA Sequencing

Quality Assurance and Quality Control of RNA Analytes

All RNA analytes were assayed for RNA integrity, concentration, and fragment size. Samples for total RNA-seq were quantified on a TapeStation system (Agilent, Inc. Santa Clara, Calif.). Samples with RINs >8.0 were considered high quality.

Total RNA-Seq Library Construction

Total RNA-seq library construction was performed from the RNA samples using the TruSeq Stranded RNA Sample Preparation Kit and bar-coded with individual tags following the manufacturer's instructions (Illumina, Inc. San Diego, Calif.). Libraries were prepared on an Agilent Bravo Automated Liquid Handling System. Quality control was performed at every step and the libraries were quantified using the TapeStation system.

Total RNA Sequencing

Indexed libraries were prepared and run on HiSeq 4000 paired end 75 base pairs to generate a minimum of 120 million reads per sample library with a target of greater than 90% mapped reads. Typically, these were pools of four samples. The raw Illumina sequence data were demultiplexed and converted to FASTQ files, and adapter and low-quality sequences were quantified. Samples were then assessed for quality by mapping reads to the hg38 human genome reference, estimating the total number of reads that mapped, amount of RNA mapping to coding regions, amount of rRNA in sample, number of genes expressed, and relative expression of housekeeping genes. Samples passing this QA/QC were then clustered with other expression data from similar and distinct tumor types to confirm expected expression patterns. Atypical samples were then SNP typed from the RNA data to confirm source analyte. FASTQ files of all reads were then uploaded to the GDC repository.

miRNA-Seq Library Construction

miRNA-seq library construction was performed from the RNA samples using the NEXTflex Small RNA-Seq Kit (v3, PerkinElmer, Waltham, Mass.) and bar-coded with individual tags following the manufacturer's instructions. Libraries were prepared on Sciclone Liquid Handling Workstation Quality control was performed at every step, and the libraries were quantified using a TapeStation system and an Agilent Bioanalyzer using the Small RNA analysis kit. Pooled libraries were then size selected according to NEXTflex Kit specifications using a Pippin Prep system (Sage Science, Beverly, Mass.).

miRNA Sequencing

Indexed libraries were loaded on the Hiseq 4000 to generate a minimum of 10 million reads per library with a minimum of 90% reads mapped. The raw Illumina sequence data were demultiplexed and converted to FASTQ files for downstream analysis. Resultant data were analyzed using a variant of the small RNA quantification pipeline developed for TCGA (Chu et al., 2016). Samples were assessed for the number of miRNAs called, species diversity, and total abundance. Samples passing quality control were uploaded to the GDC repository.

MS Sample Processing and Data Collection

Protein Extraction and Lys-C/Trypsin Tandem Digestion

Approximately 50 mg of each of the cryopulverized tumor and normal tissues were homogenized separately in 200 μL of lysis buffer (8 M urea, 75 mM NaCl, 50 mM Tris, pH 8.0, 1 mM EDTA, 2 μg/mL aprotinin, 10 μg/mL leupeptin, 1 mM PMSF, 10 mM NaF, 1:100 v/v Sigma phosphatase inhibitor cocktail 2, 1:100 v/v Sigma phosphatase inhibitor cocktail 3, 20 μM PUGNAc, and 5 mM sodium butyrate). Lysates were precleared by centrifugation at 20,000×g for 10 min at 4° C. and protein concentrations were determined by BCA assay (ThermoFisher Scientific) and adjusted to 8 μg/μL with lysis buffer. Proteins were reduced with 5 mM dithiothreitol for 1 h at 37° C. and subsequently alkylated with 10 mM iodoacetamide for 45 min at 25° C. in the dark. Samples were diluted 1:3 with 50 mM Tris, pH 8.0 and digested with Lys-C (Wako) at 1:50 enzyme-to-substrate ratio. After 2 h of digestion at 25° C., an aliquot of the same amount of sequencing-grade modified trypsin (Promega, V5117) was added to the samples and further incubated at 25° C. for 14 h. The digested samples were then acidified with 100% formic acid to 1% of the final concentration of formic acid and centrifuged for 15 min at 1,500×g at 4° C. before transferring samples into new tubes leaving the resulting pellet behind. After 3 fold dilution with 0.1% formic acid, tryptic peptides were desalted on C18 SPE (Waters tC18 SepPak, WAT054925) and dried using Speed-Vac.

TMT-11 Labeling of Peptides

Desalted peptides from each sample were labeled with 11-plex TMT reagents (ThermoFisher Scientific). Peptides (400 μg) from each of the samples were dissolved in 80 μL of 50 mM HEPES, pH 8.5 solution, and mixed with 400 μg of TMT reagent that was dissolved freshly in 20 μL of anhydrous acetonitrile according to the optimized TMT labeling protocol described previously (Zecha et al., 2019). Channel 126 was used for labeling the internal reference sample (pooled from all tumor and normal samples) throughout the sample analysis. After 1 h incubation at RT, 60 μL 50 mM HEPES pH8.5, 20% ACN solution was added to dilute the samples, and 12 μL of 5% hydroxylamine was added and incubated for 15 min at RT to quench the labeling reaction. Peptides labeled by different TMT reagents were then mixed, dried using Speed-Vac, reconstituted with 3% acetonitrile, 0.1% formic acid and desalted on tC18 SepPak SPE columns.

Peptide Fractionation by Basic Reversed-Phase Liquid Chromatography (bRPLC)

Approximately 3.5 mg of 11-plex TMT labeled sample was separated on a reversed phase Agilent Zorbax 300 Extend-C18 column (250 mm×4.6 mm column containing 3.5-μm particles) using the Agilent 1200 HPLC System. Solvent A was 4.5 mM ammonium formate, pH 10, 2% acetonitrile and solvent B was 4.5 mM ammonium formate, pH 10, 90% acetonitrile. The flow rate was 1 mL/min and the injection volume was 900 μL. The LC gradient started with a linear increase of solvent B to 16% in 6 min, then linearly increased to 40% B in 60 min, 4 min to 44% B, 5 min to 60% B and another 14 of 60% solvent B. A total of 96 fractions were collected into a 96 well plate throughout the LC gradient. These fractions were concatenated into 24 fractions by combining 4 fractions that are 24 fractions apart (i.e., combining fractions #1, #25, #49, and #73; #2, #26, #50, and #74; and so on). For proteome analysis, 5% of each concatenated fraction was dried down and re-suspended in 2% acetonitrile, 0.1% formic acid to a peptide concentration of 0.1 mg/mL for LC-MS/MS analysis. The rest of the fractions (95%) were further concatenated into 12 fractions (i.e., by combining fractions #1 and #13; #3 and #15; and so on), dried down, and subjected to immobilized metal affinity chromatography (IMAC) for phosphopeptide enrichment.

Phosphopeptide Enrichment Using IMAC

Fe³⁺-NTA-agarose beads were freshly prepared using the Ni-NTA Superflow agarose beads (QIAGEN, #30410) for phosphopeptide enrichment. For each of the 12 fractions, peptides were reconstituted in 500 μL IMAC binding/wash buffer (80% acetonitrile, 0.1% trifluoroacetic acid) and incubated with 20 μL of the 50% bead suspension for 30 min at RT. After incubation, the beads were sequentially washed with 50 μL of wash buffer (1×), 50 μL of 50% acetonitrile, 0.1% trifluoroacetic acid (1×), 50 μL of wash buffer (1×), and 50 μL of 1% formic acid (1×) on the stage tip packed with 2 discs of Empore C18 material (Empore Octadecyl C18, 47 mm; Supleco, 66883-U). Phosphopeptides were eluted from the beads on C18 using 70 μL of elution buffer (500 mM K₂HPO₄, pH 7.0). Sixty microliter of 50% acetonitrile, 0.1% formic acid was used for elution of phosphopeptides from the C18 stage tips after two washes with 100 μL of 1% formic acid. Samples were dried using Speed-Vac and later reconstituted with 12 μL of 3% acetonitrile, 0.1% formic acid for LC-MS/MS analysis.

Immunoaffinity Purification of Acetylated Peptides

Tryptic peptides from the flow-through of IMAC were combined into four samples follow concatenation scheme by combining 3 fractions that were 4 fractions apart (i.e., combining fractions #1, #5 and #9 as a new fraction) and dried down using Speed-Vac. The dried peptides were reconstituted in 1.4 mL of the immunoaffinity purification (IAP) buffer (50 mM MOPS/NaOH pH 7.2, 10 mM Na₂HPO₄ and 50 mM NaCl). After dissolving the peptide, the pH of the peptide solution was checked using pH indicator paper. The antibody beads from PTMScan® Acetyl-Lysine Motif [Ac-K] Kit (Cell Signaling, #13416) were freshly prepared. Briefly, the antibody beads were centrifuged at 2,000×g for 30 sec and all buffers from the beads were removed; the antibody beads were then washed with 1 mL of IAP buffer for four times and finally resuspend in 40 μL of IAP buffer. For each fraction, half of the antibody in each tube was transferred to the peptide solution and incubated on a rotator overnight at 4° C. After removing the supernatant, the reacted beads were washed with 1 mL of PBS buffer five times. For the elution of acetylated peptides, the antibody beads were incubated 2 times each with 50 μL of 0.15% TFA at room temperature for 10 min. The eluted peptides were transferred to the stage tip packed with two discs of Empore C18 material. The C18 stage tips were washed by 1% formic acid and 50% acetonitrile, and 0.1% formic acid was used for elution of peptides from the C18 stage tips. The eluted peptides were dried using Speed-Vac, and reconstituted with 13 μL of 2% acetonitrile, 0.1% formic acid contained 0.01% DDM (n-Dodecyl β-D-maltoside) right before the LC-MS/MS analysis.

The acetylated peptides prepared by IP from the IMAC flow-through may very well miss those peptides that are both phosphorylated and acetylated. Splitting the samples for independent IP and IMAC may improve the chance of recovering such peptides, assuming having both PTMs on the same peptide does not impact the affinity of either the IP or IMAC process. However, acetylated peptides are estimated to be 10 times lower in abundance than the phosphopeptides, hence much larger input may be needed to recover the dual-modified peptides. Given the extremely low stoichiometry of these dual-modified peptides and the sample size limitations, it was not pursued in this work.

LC-MS/MS Analysis

Fractionated samples prepared for global proteome, phosphoproteome, and acetylome analysis were separated using a nanoACQUITY UPLC system (Waters) by reversed-phase HPLC. The analytical column was manufactured in-house using ReproSil-Pur 120 C18-AQ 1.9 μm stationary phase (Dr. Maisch GmbH) and slurry packed into a 25-cm length of 360 μm o.d.×75 μm i.d. fused silica picofrit capillary tubing (New Objective). The analytical column was heated to 50° C. using an AgileSLEEVE column heater (Analytical Sales and Services). The analytical column was equilibrated to 98% Mobile Phase A (MP A, 0.1% formic acid/3% acetonitrile) and 2% Mobile Phase B (MP B, 0.1% formic acid/90% acetonitrile) and maintained at a constant column flow of 200 nL/min. The sample was injected into a 5-μL loop placed in-line with the analytical column which initiated the gradient profile (min:% MP B): 0:2, 1:6, 85:30, 94:60, 95:90, 100:90, 101:50, 110:50 (for global proteome and phosphoproteome analysis); 0:2, 1:6, 235:30, 244:60, 245:90, 250:90, 251:50, 260:50 (for acetylome analysis). The column was allowed to equilibrate at start conditions for 30 minutes between analytical runs.

MS analysis was performed using an Orbitrap Fusion Lumos mass spectrometer (ThermoFisher Scientific). The global proteome and phosphoproteome samples were analyzed under identical conditions. Electrospray voltage (1.8 kV) was applied at a carbon composite union (Valco Instruments) coupling a 360 μm o.d.×20 μm i.d. fused silica extension from the LC gradient pump to the analytical column and the ion transfer tube was set at 250° C. Following a 25-min delay from the time of sample injection, Orbitrap precursor spectra (AGC 4×10⁵) were collected from 350-1800 m/z for 110 min at a resolution of 60K along with data dependent Orbitrap HCD MS/MS spectra (centroid) at a resolution of 50K (AGC 1×10⁵) and max ion time of 105 ms for a total duty cycle of 2 seconds. Masses selected for MS/MS were isolated (quadrupole) at a width of 0.7 m/z and fragmented using a collision energy of 30%. Peptide mode was selected for monoisotopic precursor scan and charge state screening was enabled to reject unassigned 1+, 7+, 8+, and >8+ ions with a dynamic exclusion time of 45 seconds to discriminate against previously analyzed ions between ±10 ppm. The acetylome samples were analyzed under similar conditions except that the max ion time was 200 ms.

Construction and Utilization of the Comparative Reference Samples

As a quality control measure, two different types of “Comparative Reference” (“CompRef”) patient-derived xenograft (PDX) samples were generated as previously described (Li et al., 2013; Tabb et al., 2016) and used to monitor the longitudinal performance of the proteomics workflow throughout the course of this study. Briefly, the PDX tumors from established basal and luminal breast cancer intrinsic subtypes were raised subcutaneously in 8-week old NOD.Cg-Prkdc^{scid}Il2rg^{tm1Wj1}/SzJ mice (Jackson Laboratories, Bar Harbor, Me.) using procedures reviewed and approved by the Institutional Animal Care and Use Committee at Washington University in St. Louis. Xenografts were grown in multiple mice, pooled, and cryopulverized to provide a sufficient amount of uniform material for the duration of the study. Full proteome, phosphoproteome and acetylome process replicates of each of the two types of CompRef samples were prepared and analyzed as standalone 11-plex TMT experiments alongside every 4 TMT-11 experiments of the study samples, using the same analysis protocol as the patient samples. These interstitially analyzed CompRef samples were evaluated for depth of proteome, phosphoproteome, and acetylome coverage and for consistency in quantitative comparison between the basal and luminal models.

Polar Metabolites and Lipid Mass Spectrometry

Metabolite and Lipid Extraction

Lipids and metabolite extracts were generated from the same pulverized tissue with a minimum of 30 mg using a modified Folch extraction (Nakayasu et al., 2016). Additional solvent was added such that the final volume was proportionate to the mass of the sample ensuring the solvent ratio is 3:8:4 H₂O:CHCl₃:MeOH. Sample were vortexed for 30 sec, chilled in an ice block for 5 min, and vortexed again for 30 sec. The samples were then centrifuged at 10,000×g for 10 min at 4° C. The polar metabolite extract was transferred into a glass vial, dried in a speedvac, and stored at −20° C. until chemical derivatization for gas chromatography mass spectrometry (GC-MS) analysis. The total lipid extract (TLE) was transferred into a glass vial, dried in a speedvac, and then reconstituted in 500 μL 1:1 chloroform/methanol for storage at −20° C. until analysis.

Chemical Derivatization of Polar Metabolites

Polar metabolites along with 50% of the TLE were chemically derivatized prior to metabolomics analysis. Chemical derivatization of metabolites was previously detailed (Webb-Robertson et al., 2014). To protect carbonyl groups and reduce the number of tautomeric isomers, 20 μL of methoxyamine in pyridine (30 mg/mL) was added to each sample, followed by vortexing for 30 seconds and incubation at 37° C. with generous shaking for 90 minutes. To derivatize hydroxyl and amine groups to trimethylsilylated (TMS) forms, 80 μL of N-methyl-N-(trimethylsilyl)trifluoroacetamide (MSTFA) with 1% trimethylchlorosilane (TMCS) was added to each vial, followed by vortexing for 10 seconds and incubation at 37° C. with shaking for 30 minutes. The samples were allowed to cool to room temperature and were analysed on the GC-MS the same day.

GC-MS Analysis

An Agilent GC 7890A coupled with a single quadrupole MSD 5975C was used to analyze chemically derivatized metabolites. GC-MS analysis was previously detailed (Webb-Robertson et al., 2014). Briefly, 1 μL of each sample was injected onto a HP-5MS column (30 m×0.25 mm×0.25 μm; Agilent Technologies, Inc). The injection port temperature was held at 250° C. throughout the analysis. The GC oven was held at 60° C. for 1 minute after injection then increased to 325° C. by 10° C./min, followed by a 5-minute hold at 325° C. Total analysis time was 34 minutes per injection. The helium gas flow rates were determined by the Agilent Retention Time Locking function based on analysis of deuterated myristic acid. Data were collected over the mass range 50-550 m/z. A mixture of fatty acid methyl esters (C8-C28) was analyzed once per day at the beginning of each batch together with the samples for retention index alignment purposes during subsequent data analysis.

LC-MS Analysis

Stored plasma TLEs were dried in vacuo (45 min) and reconstituted in 5 μL chloroform plus 95 μL of methanol. The TLEs were analyzed as outlined in the previous study (Kyle et al., 2017). A Waters Acquity UPLC H class system interfaced with a Velos-ETD Orbitrap mass spectrometer was used for liquid chromatography tandem mass spectrometry (LC-MS/MS) analyses. 10 μL of reconstituted sample was injected onto a Waters CSH column (3.0 mm×150 mm×1.7 μm particle size) and separated over a 34-minute gradient (mobile phase A: ACN/H₂O (40:60) containing 10 mM ammonium acetate; mobile phase B: ACN/IPA (10:90) containing 10 mM ammonium acetate) at a flow rate of 250 μL/min. Eluting lipids were introduced to the MS via electrospray ionization in both positive and negative modes, and lipids were fragmented using higher-energy collision dissociation (HCD) and collision-induced dissociation (CID).

Metabolite Identification and Data Processing

Metabolite identifications and data processing were conducted as previously detailed (Webb-Robertson et al., 2014). GC-MS raw data files were processed using Metabolite Detector software v2.0.6 beta (Hiller et al., 2009). Retention indices (RI) of detected metabolites were calculated based on the analysis of the FAMEs mixture, followed by their chromatographic alignment across all analyses after deconvolution. Metabolites were identified by matching experimental spectra to an augmented version of the Agilent Fiehn Metabolomics Retention Time Locked (RTL) Library (Kind et al., 2009), containing spectra and validated retention indices. All metabolite identifications were manually validated. The NIST 08 GC-MS library was also used to cross validate the spectral matching scores obtained using the Agilent library and to provide identifications for metabolites that were initially unidentified. The three most abundant fragment ions in the spectra of each identified metabolite were automatically determined by Metabolite Detector, and their summed abundances were integrated across the GC elution profile. A matrix of identified metabolites, unidentified metabolite features, and their corresponding abundances for each sample in the batch were exported for statistics.

Lipid Identification and Data Processing

LC-MS/MS lipidomics data were analyzed using LIQUID (Lipid Informed Quantitation and Identification) (Kyle et al., 2017). Confident identifications were selected by manually evaluating the MS/MS spectra for diagnostic and corresponding acyl chain fragments of the identified lipid. In addition, the precursor isotopic profile, extracted ion chromatogram, and mass measurement error along with the elution time were evaluated. To facilitate quantification of lipids, a reference database for lipids identified from the MS/MS data was created and features from each analysis were then aligned to the reference database based on their identification, m/z and retention time using MZmine 2 (Pluskal et al., 2010). Aligned features were manually verified and peak apex intensity values were exported for subsequent statistical analysis.

Quantification and Statistical Analysis

Tumor Exclusion Criteria

One sample (C3L-03747) was excluded from the downstream analysis since it failed the expert pathology review (high necrosis) and had low correlation of RNA and protein or phosphoprotein.

Genomic Data Analysis

Harmonized Genome Alignment

WGS, WES, RNA-Seq sequence data were harmonized by NCI Genomic Data Commons (GDC) https://gdc.cancer.gov/about-data/gdc-data-harmonization, which included alignment to GDC's hg38 human reference genome (GRCh38.d1.vd1) and additional quality checks. All the downstream genomic processing was based on the GDC aligned BAMs to ensure reproducibility. However, RNA-Seq of 9 GTEx and 4 CPTAC samples didn't have the GDC harmonized BAMs available at the time of the analysis. We followed GDC's pipeline (same tool and parameters) to align those RNA-Seq samples. To ensure our alignment pipeline is identical to GDC, we randomly selected 10 samples with GDC BAMs available to apply our pipeline and obtain their gene level read count. All selected samples had identical gene counts using GDC or our BAMs.

Copy Number Variant Calling

Copy Number Variant (CNV) were detected using BIC-Seq2 (NBICseq-norm v0.2.4 and NBICseq-seg v0.7.2) (Xi et al., 2016) from WGS tumor and normal paired BAMs using Li Ding Lab's BIC-Seq2 pipeline v2.0 https://github.com/ding-lab/BICSEQ2. We used a bin size of 100 bp and a lambda of 3 (smoothing parameter for CNV segmentation). To further summarize the arm-level copy number change, we used a weighted sum approach (Vasaikar et al., 2019), in which the segment-level log₂ copy ratios for all the segments located in the given arm were added up with the length of each segment being weighted. We then used GISTIC2 v2.0.22 (Mermel et al., 2011) to integrate results from individual patients and identify genomic regions recurrently amplified or deleted in our samples. The threshold of arm-level CNV was 0.3 for gain and −0.3 for loss.

Somatic Variant Calling

Somatic variants were called from WES tumor and normal paired BAMs using TinDiasy v1.0, a modular software package designed for detection of somatic variants from tumor and normal exome data. TinDaisy merges and filters variant calls from four callers: Strelka v2.9.2 (Kim et al., 2018), VarScan v2.3.8 (Koboldt et al., 2012), Pindel v0.2.5 (Ye et al., 2009), and MuTect v1.1.7 (Cibulskis et al., 2013). SNV calls were obtained from Strelka, Varscan, and Mutect. Indel calls were obtained from Stralka2, Varscan, and Pindel. The following filters were applied to get variant calls of high confidence:
Normal VAF ≤0.02 and tumor VAF ≥0.05
Read depth in tumor ≥14 and normal ≥8
Indel length <100 bp
All variants must be called by 2 or more callers
All variants must be exonic
Exclude variants in dbSNP but not in COSMIC

We additionally called somatic whole-genome variants using WGS tumor and normal paired BAMs using somaticwrapper v1.3 with the default parameters, which ran the 4 variant callers identical to TinDaisy. The variant filtering was the same as TinDaisy except that we kept non-exonic variants.

Germline Variant Calling and Annotation

Germline variant calling was performed using Li Ding Lab's pipeline germlinewrapper v1.1, which implements multiple tools for the detection of germline INDELs and SNVs. Germline SNVs were identified using VarScan v2.3.8 (with parameters: --min-var-freq 0.10 --p-value 0.10, --min-coverage 3 --strand-filter 1) operating on a mpileup stream produced by samtools v1.2 (with parameters: -q 1 -Q 13) and GATK v4.0.0.0 (McKenna et al., 2010) using its haplotype caller in single-sample mode with duplicate and unmapped reads removed and retaining calls with a minimum quality threshold of 10. All resulting variants were limited to the coding region of the full-length transcripts obtained from Ensembl release 95 plus additional two base pairs flanking each exon to cover splice donor/acceptor sites. We required variants to have allelic depth 5 reads for the alternative allele in both tumor and normal samples. We used bam-readcount v0.8 for reference and alternative alleles quantification (with parameters: -q 10 -b 15) in both normal and tumor samples. Additionally, we filtered all variants with 0.05% frequency in gnomAD v2.1 (Karczewski et al., 2019) and The 1000 Genomes Project (The 1000 Genomes Project Consortium, 2015).

TERT Promoter Mutation Calling

We used bam-readcount program to count reads in WGS tumor and blood normal BAMs at the known hotspot positions at hg38 chr5:1295113 and chr5:1295135. We called a mutation if it was not observed in matching blood normal BAM and VAF >5%. For all tumor samples lacking a TERTp hotspot mutation, we performed the readcount across the entire TERT promoter region from chr5:1294200 to chr5:1295601. In these cases, we applied more stringent VAF cutoff to be 10%.

Structural Variant Calling

Structural Variant (SV) were called by Manta v1.6.0 (Chen et al., 2016) and DELLY v0.8.1 (Rausch et al., 2012) from WGS tumor and normal paired BAMs. We ran Manta on canonical chromosomes with the default record- and sample-level filters. For DELLY, we followed somatic SV workflow Only SV calls with PASS filter status were kept for downstream analysis. Lastly, we manually reviewed all the SV calls in the genes of interest (e.g. EGFR and PDGFRA).

DNA Methylation Microarray Processing

Raw methylation idat files were downloaded from CPTAC DCC and GDC. Beta values of CpG loci were reported after functional normalization, quality check, common SNP filtering, and probe annotation using Li Ding Lab's methylation pipeline v1.1 https://github.com/ding-lab/cptac_methylation. Resulting beta values of methylation were used for downstream analysis.

Telomere Length Quantification and Telomere Genotyping

We used Telseq v0.0.1 (Ding et al., 2014) to estimate the telomere length using WXS and WGS tumor and blood normal paired BAMs. We defined telomere length ratio as ratio between the estimated tumor telomere length and the estimated blood normal telomere length. While WXS and WGS-based telomere length ratios were well correlated, we used WGS based lengths for the downstream analysis. We defined long telomere phenotype as tumors with WGS telomere length ratio >1.2, and short telomere phenotype as WGS telomere length ratio <0.8.

We identified telomere genotypes as the following:
TERTp hotspot if tumor has TERTp hotspot mutation
ATRXmut for all remaining tumors with only ATRX mutation
ATRXmut IDH1mut for all remaining tumors with both ATRX and IDH1 mutated
IDH1mut for all remaining tumors with only IDH1 mutation
TERTp not hotspot for all remaining tumors without hotspot mutation in TERT promoter and expressing TERT
WT for the remaining tumors that do not fall into any category

RNA Quantification and Analysis

RNA Quantification

We obtained the gene-level readcount, Fragments Per Kilobase of transcript per Million mapped reads (FPKM) and FPKM Upper Quartile (FPKM-UQ) values by following the GDC's RNA-Seq pipeline (Expression mRNA Pipeline) https://docs.gdc.cancer.gov/Data/Bioinformatics_Pipelines/Expression_mRNA_P ipeline/, with the exception of running the quantification tools in the stranded mode. We used HTSeq v0.11.2 (Anders et al., 2015) to calculate the gene-level stranded readcount (parameters: -r pos -f bam -a 10 -s reverse -t exon gene_id -m intersection-nonempty --nonunique=none) using GENCODE v22 (Ensembl v79) annotation downloaded from GDC (gencode.gene.info.v22.tsv). The readcount was then converted to FPKM and FPKM-UQ using the same formula described in GDC's Expression mRNA Pipeline documentation.

RNA Fusion Detection

We used three callers, STAR-Fusion v1.5.0 (Haas et al., 2019), INTEGRATE v0.2.6 (Zhang et al., 2016b), and EricScript v0.5.5 (Benelli et al., 2012), to call consensus fusion/chimeric events in our samples. Calls by each tool using tumor and normal RNA-Seq data were then merged into a single file and extensive filtering is done. As STAR-Fusion has higher sensitivity, calls made by this tool with higher supporting evidence (defined by fusion fragments per million total reads, or FFPM>0.1) were required, or a given fusion must be reported by at least 2 callers. We then removed fusions present in our panel of blacklisted or normal fusions, which included uncharacterized genes, immunoglobulin genes, mitochondrial genes, and others, as well as fusions from the same gene or paralog genes and fusions reported in TCGA normal samples (Gao et al., 2018), GTEx tissues (reported in STAR-Fusion output), and non-cancer cell studies (Babiceanu et al., 2016). Finally, we removed normal fusions from the tumor fusions to curate the final set.

miRNA Quantification

miRNA-Seq FASTQ files were downloaded from GDC. We reported the mature miRNA and precursor miRNA expression in TPM (Transcripts Per Million) after adapter trimming, quality check, alignment, annotation, reads counting using Li Ding Lab's miRNA pipeline https://github.com/ding-lab/CPTAC_miRNA. The mature miRNA expression was calculated irrespective of its gene of origin by summing the expression from its precursor miRNAs.

Circular RNA Prediction and Quantification

The hg38 reference genome and GDC's annotations were used for the circRNA analysis. First, CIRI v2.0.6 (Gao et al., 2015) was used to call circular RNA with default parameters and BWA v0.7.17-r1188 (Li and Durbin, 2009) was used as a mapping tool. The cutoff of supporting reads for circRNA was set to 10. Then a pseudo-linear transcript strategy was used to quantify circular RNA expression (Li et al., 2017). In brief, for each sample, linear transcripts of circular RNAs were extracted and 75 bp (read length) from the 3′ end was copied to the 5′ end. The modified transcripts were called pseudo-linear transcripts. Transcripts of linear genes were also extracted and mixed with pseudo-linear transcripts. RSEM v1.3.1 (Li and Dewey, 2011) with Bowtie2 v2.3.3 (Langmead and Salzberg, 2012) as the mapping tool was used to quantify circular RNA expression based on the mixed transcripts. After quantification, the upper quantile method was applied for normalization and the normalized matrix was log₂-transformed.

MS Data Interpretation

Quantification of TMT Global Proteomics Data

LC-MS/MS analysis of the TMT11-labeled, bRPLC fractionated samples generated a total of 264 global proteomics data files. The Thermo RAW files were processed with mzRefinery to characterize and correct for any instrument calibration errors, and then with MS-GF+ v9881 (Gibbons et al., 2015; Kim and Pevzner, 2014; Kim et al., 2008) to match against the RefSeq human protein sequence database downloaded on Jun. 29, 2018 (hg38; 41,734 proteins), combined with 264 contaminants (e.g., trypsin, keratin). The partially tryptic search used a ±10 ppm parent ion tolerance, allowed for isotopic error in precursor ion selection, and searched a decoy database composed of the forward and reversed protein sequences. MS-GF+ considered static carbamidomethylation (+57.0215 Da) on Cys residues and TMT modification (+229.1629 Da) on the peptide N-terminus and Lys residues, and dynamic oxidation (+15.9949 Da) on Met residues for searching the global proteome data.

Peptide identification stringency was set at a maximum 1% FDR at peptide level using PepQValue <0.005 and parent ion mass deviation <7 ppm criteria. A minimum of 6 unique peptides per 1000 amino acids of protein length was then required for achieving 1% at the protein level within the full data set. Inference of parsimonious protein set at gene level resulted in the identification of protein groups covering 11,141 genes.

The intensities of all ten TMT reporter ions were extracted using MASIC software (Monroe et al., 2008). Next, PSMs passing the confidence thresholds described above were linked to the extracted reporter ion intensities by scan number. The reporter ion intensities from different scans and different bRPLC fractions corresponding to the same gene were grouped. Relative protein abundance was calculated as the ratio of sample abundance to reference abundance using the summed reporter ion intensities from peptides that could be uniquely mapped to a gene. The pooled reference sample was labeled with TMT 126 reagent, allowing comparison of relative protein abundances across different TMT-11 plexes. The relative abundances were log₂ transformed and zero-centered for each gene to obtain final relative abundance values.

Small differences in laboratory conditions and sample handling can result in systematic, sample-specific bias in the quantification of protein levels. In order to mitigate these effects, we computed the median, log₂ relative protein abundance for each sample and re-centered to achieve a common median of 0.

Quantification of Phosphopeptides

Phosphopeptide identification for the 132 phosphoproteomics data files were performed as in the global proteome data analysis described above (e.g., peptide level FDR <1%), with an additional dynamic phosphorylation (+79.9663 Da) on Ser, Thr, or Tyr residues. The phosphoproteome data were further processed by the Ascore algorithm (Beausoleil et al., 2006) for phosphorylation site localization, and the top-scoring sequences were reported. For phosphoproteomic datasets, the TMT-11 quantitative data were not summarized by protein but left at the phosphopeptide level. All peptides (phosphopeptides and global peptides) were labeled with TMT-11 reagent simultaneously. Separation into phospho- and non-phosphopeptides using IMAC was performed after the labeling. Thus, all the biases upstream of labeling are assumed to be identical between global and phosphoproteomic datasets. Therefore, to account for sample-specific biases in the phosphoproteome analysis, we applied the correction factors derived from median-centering the global proteomic dataset.

Quantification of Acetylated Peptides

Acetylated peptide identification for the 44 acetylome data files were performed as in the global proteome data analysis described above, with additional dynamic acetylation (+42.0105 Da) and carbamylation (+43.0058 Da) on Lys residues. The acetylation site localization, protein inference, and quantification of the acetylome data were performed in identical fashion as in the phosphoproteome data.

Preprocessing of Proteomics Tables

Due to the quantification of small values close to 0 on spectrum level, some extreme positive or negative values were generated after log₂ transform of relative protein/phosphopeptide/acetyl peptide abundance, which may have negative impact on the downstream analysis of the data sets. To identify TMT outliers with extreme values, we perform inter-TMT t-test for each individual protein/phosphopeptide/acetyl peptide. For a specific protein/phosphopeptide/acetyl peptide, relative abundance level of each TMT value was compared against all the other TMT values using Spearman two-sample test. Outlier was defined if the p value past certain threshold. In the global proteome data, 153 TMT values were identified as outliers with inter-TMT t-test p-value lower than 10e-6, as a result 1,530 data points (0.14% of all observations) were removed from the data sets. In the phosphoproteome data, 379 TMT values were identified as outliers with inter-TMT t-test p-value lower than 10e-10, resulting in 3,790 data points (0.09% of all observations) removed from the data sets. In the acetylome data, 12 TMT values were identified as outliers with inter-TMT t-test p-value lower than 10e-14, and 120 data points (0.015% of all observations) were removed from the data sets.

Batch effects were checked using the log₂ relative protein/phosphopeptide abundance or protein/acetyl peptide abundance, and removed using Combat algorithm (Beausoleil et al., 2006) after TMT outlier filtering. Imputation was performed after batch effect correction to produce a different version of the data tables for some of the data analysis tools that are sensitive to missing values. The proteins/phosphopeptide/acetyl peptide with missing rate less than 50% were selected and imputed with the DreamAI algorithm https://github.com/WangLab-MSSM/DreamAI tailored for proteomics data.

Sample Outlier Identification of Metabolome and Lipidome

A robust Mahalanobis distance based on biomolecule abundance vectors (rMd-PAV) was calculated to identify potential sample outliers in the data (Matzke et al., 2011). For proteomics data, this distance was calculated based on four metrics: average correlation with samples in the same group, skewness of biomolecule abundance distribution, the proportion of missing data, and median absolute deviation of abundances. These metrics, minus the proportion missing, were used for the metabolomics and lipidomics datasets. To confirm any sample outliers identified by rMd-PAV, a correlation heatmap was generated and sequential projection pursuit principal component analysis (PCA) was run (Webb-Robertson et al., 2013). No sample outliers were identified in the proteomics dataset. One outlier, C3N-01366, was removed from the metabolomics dataset; C3N-01370 was removed from the positive lipid dataset and C3L-03968 from the negative lipid dataset.

Normalization and Protein Quantification of Metabolome and Lipidome

Global median centering, where each sample is normalized to the median of its observed values, was used to normalize all datasets. Protein quantification was accomplished via R-rollup (Polpitiya et al., 2008), in which peptides were scaled by a reference peptide and the protein abundance was set as the median of the scaled peptides.

Other Proteogenomic Analysis

Sample Labeling Check Across Data Types

While multiple omics data enhance our understanding of complex molecular mechanisms underlying GBM, it is sometimes inevitable to have sample errors including sample swapping, shifting or data contamination. Working on error-containing data is dangerous since it could lead to a wrong scientific location. Therefore, it is required to confirm whether different types of molecular data are pertained from the same individuals prior to data integration or public sharing. For the GBM dataset, we performed sample labeling check across different types of data as described previously (Clark et al., 2019). Using MODMatcher (Yoo et al., 2014), we confirmed that all samples were well aligned among RNA-Seq, proteomics and CNV (WGS) data.NMF

Multi-Omics Subtyping Using Non-Negative Matrix Factorization (NMF)

We used non-negative matrix factorization (NMF) implemented in the NMF R-package (Gaujoux and Seoighe, 2010) to perform unsupervised clustering of tumor samples and to identify proteogenomic features (proteins, phosphosites, acetylation sites and RNA transcripts) that show characteristic expression patterns for each cluster. Briefly, given a factorization rank k (where k is the number of clusters), NMF decomposes a p×n data matrix V into two matrices Wand H such that multiplication of Wand H approximates V. Matrix H is a k×n matrix whose entries represent weights for each sample (1 to N) to contribute to each cluster (1 to k), whereas matrix W is a p×k matrix representing weights for each feature (1 to p) to contribute to each cluster (1 to k). Matrix H was used to assign samples to clusters by choosing the k with maximum score in each column of H. For each sample, we calculated a cluster membership score as the maximal fractional score of the corresponding column in matrix H. We defined a “cluster core” as the set of samples with cluster membership score >0.5. Matrix W containing the weights of each feature to a certain cluster was used to derive a list of representative features separating the clusters using the method proposed in (Kim and Park, 2007).

To enable integrative multi-omics clustering we enforced all data types (and converted if necessary) to represent log₂-ratios to either a common reference measured in each TMT plex (proteome, phosphoproteome), an in silico common reference calculated as the median abundance across all samples (RNA gene expression) or to gene copy numbers relative to matching normal blood sample (CNV). All data tables where then concatenated and all rows containing missing values were removed. To remove uninformative features from the dataset prior to NMF clustering, we removed features with the lowest standard deviation (bottom 5th percentile) across all samples. Each row in the data matrix was further scaled and standardized such that all features from different data types were represented as z-scores.

Since NMF requires a non-negative input matrix we converted the z-scores in the data matrix into a non-negative matrix as follows:
1) Create one data matrix with all negative numbers zeroed
2) Create another data matrix with all positive numbers zeroed and the signs of all negative numbers removed
3) Concatenate both matrices resulting in a data matrix twice as large as the original, but with positive values only and zeros and hence appropriate for NMF

The resulting matrix was then pass to NMF analysis in R using the factorization method described in (Brunet et al., 2004). To determine the optimal factorization rank k (number of clusters) for the multi-omic data matrix, we tested a range of clusters between k=2 and 8. For each k, we factorized matrix V using 50 iterations with random initializations of Wand H. To determine the optimal factorization rank, we calculated cophenetic correlation coefficients to measure how well the intrinsic structure of the data is recapitulated after clustering. Finally, we picked the k with maximal cophenetic correlation for cluster numbers between k=3 and 8.

To achieve robust factorization of the multi-omics data matrix V, we took the optimal factorization rank k, repeated the NMF analysis for 200 iterations with random initializations of Wand H, and partitioned the samples into clusters as described above. Due to the non-negative transformation of the z-scored data matrix, feature weight matrix W contained two separate weights for positive and negative z-scores of each feature, respectively. To revert the non-negative transformation and to derive a single signed weight for each feature, we first normalized each row in matrix W by dividing by the sum of feature weights in each row, aggregated both weights per feature and cluster by keeping the maximal normalized weight and multiplication with the sign of the z-score the initial data matrix. Thus, the resulting transformed matrix W_{signed} contained signed cluster weights for each feature in the input matrix.

For each cluster, we calculated normalized enrichment scores (NES) of cancer-relevant gene sets by projecting the matrix of signed multi-omic feature weights W_{signed} onto hallmark pathway gene sets (Liberzon et al., 2015) using ssGSEA (Barbie et al., 2009) available on https://github.com/broadinstitute/ssGSEA2.0 (parameters: gene.set.database=“h.all.v6.2.symbols.gmt” sample.norm.type=“rank” weight=1 statistic=“area.under.RES” output.score.type=“NES” nperm=1000 global.fdr=TRUE min.overlap=5 correl.type=“z.score”). To derive a single weight for each gene measured across all omics data types, we retained the weight with maximal absolute amplitude. We then associated the resulting clusters to sample-level variables by testing for overrepresentation in the cluster core sample sets using Fisher's exact test. The following clinical variables were used: expression subtype, sex, vital status, and smoking history.

The entire NMF workflow has been implemented as a module on Broad's Cloud platform Terra (https://app.terra.bio/). The docker containers encapsulating the source code and the required R packages for NMF clustering and ssGSEA were available on Dockerhub (broadcptac/pgdac_mo_nmf:9, broadcptac/pgdac_ssgsea:5).

Expression Based TCGA Subtyping

Gene expression based subtypes were based on the 150 genes created by Wang et al., the most recent TCGA subtyping effort (Wang et al., 2017), which contained 50 highly expressed genes in classical, proneural, and mesenchymal IDH wild type tumors. Tumors with recurrent mutations in IDH1/2 (IDH1 R132H specifically in our cohort) were assigned to be IDH mutant tumors. We then performed consensus clustering on all tumors based on the selected gene expression in log₂(FPKM-UQ+1) using ConsensusClusterPlus R package (parameters: maxK=10 reps=2000 pltem=0.8 pFeature=1 clusterAlg=“hc” distance=“pearson” seed=201909). We chose the total number of clusters k=5 based on the delta area plot of consensus CDF. The clusters were annotated with the TCGA subtypes based on their gene expression profiles. Three clusters (r1, r4, and r5) were merged due to their similar expression signature, which was identical to the clustering result while choosing k=3.

Unsupervised Clustering of DNA Methylation

Methylation subtypes were segregated based on the top 8,000 most variable probes using k-means consensus clustering as previously described (Sturm et al., 2012). We first removed underperforming probes (Zhou et al., 2017b), and then the samples with more than 30% missing values. Remaining missing values were imputed using the mean of the corresponding probe value. We then performed clustering 1000 times using ConsensusClusterPlus R package (parameters: maxK=10 reps=1000 pltem=0.8 pFeature=1 clusterAlg=“km” distance=“euclidean”). We choose k=6 based on the delta area plot of consensus CDF.

Classification of MGMT Promoter DNA Methylation Status

We selected the DNA methylation probes from 3 kb upstream to 500 bp downstream to the MGMT transcription start site and performed unsupervised clustering of their beta values to extract two clusters from all tumors. The cluster with the average higher MGMT methylation was considered MGMT promoter hypermethylated.

Unsupervised Clustering of miRNA Expression

Unsupervised miRNA expression subtype identification was performed on mature miRNAs expression (log₂ TPM) from 98 GBM tumors with miRNA data available using Louvain clustering (Blondel et al., 2008) implemented in louvain-igraph v0.6.1. Top 50 differentially expressed miRNAs from each miRNA-based subtype were selected.

Determination of Stemness Score

Sternness scores were calculated as previously described (Malta et al., 2018). To calculate the sternness scores based on mRNA expression, we built a predictive model using one-class logistic regression (OCLR) (Sokolov et al., 2016) on the pluripotent stem cell samples (ESC and iPSC) from the Progenitor Cell Biology Consortium (PCBC) dataset (Daily et al., 2017; Salomonis et al., 2016). For mRNA expression-based signatures, to ensure compatibility with our cohort, we first mapped the Ensembl IDs to Human Genome Organization (HUGO) gene names and dropped any genes that had no such mapping. The resulting training matrix contained 12,945 mRNA expression values measured across all available PCBC samples. To calculate mRNA-based sternness index (mRNASi), we used FPKM-UQ mRNA expression values for all CPTAC GBM tumors and GTEx samples. We used TCGAanalyze_Stemness function from the R package TCGAbiolinks (Colaprico et al., 2016) and following our previously described workflow (Silva et al., 2016), with “stemSig” argument set to PCBC stemSig.

Multi-Omics Cis Association Analysis Using iProFun

We integrated somatic mutation, CNV, DNA methylation, RNA, protein, phosphorylation (phospho) and acetylation (acetyl) levels via iProFun (Song et al., 2019) to investigate the functional impacts of DNA alterations in GBM. All data types were preprocessed to eliminate potential issues for analysis such as batch effects, missing data and major unmeasured confounding effects before the iProFun analysis. As phosphoprotein and acetylprotein were measured in a small subset of the genes in comparison with RNA and protein, we considered three sets of iProFun analysis using different combination functional outcomes (mRNA/protein, mRNA/protein/phospho, and mRNA/protein/acetyl) to include as many as possible genes and omics for investigation. For each set of outcomes (e.g. RNA and protein), we considered their levels perturbed jointly by three DNA alterations (somatic mutation, CNV, and DNA methylation). The effects of DNA methylation on molecular traits are usually smaller than mutation and CNV, and thus adjusting their effects in analysis is critical to obtain unconfounded associations for methylation. In addition, we adjusted age, sex, and tumor purity in the analysis. Turnor purity was determined using XCell (PMID: 29141660) from RNA-Seq data.

The iProFun procedure was applied to a total of 7,464 genes with measured RNA/protein, 4,433 genes with measured RNA/protein/phospho, and 1,315 genes with measured RNA/protein/acetyl data, respectively, for their cis regulatory patterns in tumors. For example, when we considered DNA methylation for its effects on RNA/protein/phospho, we started with the traditional linear regression for each of the three outcomes separately:
RNA ˜methylation+covariates
protein ˜methylation+covariates
phospho ˜methylation+covariates

The covariates here include CNV, somatic mutations (genes with mutation rate ≥10%), age, sex, and tumor purity. Then iProFun took the association summary statistics from these three regressions as input to call posterior probabilities of belonging to each of the eight possible configurations (e.g., “None”, “RNA only”, “protein only”, “phospho only” “RNA & protein”, “RNA & phospho”, “protein & phospho” and “all three”) and to determine significance associations.

A gene was identified to present significant and biologically meaningful association if the association passes three criteria: (1) the satisfaction of biological filtering procedure, (2) posterior probabilities >75%, and (3) empirical false discovery rate (eFDR)<10%. Specifically, the biological filtering criterion requires that CNV presents positive associations with all the types of molecular quantitative traits (QTs), DNA methylation presents negative associations with all the types of molecular QTs, and mutation requires the association across all outcome platforms preserve consistent directions (either positive or negative). Secondly, a significance was called only if the posterior probabilities >75% of a predictor being associated with a molecular QT, by summing over all configurations that are consistent with the association of interest. For example, the posterior probability of a methylation being associated with mRNA expression levels was obtained by summing up the posterior probabilities in the following four association patterns—“RNA only”, “RNA & protein”, “RNA & phospho” and “all three”, all of which were consistent with methylation being associated with mRNA expression. Lastly, we calculated the empirical FDR (eFDR) via 100 permutations per molecular QTs by shuffling the label of the molecular QTs and required eFDR <10% by selecting a minimal cutoff value of a that 75%<α<100%. The eFDR is calculated by:

$$eFDR = \frac{\begin{matrix}Average \# genes with posterior probability > \\ α in permutated data\end{matrix}}{\begin{matrix}Average \# genes with posterior probability > \\ α in original data\end{matrix}}$$

Results of whether the DNA methylation/CNV/mutation of a gene has perturbed any of its cis QTs (mRNA, protein, phosphoprotein and acetylprotein) were obtained.

Mutation Impact on the RNA, Proteome, Phosphoproteome, Lipidome and Metabolome

We aggregated a set of interacting proteins (e.g. kinase/phosphatase-substrate or complex partners) from OmniPath (downloaded on 2018-03-29) (Turei et al., 2016), DEPOD (downloaded on 2018-03-29) (Duan et al., 2015), CORUM (downloaded on 2018-06-29) (Ruepp et al., 2010), Signor2 (downloaded on 2018-10-29) (Perfetto et al., 2016), and Reactome (downloaded on 2018-11-01) (Fabregat et al., 2018). We focused our analyses on 18 GBM SMGs previously reported in the literature: PIK3R1, PIK3CA, PTEN, RB1, TP53, EGFR, IDH1, BRAF, NF1, PDGFRA, ATRX, and TERTp) (Bailey et al., 2018; Brennan et al., 2013).

For each interacting protein pair, we split samples with and without mutations in partner A and compare expression levels (RNA, protein and phosphosites) both in cis (partner A) and in trans (partner B), calculating a median difference in expression and testing for significance with the Wilcoxon rank sum test, with the Benjamini-Hochberg multiple test correction. For mutational impact analysis on lipidome or metabolome, all possible pairs between SMGs and metabolites/lipids were tested.

Kinase-Substrate Pairs Regression Analysis

For each kinase-substrate protein pairs supported by previous experimental evidence (OmniPath, NetworKIN, DEPOD, and SIGNOR), we tested the associations between all sufficiently detected phosphosites on the substrate and the kinase. For a kinase-substrate pair to be tested, we required both kinase protein/phosphoprotein expression and phosphosite phosphorylation to be observed in at least 20 samples in the respective datasets and the overlapped dataset. We then applied the linear regression model using Im function in R to test for the relation between kinase and substrate phosphosite. For the i-th trial for kinase phosphosite abundance in the cis associations, kinase phosphosite abundance Aᵢ depends on kinase protein expression Sᵢ and error Eᵢ

Aᵢ=M₁Sᵢ+B+Eᵢ

For the i-th trial for kinase phosphosite abundance in the trans associations, substrate phosphosite abundance Aᵢ depends on kinase phosphosite expression Kᵢ substrate protein expression Sᵢ and error Eᵢ

Aᵢ=M₁Sᵢ+M₂Kᵢ+B+Eᵢ

where the regression slope M coefficients are determined by least-square calculation. The resulting p-values were adjusted for multiple testing using the Benjamini-Hochberg procedure.

For the broader investigation of signaling cascades, we included total 214 kinases and 43 phosphatases if they satisfied either of the genetic alteration criteria or at least three criteria below:
5% and more tumors with copy number alterations
2 and more tumors with somatic mutations
Top 20% variable gene expression
Top 35% variable protein expression
Significantly different RNA or protein expression between tumor and normal (FDR ≤0.01)

Differential Proteomic, Phosphosite, Metabolome and Lipidome Analysis

TMT-based global proteomic, phosphoproteomic, and acetylation, as well as metabolome and lipidome data were used to perform pairwise differential analysis between groups of samples. A Wilcoxon rank-sum test was performed to determine differential abundance of proteins, PTMs and metabolites. At least four samples in both groups were required to have non-missing values and the p-value was adjusted using the Benjamini-Hochberg procedure. For phosphorylation markers in each genomic subtype, the adjusted p-value for the protein change was required to be 0.05.

Phosphoproteome Outlier Analysis

Outlier Analysis was done using BlackSheep's DEVA analysis (Blumenberg et al., 2019). Phosphopeptide analysis was done on data that was aggregated per protein, summing together outlier values across all phosphosites. Protein analysis was performed using TMT-based global proteomic data, RNA analysis was done using FPKM-UQ normalized transcript data. The DEVA method calculates interquartile range (IQR) and median values for the given dataset, and then defines outliers as values greater than the median plus 1.5×IQR. Features were prefiltered to include an outlier value in at least 30% of samples in the group of interest and for features that had a higher proportion of features in the group of interest compared to the rest of the population. Statistics were calculated using a Fisher's exact test and p-values were corrected using the Benjamini-Hochberg procedure. Druggability of a gene/protein was performed using DGIdbR (Cotto et al., 2018).

Copy Number Impact on Transcriptome and Proteome

To evaluate copy number impact on RNA and protein expression, we applied gene-wise correlation analysis on CNV versus RNA expression and on CNV versus protein expression. Correlation was performed by Pearson's correlation method. Both correlation coefficient and p-value were computed and adjusted by the Benjamini-Hochberg procedure.

Cell Type Enrichment Deconvolution Using Gene Expression

The abundance of each cell type was inferred by xCell web tool (Aran et al., 2017), which performed the cell type enrichment analysis from gene expression data for 64 immune and stromal cell types (default xCell signature). xCell is a gene signatures-based method learned from thousands of pure cell types from various sources. We input the FPKM-UQ expression matrix of this study in xCell using the expression levels ranking.

Immune Clustering Using Cell Type Enrichment Scores

Immune subtypes of the GBM tumors was generated on the consensus clustering of the cell type enrichment scores by xCell (Wilkerson and Hayes, 2010). Among the 64 cell types tested in xCell, we selected 43 cell types with at least 2 samples with xCell enrichment p<0.01, which filtered out the cell types that not presented in brain. xCell generated an immune score per sample that integrates the enrichment scores B cells, CD4+ T-cells, CD8+ T-cells, DC, eosinophils, macrophages, monocytes, mast cells, neutrophils, and NK cells. In addition, we included microglia using the scores by ssGSEA based on its marker genes: P2RY12, TMEM119, SLC2A5, TGFBR1, GPR34, SALL1, GAS6, MERTK, C1QA, PROS1, CD68, ADGRE1, AIF1, CX3CR1, TREM2, and ITGAM. The microglia ssGSEA score was computed using the R package GSVA (gsva function with method=‘ssgsea’). We performed consensus immune clustering based on the z-score normalized xCell and microglia scores. The consensus clustering was determined by the R package ConsensusClusterPlus (parameters: clusterAlg=‘kmdist’ method=‘spearman’).

Deep Learning Histopathology Image Analysis

We trained deep learning models for 3 different prediction tasks based on histopathology images, including the G-CIMP phenotype (positive and negative), immune response (low and high), and telomere length (short, normal, and long). Digital histopathology slides and associated quantified features (cellularity, necrosis, tumor nuclei, age, tumor weight) of samples used in proteomics analysis were downloaded from The Cancer Imaging Archive (TCIA) database. Labels were at per-case (patient) level. The images and their corresponding labels were then divided into 3 datasets at per-case level with 70% of cases in training set, 15% of cases in validation set, and 15% of cases in testing set. Due to the large size of the scanned histopathology slides, they were tiled into 299-by-299-pixel pieces with overlapping area of 49 pixels from each edge at 20×, 10×, and 5× resolution. In this process, tiles with over 30% of background pixels were removed. Qualified tiles, quantified features, and labels of each set were then loaded into a designated TFrecords file. After the data preparation, convolutional neural network (CNN) architectures, including InceptionV1 to V4, InceptionResNetV1 and V2, and self-designed simple CNNs, were trained from scratch. Statistical metrics, such as area under ROC, area under PRC, and top-1 accuracy, were used to evaluate the performance. The best model for each task was picked at the minimum validation loss point. Trained models were tested on the testing set and the statistical metrics of the testing set were used to compare the performance of different models on the same tasks.

A visualization method designed to unveil the features learned by the models was applied to discover histological features associated with G-CIMP phenotype, telomere length, and immune response in the cohort. Firstly, the activation score vectors of each tile from the fully connected layer immediately before output layer in the testing set were extracted as representation of the input samples. Then a randomly sampled subset of these activation score vectors was dimensionally reduced into 2-dimensional space by tSNE with each point representing an image tile. Overlay of prediction scores on these points revealed clusters corresponding to the labels. Finally, experienced pathologists examined the tiles in each of these clusters and summarized the general histological features in these clusters, which served as the representation of the histological features of these subgroups.

Gene Set Enrichment Analysis

Differential Expressed Genes (DEGs) were identified using DESeq2 (Love et al., 2014) by applying the minimal pre-filtering to keep only genes that have at least 10 reads in total. We selected the genes which had FDR ≤0.01 and absolute fold change larger than 2. To designate the representative pathways of immune subtypes, we selected the DEGs between the two immune subtypes and then underwent a pathway enrichment analysis of Hallmark, KEGG, and Reactome. The overrepresented pathways were selected (FDR <0.1, only pathways with at least 10 genes observed in each data type are considered).

To identify significantly enriched Hallmark, KEGG, PID, and REACTOME gene sets of each immune cluster, we applied the ssGSEA on all the protein to calculate the normalized enrichment score (NES) for each gene set in each sample. Then we performed the pairwise t-test of NES among the 2 immune clusters and adjusted the p-values by FDR. We ranked gene sets by FDR and selected the top 50 gene sets (all FDR <0.01) of each immune cluster.

Histone Protein and Acetylation Calculation

Core histones H2A, H2B, H3 and H4, and linker histone H1 are encoded by multiple genes with minor changes in their sequence. Accordingly, we detected a number of peptides and acetylated peptides corresponding to either of the core histones and H1 histone. To facilitate the interpretation of histone acetylation events, we averaged acetylation values for peptides mapped on different gene encoding practically the same histone protein.

Histone Acetylation Association with HATs and HDACs

To test the association between HATs/HDACs protein and acetylation levels of histone sites, we fitted Lasso regression model with HATs/HDACs and histone protein expression as independent variables and a histone acetylation site as a dependent variable. Lasso regression has been chosen because it takes expression of all enzymes into account simultaneously and is insensitive to highly correlated dependent variables. We performed 300 bootstraps with 80% training data and 20% testing data, and reported averaged coefficients returned by the model across 300 iterations.

Pathway Enrichment Analysis Along Histones H2B and H3/H4 Acetylation Axes

We investigated pathways from Hallmark, KEGG, WIkipathways, and REACTOME, positively or negatively aligned with averaged H2B and H3/H4 acetylation level. H2B acetylation was calculated by averaging acetylation of all H2B peptides detected. Since H3 and H4 histones are strongly correlated with each other, we averaged acetylation of histones H3 and H4 peptides together to obtain H3/H4 acetylation value.

We assumed that true biological activity of a pathway is regulated by collective changes of expression levels of majority of proteins involved in this pathway; then a difference in a pathway activity between tumors can be assessed by a difference in positioning of expression levels of proteins involved in this pathway in ranked list of expression levels of all proteins in each of tumors. Following this idea, we assessed relative positioning of pathway proteins between tumor by determining two probabilities: (1) probability of pathway proteins to occupy by random chance the observed positions in a list of tumor proteins ranked by expression level from the top to the bottom and, similarly, (2) probability to occupy by random the observed positions in a list of expression levels ranked from the bottom to the top. Then, the inferred relative activation of a given pathway across tumors was assessed as negative logarithm of the ratio of the above “top” and “bottom” probabilities. Thus, for a pathway of a single protein, its relative activity across tumors was assessed as a negative log of ratio of two numbers: a number of proteins with expression level bigger than an expression level of given protein, and a number of proteins with expression levels less than an expression level of given protein. For pathways of multiple proteins, the “top” and “bottom” probabilities were computed as geometrically averaged P values computed for each of proteins using Fisher's exact test, given protein's ranks in a list of pathway proteins and in a list of ranked proteins of a tumor, a number of proteins in a pathway, and the total number of proteins with the assessed expression level in a given tumor. The thermodynamic interpretation of the inferred pathway activity scoring function is a free energy associated with deviation of the system from equilibrium either as a result of activation or suppression. Thus, the scoring function is positive, when expression levels of pathway's proteins are overrepresented among top expressed proteins of a tumor, and it is negative, when pathway's proteins are at the bottom of expressed proteins of a tumor; the scoring function is close to zero, when expression levels are distributed by random. Given, any biological axis, e.g. histone acetylation levels in each of tumors, one can determine pathways which are significantly correlated or anti correlated with the axis.

Causative Pathway Interaction Discovery Using CausalPath

To discover the causative pathway interactions in our proteomic and phosphoproteomic data, we took the normalized expression of protein with <10% missing values and phosphoprotein with <25% missing values across all tumor and normal samples as the input to CausalPath (commit 7c5b934). We ran CausalPath in the mode that tests the mean values between test and control groups (value-transformation=significant-change-of-mean), where test group being the tumors of one subtype and control group being the rest of the tumors. The pathway interaction discovery data source was Pathway Commons v9 (built-in-network-resource-selection=PC). Additionally, we enabled the causal reasoning if all the downstream targets of a gene was active or inactive (calculate-network-significance=true, use-network-significance-for-causal-reasoning=true, permutations-for-significance=10000). The causative interactions with FDR <0.05 were extracted and visualized (fdr-threshold-for-data-significance=0.05 phosphoprotein, fdr-threshold-for-data-significance=0.05 protein, fdr-threshold-for-network-significance=0.05).

Data and Code Availability

Clinical data and raw proteomic data reported in this paper can be accessed via the CPTAC Data Portal at: https://cptac-data-portal.georgetown.edu/cptac/s/5048. Genomic and transcriptomic data files can be accessed via Genomic Data Commons (GDC) at: https://portal.gdc.cancer.gov/projects/CPTAC-3. The cptac Python package, and LinkedOmics (Vasaikar et al., 2018).

What is claimed is:

1. A method of inhibiting PTPN11 in a tumor or tumor cell comprising:
detecting upregulated PTPN11 in the tumor or tumor cell; and
administering a PTPN11 inhibitor to the tumor or tumor cell in an amount effective to inhibit PTPN11.

2. The method of claim 1, wherein the PTPN11 inhibitor is selected from a Shp2 inhibitor.

3. The method of claim 2, wherein the Shp2 inhibitor is selected from SHP099, TNO155, JAB-3068, RMC-4630, BBP 398, RLY1971, or combinations thereof.

4. The method of claim 1, wherein the tumor is or tumor cell is from lung cancer, brain cancer, breast cancer, or pancreatic cancer.

5. The method of claim 1, wherein upregulated PTPN11 is detected by measuring increased expression levels of PTPN11 compared to a tumor sample that is not EGFR, ALK1, or PDGFRA-activated.

6. The method of claim 1, wherein the tumor is or the tumor cell is a cell from an EGFR, ALK1, or PDGFRA-activated tumor.

7. The method of claim 1, wherein if the tumor or tumor cell is from lung cancer or a lung tumor cell and is:
EGFR mutated- and ALK fusion-driven, a subject having a tumor comprising the tumor cell is treated with a Shp2 inhibitor, optionally selected from SHP099, TNO155, JAB-3068, RMC-4630, BBP 398, or RLY1971 and an ALK inhibitor, optionally selected from ceritinib;
KRAS mutated, the subject or tumor cell is treated with SOS1 inhibition;
KRAS and EGFR mutated, the subject or tumor cell is treated with SOS1 inhibition and PTPN11 (Shp2) inhibitor;
EGFR mutated, the subject or tumor cell is treated with a PTPN11 (Shp2) inhibitor and an EGFR tyrosine kinase inhibitor;
EGFR mutated, the subject or tumor cell is treated with a modulator or inhibitor of: PHLDA1, PHLDA3, SOX9, CTNND2 (δ-catenin), CDK6, or CDKN2C;
EML4-ALK mutated, the subject or tumor cell is treated with a WEE1 inhibitor;
immune hot, the subject or tumor cell is treated with a combination of PD-1/PD-L1 blockade and 001 inhibitor;
KEAP mutated, the subject or tumor cell is treated with a modulator or inhibitor of: TXNRD1, SRXN1, NQO1, ARK1C1, ARK1C3, GPX2, AKR1C2, BAG2, UGHD, ARK1B10, ARK1C4, TALDO1, GCLC, GCLM, UCHL1, AKR1B10, RMND1, PGD, GSR, or CYP4E11;
KEAP mutated, the subject or tumor cell is treated with a modulator or inhibitor of: NFE2L2, AKR1C1, AKR1C1, NQO1, NFE2L2, CWC22, or MAP2;
KEAP mutated, the subject or tumor cell is treated with a KEAP1/NFE2L2 interaction inhibitor;
cancer testis (CT) antigen or neoantigen positive the subject or tumor cell is treated with immunotherapy or an inhibitor or modulator of KIF2C, IGF2BP3, PBK, PIWIL, BRDT, TEX15, or AKAP4;
TP53 mutated, the subject or tumor cell is treated with a BRAF inhibitor or a EZH2 inhibitor;
Treg upregulated, the subject or tumor cell is treated with anti-CTLA4 therapy;
EGFR phosphoprotein positive (enriched), the subject or tumor is treated with a modulator or inhibitor of: WNK1, EGFR, CDK18, CSNK1D, NEK1, PDPK1, RIPK2, PI4KB, STK3, PAK4, DCLK1, DAPK1, NEK4, or STK25;
KRAS phosphoprotein positive (enriched), the subject or tumor is treated with a modulator or inhibitor of: SLK, PRKCD, PRKCA, PRKD2, MAP3K2, KIT, MAST3, RPS6KC1, NRBP1, PRKAB2, FN3K, AATK, or NME1;
TP53 phosphoprotein positive (enriched), the subject or tumor is treated with a modulator or inhibitor of: EGFR, RIPK2, DCLK1, CDK12, BRD2, MELK, BRAF, PKN1, NRBP1, TRIO, TLK2, PRPF4B, or RIOK1;
STK11 phosphoprotein positive (enriched), the subject or tumor is treated with a modulator or inhibitor of: SLK, PKN2, TGFBR1, DYRK1B, or PRPF4B;
KEAP phosphoprotein positive (enriched), the subject or tumor is treated with a modulator or inhibitor of: PAK4, DCLK1, TRIO, TLK2, BRD2, TGFBR1, TRIM28, MAPKAPK5, MAP3K7, DYRK2, CDK7, PAK2, TRIO, TLK2, TRIM28, BUB1B, ITPK1, or TRPM7; or
ALK phosphoprotein positive (enriched), the subject or tumor is treated with a modulator or inhibitor of: PTK7, PRKAG2, WEE1, LRRK2, NEK6, PTK7, PRKAG2, PKM, or RIPK3; or
combinations thereof.

8. The method of claim 1, wherein PTPN11 and PLCG1 are hyperphosphorylated and mediate RAS pathway activation in the tumor.

9. The method of claim 1, wherein the tumor is an EGFR-altered or RB-1 altered tumor.

10. The method of claim 1, wherein if the tumor is from brain cancer or the tumor is
RB1-altered, a subject having a tumor comprising the tumor cell is treated with an MCM inhibitor, optionally, ciprofloxacin;
hypermethylated in MGMT promoter region, the subject or tumor cell is treated with temozolomide chemotherapy and radiotherapy;
EGFR-altered having co-upregulation of CDK6 and EGFR, the subject or tumor cell is treated with CDK6 and EGFR inhibitors;
BRAF V600E mutated having high H2B acetylation, the subject or tumor cell is treated with UBE2I inhibitors;
CDK6 and UBE2I protein upregulated, the subject or tumor cell is treated with UBE2I inhibitors;
RTK-altered, the subject is treated with anti-Shp2 therapy;
ATRX and IDH1 mutated, the subject or tumor cell is treated with a TNIK inhibitor;
immune cold (immune-low), the subject is treated with a modulator or inhibitor of: PTGR2, PDE4D, MAOA, MUC5B, or WFDC2 (HE4);
immune hot (immune-high), the subject or tumor cell is treated with a CSF-1 or CSF-1R inhibitor, optionally, a CSF-1 receptor inhibitor, PLX3397;
immune hot (immune-high), the subject or tumor cell is treated with a TAM infiltration inhibiting agent capable of targeting monocyte chemoattractant proteins (MCPs) family members in humans or an MCP inhibitor, wherein the MCPs targeted are optionally, CCL2, CCL7, CCL8, CCL13;
immune hot (immune-high), the subject or tumor cell is treated with a combination of PD-1/PD-L1 blockade and 001 inhibitor;
immune hot (immune-high), the subject or tumor cell is treated with a modulator or inhibitor of: IDO1 immune checkpoint proteins, optionally, CTLA-4, PD-1, or TIM-3; WARS; LCK; CD4, TYMP; or B2M;
mesenchymal-like GBM, the subject or tumor cell is treated with anti-angiogenic agents, optionally, bevacizumab, and immunotherapy;
mesenchymal GBM having elevated ALOX5 expression, the subject or tumor is treated with a GPX4 inhibitor, optionally, the GPX4 inhibitor is RSL3;
hyperacetylation of H2B, the subject or tumor cell is treated with CREBBP/EP300 inhibitors;
IDH mutated, the subject or tumor cell is treated with a GLUD1 inhibitor;
IDH mutated or proneural tumor, having upregulated AKT3, the subject or tumor is treated with an AKT3 inhibitor;
PTEN downregulated, the subject or tumor cell is treated with a modulator or inhibitor of: AKT1 or AKT2;
proneural and IDH-mutated, having amplified PDGFRA, higher RNA, protein, and phosphosite abundance of PDGFRA, the subject or tumor is treated with a PDGFRA inhibitor;
GSK3B, AKT1, or MAPK1 phosphorylation upregulated, the subject or tumor is treated with a GSK3B inhibitor, an AKT1 inhibitor, or a MAPK1 inhibitor, respectively;
ERBB2 or SHC1 phosphorylation upregulated, the subject or tumor cell is treated with a ERBB2 inhibitor or SHC1 inhibitor, respectively;
GSK3B, AKT1, MAPK1, MAPK3 and EGFR phosphorylation upregulated, the subject or tumor cell is treated with GSK3B, AKT1, MAPK1, MAPK3, and EGFR inhibitors, respectively;
ABL1-HDAC2 phosphorylation upregulated, the subject or tumor cell is treated with an ABL1 or HDAC-inhibitor;
FLT1, MMP14, ENG, and SERPINE1 upregulated in mesenchymal tumors, the subject or tumor cell is treated with a modulator or inhibitor of: HIF-1 and downstream targets;
long telomere enriched in hyperphosphorylated genes, the subject or tumor cell is treated with a modulator or inhibitor of: RFTN2, EML4, MLIP, TNIK, DEPTOR, ABLIM3, PLEKHA7, RRAS2, LARP4B, NHS, CTNND2, TP53BP1, IRS2, GJA1, EIF4EBP1, ZNF423, BLNK, LARP4, AKAP6, PARD3, PDCD4, SORBS1, TMPO, HEPACAM, SNTB2, GBF1, SPEG, EDNRA, TCEAL3, CANX, CCDC6, ARHGAP45, INPP5D, MAP7D1, or TNS1;
long versus short telomere enriched in hyperphosphorylated genes, the subject or tumor is treated with a modulator or inhibitor of: RFTN2, EML4, MLIP, TNIK, DEPTOR, ABLIM3, PLEKHA7, RRAS2, LARP4B, NHS, CTNND2, TP53BP1, IRS2, GJA1, EIF4EBP1, ZNF423, BLNK, LARP4, AKAP6, PARD3, PDCD4, SORBS1, TMPO, HEPACAM, SNTB2, GBF1, SPEG, EDNRA, TCEAL3, CANX, CCDC6, ARHGAP45, INPP5D, MAP7D1, or TNS1; or
short telomere enriched in hyperphosphorylated genes, the subject or tumor is treated with a modulator or inhibitor of: TP53BP1, IRS2, EIF4EBP1, PARD3, SORBS1, SPEG, CANX, SLTM, PRAG1, ZMYND8, RANBP2, GRAMD1A, ADGRL2, BBX, PCF11, TOP2B, SCAF1, TJP1, or FIP1L1; or
combinations thereof.

11. A method of treating a subject having lung cancer comprising:
obtaining or having obtained a tumor sample;
detecting upregulation of PTPN11 phosphorylation; and
administering an amount of a PTPN11 inhibitor sufficient to inhibit PTPN11 in a tumor of a subject if PTPN11 phosphorylation is upregulated.

12. The method of claim 11, wherein the PTPN11 inhibitor is a Shp2 inhibitor.

13. The method of claim 12, wherein the Shp2 inhibitor is selected from SHP099, TNO155, JAB-3068, RMC-4630, BBP 398, or RLY1971, or combinations thereof.

14. A method of treating a subject having brain cancer comprising:
obtaining or having obtained a tumor sample;
detecting upregulation of PTPN11 or PTPN11 phosphorylation; and
administering an amount of a PTPN11 inhibitor sufficient to inhibit PTPN11 in a tumor of a subject if PTPN11 phosphorylation, and optionally, a PLC inhibitor if PLCG1 phosphorylation is upregulated.

15. The method of claim 14, wherein the PTPN11 inhibitor is a Shp2 inhibitor.

16. The method of claim 15, wherein the Shp2 inhibitor is selected from SHP099, TNO155, JAB-3068, RMC-4630, BBP 398, or RLY1971, or combinations thereof.

17. The method of claim 15, wherein the PLC inhibitor is selected from edelfosine or neomycin.
